(19) United States
(12) Patent Application Publication
(10) Pub. No.: US 2022/0372489 A1
(43) Pub. Date: Nov. 24, 2022

(54) PPM1A INHIBITORS AND METHODS OF USING SAME

(71) Applicant: QURALIS CORPORATION, Cambridge, MA (US)

(72) Inventors: Sandra Hinckley, Sudbury, MA (US); Duncan Brown, Berkeley, CA (US); Sudhir Agrawal, Shrewsbury, MA (US); Daniel Elbaum, Newton, MA (US)

(21) Appl. No.: 17/621,320

(22) PCT Filed: Jun. 19, 2020
(86) PCT No.: PCT/US2020/038703

§ 371 Date: Dec. 21, 2021

Related U.S. Application Data
(60) Provisional application No. 62/864,988, filed on Jun. 21, 2019.
(60) Provisional application No. 62/871,356, filed on Jul. 8, 2019.

(51) Int. Cl. C12N 15/113

(52) U.S. Cl. CPC C12N 15/1137; C12N 2310/11; C12N 2310/315; C12N 2310/341; C12N 2310/346; C12N 2310/3515; C12N 2320/11; C12N 2320/31

(57) ABSTRACT

Disclosed herein are inhibitors of PPM 1 A, including PPM1A antisense oligonucleotide sequences, and methods for treating neurological diseases, such as amyotrophic lateral sclerosis and frontotemporal dementia, associated with decreased activity or expression of TBK1. Also disclosed are pharmaceutical compositions containing a PPM1A inhibitor, including a PPM1A antisense oligonucleotide, useful for treating neurological diseases and manufacture of medicaments containing a disclosed PPM1A inhibitor, for example, a PPM1A antisense oligonucleotide, to be used in treating a neurological disease

### CROSS REFERENCE TO RELATED APPLICATIONS

This application claims the benefit of and priority to U.S. Provisional Patent Application No. 62/864,988 filed Jun. 21, 2019 and U.S. Provisional Patent Application No. 62/871,356 filed on Jul. 8, 2019, the entire disclosure of each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jun. 17, 2020, is named QRL-001WO_SL.txt and is 821,932 bytes in size.

### BACKGROUND

Motor neuron diseases are a class of neurological diseases that result in the degeneration and death of motor neurons—those neurons which coordinate voluntary movement of muscles by the brain. Motor neuron diseases may be sporadic or inherited, and may affect upper motor neurons and/or lower motor neurons. Motor neuron diseases include amyotrophic lateral sclerosis, progressive bulbar palsy, pseudobulbar palsy, primary lateral sclerosis, progressive muscular atrophy, spinal muscular atrophy, and post-polio syndrome.

Amyotrophic lateral sclerosis (ALS) is a group of motor neuron diseases affecting about 15,000 individuals in the United States of America. ALS is characterized by degeneration and death of upper and lower motor neurons, resulting in loss of voluntary muscle control. Motor neuron death is accompanied by muscular fasciculation and atrophy. Early symptoms of ALS include muscle cramps, muscle spasticity, muscle weakness (for example, affecting an arm, a leg, neck, or diaphragm), slurred and nasal speech, and difficulty chewing or swallowing. Loss of strength and control over movements, including those necessary for speech, eating, and breathing, eventually occur. Disease progression may be accompanied by weight loss, malnourishment, anxiety, depression, increased risk of pneumonia, muscle cramps, neuropathy, and possibly dementia. Most individuals diagnosed with ALS die of respiratory failure within five years of the first appearance of symptoms. Currently, there is no effective treatment for ALS.

ALS occurs in individuals of all ages, but is most common in individuals between 55 to 75 years of age, with a slightly higher incidence in males. ALS can be characterized as sporadic or familial. Sporadic ALS appears to occur at random and accounts for more than 90% of all incidences of ALS. Familial ALS accounts for 5-10% of all incidences of ALS. Genetic mutations in more than a dozen genes are associated with familial ALS, including mutations in chromosome 9 open reading frame 72 (“C9ORF72”)—which account for between 25-40% of familial ALS cases—and copper-zinc superoxide dismutase 1 (“SOD1”—which accounts for 12-20% of familial ALS cases.

Interestingly, mutations in several ALS-associated genes, such as TBK1, TARDBP, SQSTM1, VCP, FUS, CHCHD10, and C9ORF72 are also associated with frontotemporal dementia (FTD) and ALS with FTD. FTD refers to a spectrum of progressive neurodegenerative diseases caused by loss of neurons in frontal and temporal lobes of the brain. FTD is characterized by changes in behavior and personality, and language dysfunction. Forms of FTD include behavioral variant FTD (bvFTD), semantic variant primary progressive aphasia (svPPA), and nonfluent variant primary progressive aphasia (nfvPPA). ALS with FTD is characterized by symptoms associated with FTD, along with symptoms of ALS such as muscle weakness, atrophy, fasciculation, spasticity, speech impairment (dysarthia), and inability to swallow (dysphagia). Individuals usually succumb to FTD within 5 to 10 years, while ALS with FTD often results in death within 2 to 3 years of the first disease symptoms appearing.

Like ALS, there is no known cure for FTD or ALS with FTD, nor a treatment known to prevent or retard either disease's progression.

Thus, there is a pressing need to identify compounds capable of preventing, ameliorating, and treating neurological diseases such as amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease.

### SUMMARY

Described herein are Protein Phosphatase 1A (PPM1A) inhibitors. The instant application is based, in part, on the surprising discovery that PPM1A inhibitors described herein can be used in the treatment of neurological diseases, including motor neuron diseases. For example, PPM1A inhibitors described herein can be used to treat any of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease. PPM1A inhibitors described herein include PPM1A antisense oligonucleotides and other PPM1A antisense therapeutics.

Disclosed herein is a compound comprising an oligonucleotide comprising linked nucleosides with a nucleobase sequence that is at least 90% complementary to an equal length portion of a transcript that is transcribed from at least nucleotide 41,932 to nucleotide 42,787 and from nucleotide 44,874 to nucleotide 44,990 of SEQ ID NO: 1, wherein at least one nucleoside linkage of the linked nucleosides is a non-natural linkage. Additionally disclosed herein is an oligonucleotide comprising linked nucleosides with a nucleobase sequence that is at least 90% complementary to an equal length portion of a transcript that is transcribed from at least nucleotide 41,932 to nucleotide 42,787 and from nucleotide 44,874 to nucleotide 44,990 of SEQ ID NO: 1, wherein at least one nucleoside linkage of the linked nucleosides is a non-natural linkage. In various embodiments, the transcript transcribed from nucleotide 41,932 to nucleotide 42,787 and from nucleotide 44,874 to nucleotide 44,990 of SEQ ID NO: 1 comprises a sequence of any of SEQ ID NO: 2864, SEQ ID NO: 2865, or SEQ ID NO: 2866.

Additionally disclosed herein is a compound comprising an oligonucleotide comprising linked nucleosides with a nucleobase sequence that is at least 90% complementary to an equal length portion of a transcript that shares at least 90% identity to SEQ ID NO: 2864, SEQ ID NO: 2865, or SEQ ID NO: 2866, or to a contiguous 15 to 50 nucleobase portion of SEQ ID NO: 2864, SEQ ID NO: 2865, or SEQ ID NO: 2866, wherein at least one nucleoside linkage of the linked nucleosides is a non-natural linkage. Additionally disclosed herein is an oligonucleotide comprising linked nucleosides with a nucleobase sequence that is at least 90% complementary to an equal length portion of a transcript that shares at least 90% identity to SEQ ID NO: 2864, SEQ ID NO: 2865, or SEQ ID NO: 2866, or to a contiguous 15 to 50 nucleobase portion of SEQ ID NO: 2864, SEQ ID NO: 2865, or SEQ ID NO: 2866, wherein at least one nucleoside linkage of the linked nucleosides is a non-natural linkage.

In various embodiments, the nucleobase sequence comprises a portion of at least 10 contiguous nucleobases that shares at least 90% identity with an equal length portion of any one of SEQ ID NOs: 2-955, SEQ ID NOs: 1910-2863, SEQ ID NOs: 2868-2913, and SEQ ID NOs: 2914-2959. In various embodiments, the nucleobase sequence comprises a portion of at least 11, 12, 13, 14, 15, 16, 17, 18, 19, or 20 contiguous nucleobases that shares at least 90% identity with an equal length portion of any one of SEQ ID NOs: 2-955, SEQ ID NOs: 1910-2863, SEQ ID NOs: 2868-2913, and SEQ ID NOs: 2914-2959. In various embodiments, the nucleobase sequence comprises a portion of at least 10 contiguous nucleobases that shares at least 90% identity with an equal length portion of any one of SEQ ID NOs: 2868-2913 and SEQ ID NOs: 2914-2959. In various embodiments, the nucleobase sequence comprises a portion of at least 11, 12, 13, 14, 15, 16, 17, 18, 19, or 20 contiguous nucleobases that shares at least 90% identity with an equal length portion of any one of SEQ ID NOs: 2868-2913 and SEQ ID NOs: 2914-2959.

In various embodiments, the nucleobase sequence comprises a portion of at least 10 contiguous nucleobases that is at least 90% complementary to an equal length portion of nucleobases within any one of positions 457-1410 of SEQ ID NO: 2864. In various embodiments, the nucleobase sequence comprises a portion of at least 11, 12, 13, 14, 15, 16, 17, 18, 19, or 20 contiguous nucleobases that is at least 90% complementary to an equal length portion of nucleobases within any one of positions 457-1410 of SEQ ID NO: 2864. In various embodiments, the nucleobase sequence comprises a portion of at least 10 contiguous nucleobases that is at least 90% complementary to an equal length portion of nucleobases within any one of positions 542-814, 895-1006, 1025-1117, or 1361-1407 of SEQ ID NO: 2864. In various embodiments, the nucleobase sequence comprises a portion of at least 11, 12, 13, 14, 15, 16, 17, 18, 19, or 20 contiguous nucleobases that is at least 90% complementary to an equal length portion of nucleobases within any one of positions 542-814, 895-1006, 1025-1117, or 1361-1407 of SEQ ID NO: 2864.

In various embodiments, the nucleobase sequence comprises a portion of at least 10 contiguous nucleobases that is at least 90% complementary to an equal length portion of nucleobases within any one of positions 542-561, 555-574, 559-578, 599-618, 602-621, 603-622, 604-623, 605-624, 606-625, 607-626, 608-627, 609-628, 625-644, 642-661, 644-663, 646-665, 648-667, 650-669, 652-671, 655-674, 656-675, 708-727, 709-728, 794-813, 795-814, 895-914, 900-919, 905-924, 910-929, 915-934, 962-981, 967-986, 972-991, 977-996, 987-1006, 1025-1044, 1030-1049, 1034-1053, 1040-1059, 1045-1064, 1098-1117, 1361-1380, 1366-1385, 1371-1390, 1378-1397, and 1386-1405 of SEQ ID NO: 2864.

In various embodiments, the nucleobase sequence comprises a portion of at least 11, 12, 13, 14, 15, 16, 17, 18, 19, or 20 contiguous nucleobases that is at least 90% complementary to an equal length portion of nucleobases within any one of positions 542-561, 555-574, 559-578, 599-618, 602-621, 603-622, 604-623, 605-624, 606-625, 607-626, 608-627, 609-628, 625-644, 642-661, 644-663, 646-665, 648-667, 650-669, 652-671, 655-674, 656-675, 708-727, 709-728, 794-813, 795-814, 895-914, 900-919, 905-924, 910-929, 915-934, 962-981, 967-986, 972-991, 977-996, 987-1006, 1025-1044, 1030-1049, 1034-1053, 1040-1059, 1045-1064, 1098-1117, 1361-1380, 1366-1385, 1371-1390, 1378-1397, and 1386-1405 of SEQ ID NO: 2864.

In various embodiments, the oligonucleotide comprises at least one nucleoside linkage selected from the group consisting of a phosphodiester linkage, a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, or any combination(s) thereof.

In various embodiments, at least one internucleoside linkage of the nucleotide sequence is a phosphorothioate linkage. In various embodiments, the phosphorothioate internucleoside linkage is in one of a Rp configuration or a Sp configuration. In various embodiments, the oligonucleotide comprises one or more chiral centers and/or double bonds. In various embodiments, the oligonucleotide exist as stereoisomers selected from geometric isomers, enantiomers, and diastereomers. In various embodiments, all internucleoside linkages of the nucleotide sequence are phosphorothioate linkages.

In various embodiments, the oligonucleotide comprises at least one modified nucleobase. In various embodiments, the at least one modified nucleobase is 5-methylcytosine, pseudouridine, or 5-methoxyuridine.

In various embodiments, the oligonucleotide comprises at least one nucleoside with a modified sugar moiety. In various embodiments, the modified sugar moiety is one of a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA). In various embodiments, the oligonucleotide comprises two, three, four, five, six, seven, eight, nine, or ten nucleosides with modified sugar moieties. In various embodiments, the modified sugar moieties are independently any one of a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

In various embodiments, the oligonucleotide comprises ten 2′-O-(2-methoxyethyl) (2′MOE) nucleosides. In various embodiments, five of the 2′-O-(2-methoxyethyl) (2′MOE) nucleosides are located at the 3′ end of the oligonucleotide, and wherein five of the 2′-O-(2-methoxyethyl) (2′MOE) nucleosides are located at the 5′ end of the oligonucleotide. In various embodiments, the at least one nucleoside with the modified sugar moiety or the nucleosides with modified sugar moieties are ribonucleosides. In various embodiments, the oligonucleotide comprises at least one deoxyribonucleoside. In various embodiments, the oligonucleotide comprises two, three, four, five, six, seven, eight, nine, or ten deoxyribonucleosides.

In various embodiments, the oligonucleotide comprises:
a. a gap segment comprising one or more of linked deoxyribonucleosides, 2′-Fluoro Arabino Nucleic Acids (FANA), and Fluoro Cyclohexenyl nucleic acid (F-CeNA);
b. a 5′ wing region comprising linked nucleosides; and
c. a 3′ wing region comprising linked nucleosides;
d. wherein the central region comprises a region of at least 8 contiguous nucleobases having at least 80% identity to an equal length portion of any one of SEQ ID NOs: 2-955, SEQ ID NOs: 1910-2863, or SEQ ID NOs: 2868-2959 positioned between the 5′ wing segment and the 3′ wing segment; wherein the 5′ wing region and the 3′ wing region each comprises at least two linked nucleosides; and wherein at least one nucleoside of each wing region comprises a modified sugar.

In various embodiments, at least two linked nucleosides of the 5′ wing region are linked through a phosphorothioate internucleoside linkage and/or wherein the at least two linked nucleosides of the 3′ wing region are independently linked through a phosphorothioate internucleoside linkage. In various embodiments, every internucleoside linkage of the 5′ wing region and/or every internucleoside linkage of the 3′ wing region, independently are phosphorothioate internucleoside linkages. In various embodiments, the 5′ wing region further comprises at least one phosphodiester internucleoside linkage. In various embodiments, the 3′ wing region further comprises at least one phosphodiester internucleoside linkage.

In various embodiments, the at least two linked nucleosides of the 5′ wing region are linked through a phosphodiester internucleoside linkage and/or wherein the at least two linked nucleosides of the 3′ wing region are independently linked through a phosphodiester internucleoside linkage. In various embodiments, at least one of the internucleoside linkages of the central region is a phosphodiester linkage. In various embodiments, at least two, at least three, at least four, at least five, at least six, at least seven, at least eight, or at least nine of the internucleoside linkages of the central region are phosphodiester linkages.

In various embodiments, at least one of the internucleoside linkages of the central region is a phosphorothioate internucleoside linkage. In various embodiments, at least two, at least three, at least four, at least five, at least six, at least seven, at least eight, or at least nine of the internucleoside linkages of the central region are phosphorothioate internucleoside linkages. In various embodiments, all internucleoside linkages of the oligonucleotide are phosphorothioate internucleoside linkages. In various embodiments, any one or all of the phosphorothioate internucleoside linkages are in a Rp configuration, a Sp configuration, or in any combination of Rp and Sp configuration.

In various embodiments, the oligonucleotide comprises at least one modified sugar moiety. In various embodiments, the 5′ wing region or the 3′ wing region comprises the at least one modified sugar moiety. In various embodiments, the central region comprises the at least one modified sugar moiety. In various embodiments, the at least one modified sugar moiety is any one of a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

In various embodiments, the oligonucleotide comprises one or more 2′-MOE nucleosides. In various embodiments, the 5′ wing region or the 3′ wing region comprise one or more 2′-MOE nucleosides. In various embodiments, the 5′ wing region or the 3′ wing region comprise two, three, four, or five 2′-MOE nucleosides. In various embodiments, every nucleoside of the 5′ wing region or the 3′ wing region is a 2′-MOE nucleoside.

In various embodiments, the central region comprises one or more 2′-MOE nucleosides. In various embodiments, the central region comprises two, three, four, five, six, seven, eight, nine, or ten 2′-MOE nucleosides. In various embodiments, every nucleoside of the central region is a 2′-MOE nucleoside. In various embodiments, the one or more 2′-MOE nucleosides are linked through phosphorothioate internucleoside linkages.

In various embodiments, the oligonucleotide comprises sugar modifications in any of the following patterns: eeeee-d10-eeeee, eee-d8-eee, eee-d10-eee, eeee-d10-eeee, and eeee-d8-eeee, wherein e=2′-MOE nucleoside and d=a deoxyribonucleoside. In various embodiments, the oligonucleotide comprises internucleoside linkages in any of the following patterns: sssssooooooooosssss; ooooosssssssssooooo; oooooooooooooosssss; soossssssssssssssss; ssssssssssssssssoos; sssssoooooooooooooo; sssssssssssssssssss; sssooooooosss; ooosssssssooo; sssssssssssss; sosssssssssos; sosssssssssss; sssssssssssos; ssssssssssooo; ooossssssssss; sssooooooooosss; ooosssssssssooo; sssssssssssssss; ssssssssssssooo; ooossssssssssss; sosssssssssssos; sosssssssssssss; sssssssssssssos; ssssooooooooossss; oooosssssssssoooo; sssssssssssssssss; sssssssssssssoooo; soosssssssssssoos; soossssssssssssss; ssssssssssssssoos; oooosssssssssssss; ssssooooooossss; oooosssssssoooo; sssssssssssoooo; oooosssssssssss; soosssssssssoos; soossssssssssss; ssssssssssssoos; or sssssssssssssss; wherein s=a phosphorothioate linkage, and o=a phosphodiester linkage.

In various embodiments, the oligonucleotide comprises sugar modification and internucleoside linkage combinations, respectively, in any of the following patterns: ssssooooooooossss
a) eeeee-d10-eeeee and sssssooooooooosssss;
b) eeeee-d10-eeeee and ooooosssssssssooooo;
c) eeeee-d10-eeeee and sssssssssssssssssss;
d) eee-d8-eee and sssooooooosss;
e) eee-d8-eee and ooosssssssooo
f) eee-d8-eee and sssssssssssss;
g) eee-d10-eee and sssooooooooosss;
h) eee-d10-eee and ooosssssssssooo;
i) eee-d10-eee and sssssssssssssss;
j) eeee-d10-eeee and ssssooooooooossss;
k) eeee-d10-eeee and oooosssssssssoooo;
l) eeee-d10-eeee and sssssssssssssssss;
m) eeee-d8-eeee and ssssooooooossss,
n) eeee-d8-eeee and oooosssssssoooo,
o) eeee-d8-eeee and sssssssssssssss,
wherein e=2′-MOE nucleoside and d=a deoxyribonucleoside, and wherein s=a phosphorothioate linkage, and o=a phosphodiester linkage.

In various embodiments, the oligonucleotide comprises at least one modified nucleobase. In various embodiments, the 5′ wing region or the 3′ wing region comprises the at least one modified nucleobase. In various embodiments, the central region comprises the at least one modified nucleobase. In various embodiments, the at least one modified nucleobase is 5′-methylcytosine, pseudouridine, or 5-methoxyuridine. In various embodiments, every cytosine in the 5′ wing region or the 3′ wing region is a 5′-methylcytosine. In various embodiments, every cytosine in the central region is a 5′-methylcytosine.

In various embodiments, the oligonucleotide comprises sugar modification and internucleoside linkage combination of:
eeeee-d10-eeeee and sssssssssssssssssss, wherein e=2′-MOE nucleoside and d=a deoxyribonucleoside, and wherein s=a phosphorothioate linkage,

wherein each cytosine of the 2′MOE nucleosides is a 5-methylcytosine.

In various embodiments, the oligonucleotide further comprises a conjugate moiety. In various embodiments, the conjugate moiety is a cholesterol conjugate located on the 3′ end of the oligonucleotide.

Additionally disclosed herein is a pharmaceutical composition comprising any one of the oligonucleotides disclosed above, or a pharmaceutically acceptable salt thereof, and a pharmaceutically acceptable excipient.

Additionally disclosed herein is a method of treating a neurological disease in a patient in need thereof, the method comprising administering to the patient an oligonucleotide of any one of the oligonucleotides disclosed above, or a pharmaceutically acceptable salt thereof, or a pharmaceutical composition disclosed above.

In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

Additionally disclosed herein is a method of increasing autophagy in a cell, the method comprising exposing the cell to a PPM1A inhibitor. Additionally disclosed herein is a method of increasing TBK1 ser172 phosphorylation in a cell, the method comprising exposing the cell to a PPM1A inhibitor. Additionally disclosed herein is a method of increasing TBK1 function in a cell, the method comprising exposing the cell to a PPM1A inhibitor. Additionally disclosed herein is a method of inhibiting PPM1A in a cell, the method comprising exposing the cell to a PPM1A inhibitor. Additionally disclosed herein is a method of inhibiting RIPK1 activity in a cell, the method comprising exposing the cell to a PPM1A inhibitor.

In various embodiments, the cell is a cell of a patient in need of treatment of a neurological disease. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease. In various embodiments, the exposing is performed in vivo or ex vivo. In various embodiments, the exposing comprises administering the PPM1A inhibitor to a patient in need thereof.

In various embodiments, the PPM1A inhibitor is administered topically, parenterally, intrathecally, intracisternally, orally, rectally, buccally, sublingually, vaginally, pulmonarily, intratracheally, intranasally, transdermally, or intraduodenally. In various embodiments, the PPM1A inhibitor is administered intrathecally. In various embodiments, a therapeutically effective amount of the PPM1A inhibitor is administered. In various embodiments, the patient is a human.

In various embodiments, the PPM1A inhibitor comprises the PPM1A antisense oligonucleotide of any one of the oligonucleotides disclosed above, a pharmaceutically acceptable salt thereof, or a pharmaceutical composition disclosed above. In various embodiments, the pharmaceutical composition is suitable for topical, intrathecal, parenteral, oral, pulmonary, intratracheal, intranasal, transdermal, rectal, buccal, sublingual, vaginal, intracisternal, or intraduodenal administration.

Additionally disclosed herein is a use of a PPM1A inhibitor in the manufacture of a medicament for the treatment of neurological disease. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease. In various embodiments, the PPM1A inhibitor is the PPM1A antisense oligonucleotide of any one of the oligonucleotides disclosed above.

Additionally disclosed herein is a method of treating a neurological disease in a patient in need thereof, the method comprising administering to a patient in need thereof a therapeutically effective amount of a pharmaceutical composition comprising a PPM1A inhibitor, and a pharmaceutically acceptable excipient. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease. In various embodiments, the PPM1A inhibitor is the PPM1A antisense oligonucleotide of any one of the oligonucleotides disclosed above, a pharmaceutically acceptable salt thereof, or a pharmaceutical composition disclosed above.

In various embodiments, the pharmaceutical composition is administered topically, parenterally, orally, pulmonarily, rectally, buccally, sublingually, vaginally, intratracheally, intranasally, intrathecally, intracisternally, transdermally, or intraduodenally. In various embodiments, the pharmaceutical composition is administered intrathecally. In various embodiments, the patient is human.

Additionally disclosed herein is a PPM1A antisense oligonucleotide of any one of the oligonucleotides disclosed above, or a pharmaceutically acceptable salt thereof, for use as a medicament. Additionally disclosed herein is a PPM1A antisense oligonucleotide of any one of the oligonucleotides disclosed above, or a pharmaceutically acceptable salt thereof, for use in the treatment of a neurological disease. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

Additionally disclosed herein is a Protein Phosphatase 1A (PPM1A) antisense oligonucleotide selected from the group consisting of: a PPM1A antisense oligonucleotide comprising the nucleotide sequence of any one of SEQ ID NOs: 2-955, SEQ ID NOs: 1910-2863, SEQ ID NOs: 2868-2913, and SEQ ID NOs: 2914-2959, or a pharmaceutically acceptable salt thereof, wherein at least one nucleoside linkage of the nucleotide sequence is selected from the group consisting of: a phosphodiester linkage, a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage; and/or wherein at least one nucleoside of the linked nucleosides is substituted with a component selected from the group consisting of a 2′-O-(2-methoxyethyl) (2′-MOE) nucleoside, a 2′-O-methyl nucleoside, a 2′-deoxy-2′-fluoro nucleoside, a 2′-fluoro-β-D-arabinonucleoside, a locked nucleic acid (LNA), constrained methoxyethyl (cMOE), constrained ethyl (cET), and a peptide nucleic acid (PNA).

In various embodiments, at least one internucleoside linkage of the nucleotide sequence is a phosphorothioate linkage. In various embodiments, the phosphorothioate internucleoside linkage is in one of a Rp configuration or a Sp configuration. In various embodiments, all internucleoside linkages of the nucleotide sequence are phosphorothioate linkages.

Additionally disclosed herein is a pharmaceutical composition comprising the antisense oligonucleotide of any one of the oligonucleotides disclosed above, or a pharmaceutically acceptable salt thereof, and a pharmaceutically acceptable excipient. In various embodiments, the patient for treatment is identified by measuring the presence or level of expression of neurofilament light (NEFL), neurofilament heavy (NEFH), phosphorylated neurofilament heavy chain (pNFH), TDP-43, or p75^{ECD} in the plasma, the spinal cord fluid, the cerebrospinal fluid, the extracellular vesicles (for example, CSF exosomes), the blood, the urine, the lymphatic fluid, fecal matter, or a tissue of the patient. In various embodiments, the patient for treatment is identified by measuring phosphorylated neurofilament heavy chain (pNFH) in cerebrospinal fluid (CSF). In various embodiments, the pNFH in the CSF of the patient is used to predict disease status and survival in C9ORF72-associated amyotrophic lateral sclerosis (c9ALS) patients after initial administration and/or during on-going treatment.

Additionally disclosed herein is a method of treating a neurological disease and/or a neuropathy in a patient in need thereof, the method comprising administering to a patient in need thereof a therapeutically effective amount of an oligonucleotide of any one the oligonucleotides disclosed above or a pharmaceutically acceptable salt thereof, or a pharmaceutical composition disclosed above, in combination with a second therapeutic agent selected from a group comprising Riluzole (Rilutek), troriluzole, Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO®), ZILUCOPLAN (RA101495). dual AON intrathecal administration (e.g., BIIB067, BIIB078), BIIB100, levodopa/carbidopa, dopaminergic agents (e.g., ropinirole, pramipexole, rotigotine), medroxyprogesterone, KCNQ2/KCNQ3 openers, Pridopidine, PrimeC (combination of ciprofloxacin and Celebrex), lithium, anticonvulsants and psychostimulant agents, breathing care, physical therapy, occupational therapy, speech therapy, and nutritional support. In various embodiments, the neurological disease is any one of amyotrophic lateral sclerosis (ALS), frontotemporal dementia (FTD), or ALS with FTD.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising Memantine, Rivastigmine, Galantamine, Donepezil, Aricept®, Exelon® (Rivastigmine), Razadyne®, Aducanumab, BAN2401, BIIB091 (gosuranemab), BIIB076, BIIB080 (IONIS-MAPTRx), Elayta (CT1812), MK1942, allogenic hMSC, nilotinib, ABT-957, acitretin, ABT-354, GV1001, Riluzole, CAD106, CNP520, AD-35, Rilapladib, DHP1401, T-817 MA, TC-5619, TPI-287, RVT-101, LY450139, JNJ-54861911, Dapagliflozin, GSK239512, PF-04360365, ASP0777, SB-742457 (a 5-HT6 receptor antagonist), PF-03654746 (an H₃ receptor antagonist), GSK933776 (an Fc-inactivated anti-D amyloid (AD) monoclonal antibody (mAb)), Posiphen ((+)-phenserine tartrate), AMX0035 (ELYBRIO®), coenzyme Q10, or any combination thereof.

In various embodiments, the neurological disease is Alzheimer's Disease.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising Levodopa, Carbidopa-levidopa, pramipexole, ropinirole, rotigotine, apomorphine, selegiline, rasagiline, entacapone, tolcapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, or any combination thereof. In various embodiments, the neurological disease is Parkinson's Disease.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising UCB0107, ABBV-8E12, F-18 AV1451, BIIB092, C2N-8E12, tideglusib, deep transcranial magnetic stimulation, lipoic acid, tolfenamica acid, lithium, AZP2006, Glial Cell Line-Derived Neurotrophic Factor, NBMI, suvorxant, zolpidem, TPI 287, davunetide, pimavanserin, Levodopa, Carbidopa-levidopa, pramipexole, ropinirole, rotigotine, apomorphine, selegiline, rasagiline, entacapone, tolcapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, or any combination thereof. In various embodiments, the neurological disease is progressive supranuclear palsy (PSP).

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising Tetrabenazine, deutetrabenazine, physical therapy, risperidone, haloperidol, chlorpromazine, clonazepam, diazepam, benzodiazepines, selective serotonin reuptake inhibitors. quetiapine, carbatrol, valproate, lamotrigine, pridopidine, delta-9-tetrahydrocannabinol, cannabidiol, stem-cell therapy, ISIS-443139, nilotinib, resveratrol, neflamapimod, fenofibrate, creatine, RO7234292, SAGE-718, WVE-120102, WVE-120101, dimebon, minocycline, deep brain stimulation, ursodiol, coenzyme Q10, OMS643762, VX15/2503, PF-02545920, BN82451B, SEN0014196, olanzapine, tiapridal (tiapride), or any combination thereof. In various embodiments, the neurological disease is Huntington's Disease.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising anticoagulants, antidepressants, muscle relaxants, stimulants, anticonvulsants, anti-anxiety medication, erythropoietin, hyperbaric treatment, rehabilitation therapies (e.g., physical, occupational, speech, psychological, or vocational counseling), or any combination thereof. In various embodiments, the neurological disease is brain trauma.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising AXER-204, glyburide, 5-hydroxytryptophan (5-HTP), L-3,4-dihydroxyphenylalanine (L-DOPA), or rehabilitation therapies (e.g., physical therapy, occupational therapy, recreational therapy, use of assistive devices, improved strategies for exercise and healthy diets), or any combination thereof. In various embodiments, the neurological disease is spinal cord injury.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising TPI-287, lithium, occupational, physical, and speech therapy, or any combination thereof can be selected as an additional therapy. In various embodiments, the neurological disease is corticobasal degeneration.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising gabapentin, pregabalin, lamotrigine, carbamazepine, duloxetine, gabapentinoids, tricyclic antidepressants, serotonin-norepinephrine reuptake inhibitors, opioids, neurotoxin, dextromethorphan, nicotinamide riboside, auto-antibodies targeting neuronal antigens (TS-HDS and FGFR3), or any combination thereof. In various embodiments, the neuropathy is a chemotherapy induced neuropathy.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising troriluzole, BHV-4157, or a combination thereof. In various embodiments, the neurological disease is spinocerebellar ataxia.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising anti-seizure medications, speech therapy, physical therapy, occupational therapy, Adrabetadex, Arimoclomol, N-Acetyl-L-Leucine, or any combination thereof. In various embodiments, the neurological disease is Niemann-Pick disease type C.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising physical and occupational therapies, orthopedic surgery, orthopedic devices, PXT3003, or any combination thereof. In various embodiments, the neurological disease is Charcot-Marie-Tooth Disease (CMT).

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising enzyme replacement therapy: idursulfase (Elaprase), surgical intervention (tonsillectomy and/or adenoidectomy), RGX-121 gene therapy, adalimumab, MT2013-31, or any combination thereof. In various embodiments, the neurological disease is Mucopolysaccharidosis type II (MPSIIA).

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising physical, occupational, and speech therapies, contact lenses and artificial tears, genetic counseling, or any combination thereof. In various embodiments, the neurological disease is Mucolipidosis IV.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising anticonvulsants, physical and occupational therapies, galactosidase, gene delivery of galactosidase, LYS-GM101 gene therapy, or any combination thereof. In various embodiments, the neurological disease is GM1 gangliosidosis.

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising physical and occupational therapies, use of devices such as braces, walkers, wheelchairs, immunosuppressants, BYM338, or any combination thereof. In various embodiments, the neurological disease is Sporadic inclusion body myositis (sIBM).

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising corticosteroids, colchicine, dapsone, azathioprine, or any combination thereof. In various embodiments, the neurological disease is Henoch-Schonlein purpura (HSP).

Additionally disclosed is a method of treating a neurological disease and/or a neuropathy in a patient in need thereof, the method comprising administering to a patient in need thereof a therapeutically effective amount of an oligonucleotide of any one of the oligonucleotides disclosed above or a pharmaceutically acceptable salt thereof, or a pharmaceutical composition disclosed above, in combination with a second therapeutic agent selected from a group comprising enzyme replacement therapy, substrate reduction therapy, N-acetylcysteine, GZ/SAR402671, cerezyme, or any combination thereof. In various embodiments, the neurological disease is Gaucher's disease.

In various embodiments, the transcript comprises a sequence of SEQ ID NO: 2864 and is further transcribed from nucleotides 8,470-8, 926, 44,991-45,990, 49,055-49,164, 50,647-50,704, and 51,703-58,336 of SEQ ID NO: 1. In various embodiments, the transcript comprises a sequence of SEQ ID NO: 2865 and is further transcribed from nucleotides 8,470-8,926, 9,629-9,730, and 44,911-47,804 of SEQ ID NO: 1. In various embodiments, the transcript comprises a sequence of SEQ ID NO: 2866 and is further transcribed from nucleotides 4,999-5,295, 49,055-49,164, 50,647-50,704, and 51,703-58,336 of SEQ ID NO: 1.

Additionally disclosed herein is a method of treating a neurological disease in a patient, the method comprising selecting a patient for treatment with an oligonucleotide of any one of the oligonucleotides disclosed above or a pharmaceutically acceptable salt thereof, or a pharmaceutical composition disclosed above, wherein the patient for treatment is selected by a method comprising measuring a presence or level of expression of neurofilament light (NEFL), neurofilament heavy (NEFH), phosphorylated neurofilanent heavy chain (pNFH), TDP-43, or p75^{ECD} in the plasma, the spinal cord fluid, the cerebrospinal fluid, the extracellular vesicles (for example, CSF exosomes), the blood, the urine, the lymphatic fluid, fecal matter, or a tissue of the patient. In various embodiments, the patient for treatment is identified by measuring phosphorylated neurofilament heavy chain (pNFH) in cerebrospinal fluid (CSF). In various embodiments, the pNFH in the CSF of the patient is used to predict disease status and survival in C9ORF72-associated amyotrophic lateral sclerosis (c9ALS) patients after initial administration and/or during on-going treatment.

Additionally disclosed is a method of treating a neurological disease in a patient, the method comprising selecting a patient for treatment with an oligonucleotide of any one of the oligonucleotides disclosed above or a pharmaceutically acceptable salt thereof, or a pharmaceutical composition disclosed above, wherein the method comprises: determining whether the patient has a mutation in one or more ALS-associated genes selected from the group comprising TBK1, TARDBP, SQSTM1, VCP, C9orf72, FUS, and CHCHD10; identifying the patient as a candidate patient for treatment according to the determination; and optionally administering, to the candidate patient, the oligonucleotide of any one of the oligonucleotides disclosed above or a pharmaceutically acceptable salt thereof, or a pharmaceutical composition disclosed above,

Additionally disclosed is a method of treating a neurological disease in a patient, the method comprising administering to the patient an oligonucleotide of any one of the oligonucleotides disclosed above or a pharmaceutically acceptable salt thereof, or a pharmaceutical composition disclosed above, wherein the patient for treatment is selected by a method comprising measuring a presence or level of expression of neurofilament light (NEFL), neurofilament heavy (NEFH), phosphorylated neurofilament heavy chain (pNFH), TDP-43, or p75^{ECD} in the plasma, the spinal cord fluid, the cerebrospinal fluid, the extracellular vesicles (for example, CSF exosomes), the blood, the urine, the lymphatic fluid, fecal matter, or a tissue of the patient. In various embodiments, the patient for treatment is identified by measuring phosphorylated neurofilament heavy chain (pNFH) in cerebrospinal fluid (CSF). In various embodiments, the pNFH in the CSF of the patient is used to predict disease status and survival in C9ORF72-associated amyotrophic lateral sclerosis (c9ALS) patients after initial administration and/or during on-going treatment.

Additionally disclosed is a method of treating a neurological disease in a patient, the method comprising administering to the patient an oligonucleotide of any one of the oligonucleotides disclosed above or a pharmaceutically acceptable salt thereof, or a pharmaceutical composition disclosed above, wherein the patient is selected for treatment by a method comprising: determining whether the patient has a mutation in one or more ALS-associated genes selected from the group comprising TBK1, TARDBP, SQSTM1, VCP, C9orf72, FUS, and CHCHD10; identifying the patient as a candidate patient for treatment according to the determination.

### BRIEF DESCRIPTION OF THE DRAWINGS

FIG. 1 is a bar graph showing the results of RT-qPCR analysis of PPM1A levels in BP6074 cells treated with transfection reagent alone (“Lipofectamine 3000 Alone”) or transfected with varying concentrations (5 nM, 20 nM, 50 nM, 200 nM, or 500 nM) of PPM1A AON candidates (QPA-905, QPA-972, QPA-1034, QPA-1045, and QPA-1371) for 72 hours. All experiments were performed in triplicate (n=3).

FIG. 2A is a bar graph showing the amount of PPM1A, as evaluated by RT-qPCR. SY5Y cells were left untreated, treated with transfection reagent alone (“lipofectamine 3000 alone”), or transfected with various concentrations (5 nM, 20 nM, 50 nM, 200 nM, or 500 nM) of the PPM1A AON QPA-1371, an siRNA control (“siControl,” 50 nM), or a PPM1A siRNA (“siPPM1A,” 50 nM). RT-qPCR was performed 48 hours after transfection.

FIG. 2B is a bar graph showing the amount of PPM1A, as evaluated by RT-qPCR. SY5Y cells were left untreated, treated with transfection reagent alone (“endoporter alone”), or transfected with various concentrations (5 nM, 20 nM, 50 nM, 200 nM, or 500 nM) of the PPM1A AON candidates (QPA-905, QPA-972, QPA-1034, QPA-1045, QPA-1371, or QPA-895), an siRNA control (“siControl,” 50 nM), or a PPM1A siRNA (“siPPM1A,” 50 nM). RT-qPCR was performed 48 hours after transfection.

FIG. 3A is a bar graph showing the ratio of phosphorylated TBK1 to total TBK1 (“pTBK1/TBK1”) as a percent of the ratio in healthy control cells, as evaluated by Western blot. BP6074 cells were treated with RNAiMax transfection reagent alone (“patient cells”) or transfected with 5 μM of the PPM1A AON candidates QPA-1045 or QPA-1371. Cell media was changed 24 hours post-transfection and protein was collected 48 hours later (n=3; *, p<0.05; **, p<0.01). GAPDH protein levels were used to normalize pTBK1 and TBK1 protein levels.

FIG. 3B is a bar graph showing the amount of PPM1A, as evaluated by Western blot. BP6074 cells were treated with RNAiMax transfection reagent alone (“patient cells”) or transfected with 5 μM of the PPM1A AON candidates QPA-1045 or QPA-1371. Cell media was changed 24 hours post-transfection and protein was collected 48 hours later (n=3; ** p<0.01).

FIG. 4A-FIG. 4Y are line graphs of RNA-knockdown potency of various candidate antisense oligonucleotides quantifying the decrease in PPM1A RNA with increasing AON concentration in SY5Y cells. FIG. 4A represents RNA-knockdown potency of SEQ ID NO: 2898 (QPA-962); FIG. 4B represents RNA-knockdown potency of SEQ ID NO: 2899 (QPA-967); FIG. 4C represents RNA-knockdown potency of SEQ ID NO:2900 (QPA-972); FIG. 4D represents RNA-knockdown potency of SEQ ID NO: 2901 (QPA-977); FIG. 4E represents RNA-knockdown potency of SEQ ID NO: 2902 (QPA-987); FIG. 4F represents RNA-knockdown potency of SEQ ID NO: 2903 (QPA-1025); FIG. 4G represents RNA-knockdown potency of SEQ ID NO: 2904 (QPA-1030); FIG. 4H represents RNA-knockdown potency of SEQ ID NO: 2905 (QPA-1034); FIG. 4I represents RNA-knockdown potency of SEQ ID NO: 2906 (QPA-1040); FIG. 4J represents RNA-knockdown potency of SEQ ID NO: 2907 (QPA-1045); FIG. 4K represents RNA-knockdown potency of SEQ ID NO: 2909 (QPA-1361); FIG. 4L represents RNA-knockdown potency of SEQ ID NO: 2910 (QPA-1366); FIG. 4M represents RNA-knockdown potency of SEQ ID NO: 2911 (QPA-1371); FIG. 4N represents RNA-knockdown potency of SEQ ID NO: 2912 (QPA-1378); FIG. 4O represents RNA-knockdown potency of SEQ ID NO: 2913 (QPA-1386); FIG. 4P represents RNA-knockdown potency of SEQ ID NO: 2868 (QPA-542); FIG. 4Q represents RNA-knockdown potency of SEQ ID NO: 2869 (QPA-555); FIG. 4R represents RNA-knockdown potency of SEQ ID NO: 2883 (QPA-646); FIG. 4S represents RNA-knockdown potency of SEQ ID NO: 2870 (QPA-559); FIG. 4T represents RNA-knockdown potency of SEQ ID NO: 2908 (QPA-1098); FIG. 4U represents RNA-knockdown potency of SEQ ID NO: 2893 (QPA-895); FIG. 4V represents RNA-knockdown potency of SEQ ID NO: 2894 (QPA-900); FIG. 4W represents RNA-knockdown potency of SEQ ID NO: 2895 (QPA-905); FIG. 4X represents RNA-knockdown potency of SEQ ID NO: 2896 (QPA-910); and FIG. 4Y represents RNA-knockdown potency of SEQ ID NO: 2897 (QPA-915).

FIGS. 5A-5T and FIGS. 6A-6K are line graphs of RNA-knockdown potency of various candidate antisense oligonucleotides quantifying the decrease in PPM1A RNA with increasing AON concentration in human motor neurons. FIG. 5A represents RNA-knockdown potency of SEQ ID NO: 2883 (QPA-646); FIG. 5B represents RNA-knockdown potency of SEQ ID NO: 2893 (QPA-895); FIG. 5C represents RNA-knockdown potency of SEQ ID NO: 2895 (QPA-905); FIG. 5D represents RNA-knockdown potency of SEQ ID NO: 2911 (QPA-1371); FIG. 5E represents RNA-knockdown potency of SEQ ID NO: 2896 (QPA-910); FIG. 5F represents RNA-knockdown potency of SEQ ID NO: 2897 (QPA-915); FIG. 5G represents RNA-knockdown potency of SEQ ID NO: 2900 (QPA-972); FIG. 5H represents RNA-knockdown potency of SEQ ID NO: 2905 (QPA-1034); FIG. 5I represents RNA-knockdown potency of SEQ ID NO: 2906 (QPA-1040); FIG. 5J represents RNA-knockdown potency of SEQ ID NO: 2907 (QPA-1045); FIG. 5K represents RNA-knockdown potency of SEQ ID NO: 2871 (QPA-599); FIG. 5L represents RNA-knockdown potency of SEQ ID NO: 2876 (QPA-606); FIG. 5M represents RNA-knockdown potency of SEQ ID NO: 2880 (QPA-625); FIG. 5N represents RNA-knockdown potency of SEQ ID NO: 2881 (QPA-642); FIG. 5O represents RNA-knockdown potency of SEQ ID NO: 2882 (QPA-644); FIG. 5P represents RNA-knockdown potency of SEQ ID NO: 2884 (QPA-648); FIG. 5Q represents RNA-knockdown potency of SEQ ID NO: 2885 (QPA-650); FIG. 5R represents RNA-knockdown potency of SEQ ID NO: 2886 (QPA-652); FIG. 5S represents RNA-knockdown potency of SEQ ID NO: 2887 (QPA-655);

FIG. 5T represents RNA-knockdown potency of SEQ ID NO: 2888 (QPA-656); FIG. 6A represents RNA-knockdown potency of SEQ ID NO: 2872 (QPA-602); FIG. 6B represents RNA-knockdown potency of SEQ ID NO: 2873 (QPA-603); FIG. 6C represents RNA-knockdown potency of SEQ ID NO: 2874 (QPA-604); FIG. 6D represents RNA-knockdown potency of SEQ ID NO: 2875 (QPA-605); FIG. 6E represents RNA-knockdown potency of SEQ ID NO: 2877 (QPA-607); FIG. 6F represents RNA-knockdown potency of SEQ ID NO: 2878 (QPA-608); FIG. 6G represents RNA-knockdown potency of SEQ ID NO: 2879 (QPA-609);

FIG. 6H represents RNA-knockdown potency of SEQ ID NO: 2889 (QPA-708); FIG. 6I represents RNA-knockdown potency of SEQ ID NO: 2890 (QPA-709); FIG. 6J represents RNA-knockdown potency of SEQ ID NO: 2891 (QPA-794); and FIG. 6K represents RNA-knockdown potency of SEQ ID NO: 2892 (QPA-795).

FIGS. 7A and 7B show reduction of PPM1A expression in two ALS iPSC lines (TBK1 and C9orf72) following treatment using PPM1A AONs (QPA-895, QPA-905, QPA-915, QPA-1045, QPA-1371, AND QPA-646).

FIG. 8 shows the decreased PPM1A relative quantity in human motor neurons in response to treatment using PPM1A AONs with a cholesterol conjugate group (QPA-606-C, QPA-642-C, QPA-644-C).

FIG. 9 shows the reduction in PPM1A protein in response to treatment using PPM1A AONs (QPA-646 and QPA-915).

FIG. 10 shows the decrease in PPM1A protein levels in wildtype iPSC-derived motor neurons in response to treatment using PPM1A AONs (QPA-642, QPA-646, QPA-1371, QPA-905, and QPA-915).

FIGS. 11A-11C show the qualitative and quantitative results of the Western blot analysis in human motor neurons treated using PPM1A AONs (QPA-646 and QPA-905).

FIGS. 12A-12D show the qualitative and quantitative results of the Western blot analysis in wildtype iPSC-derived human motor neurons treated using PPM1A AON (QPA-646).

FIG. 13 shows the percent rescue of cell survival in a proteotoxic stress neurodegeneration model in response to treatment using PPM1A AONs (QPA-905, QPA-1045, and QPA-895).

### DETAILED DESCRIPTION

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

The terms “treat,” “treatment,” “treating,” and the like are used herein to generally mean obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, e.g., preventing the disease from increasing in severity or scope; (b) relieving the disease, e.g., causing partial or complete amelioration of the disease; or (c) preventing relapse of the disease, e.g., preventing the disease from returning to an active state following previous successful treatment of symptoms of the disease or treatment of the disease.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one biologically active compound, for example, a PPM1A antisense oligonucleotide (AON), as disclosed herein formulated together with one or more pharmaceutically acceptable excipients.

“Individual,” “patient,” or “subject” are used interchangeably and include to any animal, including mammals, preferably mice, rats, other rodents, rabbits, dogs, cats, swine, cattle, sheep, horses, or non-human primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, non-human primates, and the like). In some embodiments, the mammal treated in the methods of the invention is desirably a mammal in whom modulation of PPM1A expression and/or activity is desired.

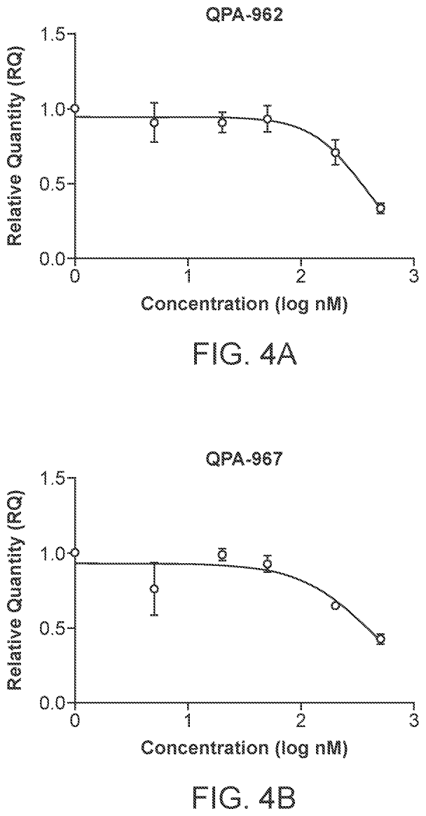

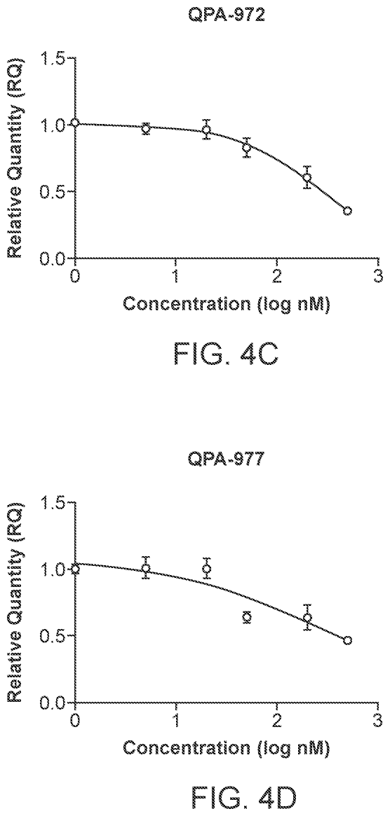

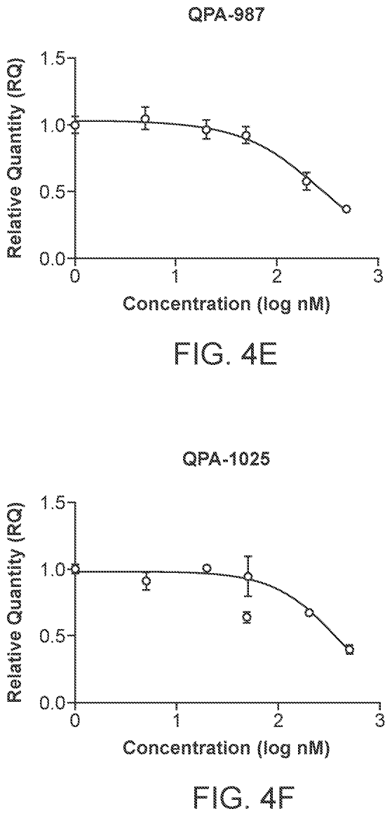

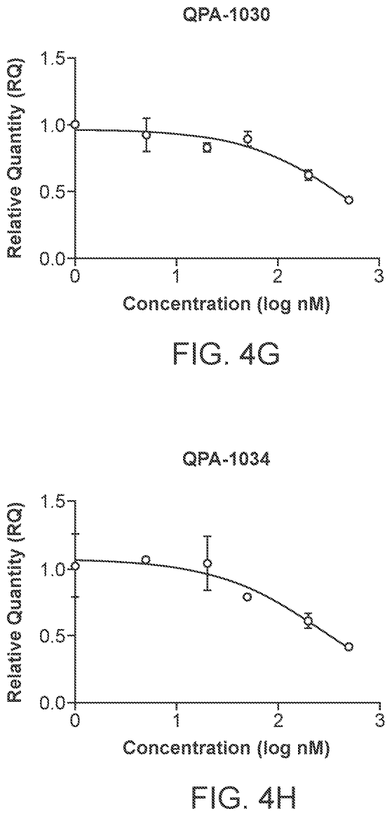

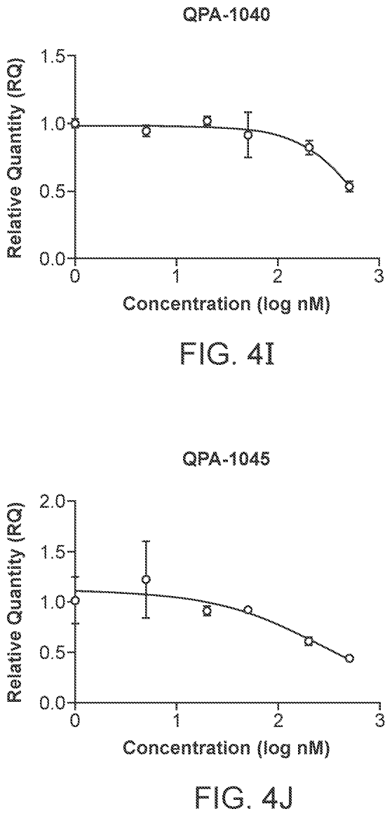

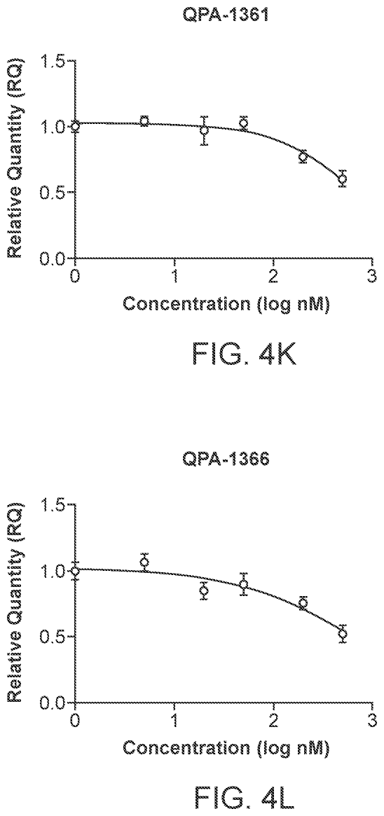

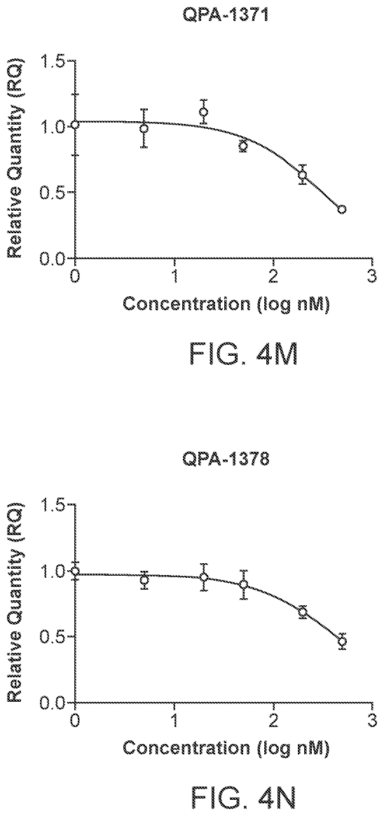

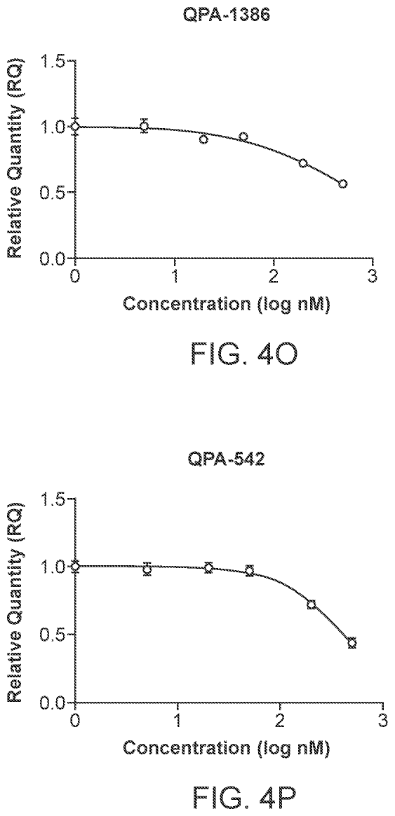

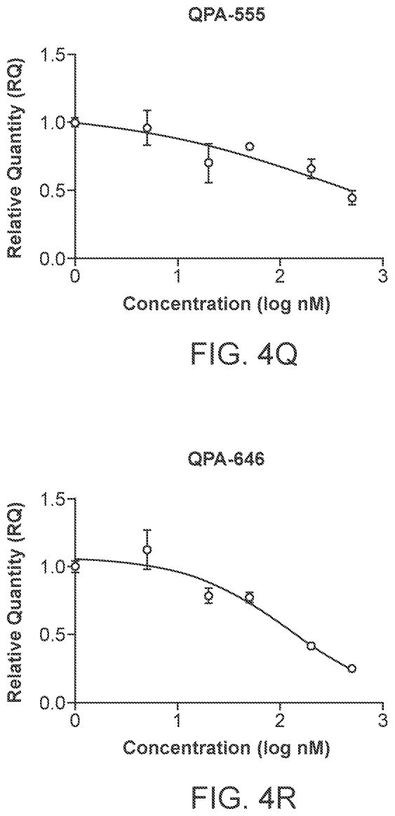

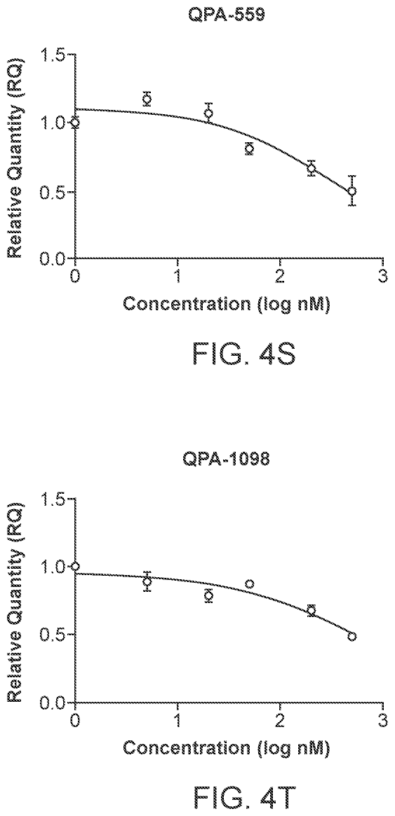

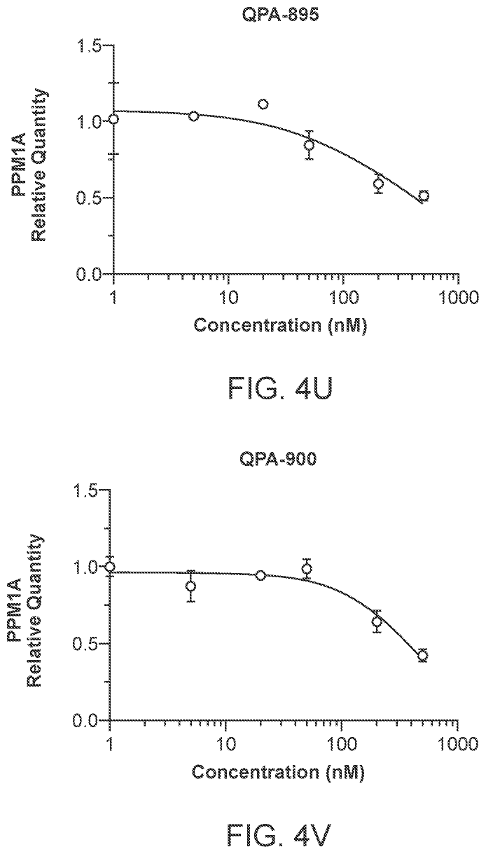

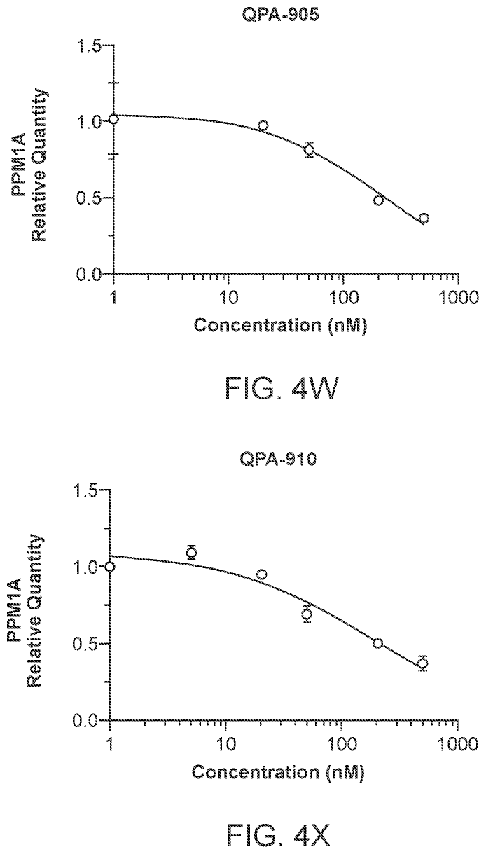

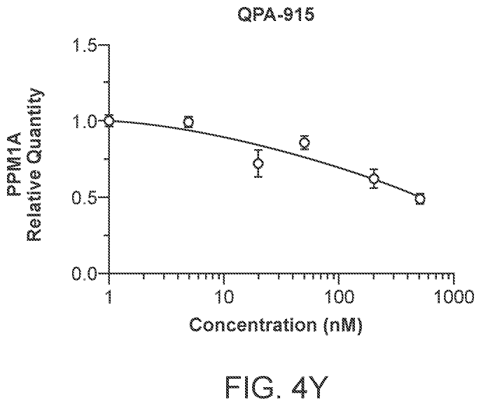

A patient suffering from ALS, FTD, ALS with FTD, or another neurological or motor neuron disease can be a patient that is diagnosed with the disease or that displays symptoms of the disease. A patient suffering from ALS, FTD, ALS with FTD, or another neurological or motor neuron disease can be a patient that previously suffered from the disease and, after recovering or experiencing complete or partial amelioration of the disease and/or disease symptoms, experiences a complete or partial relapse of the disease or disease symptoms. A patient suffering from ALS, FTD, ALS with FTD, or another neurological or motor neuron disease or condition can be a patient that harbors a genetic mutation associated with manifestation of the disease or condition. For example, a patient suffering from ALS can be a patient that harbors a genetic mutation in any of SOD1, C9orf72, Ataxin 2 (ATXN2), Charged Multivesicular Body Protein 2B (CHMP2B), Dynactin 1 (DCTN1), Human Epidermal Growth Factor Receptor 4 (ERBB4), FIG. 4 phosphoinositide 5-phosphatase (FIG. 4), NIMA related kinase 1 (NEK1), Heterogeneous nuclear ribonucleoprotein A1 (HNRNPA1), Neurofilament Heavy (NEFH), Peripherin (PRPH), TAR DNA binding protein 43 (TDP43 or TARDP), Fused in Sarcoma (FUS), Ubiquilin-2 (UBQLN2), Kinesin Family Member 5A (KIF5A), Valosin-Containing Protein (VCP), Alsin (ALS2), Senataxin (SETX), Sigma Non-Opioid Intracellular Receptor 1 (SIGMAR1), Survival of Motor Neuron 1, Telomeric (SMN1), Spastic Paraplegia 11, Autosomal Recessive (SPG11), Transient Receptor Potential Cation Channel Subfamily M Member 7 (TRPM7), Vesicle-Associated Membrane Protein-Associated Protein B/C (VAPB), Angiogenin (ANG), Profilin-1 (PFN1), Matrin-3 (MATR3), Coiled-coil-helix-coiled-coil-helix domain Containing 10 (CHCHD10), Tubulin, Alpha 4A (TUBA4A), TBK1, C21orf2, Sequestosome-1 (SQSTM1, also known as Ubiquitin-binding protein p62), and/or optineurin (OPTN), in particular, where the mutation is associated with ALS or a high risk of developing ALS.

A patient at risk of ALS, FTD, ALS with FTD, or another neurological or motor neuron disease can include those patients with a familial history of the disease or a genetic predisposition to the disease (e.g., a patient that harbors a genetic mutation associated with high disease risk, for example), or patients exposed to environmental factors that increase disease risk. For example, a patient may be at risk of ALS if the patient harbors a mutation in any of SOD1, C9orf72, ATXN2, CHMP2B, DCTN1, ERBB4, FIG. 4, HNRNPA1, NEFH, PRPH, NEK1, TDP43, FUS, UBQLN2, KIF5A, VCP, ALS2, SETX, SIGMAR1, SMN1, SPG11, TRPM7, VAPB, ANG, PFN1, MATR3, CHCHD10, TUBA4A, TBK1, SQSTM1, C21orf2, and/or OPTN, in particular, where the mutation is associated with ALS or high risk of developing ALS. A patient at risk may also include those patients diagnosed with a disease or condition that has a high comorbidity with ALS, FTD, ALS with FTD, or another neurological or motor neuron disease (for example, a patient suffering from dementia, which is significantly associated with higher odds of a family history of ALS, FTD, and of bulbar onset ALS (see Trojsi, F., et al. (2017) “Comorbidity of dementia with amyotrophic lateral sclerosis (ALS): insights from a large multicenter Italian cohort” J Neurol 264: 2224-31)).

As used herein, “PPM1A” (also known as Protein Phosphatase, Mg²⁺/Mn²⁺ Dependent 1A, Protein Phosphatase 1A (Formerly 2C), Magnesium-Dependent, Alpha Isoform, Protein Phosphatase 1A, EC 3.1.3.16, Protein Phosphatase 2C Isoform Alpha, Protein Phosphatase IA, Phosphatase 2C Alpha, PP2C-Alpha, PPPM1A, and PP2CA) refers to the gene or gene products (e.g., protein or mRNA transcript (including pre-mRNA) encoded by the gene) identified by Entrez Gene ID No. 5494 and allelic variants thereof, as well as orthologs found in non-human species (e.g., non-human primates or mice).

As used herein, “TBK1” (also known as Serine/threonine-protein kinase TBK1, NF-kappa-B-activating kinase, T2K, NAK, EC 2.7.11, FTDALS4 3, IIAE8, and TANK-binding kinase 1) refers to the gene or gene products (e.g., protein or mRNA transcript (including pre-mRNA) encoded by the gene) identified by Entrez Gene ID No. 29110 and allelic variants thereof, as well as orthologs found in non-human species (e.g., non-human primates or mice).

In the present specification, the term “therapeutically effective amount” means the amount of the subject PPM1A inhibitor that will elicit the biological or medical response of a tissue, system, animal or human that is being sought by the researcher, veterinarian, medical doctor, or other clinician. The PPM1A inhibitors of the invention are administered in therapeutically effective amounts to treat and/or prevent a disease, condition, disorder, or state, for example, ALS, FTD, ALS with FTD, or another motor neuron disease or neurological disease or condition. Alternatively, a therapeutically effective amount of a PPM1A inhibitor is the quantity required to achieve a desired therapeutic and/or prophylactic effect, such as an amount which results in the prevention of or a decrease in the symptoms associated with a disease associated with TBK1 inhibition, decreased TBK1 activity, or unwanted or deleterious PPM1A activity.

The terms “PPM1A AON” or “PPM1A antisense oligonucleotide” refers to an antisense oligonucleotide that is complementary to a portion of a PPM1A gene product, such as a PPM1A mRNA transcript. Examples of PPM1A AONs include PPM1A AONs with a sequence of any one of SEQ ID NOs: 2-955 or SEQ ID NOs: 1910-2863 or PPM1A Gapmer AONs with a sequence of any one of SEQ ID NOs: 2868-2959. “PPM1A AON” further includes PPM1A gapmer AONs.

The term “PPM1A gapmer AON” refers to a PPM1A AON with at least three distinct structural regions including a 5′-wing region, a central region, and a 3′-wing region, in ‘5→3’ orientation. The central region comprises a stretch of nucleosides that enable recruitment and activation of RNAseH. For example, the central region comprises linked DNA nucleosides, 2′-Fluoro Arabino Nucleic Acids (FANA), and Fluoro Cyclohexenyl nucleic acid (F-CeNA).

The term “pharmaceutically acceptable salt(s)” as used herein refers to salts of acidic or basic groups that may be present in PPM1A inhibitors used in the present compositions. PPM1A inhibitors included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, e.g., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (e.g., 1,1′-methylene-bis-(2-hydroxy-3-naphthoate)) salts. PPM1A inhibitors included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Pharmaceutically acceptable salts of the disclosure include, for example, pharmaceutically acceptable salts of PPM1A AONs that include a nucleotide sequence of any of SEQ ID NOs: 2-955, SEQ ID NOs: 1910-2863, SEQ ID NOs: 2868-2913, and SEQ ID NOs: 2914-2959.

PPM1A inhibitors of the disclosure may contain one or more chiral centers, groups, linkages, and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S” (or “Rp” or “Sp”) depending on the configuration of substituents around the stereogenic atom, for example, a stereogenic carbon, phosphorus, or sulfur atom. In some embodiments, one or more linkages of the compound may have a Rp or Sp configuration (e.g., one or more phosphorothioate linkages have either a Rp or Sp configuration). The configuration of each phosphorothioate linkage may be independent of another phosphorothioate linkage (e.g., one phosphorothioate linkage has a Rp configuration and a second phosphorothioate linkage has a Sp configuration). The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. Individual stereoisomers of PPM1A inhibitors of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase super critical fluid chromatography, chiral-phase simulated moving bed chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Individual stereoisomers of PPM1A inhibitors of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase super critical fluid chromatography, chiral-phase simulated moving bed chromatography, chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The PPM1A inhibitors disclosed herein can exist in solvated as well as unsolvated forms with pharmaceutically acceptable solvents such as water, ethanol, and the like, and it is intended that the invention embrace both solvated and unsolvated forms.

The invention also embraces isotopically labeled compounds of the invention (e.g., isotopically labeled PPM1A inhibitors) which are identical to those recited herein, except that one or more atoms are replaced by an atom having an atomic mass or mass number different from the atomic mass or mass number most abundantly found in nature. Examples of isotopes that can be incorporated into compounds of the invention include isotopes of hydrogen, carbon, nitrogen, oxygen, phosphorus, fluorine and chlorine, such as ²H, ³H, ¹¹C, ¹³C, ¹⁴C, ¹⁵N, ¹⁸O, ¹⁷O, ³¹P ³²P, ³⁵S, ¹⁸F, and ³⁶Cl, respectively.

Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances.

As used herein, “2′-O-(2-methoxyethyl)” (also 2′-MOE and 2′-O(CH₂)₂OCH₃ and MOE) refers to an O-methoxyethyl modification of the 2′ position of a furanose ring. A 2′-O-(2-methoxyethyl) modified sugar is a modified sugar.

As used herein, “2′-MOE nucleoside” (also 2′-O-(2-methoxyethyl) nucleoside) means a nucleoside comprising a 2′-MOE modified sugar moiety.

As used herein, “2′-substituted nucleoside” means a nucleoside comprising a substituent at the 2′-position of the furanose ring other than H or OH. In certain embodiments, 2′ substituted nucleosides include nucleosides with bicyclic sugar modifications.

As used herein, “bicyclic sugar” means a furanose ring modified by the bridging of two atoms. A bicyclic sugar is a modified sugar.

As used herein, “bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

As used herein, “cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

As used herein, “constrained ethyl nucleoside” (also cEt nucleoside) means a nucleoside comprising a bicyclic sugar moiety comprising a 4′-CH(CH₃)—O-2′ bridge. In some embodiments, cEt can be modified. In some embodiments, the cEt can be S-cEt. In some other embodiments, the cEt can be R-cEt.

As used herein, “internucleoside linkage” refers to the atom or group that links the 3′ and 5′ position of the sugar or corresponding positions of a sugar mimetic. In some embodiments, as used herein, “non-natural linkage” refers to a “modified internucleoside linkage.”

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “modified nucleobase” means any nucleobase other than adenine, cytosine, guanine, thymine, or uracil. Examples of a modified nucleobase include 5-methylcytosine, pseudouridine, or 5-methoxyuridine. An “unmodified nucleobase” means the purine bases adenine (A) and guanine (G), and the pyrimidine bases thymine (T), cytosine (C), and uracil (U).

As used herein, “5-methylcytosine” means a cytosine modified with a methyl group attached to the 5 position. A 5-methylcytosine is a modified nucleobase.

As used herein, a “modified nucleoside” means a nucleoside having, independently, a modified sugar moiety and/or modified nucleobase. A universal base is a modified nucleobase that can pair with any one. of the five unmodified nucleobases. Modified nucleosides include abasic nucleosides, which lack a nucleobase.

As used herein, “linked nucleosides” are nucleosides that are connected in a contiguous sequence (I.e., no additional nucleosides are presented between those that are linked).

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoosteen or reversed Hoosteen hydrogen bonding between complementary nucleobases.

As used herein, “increasing the amount of activity” refers to increased activity relative to the transcriptional expression or activity in an untreated or control sample.

As used herein, “mismatch” or “non-complementary nucleobase” refers to the case when a nucleobase of a first nucleic acid is not capable of pairing with the corresponding nucleobase of a second or target nucleic acid.

As used herein, “modified internucleoside linkage” refers to a substitution or any change from a naturally occurring internucleoside linkage (e.g., a phosphodiester internucleoside bond). “Phosphorothioate linkage” is a modified internucleoside linkage in which one of the non-bridging oxygen atoms of a phosphodiester internucleoside linkage is replaced with a sulfur atom.

As used herein, “modified oligonucleotide” means an oligonucleotide comprising at least one modified internucleoside linkage, modified sugar, and/or modified nucleobase.

As used herein, “modified sugar” or “modified sugar moiety” means a modified furanosyl sugar moiety or a modified sugar moiety having other than a furanosyl moiety that can link a nucleobase to another group, such as an internucleoside linkage, conjugate group, or terminal group in an oligonucleotide.

As used herein, “monomer” means a single unit of an oligomer. Monomers include, but are not limited to, nucleosides and nucleotides, whether naturally occurring or modified.

As used herein, “motif” means the pattern of unmodified and modified nucleosides in an antisense compound.

As used herein, “natural sugar moiety” means a sugar moiety found in DNA (2′-H) or RNA (2′-OH).

As used herein, “naturally occurring internucleoside linkage” means a 3′ to 5′ phosphodiester linkage.

As used herein, “nucleobase” means a heterocyclic moiety capable of pairing with a base of another nucleic acid.

As used herein, “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, “nucleobase sequence” means the order of contiguous nucleobases independent of any sugar, linkage, and/or nucleobase modification.

As used herein, “nucleoside” means a nucleobase linked to a sugar. The term “nucleoside” also includes a “modified nucleoside” which has independently, a modified sugar moiety and/or modified nucleobase.

As used herein, “nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O—or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

As used herein, “nucleotide” means a nucleoside having a phosphate group covalently linked to the sugar portion of the nucleoside.

As used herein, “oligomeric compound” or “oligomer” means a polymer of linked monomeric subunits which is capable of hybridizing to at least a region of a nucleic acid molecule.

As used herein, “oligonucleotide” means a polymer of linked nucleosides each of which can be modified or unmodified, independent one from another.

The disclosure provides methods for treating, ameliorating, or preventing a neurological disease such as, but not limited to, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease, in a patient, comprising administering to a patient a PPM1A inhibitor effective to inhibit PPM1A activity and/or expression and/or to increase TBK1 expression, phosphorylation, and/or activity, where the composition comprises a therapeutically effective amount of a PPM1A inhibitor, and a pharmaceutically acceptable excipient. Also provided herein are methods of treating, ameliorating, or preventing a neurological disease such as, but not limited to, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease, a condition, or a disorder characterized by symptoms associated with a neurological disease such as, but not limited to, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease, comprising administering to a patient a composition effective to inhibit PPM1A activity and/or expression and/or to increase TBK1 expression, phosphorylation, and/or activity, wherein the composition comprises a therapeutically effective amount of a PPM1A inhibitor, for example, a PPM1A AON, and a pharmaceutically acceptable excipient.

For example, in some embodiments, methods for treating, ameliorating, or preventing a neurological disease such as, but not limited to, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease, or treating, ameliorating, or preventing a neurological disease, condition, or a disorder characterized symptoms associated with a neurological disease such as, but not limited to, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease, include methods of administering a pharmaceutically acceptable composition, for example, a pharmaceutically acceptable formulation, that includes one or more PPM1A inhibitors, to a patient. PPM1A inhibitors can inhibit PPM1A activity, for example, PPM1A phosphatase activity, and/or levels of PPM1A expression, for example, PPM1A mRNA and/or protein expression. Without wishing to be bound by theory, a PPM1A inhibitor can inhibit PPM1A activity and/or expression and increase TBK1 expression, phosphorylation, and/or activity by decreasing the amount of active PPM1A, allowing a greater portion of total TBK1 to retain a phosphorylated form.

The present disclosure also provides pharmaceutical compositions comprising PPM1A inhibitors as disclosed herein formulated together with one or more pharmaceutically or cosmetically acceptable excipients. These formulations include those suitable for oral, sublingual, intratracheal, intranasal, vaginal, rectal, topical, transdermal, pulmonary, intrathecal, intracisternal, buccal, and parenteral (e.g., subcutaneous, intramuscular, intradermal, intraduodenal, or intravenous) administration, or for topical use, e.g., as part of a composition suitable for applying topically to skin and/or mucous membrane, for example, a composition in the form of a gel, a paste, a wax, a cream, a spray, a liquid, a foam, a lotion, an ointment, a topical solution, a transdermal patch, a powder, a vapor, or a tincture. Although the most suitable form of administration in any given case will depend on the degree and severity of the condition being treated and on the nature of the particular PPM1A inhibitor being used.

The present invention also provides a pharmaceutical composition comprising a PPM1A inhibitor, or a pharmaceutically acceptable salt thereof (for example, a PPM1A AON that includes a nucleotide sequence of any of SEQ ID NOs: 2-955, SEQ ID NOs: 1910-2863, SEQ ID NOs: 2868-2913, and SEQ ID NOs: 2914-2959).

The present disclosure also provides methods that include the use of pharmaceutical compositions comprising PPM1A inhibitors as disclosed herein (e.g., a PPM1A AON of any one of SEQ ID NOs: 2-955, SEQ ID NOs: 1910-2863, SEQ ID NOs: 2868-2913, and SEQ ID NOs: 2914-2959) formulated together with one or more pharmaceutically acceptable excipients. Exemplary compositions provided herein include compositions comprising essentially a PPM1A inhibitor, as described above, and one or more pharmaceutically acceptable excipients. Formulations include those suitable for oral, sublingual, intratracheal, intranasal, rectal, vaginal, topical, transdermal, pulmonary, intrathecal, intracisternal, buccal, and parenteral (e.g., subcutaneous, intramuscular, intradermal, intraduodenal, or intravenous) administration, or for topical use. The most suitable form of administration in any given case will depend on the clinical symptoms, complications, or biochemical indicia of the state, disorder, disease, or condition that one is trying to prevent in a subject; the state, disorder, disease, or condition one is trying to prevent in a subject; and/or on the nature of the particular compound and/or the composition being used.

PPM1A Inhibitors

In certain embodiments, PPM1A levels (e.g., PPM1A mRNA or protein levels) and/or activity (e.g., biological activity, for example, PPM1A phosphatase activity) can be decreased using compounds or compositions that target the PPM1A gene or a PPM1A gene product (for example, a PPM1A mRNA). Similarly, phosphorylated TBK1 (pTBK1) levels (e.g., pTBK1 protein levels) and/or activity (e.g., TBK1 biological activity, for example, kinase activity) can be increased using compounds or compositions that target the PPM1A gene or a PPM1A gene product (for example, a PPM1A mRNA or a PPM1A pre-mRNA). In various embodiments, such PPM1A inhibitors are PPM1A antisense therapeutics e.g., antisense oligonucleotides (AONs) that target the PPM1A gene or PPM1A gene product (e.g., PPM1A mRNA).

PPM1A inhibitors can be, but are not limited to, compounds such as PPM1A antibodies and antibody fragments (for example, PPM1A monoclonal antibodies, PPM1A Fab fragments (e.g., F(ab′)₂ and Fab′), PPM1A variable fragments (e.g., PPM1A single-chain variable fragments, dimeric single-chain variable fragments, and single-domain antibodies), and PPM1A bispecific monoclonal antibodies), small molecule inhibitors of PPM1A, nucleotide-based inhibitors of PPM1A (for example, PPM1A shRNAs, PPM1A siRNAs, PPM1A PNAs, PPM1A LNAs, or PPM1A morpholino oligomers), or compositions that include such compounds.

PPM1A antibodies include, for example, anti-PPM1A antibody p6c7 (Cat. No. ab14824; Abcam, Cambridge, Mass., USA), anti-PPM1A, clone 7F12 antibody (Cat. No. MAB S415; Millipore, Burlington, Mass., USA), and anti-PPM1A clone 4E11 (Cat. No. SAB1402318, Sigma-Aldrich, Burlington, Mass., USA).

PPM1A small molecule inhibitors include the plant alkaloid sanguinarine (see Aburai et al. (2010) “Sanguinarine as a potent and specific inhibitor of protein phosphatase 2C in vitro and induces apoptosis via phosphorylation of p38 in HL60 cells” Biosci Biotechnol Biochem. 74(3):548-52). Additional PPM1A small molecule inhibitors include proteolysis targeting chimera (PROTACS), such as a PROTACS that induces proteolysis of PPM1A protein.

PPM1A Antisense Therapeutics

Antisense therapeutics are a class of nucleic acid-based compounds that can be used to inhibit gene expression. Antisense therapeutics may be single- or double-stranded deoxyribonucleic acid (DNA)-based, ribonucleic acid (RNA)-based, or DNA/RNA chemical analogue compounds. In general, antisense therapeutics are designed to include a nucleotide sequence that is complementary or nearly complementary to an mRNA or pre-mRNA sequence transcribed from a given gene in order to promote binding between the antisense therapeutic and the pre-mRNA or mRNA. Without being bound by theory, it is believed that in most instances antisense therapeutics act by binding to an mRNA or pre-mRNA, thereby inhibiting protein translation, altering pre-mRNA splicing into mature mRNA, and/or causing destruction of mRNA. In most instances, the antisense therapeutic nucleotide sequence is complementary to a portion of a targeted gene's or mRNA's sense sequence. PPM1A antisense therapeutics described herein are oligonucleotide-based compounds that include an oligonucleotide sequence complementary to a PPM1A gene sense, PPM1A pre-mRNA sense, and/or PPM1A mRNA sense sequence, or a portion thereof PPM1A antisense therapeutics described herein can also be nucleotide chemical analog-based compounds capable of binding to a PPM1A gene sense, PPM1A pre-mRNA sense, and/or PPM1A mRNA sense sequence, or a portion thereof PPM1A antisense therapeutics include PPM1A antisense oligonucleotides, PPM1A shRNAs, PPM1A siRNAs, PPM1A PNAs, PPM1A LNAs, and PPM1A morpholino oligomers.

Antisense oligonucleotides (AONs) are short oligonucleotide-based sequences that include an oligonucleotide sequence complementary to a target RNA sequence. AONs are typically between 8 to 50 nucleotides in length, for example, 20 nucleotides in length. AONs may include chemically modified nucleosides (for example, 2′-O-methylated nucleosides or 2′-O-(2-methoxyethyl) nucleosides) as well as modified internucleoside linkages (for example, phosphorothioate linkages). PPM1A AONs described herein include oligonucleotide sequences that are complementary to PPM1A RNA sequences, such as PPM1A mRNA transcripts. PPM1A AONs described herein can include chemically modified nucleosides and modified internucleoside linkages (for example, phosphorothioate linkages).

Peptide nucleic acids (PNAs) are short, artificially synthesized polymers with a structure that mimics DNA or RNA. PNAs include a backbone composed of repeating N-(2-aminoethyl)-glycine units linked by peptide bonds. PPM1A PNAs described herein can be used as antisense therapeutics that bind to PPM1A RNA sequences with high specificity and inhibit PPM1A gene expression.

Locked nucleic acids (LNAs) are oligonucleotide sequences that include one or more modified RNA nucleotides in which the ribose moiety is modified with an extra bridge connecting the 2′ oxygen and 4′ carbon. LNAs are believed to have higher Tm's than analogous oligonucleotide sequences. PPM1A LNAs described herein can be used as antisense therapeutics that bind to PPM1A RNA sequences with high specificity and inhibit PPM1A gene expression.

Morpholino oligomers are oligonucleotide compounds that include DNA bases attached to a backbone of methylenemorpholine rings linked through phosphorodiamidate groups. Morpholino oligomers of the present invention can be designed to bind to specific PPM1A RNA sequences of interest (for example, PPM1A mRNA or PPM1A pre-mRNA sequences of interest), thereby preventing gene expression. PPM1A morpholino oligomers described herein can be used as antisense therapeutics that bind to PPM1A mRNA sequences with high specificity and inhibit PPM1A gene expression. PPM1A morpholino oligomers described herein can also be used to bind PPM1A pre-mRNA sequences, altering PPM1A pre-mRNA splicing and PPM1A gene expression.

Small hairpin RNAs (shRNAs) are generally RNA molecules with a hairpin-like structure that can be used to silence gene expression. shRNAs are generally expressed from plasmids encoding the shRNA sequence, and can be expressed from viral vectors to allow lentiviral, adenoviral, or adeno-associated viral expression. Without being bound by theory, it is believed that shRNA inhibits gene expression by taking advantage of RNA interference (RNAi) processes. In brief, the shRNA transcript is processed by Drosha and Dicer, and then loaded onto the RNA-induced silencing complex (RISC), allowing targeting of specific mRNA, and either mRNA degradation or repression of protein translation. PPM1A shRNAs described herein can inhibit gene expression of PPM1A.

Small interfering RNAs (siRNAs) are double-stranded RNA molecules of approximately 20-25 base pairs in length that take advantage of RNAi machinery (e.g., Drosha and RISC) to bind and target mRNA for degradation. siRNAs are not dependent upon plasmids or vectors for expression, and can generally be delivered directly to a target cell, for instance, by transfection. PPM1A siRNAs are double-stranded RNA sequences that include an RNA sequence complementary to a PPM1A mRNA sequence, and which prevent PPM1A protein translation.

The number of nucleotides included in a PPM1A antisense therapeutic, for example, a PPM1A antisense oligonucleotide described herein may vary. For example, in some embodiments, the antisense oligonucleotide is from 12 to 15 nucleotides in length. In some embodiments, the antisense oligonucleotide is from 15 to 20 nucleotides in length. In some embodiments, the antisense oligonucleotide is from 20 to 40 nucleotides in length. In some embodiments, the antisense oligonucleotide is from 20 to 22 nucleotides in length. In some embodiments, the antisense oligonucleotide is from 22 to 40 nucleotides in length. In some embodiments, the antisense oligonucleotide is from 20 to 30, 25 to 35, or 30 to 40 nucleotides in length.

PPM1A Antisense Oligonucleotides

PPM1A antisense oligonucleotides (AONs) described herein are short synthetic oligonucleotide sequence complementary to a portion of a PPM1A gene product, such as a PPM1A transcript (for example, a PPM1A mRNA transcript).

In various embodiments, PPM1A AONs include linked nucleosides with a nucleobase sequence that is at least 90%, at least 91%, at least 92%, at least 93%, at least 94%, at least 95%, at least 96%, at least 97%, at least 98%, at least 99%, or that is 100% complementary to a portion of a PPM1A gene product, such as a PPM1A mRNA sequence. In some embodiments, a PPM1A AON can include a non-duplexed oligonucleotide. In some embodiments, a PPM1A AON can include a duplex of two oligonucleotides where the first oligonucleotide includes a nucleotide sequence that is completely or almost completely complementary to a PPM1A mRNA sequence and the second oligonucleotide includes a nucleotide sequence that is complementary to the nucleotide sequence of the first oligonucleotide. AON binding specificity can be assessed via measurement of parameters such as dissociation constant, melting temperature (Tm), or other criteria such as changes in protein or RNA expression levels or other assays that measure PPM1A activity or expression.

A PPM1A AON, such as disclosed herein, may be an oligonucleotide sequence of 5 to 100 nucleotides in length, for example, 10 to 40 nucleotides in length, for example, 14 to 40 nucleotides in length, 10 to 30 nucleotides in length, for example, 14 to 30 nucleotides in length, for example, 14 to 25 nucleotides in length, 15 to 22 nucleotides in length, 18 to 21 nucleotides in length, or 18, 19, 20, 21, 22, 23, 24, or 25 nucleotides in length.

PPM1A AONs described herein also include antisense oligonucleotides comprising the oligonucleotide sequences listed in Table 1 below. The “Start Position” column in Table 1 refers to the first position in the PPM1A mRNA transcript (SEQ ID NO: 2864) that the PPM1A AON sequence is complementary to. As an example, oligonucleotide sequence with a “Start Position” of 457 is complementary to a first nucleotide at position 457 of SEQ ID NO: 2864.

TABLE 1
PPM1A AON Sequences and Corresponding
Target Sequences

| SEQ | Start | PPM1A AON | SEQ | |
|---|---|---|---|---|
| ID | Posi- | sequence | ID | Target Sequence |
| NO: | tion | (5′→3′)* | NO: | (5′→3′) |
| 2 | 457 | ATGTCTTGATCCTCTAGGTC | 956 | GACCTAGAGGATCAAGACAT |
| 3 | 458 | TATGTCTTGATCCTCTAGGT | 957 | ACCTAGAGGATCAAGACATA |
| 4 | 459 | TTATGTCTTGATCCTCTAGG | 958 | CCTAGAGGATCAAGACATAA |
| 5 | 460 | ATTATGTCTTGATCCTCTAG | 959 | CTAGAGGATCAAGACATAAT |
| 6 | 461 | CATTATGTCTTGATCCTCTA | 960 | TAGAGGATCAAGACATAATG |
| 7 | 462 | CCATTATGTCTTGATCCTCT | 961 | AGAGGATCAAGACATAATGG |
| 8 | 463 | CCCATTATGTCTTGATCCTC | 962 | GAGGATCAAGACATAATGGG |
| 9 | 464 | TCCCATTATGTCTTGATCCT | 963 | AGGATCAAGACATAATGGGA |
| 10 | 465 | CTCCCATTATGTCTTGATCC | 964 | GGATCAAGACATAATGGGAG |
| 11 | 466 | GCTCCCATTATGTCTTGATC | 965 | GATCAAGACATAATGGGAGC |
| 12 | 467 | TGCTCCCATTATGTCTTGAT | 966 | ATCAAGACATAATGGGAGCA |
| 13 | 468 | ATGCTCCCATTATGTCTTGA | 967 | TCAAGACATAATGGGAGCAT |
| 14 | 469 | AATGCTCCCATTATGTCTTG | 968 | CAAGACATAATGGGAGCATT |
| 15 | 470 | AAATGCTCCCATTATGTCTT | 969 | AAGACATAATGGGAGCATTT |
| 16 | 471 | AAAATGCTCCCATTATGTCT | 970 | AGACATAATGGGAGCATTTT |
| 17 | 472 | AAAAATGCTCCCATTATGTC | 971 | GACATAATGGGAGCATTTTT |
| 18 | 473 | TAAAAATGCTCCCATTATGT | 972 | ACATAATGGGAGCATTTTTA |
| 19 | 474 | CTAAAAATGCTCCCATTATG | 973 | CATAATGGGAGCATTTTTAG |
| 20 | 475 | TCTAAAAATGCTCCCATTAT | 974 | ATAATGGGAGCATTTTTAGA |
| 21 | 476 | GTCTAAAAATGCTCCCATTA | 975 | TAATGGGAGCATTTTTAGAC |
| 22 | 477 | TGTCTAAAAATGCTCCCATT | 976 | AATGGGAGCATTTTTAGACA |
| 23 | 478 | TTGTCTAAAAATGCTCCCAT | 977 | ATGGGAGCATTTTTAGACAA |
| 24 | 479 | CTTGTCTAAAAATGCTCCCA | 978 | TGGGAGCATTTTTAGACAAG |
| 25 | 480 | GCTTGTCTAAAAATGCTCCC | 979 | GGGAGCATTTTTAGACAAGC |
| 26 | 481 | GGCTTGTCTAAAAATGCTCC | 980 | GGAGCATTTTTAGACAAGCC |
| 27 | 482 | TGGCTTGTCTAAAAATGCTC | 981 | GAGCATTTTTAGACAAGCCA |
| 28 | 483 | TTGGCTTGTCTAAAAATGCT | 982 | AGCATTTTTAGACAAGCCAA |
| 29 | 484 | TTTGGCTTGTCTAAAAATGC | 983 | GCATTTTTAGACAAGCCAAA |
| 30 | 485 | CTTTGGCTTGTCTAAAAATG | 984 | CATTTTTAGACAAGCCAAAG |
| 31 | 486 | TCTTTGGCTTGTCTAAAAAT | 985 | ATTTTTAGACAAGCCAAAGA |
| 32 | 487 | ATCTTTGGCTTGTCTAAAAA | 986 | TTTTTAGACAAGCCAAAGAT |
| 33 | 488 | CATCTTTGGCTTGTCTAAAA | 987 | TTTTAGACAAGCCAAAGATG |
| 34 | 489 | CCATCTTTGGCTTGTCTAAA | 988 | TTTAGACAAGCCAAAGATGG |
| 35 | 490 | TCCATCTTTGGCTTGTCTAA | 989 | TTAGACAAGCCAAAGATGGA |
| 36 | 491 | TTCCATCTTTGGCTTGTCTA | 990 | TAGACAAGCCAAAGATGGAA |
| 37 | 492 | TTTCCATCTTTGGCTTGTCT | 991 | AGACAAGCCAAAGATGGAAA |
| 38 | 493 | TTTTCCATCTTTGGCTTGTC | 992 | GACAAGCCAAAGATGGAAAA |
| 39 | 494 | CTTTTCCATCTTTGGCTTGT | 993 | ACAAGCCAAAGATGGAAAAG |
| 40 | 495 | GCTTTTCCATCTTTGGCTTG | 994 | CAAGCCAAAGATGGAAAAGC |
| 41 | 496 | TGCTTTTCCATCTTTGGCTT | 995 | AAGCCAAAGATGGAAAAGCA |
| 42 | 497 | ATGCTTTTCCATCTTTGGCT | 996 | AGCCAAAGATGGAAAAGCAT |
| 43 | 498 | TATGCTTTTCCATCTTTGGC | 997 | GCCAAAGATGGAAAAGCATA |
| 44 | 499 | TTATGCTTTTCCATCTTTGG | 998 | CCAAAGATGGAAAAGCATAA |
| 45 | 500 | ATTATGCTTTTCCATCTTTG | 999 | CAAAGATGGAAAAGCATAAT |
| 46 | 501 | CATTATGCTTTTCCATCTTT | 1000 | AAAGATGGAAAAGCATAATG |
| 47 | 502 | GCATTATGCTTTTCCATCTT | 1001 | AAGATGGAAAAGCATAATGC |
| 48 | 503 | GGCATTATGCTTTTCCATCT | 1002 | AGATGGAAAAGCATAATGCC |
| 49 | 504 | GGGCATTATGCTTTTCCATC | 1003 | GATGGAAAAGCATAATGCCC |
| 50 | 505 | TGGGCATTATGCTTTTCCAT | 1004 | ATGGAAAAGCATAATGCCCA |
| 51 | 506 | CTGGGCATTATGCTTTTCCA | 1005 | TGGAAAAGCATAATGCCCAG |
| 52 | 507 | CCTGGGCATTATGCTTTTCC | 1006 | GGAAAAGCATAATGCCCAGG |
| 53 | 508 | CCCTGGGCATTATGCTTTTC | 1007 | GAAAAGCATAATGCCCAGGG |
| 54 | 509 | CCCCTGGGCATTATGCTTTT | 1008 | AAAAGCATAATGCCCAGGGG |
| 55 | 510 | GCCCCTGGGCATTATGCTTT | 1009 | AAAGCATAATGCCCAGGGGC |
| 56 | 511 | TGCCCCTGGGCATTATGCTT | 1010 | AAGCATAATGCCCAGGGGCA |
| 57 | 512 | CTGCCCCTGGGCATTATGCT | 1011 | AGCATAATGCCCAGGGGCAG |
| 58 | 513 | CCTGCCCCTGGGCATTATGC | 1012 | GCATAATGCCCAGGGGCAGG |
| 59 | 514 | CCCTGCCCCTGGGCATTATG | 1013 | CATAATGCCCAGGGGCAGGG |
| 60 | 515 | ACCCTGCCCCTGGGCATTAT | 1014 | ATAATGCCCAGGGGCAGGGT |
| 61 | 516 | TACCCTGCCCCTGGGCATTA | 1015 | TAATGCCCAGGGGCAGGGTA |
| 62 | 517 | TTACCCTGCCCCTGGGCATT | 1016 | AATGCCCAGGGGCAGGGTAA |
| 63 | 518 | ATTACCCTGCCCCTGGGCAT | 1017 | ATGCCCAGGGGCAGGGTAAT |
| 64 | 519 | CATTACCCTGCCCCTGGGCA | 1018 | TGCCCAGGGGCAGGGTAATG |
| 65 | 520 | CCATTACCCTGCCCCTGGGC | 1019 | GCCCAGGGGCAGGGTAATGG |
| 66 | 521 | CCCATTACCCTGCCCCTGGG | 1020 | CCCAGGGGCAGGGTAATGGG |
| 67 | 522 | ACCCATTACCCTGCCCCTGG | 1021 | CCAGGGGCAGGGTAATGGGT |
| 68 | 523 | AACCCATTACCCTGCCCCTG | 1022 | CAGGGGCAGGGTAATGGGTT |
| 69 | 524 | CAACCCATTACCCTGCCCCT | 1023 | AGGGGCAGGGTAATGGGTTG |
| 70 | 525 | GCAACCCATTACCCTGCCCC | 1024 | GGGGCAGGGTAATGGGTTGC |
| 71 | 526 | CGCAACCCATTACCCTGCCC | 1025 | GGGCAGGGTAATGGGTTGCG |
| 72 | 527 | TCGCAACCCATTACCCTGCC | 1026 | GGCAGGGTAATGGGTTGCGA |
| 73 | 528 | ATCGCAACCCATTACCCTGC | 1027 | GCAGGGTAATGGGTTGCGAT |
| 74 | 529 | TATCGCAACCCATTACCCTG | 1028 | CAGGGTAATGGGTTGCGATA |
| 75 | 530 | ATATCGCAACCCATTACCCT | 1029 | AGGGTAATGGGTTGCGATAT |
| 76 | 531 | CATATCGCAACCCATTACCC | 1030 | GGGTAATGGGTTGCGATATG |
| 77 | 532 | CCATATCGCAACCCATTACC | 1031 | GGTAATGGGTTGCGATATGG |
| 78 | 533 | CCCATATCGCAACCCATTAC | 1032 | GTAATGGGTTGCGATATGGG |
| 79 | 534 | GCCCATATCGCAACCCATTA | 1033 | TAATGGGTTGCGATATGGGC |
| 80 | 535 | AGCCCATATCGCAACCCATT | 1034 | AATGGGTTGCGATATGGGCT |
| 81 | 536 | TAGCCCATATCGCAACCCAT | 1035 | ATGGGTTGCGATATGGGCTA |
| 82 | 537 | TTAGCCCATATCGCAACCCA | 1036 | TGGGTTGCGATATGGGCTAA |
| 83 | 538 | CTTAGCCCATATCGCAACCC | 1037 | GGGTTGCGATATGGGCTAAG |
| 84 | 539 | GCTTAGCCCATATCGCAACC | 1038 | GGTTGCGATATGGGCTAAGC |
| 85 | 540 | TGCTTAGCCCATATCGCAAC | 1039 | GTTGCGATATGGGCTAAGCA |
| 86 | 541 | CTGCTTAGCCCATATCGCAA | 1040 | TTGCGATATGGGCTAAGCAG |
| 87 | 542 | GCTGCTTAGCCCATATCGCA | 1041 | TGCGATATGGGCTAAGCAGC |
| 88 | 543 | TGCTGCTTAGCCCATATCGC | 1042 | GCGATATGGGCTAAGCAGCA |
| 89 | 544 | ATGCTGCTTAGCCCATATCG | 1043 | CGATATGGGCTAAGCAGCAT |
| 90 | 545 | CATGCTGCTTAGCCCATATC | 1044 | GATATGGGCTAAGCAGCATG |
| 91 | 546 | GCATGCTGCTTAGCCCATAT | 1045 | ATATGGGCTAAGCAGCATGC |
| 92 | 547 | TGCATGCTGCTTAGCCCATA | 1046 | TATGGGCTAAGCAGCATGCA |
| 93 | 548 | TTGCATGCTGCTTAGCCCAT | 1047 | ATGGGCTAAGCAGCATGCAA |
| 94 | 549 | CTTGCATGCTGCTTAGCCCA | 1048 | TGGGCTAAGCAGCATGCAAG |
| 95 | 550 | CCTTGCATGCTGCTTAGCCC | 1049 | GGGCTAAGCAGCATGCAAGG |
| 96 | 551 | GCCTTGCATGCTGCTTAGCC | 1050 | GGCTAAGCAGCATGCAAGGC |
| 97 | 552 | AGCCTTGCATGCTGCTTAGC | 1051 | GCTAAGCAGCATGCAAGGCT |
| 98 | 553 | CAGCCTTGCATGCTGCTTAG | 1052 | CTAAGCAGCATGCAAGGCTG |
| 99 | 554 | CCAGCCTTGCATGCTGCTTA | 1053 | TAAGCAGCATGCAAGGCTGG |
| 100 | 555 | GCCAGCCTTGCATGCTGCTT | 1054 | AAGCAGCATGCAAGGCTGGC |
| 101 | 556 | CGCCAGCCTTGCATGCTGCT | 1055 | AGCAGCATGCAAGGCTGGCG |
| 102 | 557 | ACGCCAGCCTTGCATGCTGC | 1056 | GCAGCATGCAAGGCTGGCGT |
| 103 | 558 | CACGCCAGCCTTGCATGCTG | 1057 | CAGCATGCAAGGCTGGCGTG |
| 104 | 559 | ACACGCCAGCCTTGCATGCT | 1058 | AGCATGCAAGGCTGGCGTGT |
| 105 | 560 | AACACGCCAGCCTTGCATGC | 1059 | GCATGCAAGGCTGGCGTGTT |
| 106 | 561 | CAACACGCCAGCCTTGCATG | 1060 | CATGCAAGGCTGGCGTGTTG |
| 107 | 562 | TCAACACGCCAGCCTTGCAT | 1061 | ATGCAAGGCTGGCGTGTTGA |
| 108 | 563 | TTCAACACGCCAGCCTTGCA | 1062 | TGCAAGGCTGGCGTGTTGAA |
| 109 | 564 | TTTCAACACGCCAGCCTTGC | 1063 | GCAAGGCTGGCGTGTTGAAA |
| 110 | 565 | ATTTCAACACGCCAGCCTTG | 1064 | CAAGGCTGGCGTGTTGAAAT |
| 111 | 566 | CATTTCAACACGCCAGCCTT | 1065 | AAGGCTGGCGTGTTGAAATG |
| 112 | 567 | CCATTTCAACACGCCAGCCT | 1066 | AGGCTGGCGTGTTGAAATGG |
| 113 | 568 | TCCATTTCAACACGCCAGCC | 1067 | GGCTGGCGTGTTGAAATGGA |
| 114 | 569 | CTCCATTTCAACACGCCAGC | 1068 | GCTGGCGTGTTGAAATGGAG |
| 115 | 570 | CCTCCATTTCAACACGCCAG | 1069 | CTGGCGTGTTGAAATGGAGG |
| 116 | 571 | TCCTCCATTTCAACACGCCA | 1070 | TGGCGTGTTGAAATGGAGGA |
| 117 | 572 | ATCCTCCATTTCAACACGCC | 1071 | GGCGTGTTGAAATGGAGGAT |
| 118 | 573 | CATCCTCCATTTCAACACGC | 1072 | GCGTGTTGAAATGGAGGATG |
| 119 | 574 | GCATCCTCCATTTCAACACG | 1073 | CGTGTTGAAATGGAGGATGC |
| 120 | 575 | TGCATCCTCCATTTCAACAC | 1074 | GTGTTGAAATGGAGGATGCA |
| 121 | 576 | GTGCATCCTCCATTTCAACA | 1075 | TGTTGAAATGGAGGATGCAC |
| 122 | 577 | TGTGCATCCTCCATTTCAAC | 1076 | GTTGAAATGGAGGATGCACA |
| 123 | 578 | ATGTGCATCCTCCATTTCAA | 1077 | TTGAAATGGAGGATGCACAT |
| 124 | 579 | TATGTGCATCCTCCATTTCA | 1078 | TGAAATGGAGGATGCACATA |
| 125 | 580 | GTATGTGCATCCTCCATTTC | 1079 | GAAATGGAGGATGCACATAC |
| 126 | 581 | CGTATGTGCATCCTCCATTT | 1080 | AAATGGAGGATGCACATACG |
| 127 | 582 | CCGTATGTGCATCCTCCATT | 1081 | AATGGAGGATGCACATACGG |
| 128 | 583 | GCCGTATGTGCATCCTCCAT | 1082 | ATGGAGGATGCACATACGGC |
| 129 | 584 | AGCCGTATGTGCATCCTCCA | 1083 | TGGAGGATGCACATACGGCT |
| 130 | 585 | CAGCCGTATGTGCATCCTCC | 1084 | GGAGGATGCACATACGGCTG |
| 131 | 586 | ACAGCCGTATGTGCATCCTC | 1085 | GAGGATGCACATACGGCTGT |
| 132 | 587 | CACAGCCGTATGTGCATCCT | 1086 | AGGATGCACATACGGCTGTG |
| 133 | 588 | TCACAGCCGTATGTGCATCC | 1087 | GGATGCACATACGGCTGTGA |
| 134 | 589 | ATCACAGCCGTATGTGCATC | 1088 | GATGCACATACGGCTGTGAT |
| 135 | 590 | GATCACAGCCGTATGTGCAT | 1089 | ATGCACATACGGCTGTGATC |
| 136 | 591 | CGATCACAGCCGTATGTGCA | 1090 | TGCACATACGGCTGTGATCG |
| 137 | 592 | CCGATCACAGCCGTATGTGC | 1091 | GCACATACGGCTGTGATCGG |
| 138 | 593 | ACCGATCACAGCCGTATGTG | 1092 | CACATACGGCTGTGATCGGT |
| 139 | 594 | AACCGATCACAGCCGTATGT | 1093 | ACATACGGCTGTGATCGGTT |
| 140 | 595 | AAACCGATCACAGCCGTATG | 1094 | CATACGGCTGTGATCGGTTT |
| 141 | 596 | CAAACCGATCACAGCCGTAT | 1095 | ATACGGCTGTGATCGGTTTG |
| 142 | 597 | GCAAACCGATCACAGCCGTA | 1096 | TACGGCTGTGATCGGTTTGC |
| 143 | 598 | GGCAAACCGATCACAGCCGT | 1097 | ACGGCTGTGATCGGTTTGCC |
| 144 | 599 | TGGCAAACCGATCACAGCCG | 1098 | CGGCTGTGATCGGTTTGCCA |
| 145 | 600 | TTGGCAAACCGATCACAGCC | 1099 | GGCTGTGATCGGTTTGCCAA |
| 146 | 601 | CTTGGCAAACCGATCACAGC | 1100 | GCTGTGATCGGTTTGCCAAG |
| 147 | 602 | ACTTGGCAAACCGATCACAG | 1101 | CTGTGATCGGTTTGCCAAGT |
| 148 | 603 | CACTTGGCAAACCGATCACA | 1102 | TGTGATCGGTTTGCCAAGTG |
| 149 | 604 | CCACTTGGCAAACCGATCAC | 1103 | GTGATCGGTTTGCCAAGTGG |
| 150 | 605 | TCCACTTGGCAAACCGATCA | 1104 | TGATCGGTTTGCCAAGTGGA |
| 151 | 606 | GTCCACTTGGCAAACCGATC | 1105 | GATCGGTTTGCCAAGTGGAC |
| 152 | 607 | AGTCCACTTGGCAAACCGAT | 1106 | ATCGGTTTGCCAAGTGGACT |
| 153 | 608 | AAGTCCACTTGGCAAACCGA | 1107 | TCGGTTTGCCAAGTGGACTT |
| 154 | 609 | CAAGTCCACTTGGCAAACCG | 1108 | CGGTTTGCCAAGTGGACTTG |
| 155 | 610 | TCAAGTCCACTTGGCAAACC | 1109 | GGTTTGCCAAGTGGACTTGA |
| 156 | 611 | TTCAAGTCCACTTGGCAAAC | 1110 | GTTTGCCAAGTGGACTTGAA |
| 157 | 612 | ATTCAAGTCCACTTGGCAAA | 1111 | TTTGCCAAGTGGACTTGAAT |
| 158 | 613 | GATTCAAGTCCACTTGGCAA | 1112 | TTGCCAAGTGGACTTGAATC |
| 159 | 614 | CGATTCAAGTCCACTTGGCA | 1113 | TGCCAAGTGGACTTGAATCG |
| 160 | 615 | ACGATTCAAGTCCACTTGGC | 1114 | GCCAAGTGGACTTGAATCGT |
| 161 | 616 | CACGATTCAAGTCCACTTGG | 1115 | CCAAGTGGACTTGAATCGTG |
| 162 | 617 | CCACGATTCAAGTCCACTTG | 1116 | CAAGTGGACTTGAATCGTGG |
| 163 | 618 | ACCACGATTCAAGTCCACTT | 1117 | AAGTGGACTTGAATCGTGGT |
| 164 | 619 | GACCACGATTCAAGTCCACT | 1118 | AGTGGACTTGAATCGTGGTC |
| 165 | 620 | TGACCACGATTCAAGTCCAC | 1119 | GTGGACTTGAATCGTGGTCA |
| 166 | 621 | ATGACCACGATTCAAGTCCA | 1120 | TGGACTTGAATCGTGGTCAT |
| 167 | 622 | AATGACCACGATTCAAGTCC | 1121 | GGACTTGAATCGTGGTCATT |
| 168 | 623 | GAATGACCACGATTCAAGTC | 1122 | GACTTGAATCGTGGTCATTC |
| 169 | 624 | AGAATGACCACGATTCAAGT | 1123 | ACTTGAATCGTGGTCATTCT |
| 170 | 625 | AAGAATGACCACGATTCAAG | 1124 | CTTGAATCGTGGTCATTCTT |
| 171 | 626 | AAAGAATGACCACGATTCAA | 1125 | TTGAATCGTGGTCATTCTTT |
| 172 | 627 | CAAAGAATGACCACGATTCA | 1126 | TGAATCGTGGTCATTCTTTG |
| 173 | 628 | GCAAAGAATGACCACGATTC | 1127 | GAATCGTGGTCATTCTTTGC |
| 174 | 629 | AGCAAAGAATGACCACGATT | 1128 | AATCGTGGTCATTCTTTGCT |
| 175 | 630 | CAGCAAAGAATGACCACGAT | 1129 | ATCGTGGTCATTCTTTGCTG |
| 176 | 631 | ACAGCAAAGAATGACCACGA | 1130 | TCGTGGTCATTCTTTGCTGT |
| 177 | 632 | CACAGCAAAGAATGACCACG | 1131 | CGTGGTCATTCTTTGCTGTG |
| 178 | 633 | ACACAGCAAAGAATGACCAC | 1132 | GTGGTCATTCTTTGCTGTGT |
| 179 | 634 | TACACAGCAAAGAATGACCA | 1133 | TGGTCATTCTTTGCTGTGTA |
| 180 | 635 | ATACACAGCAAAGAATGACC | 1134 | GGTCATTCTTTGCTGTGTAT |
| 181 | 636 | CATACACAGCAAAGAATGAC | 1135 | GTCATTCTTTGCTGTGTATG |
| 182 | 637 | TCATACACAGCAAAGAATGA | 1136 | TCATTCTTTGCTGTGTATGA |
| 183 | 638 | ATCATACACAGCAAAGAATG | 1137 | CATTCTTTGCTGTGTATGAT |
| 184 | 639 | CATCATACACAGCAAAGAAT | 1138 | ATTCTTTGCTGTGTATGATG |
| 185 | 640 | CCATCATACACAGCAAAGAA | 1139 | TTCTTTGCTGTGTATGATGG |
| 186 | 641 | CCCATCATACACAGCAAAGA | 1140 | TCTTTGCTGTGTATGATGGG |
| 187 | 642 | GCCCATCATACACAGCAAAG | 1141 | CTTTGCTGTGTATGATGGGC |
| 188 | 643 | TGCCCATCATACACAGCAAA | 1142 | TTTGCTGTGTATGATGGGCA |
| 189 | 644 | ATGCCCATCATACACAGCAA | 1143 | TTGCTGTGTATGATGGGCAT |
| 190 | 645 | CATGCCCATCATACACAGCA | 1144 | TGCTGTGTATGATGGGCATG |
| 191 | 646 | GCATGCCCATCATACACAGC | 1145 | GCTGTGTATGATGGGCATGC |
| 192 | 647 | AGCATGCCCATCATACACAG | 1146 | CTGTGTATGATGGGCATGCT |
| 193 | 648 | CAGCATGCCCATCATACACA | 1147 | TGTGTATGATGGGCATGCTG |
| 194 | 649 | CCAGCATGCCCATCATACAC | 1148 | GTGTATGATGGGCATGCTGG |
| 195 | 650 | ACCAGCATGCCCATCATACA | 1149 | TGTATGATGGGCATGCTGGT |
| 196 | 651 | AACCAGCATGCCCATCATAC | 1150 | GTATGATGGGCATGCTGGTT |
| 197 | 652 | GAACCAGCATGCCCATCATA | 1151 | TATGATGGGCATGCTGGTTC |
| 198 | 653 | AGAACCAGCATGCCCATCAT | 1152 | ATGATGGGCATGCTGGTTCT |
| 199 | 654 | GAGAACCAGCATGCCCATCA | 1153 | TGATGGGCATGCTGGTTCTC |
| 200 | 655 | TGAGAACCAGCATGCCCATC | 1154 | GATGGGCATGCTGGTTCTCA |
| 201 | 656 | CTGAGAACCAGCATGCCCAT | 1155 | ATGGGCATGCTGGTTCTCAG |
| 202 | 657 | CCTGAGAACCAGCATGCCCA | 1156 | TGGGCATGCTGGTTCTCAGG |
| 203 | 658 | ACCTGAGAACCAGCATGCCC | 1157 | GGGCATGCTGGTTCTCAGGT |
| 204 | 659 | AACCTGAGAACCAGCATGCC | 1158 | GGCATGCTGGTTCTCAGGTT |
| 205 | 660 | CAACCTGAGAACCAGCATGC | 1159 | GCATGCTGGTTCTCAGGTTG |
| 206 | 661 | GCAACCTGAGAACCAGCATG | 1160 | CATGCTGGTTCTCAGGTTGC |
| 207 | 662 | GGCAACCTGAGAACCAGCAT | 1161 | ATGCTGGTTCTCAGGTTGCC |
| 208 | 663 | TGGCAACCTGAGAACCAGCA | 1162 | TGCTGGTTCTCAGGTTGCCA |
| 209 | 664 | TTGGCAACCTGAGAACCAGC | 1163 | GCTGGTTCTCAGGTTGCCAA |
| 210 | 665 | TTTGGCAACCTGAGAACCAG | 1164 | CTGGTTCTCAGGTTGCCAAA |
| 211 | 666 | ATTTGGCAACCTGAGAACCA | 1165 | TGGTTCTCAGGTTGCCAAAT |
| 212 | 667 | TATTTGGCAACCTGAGAACC | 1166 | GGTTCTCAGGTTGCCAAATA |
| 213 | 668 | GTATTTGGCAACCTGAGAAC | 1167 | GTTCTCAGGTTGCCAAATAC |
| 214 | 669 | AGTATTTGGCAACCTGAGAA | 1168 | TTCTCAGGTTGCCAAATACT |
| 215 | 670 | CAGTATTTGGCAACCTGAGA | 1169 | TCTCAGGTTGCCAAATACTG |
| 216 | 671 | GCAGTATTTGGCAACCTGAG | 1170 | CTCAGGTTGCCAAATACTGC |
| 217 | 672 | AGCAGTATTTGGCAACCTGA | 1171 | TCAGGTTGCCAAATACTGCT |
| 218 | 673 | CAGCAGTATTTGGCAACCTG | 1172 | CAGGTTGCCAAATACTGCTG |
| 219 | 674 | ACAGCAGTATTTGGCAACCT | 1173 | AGGTTGCCAAATACTGCTGT |
| 220 | 675 | CACAGCAGTATTTGGCAACC | 1174 | GGTTGCCAAATACTGCTGTG |
| 221 | 676 | TCACAGCAGTATTTGGCAAC | 1175 | GTTGCCAAATACTGCTGTGA |
| 222 | 677 | CTCACAGCAGTATTTGGCAA | 1176 | TTGCCAAATACTGCTGTGAG |
| 223 | 678 | GCTCACAGCAGTATTTGGCA | 1177 | TGCCAAATACTGCTGTGAGC |
| 224 | 679 | TGCTCACAGCAGTATTTGGC | 1178 | GCCAAATACTGCTGTGAGCA |
| 225 | 680 | ATGCTCACAGCAGTATTTGG | 1179 | CCAAATACTGCTGTGAGCAT |
| 226 | 681 | AATGCTCACAGCAGTATTTG | 1180 | CAAATACTGCTGTGAGCATT |
| 227 | 682 | AAATGCTCACAGCAGTATTT | 1181 | AAATACTGCTGTGAGCATTT |
| 228 | 683 | CAAATGCTCACAGCAGTATT | 1182 | AATACTGCTGTGAGCATTTG |
| 229 | 684 | ACAAATGCTCACAGCAGTAT | 1183 | ATACTGCTGTGAGCATTTGT |
| 230 | 685 | AACAAATGCTCACAGCAGTA | 1184 | TACTGCTGTGAGCATTTGTT |
| 231 | 686 | TAACAAATGCTCACAGCAGT | 1185 | ACTGCTGTGAGCATTTGTTA |
| 232 | 687 | CTAACAAATGCTCACAGCAG | 1186 | CTGCTGTGAGCATTTGTTAG |
| 233 | 688 | TCTAACAAATGCTCACAGCA | 1187 | TGCTGTGAGCATTTGTTAGA |
| 234 | 689 | ATCTAACAAATGCTCACAGC | 1188 | GCTGTGAGCATTTGTTAGAT |
| 235 | 690 | GATCTAACAAATGCTCACAG | 1189 | CTGTGAGCATTTGTTAGATC |
| 236 | 691 | TGATCTAACAAATGCTCACA | 1190 | TGTGAGCATTTGTTAGATCA |
| 237 | 692 | GTGATCTAACAAATGCTCAC | 1191 | GTGAGCATTTGTTAGATCAC |
| 238 | 693 | TGTGATCTAACAAATGCTCA | 1192 | TGAGCATTTGTTAGATCACA |
| 239 | 694 | ATGTGATCTAACAAATGCTC | 1193 | GAGCATTTGTTAGATCACAT |
| 240 | 695 | GATGTGATCTAACAAATGCT | 1194 | AGCATTTGTTAGATCACATC |
| 241 | 696 | TGATGTGATCTAACAAATGC | 1195 | GCATTTGTTAGATCACATCA |
| 242 | 697 | GTGATGTGATCTAACAAATG | 1196 | CATTTGTTAGATCACATCAC |
| 243 | 698 | GGTGATGTGATCTAACAAAT | 1197 | ATTTGTTAGATCACATCACC |
| 244 | 699 | TGGTGATGTGATCTAACAAA | 1198 | TTTGTTAGATCACATCACCA |
| 245 | 700 | TTGGTGATGTGATCTAACAA | 1199 | TTGTTAGATCACATCACCAA |
| 246 | 701 | ATTGGTGATGTGATCTAACA | 1200 | TGTTAGATCACATCACCAAT |
| 247 | 702 | TATTGGTGATGTGATCTAAC | 1201 | GTTAGATCACATCACCAATA |
| 248 | 703 | TTATTGGTGATGTGATCTAA | 1202 | TTAGATCACATCACCAATAA |
| 249 | 704 | GTTATTGGTGATGTGATCTA | 1203 | TAGATCACATCACCAATAAC |
| 250 | 705 | GGTTATTGGTGATGTGATCT | 1204 | AGATCACATCACCAATAACC |
| 251 | 706 | TGGTTATTGGTGATGTGATC | 1205 | GATCACATCACCAATAACCA |
| 252 | 707 | CTGGTTATTGGTGATGTGAT | 1206 | ATCACATCACCAATAACCAG |
| 253 | 708 | CCTGGTTATTGGTGATGTGA | 1207 | TCACATCACCAATAACCAGG |
| 254 | 709 | TCCTGGTTATTGGTGATGTG | 1208 | CACATCACCAATAACCAGGA |
| 255 | 710 | ATCCTGGTTATTGGTGATGT | 1209 | ACATCACCAATAACCAGGAT |
| 256 | 711 | AATCCTGGTTATTGGTGATG | 1210 | CATCACCAATAACCAGGATT |
| 257 | 712 | AAATCCTGGTTATTGGTGAT | 1211 | ATCACCAATAACCAGGATTT |
| 258 | 713 | AAAATCCTGGTTATTGGTGA | 1212 | TCACCAATAACCAGGATTTT |
| 259 | 714 | TAAAATCCTGGTTATTGGTG | 1213 | CACCAATAACCAGGATTTTA |
| 260 | 715 | TTAAAATCCTGGTTATTGGT | 1214 | ACCAATAACCAGGATTTTAA |
| 261 | 716 | TTTAAAATCCTGGTTATTGG | 1215 | CCAATAACCAGGATTTTAAA |
| 262 | 717 | CTTTAAAATCCTGGTTATTG | 1216 | CAATAACCAGGATTTTAAAG |
| 263 | 718 | CCTTTAAAATCCTGGTTATT | 1217 | AATAACCAGGATTTTAAAGG |
| 264 | 719 | CCCTTTAAAATCCTGGTTAT | 1218 | ATAACCAGGATTTTAAAGGG |
| 265 | 720 | ACCCTTTAAAATCCTGGTTA | 1219 | TAACCAGGATTTTAAAGGGT |
| 266 | 721 | GACCCTTTAAAATCCTGGTT | 1220 | AACCAGGATTTTAAAGGGTC |
| 267 | 722 | AGACCCTTTAAAATCCTGGT | 1221 | ACCAGGATTTTAAAGGGTCT |
| 268 | 723 | CAGACCCTTTAAAATCCTGG | 1222 | CCAGGATTTTAAAGGGTCTG |
| 269 | 724 | GCAGACCCTTTAAAATCCTG | 1223 | CAGGATTTTAAAGGGTCTGC |
| 270 | 725 | TGCAGACCCTTTAAAATCCT | 1224 | AGGATTTTAAAGGGTCTGCA |
| 271 | 726 | CTGCAGACCCTTTAAAATCC | 1225 | GGATTTTAAAGGGTCTGCAG |
| 272 | 727 | CCTGCAGACCCTTTAAAATC | 1226 | GATTTTAAAGGGTCTGCAGG |
| 273 | 728 | TCCTGCAGACCCTTTAAAAT | 1227 | ATTTTAAAGGGTCTGCAGGA |
| 274 | 729 | CTCCTGCAGACCCTTTAAAA | 1228 | TTTTAAAGGGTCTGCAGGAG |
| 275 | 730 | GCTCCTGCAGACCCTTTAAA | 1229 | TTTAAAGGGTCTGCAGGAGC |
| 276 | 731 | TGCTCCTGCAGACCCTTTAA | 1230 | TTAAAGGGTCTGCAGGAGCA |
| 277 | 732 | GTGCTCCTGCAGACCCTTTA | 1231 | TAAAGGGTCTGCAGGAGCAC |
| 278 | 733 | GGTGCTCCTGCAGACCCTTT | 1232 | AAAGGGTCTGCAGGAGCACC |
| 279 | 734 | AGGTGCTCCTGCAGACCCTT | 1233 | AAGGGTCTGCAGGAGCACCT |
| 280 | 735 | AAGGTGCTCCTGCAGACCCT | 1234 | AGGGTCTGCAGGAGCACCTT |
| 281 | 736 | GAAGGTGCTCCTGCAGACCC | 1235 | GGGTCTGCAGGAGCACCTTC |
| 282 | 737 | AGAAGGTGCTCCTGCAGACC | 1236 | GGTCTGCAGGAGCACCTTCT |
| 283 | 738 | CAGAAGGTGCTCCTGCAGAC | 1237 | GTCTGCAGGAGCACCTTCTG |
| 284 | 739 | ACAGAAGGTGCTCCTGCAGA | 1238 | TCTGCAGGAGCACCTTCTGT |
| 285 | 740 | CACAGAAGGTGCTCCTGCAG | 1239 | CTGCAGGAGCACCTTCTGTG |
| 286 | 741 | CCACAGAAGGTGCTCCTGCA | 1240 | TGCAGGAGCACCTTCTGTGG |
| 287 | 742 | TCCACAGAAGGTGCTCCTGC | 1241 | GCAGGAGCACCTTCTGTGGA |
| 288 | 743 | TTCCACAGAAGGTGCTCCTG | 1242 | CAGGAGCACCTTCTGTGGAA |
| 289 | 744 | TTTCCACAGAAGGTGCTCCT | 1243 | AGGAGCACCTTCTGTGGAAA |
| 290 | 745 | TTTTCCACAGAAGGTGCTCC | 1244 | GGAGCACCTTCTGTGGAAAA |
| 291 | 746 | ATTTTCCACAGAAGGTGCTC | 1245 | GAGCACCTTCTGTGGAAAAT |
| 292 | 747 | CATTTTCCACAGAAGGTGCT | 1246 | AGCACCTTCTGTGGAAAATG |
| 293 | 748 | ACATTTTCCACAGAAGGTGC | 1247 | GCACCTTCTGTGGAAAATGT |
| 294 | 749 | TACATTTTCCACAGAAGGTG | 1248 | CACCTTCTGTGGAAAATGTA |
| 295 | 750 | TTACATTTTCCACAGAAGGT | 1249 | ACCTTCTGTGGAAAATGTAA |
| 296 | 751 | TTTACATTTTCCACAGAAGG | 1250 | CCTTCTGTGGAAAATGTAAA |
| 297 | 752 | CTTTACATTTTCCACAGAAG | 1251 | CTTCTGTGGAAAATGTAAAG |
| 298 | 753 | TCTTTACATTTTCCACAGAA | 1252 | TTCTGTGGAAAATGTAAAGA |
| 299 | 754 | TTCTTTACATTTTCCACAGA | 1253 | TCTGTGGAAAATGTAAAGAA |
| 300 | 755 | ATTCTTTACATTTTCCACAG | 1254 | CTGTGGAAAATGTAAAGAAT |
| 301 | 756 | CATTCTTTACATTTTCCACA | 1255 | TGTGGAAAATGTAAAGAATG |
| 302 | 757 | CCATTCTTTACATTTTCCAC | 1256 | GTGGAAAATGTAAAGAATGG |
| 303 | 758 | TCCATTCTTTACATTTTCCA | 1257 | TGGAAAATGTAAAGAATGGA |
| 304 | 759 | TTCCATTCTTTACATTTTCC | 1258 | GGAAAATGTAAAGAATGGAA |
| 305 | 760 | ATTCCATTCTTTACATTTTC | 1259 | GAAAATGTAAAGAATGGAAT |
| 306 | 761 | GATTCCATTCTTTACATTTT | 1260 | AAAATGTAAAGAATGGAATC |
| 307 | 762 | TGATTCCATTCTTTACATTT | 1261 | AAATGTAAAGAATGGAATCA |
| 308 | 763 | CTGATTCCATTCTTTACATT | 1262 | AATGTAAAGAATGGAATCAG |
| 309 | 764 | TCTGATTCCATTCTTTACAT | 1263 | ATGTAAAGAATGGAATCAGA |
| 310 | 765 | TTCTGATTCCATTCTTTACA | 1264 | TGTAAAGAATGGAATCAGAA |
| 311 | 766 | GTTCTGATTCCATTCTTTAC | 1265 | GTAAAGAATGGAATCAGAAC |
| 312 | 767 | TGTTCTGATTCCATTCTTTA | 1266 | TAAAGAATGGAATCAGAACA |
| 313 | 768 | CTGTTCTGATTCCATTCTTT | 1267 | AAAGAATGGAATCAGAACAG |
| 314 | 769 | CCTGTTCTGATTCCATTCTT | 1268 | AAGAATGGAATCAGAACAGG |
| 315 | 770 | ACCTGTTCTGATTCCATTCT | 1269 | AGAATGGAATCAGAACAGGT |
| 316 | 771 | AACCTGTTCTGATTCCATTC | 1270 | GAATGGAATCAGAACAGGTT |
| 317 | 772 | AAACCTGTTCTGATTCCATT | 1271 | AATGGAATCAGAACAGGTTT |
| 318 | 773 | AAAACCTGTTCTGATTCCAT | 1272 | ATGGAATCAGAACAGGTTTT |
| 319 | 774 | GAAAACCTGTTCTGATTCCA | 1273 | TGGAATCAGAACAGGTTTTC |
| 320 | 775 | AGAAAACCTGTTCTGATTCC | 1274 | GGAATCAGAACAGGTTTTCT |
| 321 | 776 | CAGAAAACCTGTTCTGATTC | 1275 | GAATCAGAACAGGTTTTCTG |
| 322 | 777 | CCAGAAAACCTGTTCTGATT | 1276 | AATCAGAACAGGTTTTCTGG |
| 323 | 778 | TCCAGAAAACCTGTTCTGAT | 1277 | ATCAGAACAGGTTTTCTGGA |
| 324 | 779 | CTCCAGAAAACCTGTTCTGA | 1278 | TCAGAACAGGTTTTCTGGAG |
| 325 | 780 | TCTCCAGAAAACCTGTTCTG | 1279 | CAGAACAGGTTTTCTGGAGA |
| 326 | 781 | ATCTCCAGAAAACCTGTTCT | 1280 | AGAACAGGTTTTCTGGAGAT |
| 327 | 782 | AATCTCCAGAAAACCTGTTC | 1281 | GAACAGGTTTTCTGGAGATT |
| 328 | 783 | CAATCTCCAGAAAACCTGTT | 1282 | AACAGGTTTTCTGGAGATTG |
| 329 | 784 | TCAATCTCCAGAAAACCTGT | 1283 | ACAGGTTTTCTGGAGATTGA |
| 330 | 785 | ATCAATCTCCAGAAAACCTG | 1284 | CAGGTTTTCTGGAGATTGAT |
| 331 | 786 | CATCAATCTCCAGAAAACCT | 1285 | AGGTTTTCTGGAGATTGATG |
| 332 | 787 | TCATCAATCTCCAGAAAACC | 1286 | GGTTTTCTGGAGATTGATGA |
| 333 | 788 | TTCATCAATCTCCAGAAAAC | 1287 | GTTTTCTGGAGATTGATGAA |
| 334 | 789 | GTTCATCAATCTCCAGAAAA | 1288 | TTTTCTGGAGATTGATGAAC |
| 335 | 790 | TGTTCATCAATCTCCAGAAA | 1289 | TTTCTGGAGATTGATGAACA |
| 336 | 791 | GTGTTCATCAATCTCCAGAA | 1290 | TTCTGGAGATTGATGAACAC |
| 337 | 792 | TGTGTTCATCAATCTCCAGA | 1291 | TCTGGAGATTGATGAACACA |
| 338 | 793 | ATGTGTTCATCAATCTCCAG | 1292 | CTGGAGATTGATGAACACAT |
| 339 | 794 | CATGTGTTCATCAATCTCCA | 1293 | TGGAGATTGATGAACACATG |
| 340 | 795 | TCATGTGTTCATCAATCTCC | 1294 | GGAGATTGATGAACACATGA |
| 341 | 796 | CTCATGTGTTCATCAATCTC | 1295 | GAGATTGATGAACACATGAG |
| 342 | 797 | TCTCATGTGTTCATCAATCT | 1296 | AGATTGATGAACACATGAGA |
| 343 | 798 | CTCTCATGTGTTCATCAATC | 1297 | GATTGATGAACACATGAGAG |
| 344 | 799 | ACTCTCATGTGTTCATCAAT | 1298 | ATTGATGAACACATGAGAGT |
| 345 | 800 | AACTCTCATGTGTTCATCAA | 1299 | TTGATGAACACATGAGAGTT |
| 346 | 801 | TAACTCTCATGTGTTCATCA | 1300 | TGATGAACACATGAGAGTTA |
| 347 | 802 | ATAACTCTCATGTGTTCATC | 1301 | GATGAACACATGAGAGTTAT |
| 348 | 803 | CATAACTCTCATGTGTTCAT | 1302 | ATGAACACATGAGAGTTATG |
| 349 | 804 | ACATAACTCTCATGTGTTCA | 1303 | TGAACACATGAGAGTTATGT |
| 350 | 805 | GACATAACTCTCATGTGTTC | 1304 | GAACACATGAGAGTTATGTC |
| 351 | 806 | TGACATAACTCTCATGTGTT | 1305 | AACACATGAGAGTTATGTCA |
| 352 | 807 | CTGACATAACTCTCATGTGT | 1306 | ACACATGAGAGTTATGTCAG |
| 353 | 808 | TCTGACATAACTCTCATGTG | 1307 | CACATGAGAGTTATGTCAGA |
| 354 | 809 | CTCTGACATAACTCTCATGT | 1308 | ACATGAGAGTTATGTCAGAG |
| 355 | 810 | TCTCTGACATAACTCTCATG | 1309 | CATGAGAGTTATGTCAGAGA |
| 356 | 811 | TTCTCTGACATAACTCTCAT | 1310 | ATGAGAGTTATGTCAGAGAA |
| 357 | 812 | CTTCTCTGACATAACTCTCA | 1311 | TGAGAGTTATGTCAGAGAAG |
| 358 | 813 | TCTTCTCTGACATAACTCTC | 1312 | GAGAGTTATGTCAGAGAAGA |
| 359 | 814 | TTCTTCTCTGACATAACTCT | 1313 | AGAGTTATGTCAGAGAAGAA |
| 360 | 815 | TTTCTTCTCTGACATAACTC | 1314 | GAGTTATGTCAGAGAAGAAA |
| 361 | 816 | GTTTCTTCTCTGACATAACT | 1315 | AGTTATGTCAGAGAAGAAAC |
| 362 | 817 | TGTTTCTTCTCTGACATAAC | 1316 | GTTATGTCAGAGAAGAAACA |
| 363 | 818 | ATGTTTCTTCTCTGACATAA | 1317 | TTATGTCAGAGAAGAAACAT |
| 364 | 819 | CATGTTTCTTCTCTGACATA | 1318 | TATGTCAGAGAAGAAACATG |
| 365 | 820 | CCATGTTTCTTCTCTGACAT | 1319 | ATGTCAGAGAAGAAACATGG |
| 366 | 821 | ACCATGTTTCTTCTCTGACA | 1320 | TGTCAGAGAAGAAACATGGT |
| 367 | 822 | CACCATGTTTCTTCTCTGAC | 1321 | GTCAGAGAAGAAACATGGTG |
| 368 | 823 | GCACCATGTTTCTTCTCTGA | 1322 | TCAGAGAAGAAACATGGTGC |
| 369 | 824 | TGCACCATGTTTCTTCTCTG | 1323 | CAGAGAAGAAACATGGTGCA |
| 370 | 825 | CTGCACCATGTTTCTTCTCT | 1324 | AGAGAAGAAACATGGTGCAG |
| 371 | 826 | TCTGCACCATGTTTCTTCTC | 1325 | GAGAAGAAACATGGTGCAGA |
| 372 | 827 | ATCTGCACCATGTTTCTTCT | 1326 | AGAAGAAACATGGTGCAGAT |
| 373 | 828 | TATCTGCACCATGTTTCTTC | 1327 | GAAGAAACATGGTGCAGATA |
| 374 | 829 | CTATCTGCACCATGTTTCTT | 1328 | AAGAAACATGGTGCAGATAG |
| 375 | 830 | TCTATCTGCACCATGTTTCT | 1329 | AGAAACATGGTGCAGATAGA |
| 376 | 831 | TTCTATCTGCACCATGTTTC | 1330 | GAAACATGGTGCAGATAGAA |
| 377 | 832 | CTTCTATCTGCACCATGTTT | 1331 | AAACATGGTGCAGATAGAAG |
| 378 | 833 | ACTTCTATCTGCACCATGTT | 1332 | AACATGGTGCAGATAGAAGT |
| 379 | 834 | CACTTCTATCTGCACCATGT | 1333 | ACATGGTGCAGATAGAAGTG |
| 380 | 835 | CCACTTCTATCTGCACCATG | 1334 | CATGGTGCAGATAGAAGTGG |
| 381 | 836 | CCCACTTCTATCTGCACCAT | 1335 | ATGGTGCAGATAGAAGTGGG |
| 382 | 837 | ACCCACTTCTATCTGCACCA | 1336 | TGGTGCAGATAGAAGTGGGT |
| 383 | 838 | GACCCACTTCTATCTGCACC | 1337 | GGTGCAGATAGAAGTGGGTC |
| 384 | 839 | TGACCCACTTCTATCTGCAC | 1338 | GTGCAGATAGAAGTGGGTCA |
| 385 | 840 | TTGACCCACTTCTATCTGCA | 1339 | TGCAGATAGAAGTGGGTCAA |
| 386 | 841 | GTTGACCCACTTCTATCTGC | 1340 | GCAGATAGAAGTGGGTCAAC |
| 387 | 842 | TGTTGACCCACTTCTATCTG | 1341 | CAGATAGAAGTGGGTCAACA |
| 388 | 843 | CTGTTGACCCACTTCTATCT | 1342 | AGATAGAAGTGGGTCAACAG |
| 389 | 844 | GCTGTTGACCCACTTCTATC | 1343 | GATAGAAGTGGGTCAACAGC |
| 390 | 845 | AGCTGTTGACCCACTTCTAT | 1344 | ATAGAAGTGGGTCAACAGCT |
| 391 | 846 | CAGCTGTTGACCCACTTCTA | 1345 | TAGAAGTGGGTCAACAGCTG |
| 392 | 847 | ACAGCTGTTGACCCACTTCT | 1346 | AGAAGTGGGTCAACAGCTGT |
| 393 | 848 | TACAGCTGTTGACCCACTTC | 1347 | GAAGTGGGTCAACAGCTGTA |
| 394 | 849 | CTACAGCTGTTGACCCACTT | 1348 | AAGTGGGTCAACAGCTGTAG |
| 395 | 850 | CCTACAGCTGTTGACCCACT | 1349 | AGTGGGTCAACAGCTGTAGG |
| 396 | 851 | ACCTACAGCTGTTGACCCAC | 1350 | GTGGGTCAACAGCTGTAGGT |
| 397 | 852 | CACCTACAGCTGTTGACCCA | 1351 | TGGGTCAACAGCTGTAGGTG |
| 398 | 853 | ACACCTACAGCTGTTGACCC | 1352 | GGGTCAACAGCTGTAGGTGT |
| 399 | 854 | GACACCTACAGCTGTTGACC | 1353 | GGTCAACAGCTGTAGGTGTC |
| 400 | 855 | AGACACCTACAGCTGTTGAC | 1354 | GTCAACAGCTGTAGGTGTCT |
| 401 | 856 | AAGACACCTACAGCTGTTGA | 1355 | TCAACAGCTGTAGGTGTCTT |
| 402 | 857 | TAAGACACCTACAGCTGTTG | 1356 | CAACAGCTGTAGGTGTCTTA |
| 403 | 858 | TTAAGACACCTACAGCTGTT | 1357 | AACAGCTGTAGGTGTCTTAA |
| 404 | 859 | ATTAAGACACCTACAGCTGT | 1358 | ACAGCTGTAGGTGTCTTAAT |
| 405 | 860 | AATTAAGACACCTACAGCTG | 1359 | CAGCTGTAGGTGTCTTAATT |
| 406 | 861 | AAATTAAGACACCTACAGCT | 1360 | AGCTGTAGGTGTCTTAATTT |
| 407 | 862 | GAAATTAAGACACCTACAGC | 1361 | GCTGTAGGTGTCTTAATTTC |
| 408 | 863 | AGAAATTAAGACACCTACAG | 1362 | CTGTAGGTGTCTTAATTTCT |
| 409 | 864 | GAGAAATTAAGACACCTACA | 1363 | TGTAGGTGTCTTAATTTCTC |
| 410 | 865 | GGAGAAATTAAGACACCTAC | 1364 | GTAGGTGTCTTAATTTCTCC |
| 411 | 866 | GGGAGAAATTAAGACACCTA | 1365 | TAGGTGTCTTAATTTCTCCC |
| 412 | 867 | GGGGAGAAATTAAGACACCT | 1366 | AGGTGTCTTAATTTCTCCCC |
| 413 | 868 | TGGGGAGAAATTAAGACACC | 1367 | GGTGTCTTAATTTCTCCCCA |
| 414 | 869 | TTGGGGAGAAATTAAGACAC | 1368 | GTGTCTTAATTTCTCCCCAA |
| 415 | 870 | GTTGGGGAGAAATTAAGACA | 1369 | TGTCTTAATTTCTCCCCAAC |
| 416 | 871 | TGTTGGGGAGAAATTAAGAC | 1370 | GTCTTAATTTCTCCCCAACA |
| 417 | 872 | ATGTTGGGGAGAAATTAAGA | 1371 | TCTTAATTTCTCCCCAACAT |
| 418 | 873 | TATGTTGGGGAGAAATTAAG | 1372 | CTTAATTTCTCCCCAACATA |
| 419 | 874 | GTATGTTGGGGAGAAATTAA | 1373 | TTAATTTCTCCCCAACATAC |
| 420 | 875 | AGTATGTTGGGGAGAAATTA | 1374 | TAATTTCTCCCCAACATACT |
| 421 | 876 | AAGTATGTTGGGGAGAAATT | 1375 | AATTTCTCCCCAACATACTT |
| 422 | 877 | TAAGTATGTTGGGGAGAAAT | 1376 | ATTTCTCCCCAACATACTTA |
| 423 | 878 | ATAAGTATGTTGGGGAGAAA | 1377 | TTTCTCCCCAACATACTTAT |
| 424 | 879 | AATAAGTATGTTGGGGAGAA | 1378 | TTCTCCCCAACATACTTATT |
| 425 | 880 | AAATAAGTATGTTGGGGAGA | 1379 | TCTCCCCAACATACTTATTT |
| 426 | 881 | GAAATAAGTATGTTGGGGAG | 1380 | CTCCCCAACATACTTATTTC |
| 427 | 882 | TGAAATAAGTATGTTGGGGA | 1381 | TCCCCAACATACTTATTTCA |
| 428 | 883 | ATGAAATAAGTATGTTGGGG | 1382 | CCCCAACATACTTATTTCAT |
| 429 | 884 | AATGAAATAAGTATGTTGGG | 1383 | CCCAACATACTTATTTCATT |
| 430 | 885 | TAATGAAATAAGTATGTTGG | 1384 | CCAACATACTTATTTCATTA |
| 431 | 886 | TTAATGAAATAAGTATGTTG | 1385 | CAACATACTTATTTCATTAA |
| 432 | 887 | GTTAATGAAATAAGTATGTT | 1386 | AACATACTTATTTCATTAAC |
| 433 | 888 | AGTTAATGAAATAAGTATGT | 1387 | ACATACTTATTTCATTAACT |
| 434 | 889 | CAGTTAATGAAATAAGTATG | 1388 | CATACTTATTTCATTAACTG |
| 435 | 890 | ACAGTTAATGAAATAAGTAT | 1389 | ATACTTATTTCATTAACTGT |
| 436 | 891 | CACAGTTAATGAAATAAGTA | 1390 | TACTTATTTCATTAACTGTG |
| 437 | 892 | CCACAGTTAATGAAATAAGT | 1391 | ACTTATTTCATTAACTGTGG |
| 438 | 893 | TCCACAGTTAATGAAATAAG | 1392 | CTTATTTCATTAACTGTGGA |
| 439 | 894 | CTCCACAGTTAATGAAATAA | 1393 | TTATTTCATTAACTGTGGAG |
| 440 | 895 | TCTCCACAGTTAATGAAATA | 1394 | TATTTCATTAACTGTGGAGA |
| 441 | 896 | GTCTCCACAGTTAATGAAAT | 1395 | ATTTCATTAACTGTGGAGAC |
| 442 | 897 | AGTCTCCACAGTTAATGAAA | 1396 | TTTCATTAACTGTGGAGACT |
| 443 | 898 | GAGTCTCCACAGTTAATGAA | 1397 | TTCATTAACTGTGGAGACTC |
| 444 | 899 | TGAGTCTCCACAGTTAATGA | 1398 | TCATTAACTGTGGAGACTCA |
| 445 | 900 | TTGAGTCTCCACAGTTAATG | 1399 | CATTAACTGTGGAGACTCAA |
| 446 | 901 | CTTGAGTCTCCACAGTTAAT | 1400 | ATTAACTGTGGAGACTCAAG |
| 447 | 902 | TCTTGAGTCTCCACAGTTAA | 1401 | TTAACTGTGGAGACTCAAGA |
| 448 | 903 | CTCTTGAGTCTCCACAGTTA | 1402 | TAACTGTGGAGACTCAAGAG |
| 449 | 904 | CCTCTTGAGTCTCCACAGTT | 1403 | AACTGTGGAGACTCAAGAGG |
| 450 | 905 | ACCTCTTGAGTCTCCACAGT | 1404 | ACTGTGGAGACTCAAGAGGT |
| 451 | 906 | AACCTCTTGAGTCTCCACAG | 1405 | CTGTGGAGACTCAAGAGGTT |
| 452 | 907 | AAACCTCTTGAGTCTCCACA | 1406 | TGTGGAGACTCAAGAGGTTT |
| 453 | 908 | TAAACCTCTTGAGTCTCCAC | 1407 | GTGGAGACTCAAGAGGTTTA |
| 454 | 909 | GTAAACCTCTTGAGTCTCCA | 1408 | TGGAGACTCAAGAGGTTTAC |
| 455 | 910 | AGTAAACCTCTTGAGTCTCC | 1409 | GGAGACTCAAGAGGTTTACT |
| 456 | 911 | AAGTAAACCTCTTGAGTCTC | 1410 | GAGACTCAAGAGGTTTACTT |
| 457 | 912 | AAAGTAAACCTCTTGAGTCT | 1411 | AGACTCAAGAGGTTTACTTT |
| 458 | 913 | CAAAGTAAACCTCTTGAGTC | 1412 | GACTCAAGAGGTTTACTTTG |
| 459 | 914 | ACAAAGTAAACCTCTTGAGT | 1413 | ACTCAAGAGGTTTACTTTGT |
| 460 | 915 | TACAAAGTAAACCTCTTGAG | 1414 | CTCAAGAGGTTTACTTTGTA |
| 461 | 916 | CTACAAAGTAAACCTCTTGA | 1415 | TCAAGAGGTTTACTTTGTAG |
| 462 | 917 | CCTACAAAGTAAACCTCTTG | 1416 | CAAGAGGTTTACTTTGTAGG |
| 463 | 918 | TCCTACAAAGTAAACCTCTT | 1417 | AAGAGGTTTACTTTGTAGGA |
| 464 | 919 | TTCCTACAAAGTAAACCTCT | 1418 | AGAGGTTTACTTTGTAGGAA |
| 465 | 920 | GTTCCTACAAAGTAAACCTC | 1419 | GAGGTTTACTTTGTAGGAAC |
| 466 | 921 | TGTTCCTACAAAGTAAACCT | 1420 | AGGTTTACTTTGTAGGAACA |
| 467 | 922 | CTGTTCCTACAAAGTAAACC | 1421 | GGTTTACTTTGTAGGAACAG |
| 468 | 923 | CCTGTTCCTACAAAGTAAAC | 1422 | GTTTACTTTGTAGGAACAGG |
| 469 | 924 | TCCTGTTCCTACAAAGTAAA | 1423 | TTTACTTTGTAGGAACAGGA |
| 470 | 925 | TTCCTGTTCCTACAAAGTAA | 1424 | TTACTTTGTAGGAACAGGAA |
| 471 | 926 | TTTCCTGTTCCTACAAAGTA | 1425 | TACTTTGTAGGAACAGGAAA |
| 472 | 927 | CTTTCCTGTTCCTACAAAGT | 1426 | ACTTTGTAGGAACAGGAAAG |
| 473 | 928 | ACTTTCCTGTTCCTACAAAG | 1427 | CTTTGTAGGAACAGGAAAGT |
| 474 | 929 | AACTTTCCTGTTCCTACAAA | 1428 | TTTGTAGGAACAGGAAAGTT |
| 475 | 930 | GAACTTTCCTGTTCCTACAA | 1429 | TTGTAGGAACAGGAAAGTTC |
| 476 | 931 | TGAACTTTCCTGTTCCTACA | 1430 | TGTAGGAACAGGAAAGTTCA |
| 477 | 932 | ATGAACTTTCCTGTTCCTAC | 1431 | GTAGGAACAGGAAAGTTCAT |
| 478 | 933 | AATGAACTTTCCTGTTCCTA | 1432 | TAGGAACAGGAAAGTTCATT |
| 479 | 934 | AAATGAACTTTCCTGTTCCT | 1433 | AGGAACAGGAAAGTTCATTT |
| 480 | 935 | GAAATGAACTTTCCTGTTCC | 1434 | GGAACAGGAAAGTTCATTTC |
| 481 | 936 | AGAAATGAACTTTCCTGTTC | 1435 | GAACAGGAAAGTTCATTTCT |
| 482 | 937 | AAGAAATGAACTTTCCTGTT | 1436 | AACAGGAAAGTTCATTTCTT |
| 483 | 938 | GAAGAAATGAACTTTCCTGT | 1437 | ACAGGAAAGTTCATTTCTTC |
| 484 | 939 | TGAAGAAATGAACTTTCCTG | 1438 | CAGGAAAGTTCATTTCTTCA |
| 485 | 940 | GTGAAGAAATGAACTTTCCT | 1439 | AGGAAAGTTCATTTCTTCAC |
| 486 | 941 | TGTGAAGAAATGAACTTTCC | 1440 | GGAAAGTTCATTTCTTCACA |
| 487 | 942 | GTGTGAAGAAATGAACTTTC | 1441 | GAAAGTTCATTTCTTCACAC |
| 488 | 943 | TGTGTGAAGAAATGAACTTT | 1442 | AAAGTTCATTTCTTCACACA |
| 489 | 944 | TTGTGTGAAGAAATGAACTT | 1443 | AAGTTCATTTCTTCACACAA |
| 490 | 945 | CTTGTGTGAAGAAATGAACT | 1444 | AGTTCATTTCTTCACACAAG |
| 491 | 946 | TCTTGTGTGAAGAAATGAAC | 1445 | GTTCATTTCTTCACACAAGA |
| 492 | 947 | ATCTTGTGTGAAGAAATGAA | 1446 | TTCATTTCTTCACACAAGAT |
| 493 | 948 | GATCTTGTGTGAAGAAATGA | 1447 | TCATTTCTTCACACAAGATC |
| 494 | 949 | TGATCTTGTGTGAAGAAATG | 1448 | CATTTCTTCACACAAGATCA |
| 495 | 950 | GTGATCTTGTGTGAAGAAAT | 1449 | ATTTCTTCACACAAGATCAC |
| 496 | 951 | TGTGATCTTGTGTGAAGAAA | 1450 | TTTCTTCACACAAGATCACA |
| 497 | 952 | TTGTGATCTTGTGTGAAGAA | 1451 | TTCTTCACACAAGATCACAA |
| 498 | 953 | TTTGTGATCTTGTGTGAAGA | 1452 | TCTTCACACAAGATCACAAA |
| 499 | 954 | GTTTGTGATCTTGTGTGAAG | 1453 | CTTCACACAAGATCACAAAC |
| 500 | 955 | GGTTTGTGATCTTGTGTGAA | 1454 | TTCACACAAGATCACAAACC |
| 501 | 956 | TGGTTTGTGATCTTGTGTGA | 1455 | TCACACAAGATCACAAACCA |
| 502 | 957 | TTGGTTTGTGATCTTGTGTG | 1456 | CACACAAGATCACAAACCAA |
| 503 | 958 | CTTGGTTTGTGATCTTGTGT | 1457 | ACACAAGATCACAAACCAAG |
| 504 | 959 | ACTTGGTTTGTGATCTTGTG | 1458 | CACAAGATCACAAACCAAGT |
| 505 | 960 | TACTTGGTTTGTGATCTTGT | 1459 | ACAAGATCACAAACCAAGTA |
| 506 | 961 | TTACTTGGTTTGTGATCTTG | 1460 | CAAGATCACAAACCAAGTAA |
| 507 | 962 | ATTACTTGGTTTGTGATCTT | 1461 | AAGATCACAAACCAAGTAAT |
| 508 | 963 | GATTACTTGGTTTGTGATCT | 1462 | AGATCACAAACCAAGTAATC |
| 509 | 964 | GGATTACTTGGTTTGTGATC | 1463 | GATCACAAACCAAGTAATCC |
| 510 | 965 | CGGATTACTTGGTTTGTGAT | 1464 | ATCACAAACCAAGTAATCCG |
| 511 | 966 | GCGGATTACTTGGTTTGTGA | 1465 | TCACAAACCAAGTAATCCGC |
| 512 | 967 | AGCGGATTACTTGGTTTGTG | 1466 | CACAAACCAAGTAATCCGCT |
| 513 | 968 | CAGCGGATTACTTGGTTTGT | 1467 | ACAAACCAAGTAATCCGCTG |
| 514 | 969 | CCAGCGGATTACTTGGTTTG | 1468 | CAAACCAAGTAATCCGCTGG |
| 515 | 970 | TCCAGCGGATTACTTGGTTT | 1469 | AAACCAAGTAATCCGCTGGA |
| 516 | 971 | CTCCAGCGGATTACTTGGTT | 1470 | AACCAAGTAATCCGCTGGAG |
| 517 | 972 | TCTCCAGCGGATTACTTGGT | 1471 | ACCAAGTAATCCGCTGGAGA |
| 518 | 973 | TTCTCCAGCGGATTACTTGG | 1472 | CCAAGTAATCCGCTGGAGAA |
| 519 | 974 | TTTCTCCAGCGGATTACTTG | 1473 | CAAGTAATCCGCTGGAGAAA |
| 520 | 975 | CTTTCTCCAGCGGATTACTT | 1474 | AAGTAATCCGCTGGAGAAAG |
| 521 | 976 | TCTTTCTCCAGCGGATTACT | 1475 | AGTAATCCGCTGGAGAAAGA |
| 522 | 977 | TTCTTTCTCCAGCGGATTAC | 1476 | GTAATCCGCTGGAGAAAGAA |
| 523 | 978 | GTTCTTTCTCCAGCGGATTA | 1477 | TAATCCGCTGGAGAAAGAAC |
| 524 | 979 | CGTTCTTTCTCCAGCGGATT | 1478 | AATCCGCTGGAGAAAGAACG |
| 525 | 980 | TCGTTCTTTCTCCAGCGGAT | 1479 | ATCCGCTGGAGAAAGAACGA |
| 526 | 981 | TTCGTTCTTTCTCCAGCGGA | 1480 | TCCGCTGGAGAAAGAACGAA |
| 527 | 982 | ATTCGTTCTTTCTCCAGCGG | 1481 | CCGCTGGAGAAAGAACGAAT |
| 528 | 983 | AATTCGTTCTTTCTCCAGCG | 1482 | CGCTGGAGAAAGAACGAATT |
| 529 | 984 | GAATTCGTTCTTTCTCCAGC | 1483 | GCTGGAGAAAGAACGAATTC |
| 530 | 985 | TGAATTCGTTCTTTCTCCAG | 1484 | CTGGAGAAAGAACGAATTCA |
| 531 | 986 | CTGAATTCGTTCTTTCTCCA | 1485 | TGGAGAAAGAACGAATTCAG |
| 532 | 987 | TCTGAATTCGTTCTTTCTCC | 1486 | GGAGAAAGAACGAATTCAGA |
| 533 | 988 | TTCTGAATTCGTTCTTTCTC | 1487 | GAGAAAGAACGAATTCAGAA |
| 534 | 989 | ATTCTGAATTCGTTCTTTCT | 1488 | AGAAAGAACGAATTCAGAAT |
| 535 | 990 | CATTCTGAATTCGTTCTTTC | 1489 | GAAAGAACGAATTCAGAATG |
| 536 | 991 | GCATTCTGAATTCGTTCTTT | 1490 | AAAGAACGAATTCAGAATGC |
| 537 | 992 | TGCATTCTGAATTCGTTCTT | 1491 | AAGAACGAATTCAGAATGCA |
| 538 | 993 | CTGCATTCTGAATTCGTTCT | 1492 | AGAACGAATTCAGAATGCAG |
| 539 | 994 | CCTGCATTCTGAATTCGTTC | 1493 | GAACGAATTCAGAATGCAGG |
| 540 | 995 | ACCTGCATTCTGAATTCGTT | 1494 | AACGAATTCAGAATGCAGGT |
| 541 | 996 | CACCTGCATTCTGAATTCGT | 1495 | ACGAATTCAGAATGCAGGTG |
| 542 | 997 | CCACCTGCATTCTGAATTCG | 1496 | CGAATTCAGAATGCAGGTGG |
| 543 | 998 | GCCACCTGCATTCTGAATTC | 1497 | GAATTCAGAATGCAGGTGGC |
| 544 | 999 | AGCCACCTGCATTCTGAATT | 1498 | AATTCAGAATGCAGGTGGCT |
| 545 | 1000 | GAGCCACCTGCATTCTGAAT | 1499 | ATTCAGAATGCAGGTGGCTC |
| 546 | 1001 | AGAGCCACCTGCATTCTGAA | 1500 | TTCAGAATGCAGGTGGCTCT |
| 547 | 1002 | CAGAGCCACCTGCATTCTGA | 1501 | TCAGAATGCAGGTGGCTCTG |
| 548 | 1003 | ACAGAGCCACCTGCATTCTG | 1502 | CAGAATGCAGGTGGCTCTGT |
| 549 | 1004 | TACAGAGCCACCTGCATTCT | 1503 | AGAATGCAGGTGGCTCTGTA |
| 550 | 1005 | TTACAGAGCCACCTGCATTC | 1504 | GAATGCAGGTGGCTCTGTAA |
| 551 | 1006 | ATTACAGAGCCACCTGCATT | 1505 | AATGCAGGTGGCTCTGTAAT |
| 552 | 1007 | CATTACAGAGCCACCTGCAT | 1506 | ATGCAGGTGGCTCTGTAATG |
| 553 | 1008 | TCATTACAGAGCCACCTGCA | 1507 | TGCAGGTGGCTCTGTAATGA |
| 554 | 1009 | ATCATTACAGAGCCACCTGC | 1508 | GCAGGTGGCTCTGTAATGAT |
| 555 | 1010 | AATCATTACAGAGCCACCTG | 1509 | CAGGTGGCTCTGTAATGATT |
| 556 | 1011 | GAATCATTACAGAGCCACCT | 1510 | AGGTGGCTCTGTAATGATTC |
| 557 | 1012 | TGAATCATTACAGAGCCACC | 1511 | GGTGGCTCTGTAATGATTCA |
| 558 | 1013 | CTGAATCATTACAGAGCCAC | 1512 | GTGGCTCTGTAATGATTCAG |
| 559 | 1014 | GCTGAATCATTACAGAGCCA | 1513 | TGGCTCTGTAATGATTCAGC |
| 560 | 1015 | CGCTGAATCATTACAGAGCC | 1514 | GGCTCTGTAATGATTCAGCG |
| 561 | 1016 | ACGCTGAATCATTACAGAGC | 1515 | GCTCTGTAATGATTCAGCGT |
| 562 | 1017 | CACGCTGAATCATTACAGAG | 1516 | CTCTGTAATGATTCAGCGTG |
| 563 | 1018 | ACACGCTGAATCATTACAGA | 1517 | TCTGTAATGATTCAGCGTGT |
| 564 | 1019 | CACACGCTGAATCATTACAG | 1518 | CTGTAATGATTCAGCGTGTG |
| 565 | 1020 | TCACACGCTGAATCATTACA | 1519 | TGTAATGATTCAGCGTGTGA |
| 566 | 1021 | TTCACACGCTGAATCATTAC | 1520 | GTAATGATTCAGCGTGTGAA |
| 567 | 1022 | ATTCACACGCTGAATCATTA | 1521 | TAATGATTCAGCGTGTGAAT |
| 568 | 1023 | CATTCACACGCTGAATCATT | 1522 | AATGATTCAGCGTGTGAATG |
| 569 | 1024 | CCATTCACACGCTGAATCAT | 1523 | ATGATTCAGCGTGTGAATGG |
| 570 | 1025 | GCCATTCACACGCTGAATCA | 1524 | TGATTCAGCGTGTGAATGGC |
| 571 | 1026 | AGCCATTCACACGCTGAATC | 1525 | GATTCAGCGTGTGAATGGCT |
| 572 | 1027 | GAGCCATTCACACGCTGAAT | 1526 | ATTCAGCGTGTGAATGGCTC |
| 573 | 1028 | AGAGCCATTCACACGCTGAA | 1527 | TTCAGCGTGTGAATGGCTCT |
| 574 | 1029 | GAGAGCCATTCACACGCTGA | 1528 | TCAGCGTGTGAATGGCTCTC |
| 575 | 1030 | AGAGAGCCATTCACACGCTG | 1529 | CAGCGTGTGAATGGCTCTCT |
| 576 | 1031 | CAGAGAGCCATTCACACGCT | 1530 | AGCGTGTGAATGGCTCTCTG |
| 577 | 1032 | CCAGAGAGCCATTCACACGC | 1531 | GCGTGTGAATGGCTCTCTGG |
| 578 | 1033 | GCCAGAGAGCCATTCACACG | 1532 | CGTGTGAATGGCTCTCTGGC |
| 579 | 1034 | AGCCAGAGAGCCATTCACAC | 1533 | GTGTGAATGGCTCTCTGGCT |
| 580 | 1035 | CAGCCAGAGAGCCATTCACA | 1534 | TGTGAATGGCTCTCTGGCTG |
| 581 | 1036 | ACAGCCAGAGAGCCATTCAC | 1535 | GTGAATGGCTCTCTGGCTGT |
| 582 | 1037 | TACAGCCAGAGAGCCATTCA | 1536 | TGAATGGCTCTCTGGCTGTA |
| 583 | 1038 | ATACAGCCAGAGAGCCATTC | 1537 | GAATGGCTCTCTGGCTGTAT |
| 584 | 1039 | GATACAGCCAGAGAGCCATT | 1538 | AATGGCTCTCTGGCTGTATC |
| 585 | 1040 | CGATACAGCCAGAGAGCCAT | 1539 | ATGGCTCTCTGGCTGTATCG |
| 586 | 1041 | TCGATACAGCCAGAGAGCCA | 1540 | TGGCTCTCTGGCTGTATCGA |
| 587 | 1042 | CTCGATACAGCCAGAGAGCC | 1541 | GGCTCTCTGGCTGTATCGAG |
| 588 | 1043 | CCTCGATACAGCCAGAGAGC | 1542 | GCTCTCTGGCTGTATCGAGG |
| 589 | 1044 | CCCTCGATACAGCCAGAGAG | 1543 | CTCTCTGGCTGTATCGAGGG |
| 590 | 1045 | GCCCTCGATACAGCCAGAGA | 1544 | TCTCTGGCTGTATCGAGGGC |
| 591 | 1046 | GGCCCTCGATACAGCCAGAG | 1545 | CTCTGGCTGTATCGAGGGCC |
| 592 | 1047 | GGGCCCTCGATACAGCCAGA | 1546 | TCTGGCTGTATCGAGGGCCC |
| 593 | 1048 | AGGGCCCTCGATACAGCCAG | 1547 | CTGGCTGTATCGAGGGCCCT |
| 594 | 1049 | AAGGGCCCTCGATACAGCCA | 1548 | TGGCTGTATCGAGGGCCCTT |
| 595 | 1050 | CAAGGGCCCTCGATACAGCC | 1549 | GGCTGTATCGAGGGCCCTTG |
| 596 | 1051 | CCAAGGGCCCTCGATACAGC | 1550 | GCTGTATCGAGGGCCCTTGG |
| 597 | 1052 | CCCAAGGGCCCTCGATACAG | 1551 | CTGTATCGAGGGCCCTTGGG |
| 598 | 1053 | CCCCAAGGGCCCTCGATACA | 1552 | TGTATCGAGGGCCCTTGGGG |
| 599 | 1054 | TCCCCAAGGGCCCTCGATAC | 1553 | GTATCGAGGGCCCTTGGGGA |
| 600 | 1055 | ATCCCCAAGGGCCCTCGATA | 1554 | TATCGAGGGCCCTTGGGGAT |
| 601 | 1056 | AATCCCCAAGGGCCCTCGAT | 1555 | ATCGAGGGCCCTTGGGGATT |
| 602 | 1057 | AAATCCCCAAGGGCCCTCGA | 1556 | TCGAGGGCCCTTGGGGATTT |
| 603 | 1058 | AAAATCCCCAAGGGCCCTCG | 1557 | CGAGGGCCCTTGGGGATTTT |
| 604 | 1059 | CAAAATCCCCAAGGGCCCTC | 1558 | GAGGGCCCTTGGGGATTTTG |
| 605 | 1060 | TCAAAATCCCCAAGGGCCCT | 1559 | AGGGCCCTTGGGGATTTTGA |
| 606 | 1061 | ATCAAAATCCCCAAGGGCCC | 1560 | GGGCCCTTGGGGATTTTGAT |
| 607 | 1062 | AATCAAAATCCCCAAGGGCC | 1561 | GGCCCTTGGGGATTTTGATT |
| 608 | 1063 | TAATCAAAATCCCCAAGGGC | 1562 | GCCCTTGGGGATTTTGATTA |
| 609 | 1064 | GTAATCAAAATCCCCAAGGG | 1563 | CCCTTGGGGATTTTGATTAC |
| 610 | 1065 | TGTAATCAAAATCCCCAAGG | 1564 | CCTTGGGGATTTTGATTACA |
| 611 | 1066 | TTGTAATCAAAATCCCCAAG | 1565 | CTTGGGGATTTTGATTACAA |
| 612 | 1067 | TTTGTAATCAAAATCCCCAA | 1566 | TTGGGGATTTTGATTACAAA |
| 613 | 1068 | ATTTGTAATCAAAATCCCCA | 1567 | TGGGGATTTTGATTACAAAT |
| 614 | 1069 | CATTTGTAATCAAAATCCCC | 1568 | GGGGATTTTGATTACAAATG |
| 615 | 1070 | ACATTTGTAATCAAAATCCC | 1569 | GGGATTTTGATTACAAATGT |
| 616 | 1071 | CACATTTGTAATCAAAATCC | 1570 | GGATTTTGATTACAAATGTG |
| 617 | 1072 | ACACATTTGTAATCAAAATC | 1571 | GATTTTGATTACAAATGTGT |
| 618 | 1073 | GACACATTTGTAATCAAAAT | 1572 | ATTTTGATTACAAATGTGTC |
| 619 | 1074 | GGACACATTTGTAATCAAAA | 1573 | TTTTGATTACAAATGTGTCC |
| 620 | 1075 | TGGACACATTTGTAATCAAA | 1574 | TTTGATTACAAATGTGTCCA |
| 621 | 1076 | ATGGACACATTTGTAATCAA | 1575 | TTGATTACAAATGTGTCCAT |
| 622 | 1077 | CATGGACACATTTGTAATCA | 1576 | TGATTACAAATGTGTCCATG |
| 623 | 1078 | CCATGGACACATTTGTAATC | 1577 | GATTACAAATGTGTCCATGG |
| 624 | 1079 | TCCATGGACACATTTGTAAT | 1578 | ATTACAAATGTGTCCATGGA |
| 625 | 1080 | TTCCATGGACACATTTGTAA | 1579 | TTACAAATGTGTCCATGGAA |
| 626 | 1081 | TTTCCATGGACACATTTGTA | 1580 | TACAAATGTGTCCATGGAAA |
| 627 | 1082 | TTTTCCATGGACACATTTGT | 1581 | ACAAATGTGTCCATGGAAAA |
| 628 | 1083 | CTTTTCCATGGACACATTTG | 1582 | CAAATGTGTCCATGGAAAAG |
| 629 | 1084 | CCTTTTCCATGGACACATTT | 1583 | AAATGTGTCCATGGAAAAGG |
| 630 | 1085 | ACCTTTTCCATGGACACATT | 1584 | AATGTGTCCATGGAAAAGGT |
| 631 | 1086 | GACCTTTTCCATGGACACAT | 1585 | ATGTGTCCATGGAAAAGGTC |
| 632 | 1087 | GGACCTTTTCCATGGACACA | 1586 | TGTGTCCATGGAAAAGGTCC |
| 633 | 1088 | AGGACCTTTTCCATGGACAC | 1587 | GTGTCCATGGAAAAGGTCCT |
| 634 | 1089 | TAGGACCTTTTCCATGGACA | 1588 | TGTCCATGGAAAAGGTCCTA |
| 635 | 1090 | GTAGGACCTTTTCCATGGAC | 1589 | GTCCATGGAAAAGGTCCTAC |
| 636 | 1091 | AGTAGGACCTTTTCCATGGA | 1590 | TCCATGGAAAAGGTCCTACT |
| 637 | 1092 | CAGTAGGACCTTTTCCATGG | 1591 | CCATGGAAAAGGTCCTACTG |
| 638 | 1093 | TCAGTAGGACCTTTTCCATG | 1592 | CATGGAAAAGGTCCTACTGA |
| 639 | 1094 | CTCAGTAGGACCTTTTCCAT | 1593 | ATGGAAAAGGTCCTACTGAG |
| 640 | 1095 | GCTCAGTAGGACCTTTTCCA | 1594 | TGGAAAAGGTCCTACTGAGC |
| 641 | 1096 | TGCTCAGTAGGACCTTTTCC | 1595 | GGAAAAGGTCCTACTGAGCA |
| 642 | 1097 | CTGCTCAGTAGGACCTTTTC | 1596 | GAAAAGGTCCTACTGAGCAG |
| 643 | 1098 | GCTGCTCAGTAGGACCTTTT | 1597 | AAAAGGTCCTACTGAGCAGC |
| 644 | 1099 | AGCTGCTCAGTAGGACCTTT | 1598 | AAAGGTCCTACTGAGCAGCT |
| 645 | 1100 | AAGCTGCTCAGTAGGACCTT | 1599 | AAGGTCCTACTGAGCAGCTT |
| 646 | 1101 | CAAGCTGCTCAGTAGGACCT | 1600 | AGGTCCTACTGAGCAGCTTG |
| 647 | 1102 | ACAAGCTGCTCAGTAGGACC | 1601 | GGTCCTACTGAGCAGCTTGT |
| 648 | 1103 | GACAAGCTGCTCAGTAGGAC | 1602 | GTCCTACTGAGCAGCTTGTC |
| 649 | 1104 | AGACAAGCTGCTCAGTAGGA | 1603 | TCCTACTGAGCAGCTTGTCT |
| 650 | 1105 | GAGACAAGCTGCTCAGTAGG | 1604 | CCTACTGAGCAGCTTGTCTC |
| 651 | 1106 | TGAGACAAGCTGCTCAGTAG | 1605 | CTACTGAGCAGCTTGTCTCA |
| 652 | 1107 | GTGAGACAAGCTGCTCAGTA | 1606 | TACTGAGCAGCTTGTCTCAC |
| 653 | 1108 | GGTGAGACAAGCTGCTCAGT | 1607 | ACTGAGCAGCTTGTCTCACC |
| 654 | 1109 | TGGTGAGACAAGCTGCTCAG | 1608 | CTGAGCAGCTTGTCTCACCA |
| 655 | 1110 | CTGGTGAGACAAGCTGCTCA | 1609 | TGAGCAGCTTGTCTCACCAG |
| 656 | 1111 | TCTGGTGAGACAAGCTGCTC | 1610 | GAGCAGCTTGTCTCACCAGA |
| 657 | 1112 | CTCTGGTGAGACAAGCTGCT | 1611 | AGCAGCTTGTCTCACCAGAG |
| 658 | 1113 | GCTCTGGTGAGACAAGCTGC | 1612 | GCAGCTTGTCTCACCAGAGC |
| 659 | 1114 | GGCTCTGGTGAGACAAGCTG | 1613 | CAGCTTGTCTCACCAGAGCC |
| 660 | 1115 | AGGCTCTGGTGAGACAAGCT | 1614 | AGCTTGTCTCACCAGAGCCT |
| 661 | 1116 | CAGGCTCTGGTGAGACAAGC | 1615 | GCTTGTCTCACCAGAGCCTG |
| 662 | 1117 | TCAGGCTCTGGTGAGACAAG | 1616 | CTTGTCTCACCAGAGCCTGA |
| 663 | 1118 | TTCAGGCTCTGGTGAGACAA | 1617 | TTGTCTCACCAGAGCCTGAA |
| 664 | 1119 | CTTCAGGCTCTGGTGAGACA | 1618 | TGTCTCACCAGAGCCTGAAG |
| 665 | 1120 | ACTTCAGGCTCTGGTGAGAC | 1619 | GTCTCACCAGAGCCTGAAGT |
| 666 | 1121 | GACTTCAGGCTCTGGTGAGA | 1620 | TCTCACCAGAGCCTGAAGTC |
| 667 | 1122 | GGACTTCAGGCTCTGGTGAG | 1621 | CTCACCAGAGCCTGAAGTCC |
| 668 | 1123 | TGGACTTCAGGCTCTGGTGA | 1622 | TCACCAGAGCCTGAAGTCCA |
| 669 | 1124 | ATGGACTTCAGGCTCTGGTG | 1623 | CACCAGAGCCTGAAGTCCAT |
| 670 | 1125 | CATGGACTTCAGGCTCTGGT | 1624 | ACCAGAGCCTGAAGTCCATG |
| 671 | 1126 | TCATGGACTTCAGGCTCTGG | 1625 | CCAGAGCCTGAAGTCCATGA |
| 672 | 1127 | ATCATGGACTTCAGGCTCTG | 1626 | CAGAGCCTGAAGTCCATGAT |
| 673 | 1128 | TATCATGGACTTCAGGCTCT | 1627 | AGAGCCTGAAGTCCATGATA |
| 674 | 1129 | ATATCATGGACTTCAGGCTC | 1628 | GAGCCTGAAGTCCATGATAT |
| 675 | 1130 | AATATCATGGACTTCAGGCT | 1629 | AGCCTGAAGTCCATGATATT |
| 676 | 1131 | CAATATCATGGACTTCAGGC | 1630 | GCCTGAAGTCCATGATATTG |
| 677 | 1132 | TCAATATCATGGACTTCAGG | 1631 | CCTGAAGTCCATGATATTGA |
| 678 | 1133 | TTCAATATCATGGACTTCAG | 1632 | CTGAAGTCCATGATATTGAA |
| 679 | 1134 | TTTCAATATCATGGACTTCA | 1633 | TGAAGTCCATGATATTGAAA |
| 680 | 1135 | CTTTCAATATCATGGACTTC | 1634 | GAAGTCCATGATATTGAAAG |
| 681 | 1136 | TCTTTCAATATCATGGACTT | 1635 | AAGTCCATGATATTGAAAGA |
| 682 | 1137 | ATCTTTCAATATCATGGACT | 1636 | AGTCCATGATATTGAAAGAT |
| 683 | 1138 | GATCTTTCAATATCATGGAC | 1637 | GTCCATGATATTGAAAGATC |
| 684 | 1139 | AGATCTTTCAATATCATGGA | 1638 | TCCATGATATTGAAAGATCT |
| 685 | 1140 | CAGATCTTTCAATATCATGG | 1639 | CCATGATATTGAAAGATCTG |
| 686 | 1141 | TCAGATCTTTCAATATCATG | 1640 | CATGATATTGAAAGATCTGA |
| 687 | 1142 | TTCAGATCTTTCAATATCAT | 1641 | ATGATATTGAAAGATCTGAA |
| 688 | 1143 | CTTCAGATCTTTCAATATCA | 1642 | TGATATTGAAAGATCTGAAG |
| 689 | 1144 | TCTTCAGATCTTTCAATATC | 1643 | GATATTGAAAGATCTGAAGA |
| 690 | 1145 | TTCTTCAGATCTTTCAATAT | 1644 | ATATTGAAAGATCTGAAGAA |
| 691 | 1146 | CTTCTTCAGATCTTTCAATA | 1645 | TATTGAAAGATCTGAAGAAG |
| 692 | 1147 | TCTTCTTCAGATCTTTCAAT | 1646 | ATTGAAAGATCTGAAGAAGA |
| 693 | 1148 | ATCTTCTTCAGATCTTTCAA | 1647 | TTGAAAGATCTGAAGAAGAT |
| 694 | 1149 | CATCTTCTTCAGATCTTTCA | 1648 | TGAAAGATCTGAAGAAGATG |
| 695 | 1150 | TCATCTTCTTCAGATCTTTC | 1649 | GAAAGATCTGAAGAAGATGA |
| 696 | 1151 | ATCATCTTCTTCAGATCTTT | 1650 | AAAGATCTGAAGAAGATGAT |
| 697 | 1152 | GATCATCTTCTTCAGATCTT | 1651 | AAGATCTGAAGAAGATGATC |
| 698 | 1153 | TGATCATCTTCTTCAGATCT | 1652 | AGATCTGAAGAAGATGATCA |
| 699 | 1154 | CTGATCATCTTCTTCAGATC | 1653 | GATCTGAAGAAGATGATCAG |
| 700 | 1155 | ACTGATCATCTTCTTCAGAT | 1654 | ATCTGAAGAAGATGATCAGT |
| 701 | 1156 | AACTGATCATCTTCTTCAGA | 1655 | TCTGAAGAAGATGATCAGTT |
| 702 | 1157 | GAACTGATCATCTTCTTCAG | 1656 | CTGAAGAAGATGATCAGTTC |
| 703 | 1158 | TGAACTGATCATCTTCTTCA | 1657 | TGAAGAAGATGATCAGTTCA |
| 704 | 1159 | ATGAACTGATCATCTTCTTC | 1658 | GAAGAAGATGATCAGTTCAT |
| 705 | 1160 | AATGAACTGATCATCTTCTT | 1659 | AAGAAGATGATCAGTTCATT |
| 706 | 1161 | TAATGAACTGATCATCTTCT | 1660 | AGAAGATGATCAGTTCATTA |
| 707 | 1162 | ATAATGAACTGATCATCTTC | 1661 | GAAGATGATCAGTTCATTAT |
| 708 | 1163 | GATAATGAACTGATCATCTT | 1662 | AAGATGATCAGTTCATTATC |
| 709 | 1164 | GGATAATGAACTGATCATCT | 1663 | AGATGATCAGTTCATTATCC |
| 710 | 1165 | AGGATAATGAACTGATCATC | 1664 | GATGATCAGTTCATTATCCT |
| 711 | 1166 | AAGGATAATGAACTGATCAT | 1665 | ATGATCAGTTCATTATCCTT |
| 712 | 1167 | CAAGGATAATGAACTGATCA | 1666 | TGATCAGTTCATTATCCTTG |
| 713 | 1168 | GCAAGGATAATGAACTGATC | 1667 | GATCAGTTCATTATCCTTGC |
| 714 | 1169 | TGCAAGGATAATGAACTGAT | 1668 | ATCAGTTCATTATCCTTGCA |
| 715 | 1170 | ATGCAAGGATAATGAACTGA | 1669 | TCAGTTCATTATCCTTGCAT |
| 716 | 1171 | CATGCAAGGATAATGAACTG | 1670 | CAGTTCATTATCCTTGCATG |
| 717 | 1172 | ACATGCAAGGATAATGAACT | 1671 | AGTTCATTATCCTTGCATGT |
| 718 | 1173 | CACATGCAAGGATAATGAAC | 1672 | GTTCATTATCCTTGCATGTG |
| 719 | 1174 | TCACATGCAAGGATAATGAA | 1673 | TTCATTATCCTTGCATGTGA |
| 720 | 1175 | ATCACATGCAAGGATAATGA | 1674 | TCATTATCCTTGCATGTGAT |
| 721 | 1176 | CATCACATGCAAGGATAATG | 1675 | CATTATCCTTGCATGTGATG |
| 722 | 1177 | CCATCACATGCAAGGATAAT | 1676 | ATTATCCTTGCATGTGATGG |
| 723 | 1178 | ACCATCACATGCAAGGATAA | 1677 | TTATCCTTGCATGTGATGGT |
| 724 | 1179 | TACCATCACATGCAAGGATA | 1678 | TATCCTTGCATGTGATGGTA |
| 725 | 1180 | ATACCATCACATGCAAGGAT | 1679 | ATCCTTGCATGTGATGGTAT |
| 726 | 1181 | GATACCATCACATGCAAGGA | 1680 | TCCTTGCATGTGATGGTATC |
| 727 | 1182 | AGATACCATCACATGCAAGG | 1681 | CCTTGCATGTGATGGTATCT |
| 728 | 1183 | CAGATACCATCACATGCAAG | 1682 | CTTGCATGTGATGGTATCTG |
| 729 | 1184 | CCAGATACCATCACATGCAA | 1683 | TTGCATGTGATGGTATCTGG |
| 730 | 1185 | CCCAGATACCATCACATGCA | 1684 | TGCATGTGATGGTATCTGGG |
| 731 | 1186 | TCCCAGATACCATCACATGC | 1685 | GCATGTGATGGTATCTGGGA |
| 732 | 1187 | ATCCCAGATACCATCACATG | 1686 | CATGTGATGGTATCTGGGAT |
| 733 | 1188 | CATCCCAGATACCATCACAT | 1687 | ATGTGATGGTATCTGGGATG |
| 734 | 1189 | ACATCCCAGATACCATCACA | 1688 | TGTGATGGTATCTGGGATGT |
| 735 | 1190 | AACATCCCAGATACCATCAC | 1689 | GTGATGGTATCTGGGATGTT |
| 736 | 1191 | TAACATCCCAGATACCATCA | 1690 | TGATGGTATCTGGGATGTTA |
| 737 | 1192 | ATAACATCCCAGATACCATC | 1691 | GATGGTATCTGGGATGTTAT |
| 738 | 1193 | CATAACATCCCAGATACCAT | 1692 | ATGGTATCTGGGATGTTATG |
| 739 | 1194 | CCATAACATCCCAGATACCA | 1693 | TGGTATCTGGGATGTTATGG |
| 740 | 1195 | CCCATAACATCCCAGATACC | 1694 | GGTATCTGGGATGTTATGGG |
| 741 | 1196 | TCCCATAACATCCCAGATAC | 1695 | GTATCTGGGATGTTATGGGA |
| 742 | 1197 | TTCCCATAACATCCCAGATA | 1696 | TATCTGGGATGTTATGGGAA |
| 743 | 1198 | TTTCCCATAACATCCCAGAT | 1697 | ATCTGGGATGTTATGGGAAA |
| 744 | 1199 | ATTTCCCATAACATCCCAGA | 1698 | TCTGGGATGTTATGGGAAAT |
| 745 | 1200 | CATTTCCCATAACATCCCAG | 1699 | CTGGGATGTTATGGGAAATG |
| 746 | 1201 | TCATTTCCCATAACATCCCA | 1700 | TGGGATGTTATGGGAAATGA |
| 747 | 1202 | TTCATTTCCCATAACATCCC | 1701 | GGGATGTTATGGGAAATGAA |
| 748 | 1203 | CTTCATTTCCCATAACATCC | 1702 | GGATGTTATGGGAAATGAAG |
| 749 | 1204 | TCTTCATTTCCCATAACATC | 1703 | GATGTTATGGGAAATGAAGA |
| 750 | 1205 | CTCTTCATTTCCCATAACAT | 1704 | ATGTTATGGGAAATGAAGAG |
| 751 | 1206 | GCTCTTCATTTCCCATAACA | 1705 | TGTTATGGGAAATGAAGAGC |
| 752 | 1207 | AGCTCTTCATTTCCCATAAC | 1706 | GTTATGGGAAATGAAGAGCT |
| 753 | 1208 | GAGCTCTTCATTTCCCATAA | 1707 | TTATGGGAAATGAAGAGCTC |
| 754 | 1209 | AGAGCTCTTCATTTCCCATA | 1708 | TATGGGAAATGAAGAGCTCT |
| 755 | 1210 | CAGAGCTCTTCATTTCCCAT | 1709 | ATGGGAAATGAAGAGCTCTG |
| 756 | 1211 | ACAGAGCTCTTCATTTCCCA | 1710 | TGGGAAATGAAGAGCTCTGT |
| 757 | 1212 | CACAGAGCTCTTCATTTCCC | 1711 | GGGAAATGAAGAGCTCTGTG |
| 758 | 1213 | TCACAGAGCTCTTCATTTCC | 1712 | GGAAATGAAGAGCTCTGTGA |
| 759 | 1214 | ATCACAGAGCTCTTCATTTC | 1713 | GAAATGAAGAGCTCTGTGAT |
| 760 | 1215 | AATCACAGAGCTCTTCATTT | 1714 | AAATGAAGAGCTCTGTGATT |
| 761 | 1216 | AAATCACAGAGCTCTTCATT | 1715 | AATGAAGAGCTCTGTGATTT |
| 762 | 1217 | AAAATCACAGAGCTCTTCAT | 1716 | ATGAAGAGCTCTGTGATTTT |
| 763 | 1218 | CAAAATCACAGAGCTCTTCA | 1717 | TGAAGAGCTCTGTGATTTTG |
| 764 | 1219 | ACAAAATCACAGAGCTCTTC | 1718 | GAAGAGCTCTGTGATTTTGT |
| 765 | 1220 | TACAAAATCACAGAGCTCTT | 1719 | AAGAGCTCTGTGATTTTGTA |
| 766 | 1221 | TTACAAAATCACAGAGCTCT | 1720 | AGAGCTCTGTGATTTTGTAA |
| 767 | 1222 | CTTACAAAATCACAGAGCTC | 1721 | GAGCTCTGTGATTTTGTAAG |
| 768 | 1223 | TCTTACAAAATCACAGAGCT | 1722 | AGCTCTGTGATTTTGTAAGA |
| 769 | 1224 | ATCTTACAAAATCACAGAGC | 1723 | GCTCTGTGATTTTGTAAGAT |
| 770 | 1225 | GATCTTACAAAATCACAGAG | 1724 | CTCTGTGATTTTGTAAGATC |
| 771 | 1226 | GGATCTTACAAAATCACAGA | 1725 | TCTGTGATTTTGTAAGATCC |
| 772 | 1227 | TGGATCTTACAAAATCACAG | 1726 | CTGTGATTTTGTAAGATCCA |
| 773 | 1228 | CTGGATCTTACAAAATCACA | 1727 | TGTGATTTTGTAAGATCCAG |
| 774 | 1229 | TCTGGATCTTACAAAATCAC | 1728 | GTGATTTTGTAAGATCCAGA |
| 775 | 1230 | GTCTGGATCTTACAAAATCA | 1729 | TGATTTTGTAAGATCCAGAC |
| 776 | 1231 | AGTCTGGATCTTACAAAATC | 1730 | GATTTTGTAAGATCCAGACT |
| 777 | 1232 | AAGTCTGGATCTTACAAAAT | 1731 | ATTTTGTAAGATCCAGACTT |
| 778 | 1233 | CAAGTCTGGATCTTACAAAA | 1732 | TTTTGTAAGATCCAGACTTG |
| 779 | 1234 | TCAAGTCTGGATCTTACAAA | 1733 | TTTGTAAGATCCAGACTTGA |
| 780 | 1235 | TTCAAGTCTGGATCTTACAA | 1734 | TTGTAAGATCCAGACTTGAA |
| 781 | 1236 | CTTCAAGTCTGGATCTTACA | 1735 | TGTAAGATCCAGACTTGAAG |
| 782 | 1237 | ACTTCAAGTCTGGATCTTAC | 1736 | GTAAGATCCAGACTTGAAGT |
| 783 | 1238 | GACTTCAAGTCTGGATCTTA | 1737 | TAAGATCCAGACTTGAAGTC |
| 784 | 1239 | TGACTTCAAGTCTGGATCTT | 1738 | AAGATCCAGACTTGAAGTCA |
| 785 | 1240 | GTGACTTCAAGTCTGGATCT | 1739 | AGATCCAGACTTGAAGTCAC |
| 786 | 1241 | AGTGACTTCAAGTCTGGATC | 1740 | GATCCAGACTTGAAGTCACT |
| 787 | 1242 | CAGTGACTTCAAGTCTGGAT | 1741 | ATCCAGACTTGAAGTCACTG |
| 788 | 1243 | TCAGTGACTTCAAGTCTGGA | 1742 | TCCAGACTTGAAGTCACTGA |
| 789 | 1244 | ATCAGTGACTTCAAGTCTGG | 1743 | CCAGACTTGAAGTCACTGAT |
| 790 | 1245 | CATCAGTGACTTCAAGTCTG | 1744 | CAGACTTGAAGTCACTGATG |
| 791 | 1246 | TCATCAGTGACTTCAAGTCT | 1745 | AGACTTGAAGTCACTGATGA |
| 792 | 1247 | GTCATCAGTGACTTCAAGTC | 1746 | GACTTGAAGTCACTGATGAC |
| 793 | 1248 | GGTCATCAGTGACTTCAAGT | 1747 | ACTTGAAGTCACTGATGACC |
| 794 | 1249 | AGGTCATCAGTGACTTCAAG | 1748 | CTTGAAGTCACTGATGACCT |
| 795 | 1250 | AAGGTCATCAGTGACTTCAA | 1749 | TTGAAGTCACTGATGACCTT |
| 796 | 1251 | CAAGGTCATCAGTGACTTCA | 1750 | TGAAGTCACTGATGACCTTG |
| 797 | 1252 | TCAAGGTCATCAGTGACTTC | 1751 | GAAGTCACTGATGACCTTGA |
| 798 | 1253 | CTCAAGGTCATCAGTGACTT | 1752 | AAGTCACTGATGACCTTGAG |
| 799 | 1254 | TCTCAAGGTCATCAGTGACT | 1753 | AGTCACTGATGACCTTGAGA |
| 800 | 1255 | TTCTCAAGGTCATCAGTGAC | 1754 | GTCACTGATGACCTTGAGAA |
| 801 | 1256 | TTTCTCAAGGTCATCAGTGA | 1755 | TCACTGATGACCTTGAGAAA |
| 802 | 1257 | CTTTCTCAAGGTCATCAGTG | 1756 | CACTGATGACCTTGAGAAAG |
| 803 | 1258 | ACTTTCTCAAGGTCATCAGT | 1757 | ACTGATGACCTTGAGAAAGT |
| 804 | 1259 | AACTTTCTCAAGGTCATCAG | 1758 | CTGATGACCTTGAGAAAGTT |
| 805 | 1260 | AAACTTTCTCAAGGTCATCA | 1759 | TGATGACCTTGAGAAAGTTT |
| 806 | 1261 | CAAACTTTCTCAAGGTCATC | 1760 | GATGACCTTGAGAAAGTTTG |
| 807 | 1262 | GCAAACTTTCTCAAGGTCAT | 1761 | ATGACCTTGAGAAAGTTTGC |
| 808 | 1263 | TGCAAACTTTCTCAAGGTCA | 1762 | TGACCTTGAGAAAGTTTGCA |
| 809 | 1264 | TTGCAAACTTTCTCAAGGTC | 1763 | GACCTTGAGAAAGTTTGCAA |
| 810 | 1265 | ATTGCAAACTTTCTCAAGGT | 1764 | ACCTTGAGAAAGTTTGCAAT |
| 811 | 1266 | CATTGCAAACTTTCTCAAGG | 1765 | CCTTGAGAAAGTTTGCAATG |
| 812 | 1267 | TCATTGCAAACTTTCTCAAG | 1766 | CTTGAGAAAGTTTGCAATGA |
| 813 | 1268 | TTCATTGCAAACTTTCTCAA | 1767 | TTGAGAAAGTTTGCAATGAA |
| 814 | 1269 | CTTCATTGCAAACTTTCTCA | 1768 | TGAGAAAGTTTGCAATGAAG |
| 815 | 1270 | ACTTCATTGCAAACTTTCTC | 1769 | GAGAAAGTTTGCAATGAAGT |
| 816 | 1271 | TACTTCATTGCAAACTTTCT | 1770 | AGAAAGTTTGCAATGAAGTA |
| 817 | 1272 | CTACTTCATTGCAAACTTTC | 1771 | GAAAGTTTGCAATGAAGTAG |
| 818 | 1273 | ACTACTTCATTGCAAACTTT | 1772 | AAAGTTTGCAATGAAGTAGT |
| 819 | 1274 | GACTACTTCATTGCAAACTT | 1773 | AAGTTTGCAATGAAGTAGTC |
| 820 | 1275 | CGACTACTTCATTGCAAACT | 1774 | AGTTTGCAATGAAGTAGTCG |
| 821 | 1276 | TCGACTACTTCATTGCAAAC | 1775 | GTTTGCAATGAAGTAGTCGA |
| 822 | 1277 | GTCGACTACTTCATTGCAAA | 1776 | TTTGCAATGAAGTAGTCGAC |
| 823 | 1278 | TGTCGACTACTTCATTGCAA | 1777 | TTGCAATGAAGTAGTCGACA |
| 824 | 1279 | GTGTCGACTACTTCATTGCA | 1778 | TGCAATGAAGTAGTCGACAC |
| 825 | 1280 | GGTGTCGACTACTTCATTGC | 1779 | GCAATGAAGTAGTCGACACC |
| 826 | 1281 | AGGTGTCGACTACTTCATTG | 1780 | CAATGAAGTAGTCGACACCT |
| 827 | 1282 | CAGGTGTCGACTACTTCATT | 1781 | AATGAAGTAGTCGACACCTG |
| 828 | 1283 | ACAGGTGTCGACTACTTCAT | 1782 | ATGAAGTAGTCGACACCTGT |
| 829 | 1284 | AACAGGTGTCGACTACTTCA | 1783 | TGAAGTAGTCGACACCTGTT |
| 830 | 1285 | AAACAGGTGTCGACTACTTC | 1784 | GAAGTAGTCGACACCTGTTT |
| 831 | 1286 | CAAACAGGTGTCGACTACTT | 1785 | AAGTAGTCGACACCTGTTTG |
| 832 | 1287 | ACAAACAGGTGTCGACTACT | 1786 | AGTAGTCGACACCTGTTTGT |
| 833 | 1288 | TACAAACAGGTGTCGACTAC | 1787 | GTAGTCGACACCTGTTTGTA |
| 834 | 1289 | ATACAAACAGGTGTCGACTA | 1788 | TAGTCGACACCTGTTTGTAT |
| 835 | 1290 | TATACAAACAGGTGTCGACT | 1789 | AGTCGACACCTGTTTGTATA |
| 836 | 1291 | TTATACAAACAGGTGTCGAC | 1790 | GTCGACACCTGTTTGTATAA |
| 837 | 1292 | CTTATACAAACAGGTGTCGA | 1791 | TCGACACCTGTTTGTATAAG |
| 838 | 1293 | CCTTATACAAACAGGTGTCG | 1792 | CGACACCTGTTTGTATAAGG |
| 839 | 1294 | CCCTTATACAAACAGGTGTC | 1793 | GACACCTGTTTGTATAAGGG |
| 840 | 1295 | TCCCTTATACAAACAGGTGT | 1794 | ACACCTGTTTGTATAAGGGA |
| 841 | 1296 | TTCCCTTATACAAACAGGTG | 1795 | CACCTGTTTGTATAAGGGAA |
| 842 | 1297 | CTTCCCTTATACAAACAGGT | 1796 | ACCTGTTTGTATAAGGGAAG |
| 843 | 1298 | ACTTCCCTTATACAAACAGG | 1797 | CCTGTTTGTATAAGGGAAGT |
| 844 | 1299 | GACTTCCCTTATACAAACAG | 1798 | CTGTTTGTATAAGGGAAGTC |
| 845 | 1300 | CGACTTCCCTTATACAAACA | 1799 | TGTTTGTATAAGGGAAGTCG |
| 846 | 1301 | TCGACTTCCCTTATACAAAC | 1800 | GTTTGTATAAGGGAAGTCGA |
| 847 | 1302 | CTCGACTTCCCTTATACAAA | 1801 | TTTGTATAAGGGAAGTCGAG |
| 848 | 1303 | TCTCGACTTCCCTTATACAA | 1802 | TTGTATAAGGGAAGTCGAGA |
| 849 | 1304 | GTCTCGACTTCCCTTATACA | 1803 | TGTATAAGGGAAGTCGAGAC |
| 850 | 1305 | TGTCTCGACTTCCCTTATAC | 1804 | GTATAAGGGAAGTCGAGACA |
| 851 | 1306 | TTGTCTCGACTTCCCTTATA | 1805 | TATAAGGGAAGTCGAGACAA |
| 852 | 1307 | GTTGTCTCGACTTCCCTTAT | 1806 | ATAAGGGAAGTCGAGACAAC |
| 853 | 1308 | TGTTGTCTCGACTTCCCTTA | 1807 | TAAGGGAAGTCGAGACAACA |
| 854 | 1309 | ATGTTGTCTCGACTTCCCTT | 1808 | AAGGGAAGTCGAGACAACAT |
| 855 | 1310 | CATGTTGTCTCGACTTCCCT | 1809 | AGGGAAGTCGAGACAACATG |
| 856 | 1311 | TCATGTTGTCTCGACTTCCC | 1810 | GGGAAGTCGAGACAACATGA |
| 857 | 1312 | CTCATGTTGTCTCGACTTCC | 1811 | GGAAGTCGAGACAACATGAG |
| 858 | 1313 | ACTCATGTTGTCTCGACTTC | 1812 | GAAGTCGAGACAACATGAGT |
| 859 | 1314 | CACTCATGTTGTCTCGACTT | 1813 | AAGTCGAGACAACATGAGTG |
| 860 | 1315 | ACACTCATGTTGTCTCGACT | 1814 | AGTCGAGACAACATGAGTGT |
| 861 | 1316 | CACACTCATGTTGTCTCGAC | 1815 | GTCGAGACAACATGAGTGTG |
| 862 | 1317 | TCACACTCATGTTGTCTCGA | 1816 | TCGAGACAACATGAGTGTGA |
| 863 | 1318 | ATCACACTCATGTTGTCTCG | 1817 | CGAGACAACATGAGTGTGAT |
| 864 | 1319 | AATCACACTCATGTTGTCTC | 1818 | GAGACAACATGAGTGTGATT |
| 865 | 1320 | AAATCACACTCATGTTGTCT | 1819 | AGACAACATGAGTGTGATTT |
| 866 | 1321 | AAAATCACACTCATGTTGTC | 1820 | GACAACATGAGTGTGATTTT |
| 867 | 1322 | CAAAATCACACTCATGTTGT | 1821 | ACAACATGAGTGTGATTTTG |
| 868 | 1323 | TCAAAATCACACTCATGTTG | 1822 | CAACATGAGTGTGATTTTGA |
| 869 | 1324 | ATCAAAATCACACTCATGTT | 1823 | AACATGAGTGTGATTTTGAT |
| 870 | 1325 | GATCAAAATCACACTCATGT | 1824 | ACATGAGTGTGATTTTGATC |
| 871 | 1326 | AGATCAAAATCACACTCATG | 1825 | CATGAGTGTGATTTTGATCT |
| 872 | 1327 | CAGATCAAAATCACACTCAT | 1826 | ATGAGTGTGATTTTGATCTG |
| 873 | 1328 | ACAGATCAAAATCACACTCA | 1827 | TGAGTGTGATTTTGATCTGT |
| 874 | 1329 | AACAGATCAAAATCACACTC | 1828 | GAGTGTGATTTTGATCTGTT |
| 875 | 1330 | AAACAGATCAAAATCACACT | 1829 | AGTGTGATTTTGATCTGTTT |
| 876 | 1331 | AAAACAGATCAAAATCACAC | 1830 | GTGTGATTTTGATCTGTTTT |
| 877 | 1332 | GAAAACAGATCAAAATCACA | 1831 | TGTGATTTTGATCTGTTTTC |
| 878 | 1333 | GGAAAACAGATCAAAATCAC | 1832 | GTGATTTTGATCTGTTTTCC |
| 879 | 1334 | TGGAAAACAGATCAAAATCA | 1833 | TGATTTTGATCTGTTTTCCA |
| 880 | 1335 | TTGGAAAACAGATCAAAATC | 1834 | GATTTTGATCTGTTTTCCAA |
| 881 | 1336 | TTTGGAAAACAGATCAAAAT | 1835 | ATTTTGATCTGTTTTCCAAA |
| 882 | 1337 | ATTTGGAAAACAGATCAAAA | 1836 | TTTTGATCTGTTTTCCAAAT |
| 883 | 1338 | CATTTGGAAAACAGATCAAA | 1837 | TTTGATCTGTTTTCCAAATG |
| 884 | 1339 | GCATTTGGAAAACAGATCAA | 1838 | TTGATCTGTTTTCCAAATGC |
| 885 | 1340 | TGCATTTGGAAAACAGATCA | 1839 | TGATCTGTTTTCCAAATGCA |
| 886 | 1341 | GTGCATTTGGAAAACAGATC | 1840 | GATCTGTTTTCCAAATGCAC |
| 887 | 1342 | GGTGCATTTGGAAAACAGAT | 1841 | ATCTGTTTTCCAAATGCACC |
| 888 | 1343 | GGGTGCATTTGGAAAACAGA | 1842 | TCTGTTTTCCAAATGCACCC |
| 889 | 1344 | TGGGTGCATTTGGAAAACAG | 1843 | CTGTTTTCCAAATGCACCCA |
| 890 | 1345 | TTGGGTGCATTTGGAAAACA | 1844 | TGTTTTCCAAATGCACCCAA |
| 891 | 1346 | TTTGGGTGCATTTGGAAAAC | 1845 | GTTTTCCAAATGCACCCAAA |
| 892 | 1347 | CTTTGGGTGCATTTGGAAAA | 1846 | TTTTCCAAATGCACCCAAAG |
| 893 | 1348 | ACTTTGGGTGCATTTGGAAA | 1847 | TTTCCAAATGCACCCAAAGT |
| 894 | 1349 | TACTTTGGGTGCATTTGGAA | 1848 | TTCCAAATGCACCCAAAGTA |
| 895 | 1350 | ATACTTTGGGTGCATTTGGA | 1849 | TCCAAATGCACCCAAAGTAT |
| 896 | 1351 | GATACTTTGGGTGCATTTGG | 1850 | CCAAATGCACCCAAAGTATC |
| 897 | 1352 | CGATACTTTGGGTGCATTTG | 1851 | CAAATGCACCCAAAGTATCG |
| 898 | 1353 | GCGATACTTTGGGTGCATTT | 1852 | AAATGCACCCAAAGTATCGC |
| 899 | 1354 | GGCGATACTTTGGGTGCATT | 1853 | AATGCACCCAAAGTATCGCC |
| 900 | 1355 | TGGCGATACTTTGGGTGCAT | 1854 | ATGCACCCAAAGTATCGCCA |
| 901 | 1356 | CTGGCGATACTTTGGGTGCA | 1855 | TGCACCCAAAGTATCGCCAG |
| 902 | 1357 | TCTGGCGATACTTTGGGTGC | 1856 | GCACCCAAAGTATCGCCAGA |
| 903 | 1358 | TTCTGGCGATACTTTGGGTG | 1857 | CACCCAAAGTATCGCCAGAA |
| 904 | 1359 | CTTCTGGCGATACTTTGGGT | 1858 | ACCCAAAGTATCGCCAGAAG |
| 905 | 1360 | GCTTCTGGCGATACTTTGGG | 1859 | CCCAAAGTATCGCCAGAAGC |
| 906 | 1361 | TGCTTCTGGCGATACTTTGG | 1860 | CCAAAGTATCGCCAGAAGCA |
| 907 | 1362 | CTGCTTCTGGCGATACTTTG | 1861 | CAAAGTATCGCCAGAAGCAG |
| 908 | 1363 | ACTGCTTCTGGCGATACTTT | 1862 | AAAGTATCGCCAGAAGCAGT |
| 909 | 1364 | CACTGCTTCTGGCGATACTT | 1863 | AAGTATCGCCAGAAGCAGTG |
| 910 | 1365 | TCACTGCTTCTGGCGATACT | 1864 | AGTATCGCCAGAAGCAGTGA |
| 911 | 1366 | TTCACTGCTTCTGGCGATAC | 1865 | GTATCGCCAGAAGCAGTGAA |
| 912 | 1367 | CTTCACTGCTTCTGGCGATA | 1866 | TATCGCCAGAAGCAGTGAAG |
| 913 | 1368 | TCTTCACTGCTTCTGGCGAT | 1867 | ATCGCCAGAAGCAGTGAAGA |
| 914 | 1369 | TTCTTCACTGCTTCTGGCGA | 1868 | TCGCCAGAAGCAGTGAAGAA |
| 915 | 1370 | CTTCTTCACTGCTTCTGGCG | 1869 | CGCCAGAAGCAGTGAAGAAG |
| 916 | 1371 | CCTTCTTCACTGCTTCTGGC | 1870 | GCCAGAAGCAGTGAAGAAGG |
| 917 | 1372 | TCCTTCTTCACTGCTTCTGG | 1871 | CCAGAAGCAGTGAAGAAGGA |
| 918 | 1373 | CTCCTTCTTCACTGCTTCTG | 1872 | CAGAAGCAGTGAAGAAGGAG |
| 919 | 1374 | CCTCCTTCTTCACTGCTTCT | 1873 | AGAAGCAGTGAAGAAGGAGG |
| 920 | 1375 | GCCTCCTTCTTCACTGCTTC | 1874 | GAAGCAGTGAAGAAGGAGGC |
| 921 | 1376 | TGCCTCCTTCTTCACTGCTT | 1875 | AAGCAGTGAAGAAGGAGGCA |
| 922 | 1377 | CTGCCTCCTTCTTCACTGCT | 1876 | AGCAGTGAAGAAGGAGGCAG |
| 923 | 1378 | TCTGCCTCCTTCTTCACTGC | 1877 | GCAGTGAAGAAGGAGGCAGA |
| 924 | 1379 | CTCTGCCTCCTTCTTCACTG | 1878 | CAGTGAAGAAGGAGGCAGAG |
| 925 | 1380 | ACTCTGCCTCCTTCTTCACT | 1879 | AGTGAAGAAGGAGGCAGAGT |
| 926 | 1381 | AACTCTGCCTCCTTCTTCAC | 1880 | GTGAAGAAGGAGGCAGAGTT |
| 927 | 1382 | CAACTCTGCCTCCTTCTTCA | 1881 | TGAAGAAGGAGGCAGAGTTG |
| 928 | 1383 | CCAACTCTGCCTCCTTCTTC | 1882 | GAAGAAGGAGGCAGAGTTGG |
| 929 | 1384 | TCCAACTCTGCCTCCTTCTT | 1883 | AAGAAGGAGGCAGAGTTGGA |
| 930 | 1385 | GTCCAACTCTGCCTCCTTCT | 1884 | AGAAGGAGGCAGAGTTGGAC |
| 931 | 1386 | TGTCCAACTCTGCCTCCTTC | 1885 | GAAGGAGGCAGAGTTGGACA |
| 932 | 1387 | TTGTCCAACTCTGCCTCCTT | 1886 | AAGGAGGCAGAGTTGGACAA |
| 933 | 1388 | CTTGTCCAACTCTGCCTCCT | 1887 | AGGAGGCAGAGTTGGACAAG |
| 934 | 1389 | ACTTGTCCAACTCTGCCTCC | 1888 | GGAGGCAGAGTTGGACAAGT |
| 935 | 1390 | TACTTGTCCAACTCTGCCTC | 1889 | GAGGCAGAGTTGGACAAGTA |
| 936 | 1391 | GTACTTGTCCAACTCTGCCT | 1890 | AGGCAGAGTTGGACAAGTAC |
| 937 | 1392 | GGTACTTGTCCAACTCTGCC | 1891 | GGCAGAGTTGGACAAGTACC |
| 938 | 1393 | AGGTACTTGTCCAACTCTGC | 1892 | GCAGAGTTGGACAAGTACCT |
| 939 | 1394 | CAGGTACTTGTCCAACTCTG | 1893 | CAGAGTTGGACAAGTACCTG |
| 940 | 1395 | CCAGGTACTTGTCCAACTCT | 1894 | AGAGTTGGACAAGTACCTGG |
| 941 | 1396 | TCCAGGTACTTGTCCAACTC | 1895 | GAGTTGGACAAGTACCTGGA |
| 942 | 1397 | TTCCAGGTACTTGTCCAACT | 1896 | AGTTGGACAAGTACCTGGAA |
| 943 | 1398 | ATTCCAGGTACTTGTCCAAC | 1897 | GTTGGACAAGTACCTGGAAT |
| 944 | 1399 | CATTCCAGGTACTTGTCCAA | 1898 | TTGGACAAGTACCTGGAATG |
| 945 | 1400 | GCATTCCAGGTACTTGTCCA | 1899 | TGGACAAGTACCTGGAATGC |
| 946 | 1401 | TGCATTCCAGGTACTTGTCC | 1900 | GGACAAGTACCTGGAATGCA |
| 947 | 1402 | CTGCATTCCAGGTACTTGTC | 1901 | GACAAGTACCTGGAATGCAG |
| 948 | 1403 | TCTGCATTCCAGGTACTTGT | 1902 | ACAAGTACCTGGAATGCAGA |
| 949 | 1404 | CTCTGCATTCCAGGTACTTG | 1903 | CAAGTACCTGGAATGCAGAG |
| 950 | 1405 | ACTCTGCATTCCAGGTACTT | 1904 | AAGTACCTGGAATGCAGAGT |
| 951 | 1406 | TACTCTGCATTCCAGGTACT | 1905 | AGTACCTGGAATGCAGAGTA |
| 952 | 1407 | CTACTCTGCATTCCAGGTAC | 1906 | GTACCTGGAATGCAGAGTAG |
| 953 | 1408 | TCTACTCTGCATTCCAGGTA | 1907 | TACCTGGAATGCAGAGTAGA |
| 954 | 1409 | TTCTACTCTGCATTCCAGGT | 1908 | ACCTGGAATGCAGAGTAGAA |
| 955 | 1410 | CTTCTACTCTGCATTCCAGG | 1909 | CCTGGAATGCAGAGTAGAAG |

*At least one nucleoside linkage of the oligonucleotide is selected from a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a sclenophosphate linkage, and a boranophosphate linkage

Table 2 below identifies PPM1A AON sequences.

TABLE 2
PPM1A AON Sequences. In comparison to Table 1,
the PPM1A AON sequences here have uracil
nucleobases in place of thymine nucleobases.

| SEQ | | SEQ | |
|---|---|---|---|
| ID | AON | ID | |
| NO: | Sequence (5′→3′) | NO: | AON Sequence (5′→3′) |
| 1910 | AUGUCUUGAUCCUCUAGGUC | 2387 | AAAUGAACUUUCCUGUUCCU |
| 1911 | UAUGUCUUGAUCCUCUAGGU | 2388 | GAAAUGAACUUUCCUGUUCC |
| 1912 | UUAUGUCUUGAUCCUCUAGG | 2389 | AGAAAUGAACUUUCCUGUUC |
| 1913 | AUUAUGUCUUGAUCCUCUAG | 2390 | AAGAAAUGAACUUUCCUGUU |
| 1914 | CAUUAUGUCUUGAUCCUCUA | 2391 | GAAGAAAUGAACUUUCCUGU |
| 1915 | CCAUUAUGUCUUGAUCCUCU | 2392 | UGAAGAAAUGAACUUUCCUG |
| 1916 | CCCAUUAUGUCUUGAUCCUC | 2393 | GUGAAGAAAUGAACUUUCCU |
| 1917 | UCCCAUUAUGUCUUGAUCCU | 2394 | UGUGAAGAAAUGAACUUUCC |
| 1918 | CUCCCAUUAUGUCUUGAUCC | 2395 | GUGUGAAGAAAUGAACUUUC |
| 1919 | GCUCCCAUUAUGUCUUGAUC | 2396 | UGUGUGAAGAAAUGAACUUU |
| 1920 | UGCUCCCAUUAUGUCUUGAU | 2397 | UUGUGUGAAGAAAUGAACUU |
| 1921 | AUGCUCCCAUUAUGUCUUGA | 2398 | CUUGUGUGAAGAAAUGAACU |
| 1922 | AAUGCUCCCAUUAUGUCUUG | 2399 | UCUUGUGUGAAGAAAUGAAC |
| 1923 | AAAUGCUCCCAUUAUGUCUU | 2400 | AUCUUGUGUGAAGAAAUGAA |
| 1924 | AAAAUGCUCCCAUUAUGUCU | 2401 | GAUCUUGUGUGAAGAAAUGA |
| 1925 | AAAAAUGCUCCCAUUAUGUC | 2402 | UGAUCUUGUGUGAAGAAAUG |
| 1926 | UAAAAAUGCUCCCAUUAUGU | 2403 | GUGAUCUUGUGUGAAGAAAU |
| 1927 | CUAAAAAUGCUCCCAUUAUG | 2404 | UGUGAUCUUGUGUGAAGAAA |
| 1928 | UCUAAAAAUGCUCCCAUUAU | 2405 | UUGUGAUCUUGUGUGAAGAA |
| 1929 | GUCUAAAAAUGCUCCCAUUA | 2406 | UUUGUGAUCUUGUGUGAAGA |
| 1930 | UGUCUAAAAAUGCUCCCAUU | 2407 | GUUUGUGAUCUUGUGUGAAG |
| 1931 | UUGUCUAAAAAUGCUCCCAU | 2408 | GGUUUGUGAUCUUGUGUGAA |
| 1932 | CUUGUCUAAAAAUGCUCCCA | 2409 | UGGUUUGUGAUCUUGUGUGA |
| 1933 | GCUUGUCUAAAAAUGCUCCC | 2410 | UUGGUUUGUGAUCUUGUGUG |
| 1934 | GGCUUGUCUAAAAAUGCUCC | 2411 | CUUGGUUUGUGAUCUUGUGU |
| 1935 | UGGCUUGUCUAAAAAUGCUC | 2412 | ACUUGGUUUGUGAUCUUGUG |
| 1936 | UUGGCUUGUCUAAAAAUGCU | 2413 | UACUUGGUUUGUGAUCUUGU |
| 1937 | UUUGGCUUGUCUAAAAAUGC | 2414 | UUACUUGGUUUGUGAUCUUG |
| 1938 | CUUUGGCUUGUCUAAAAAUG | 2415 | AUUACUUGGUUUGUGAUCUU |
| 1939 | UCUUUGGCUUGUCUAAAAAU | 2416 | GAUUACUUGGUUUGUGAUCU |
| 1940 | AUCUUUGGCUUGUCUAAAAA | 2417 | GGAUUACUUGGUUUGUGAUC |
| 1941 | CAUCUUUGGCUUGUCUAAAA | 2418 | CGGAUUACUUGGUUUGUGAU |
| 1942 | CCAUCUUUGGCUUGUCUAAA | 2419 | GCGGAUUACUUGGUUUGUGA |
| 1943 | UCCAUCUUUGGCUUGUCUAA | 2420 | AGCGGAUUACUUGGUUUGUG |
| 1944 | UUCCAUCUUUGGCUUGUCUA | 2421 | CAGCGGAUUACUUGGUUUGU |
| 1945 | UUUCCAUCUUUGGCUUGUCU | 2422 | CCAGCGGAUUACUUGGUUUG |
| 1946 | UUUUCCAUCUUUGGCUUGUC | 2423 | UCCAGCGGAUUACUUGGUUU |
| 1947 | CUUUUCCAUCUUUGGCUUGU | 2424 | CUCCAGCGGAUUACUUGGUU |
| 1948 | GCUUUUCCAUCUUUGGCUUG | 2425 | UCUCCAGCGGAUUACUUGGU |
| 1949 | UGCUUUUCCAUCUUUGGCUU | 2426 | UUCUCCAGCGGAUUACUUGG |
| 1950 | AUGCUUUUCCAUCUUUGGCU | 2427 | UUUCUCCAGCGGAUUACUUG |
| 1951 | UAUGCUUUUCCAUCUUUGGC | 2428 | CUUUCUCCAGCGGAUUACUU |
| 1952 | UUAUGCUUUUCCAUCUUUGG | 2429 | UCUUUCUCCAGCGGAUUACU |
| 1953 | AUUAUGCUUUUCCAUCUUUG | 2430 | UUCUUUCUCCAGCGGAUUAC |
| 1954 | CAUUAUGCUUUUCCAUCUUU | 2431 | GUUCUUUCUCCAGCGGAUUA |
| 1955 | GCAUUAUGCUUUUCCAUCUU | 2432 | CGUUCUUUCUCCAGCGGAUU |
| 1956 | GGCAUUAUGCUUUUCCAUCU | 2433 | UCGUUCUUUCUCCAGCGGAU |
| 1957 | GGGCAUUAUGCUUUUCCAUC | 2434 | UUCGUUCUUUCUCCAGCGGA |
| 1958 | UGGGCAUUAUGCUUUUCCAU | 2435 | AUUCGUUCUUUCUCCAGCGG |
| 1959 | CUGGGCAUUAUGCUUUUCCA | 2436 | AAUUCGUUCUUUCUCCAGCG |
| 1960 | CCUGGGCAUUAUGCUUUUCC | 2437 | GAAUUCGUUCUUUCUCCAGC |
| 1961 | CCCUGGGCAUUAUGCUUUUC | 2438 | UGAAUUCGUUCUUUCUCCAG |
| 1962 | CCCCUGGGCAUUAUGCUUUU | 2439 | CUGAAUUCGUUCUUUCUCCA |
| 1963 | GCCCCUGGGCAUUAUGCUUU | 2440 | UCUGAAUUCGUUCUUUCUCC |
| 1964 | UGCCCCUGGGCAUUAUGCUU | 2441 | UUCUGAAUUCGUUCUUUCUC |
| 1965 | CUGCCCCUGGGCAUUAUGCU | 2442 | AUUCUGAAUUCGUUCUUUCU |
| 1966 | CCUGCCCCUGGGCAUUAUGC | 2443 | CAUUCUGAAUUCGUUCUUUC |
| 1967 | CCCUGCCCCUGGGCAUUAUG | 2444 | GCAUUCUGAAUUCGUUCUUU |
| 1968 | ACCCUGCCCCUGGGCAUUAU | 2445 | UGCAUUCUGAAUUCGUUCUU |
| 1969 | UACCCUGCCCCUGGGCAUUA | 2446 | CUGCAUUCUGAAUUCGUUCU |
| 1970 | UUACCCUGCCCCUGGGCAUU | 2447 | CCUGCAUUCUGAAUUCGUUC |
| 1971 | AUUACCCUGCCCCUGGGCAU | 2448 | ACCUGCAUUCUGAAUUCGUU |
| 1972 | CAUUACCCUGCCCCUGGGCA | 2449 | CACCUGCAUUCUGAAUUCGU |
| 1973 | CCAUUACCCUGCCCCUGGGC | 2450 | CCACCUGCAUUCUGAAUUCG |
| 1974 | CCCAUUACCCUGCCCCUGGG | 2451 | GCCACCUGCAUUCUGAAUUC |
| 1975 | ACCCAUUACCCUGCCCCUGG | 2452 | AGCCACCUGCAUUCUGAAUU |
| 1976 | AACCCAUUACCCUGCCCCUG | 2453 | GAGCCACCUGCAUUCUGAAU |
| 1977 | CAACCCAUUACCCUGCCCCU | 2454 | AGAGCCACCUGCAUUCUGAA |
| 1978 | GCAACCCAUUACCCUGCCCC | 2455 | CAGAGCCACCUGCAUUCUGA |
| 1979 | CGCAACCCAUUACCCUGCCC | 2456 | ACAGAGCCACCUGCAUUCUG |
| 1980 | UCGCAACCCAUUACCCUGCC | 2457 | UACAGAGCCACCUGCAUUCU |
| 1981 | AUCGCAACCCAUUACCCUGC | 2458 | UUACAGAGCCACCUGCAUUC |
| 1982 | UAUCGCAACCCAUUACCCUG | 2459 | AUUACAGAGCCACCUGCAUU |
| 1983 | AUAUCGCAACCCAUUACCCU | 2460 | CAUUACAGAGCCACCUGCAU |
| 1984 | CAUAUCGCAACCCAUUACCC | 2461 | UCAUUACAGAGCCACCUGCA |
| 1985 | CCAUAUCGCAACCCAUUACC | 2462 | AUCAUUACAGAGCCACCUGC |
| 1986 | CCCAUAUCGCAACCCAUUAC | 2463 | AAUCAUUACAGAGCCACCUG |
| 1987 | GCCCAUAUCGCAACCCAUUA | 2464 | GAAUCAUUACAGAGCCACCU |
| 1988 | AGCCCAUAUCGCAACCCAUU | 2465 | UGAAUCAUUACAGAGCCACC |
| 1989 | UAGCCCAUAUCGCAACCCAU | 2466 | CUGAAUCAUUACAGAGCCAC |
| 1990 | UUAGCCCAUAUCGCAACCCA | 2467 | GCUGAAUCAUUACAGAGCCA |
| 1991 | CUUAGCCCAUAUCGCAACCC | 2468 | CGCUGAAUCAUUACAGAGCC |
| 1992 | GCUUAGCCCAUAUCGCAACC | 2469 | ACGCUGAAUCAUUACAGAGC |
| 1993 | UGCUUAGCCCAUAUCGCAAC | 2470 | CACGCUGAAUCAUUACAGAG |
| 1994 | CUGCUUAGCCCAUAUCGCAA | 2471 | ACACGCUGAAUCAUUACAGA |
| 1995 | GCUGCUUAGCCCAUAUCGCA | 2472 | CACACGCUGAAUCAUUACAG |
| 1996 | UGCUGCUUAGCCCAUAUCGC | 2473 | UCACACGCUGAAUCAUUACA |
| 1997 | AUGCUGCUUAGCCCAUAUCG | 2474 | UUCACACGCUGAAUCAUUAC |
| 1998 | CAUGCUGCUUAGCCCAUAUC | 2475 | AUUCACACGCUGAAUCAUUA |
| 1999 | GCAUGCUGCUUAGCCCAUAU | 2476 | CAUUCACACGCUGAAUCAUU |
| 2000 | UGCAUGCUGCUUAGCCCAUA | 2477 | CCAUUCACACGCUGAAUCAU |
| 2001 | UUGCAUGCUGCUUAGCCCAU | 2478 | GCCAUUCACACGCUGAAUCA |
| 2002 | CUUGCAUGCUGCUUAGCCCA | 2479 | AGCCAUUCACACGCUGAAUC |
| 2003 | CCUUGCAUGCUGCUUAGCCC | 2480 | GAGCCAUUCACACGCUGAAU |
| 2004 | GCCUUGCAUGCUGCUUAGCC | 2481 | AGAGCCAUUCACACGCUGAA |
| 2005 | AGCCUUGCAUGCUGCUUAGC | 2482 | GAGAGCCAUUCACACGCUGA |
| 2006 | CAGCCUUGCAUGCUGCUUAG | 2483 | AGAGAGCCAUUCACACGCUG |
| 2007 | CCAGCCUUGCAUGCUGCUUA | 2484 | CAGAGAGCCAUUCACACGCU |
| 2008 | GCCAGCCUUGCAUGCUGCUU | 2485 | CCAGAGAGCCAUUCACACGC |
| 2009 | CGCCAGCCUUGCAUGCUGCU | 2486 | GCCAGAGAGCCAUUCACACG |
| 2010 | ACGCCAGCCUUGCAUGCUGC | 2487 | AGCCAGAGAGCCAUUCACAC |
| 2011 | CACGCCAGCCUUGCAUGCUG | 2488 | CAGCCAGAGAGCCAUUCACA |
| 2012 | ACACGCCAGCCUUGCAUGCU | 2489 | ACAGCCAGAGAGCCAUUCAC |
| 2013 | AACACGCCAGCCUUGCAUGC | 2490 | UACAGCCAGAGAGCCAUUCA |
| 2014 | CAACACGCCAGCCUUGCAUG | 2491 | AUACAGCCAGAGAGCCAUUC |
| 2015 | UCAACACGCCAGCCUUGCAU | 2492 | GAUACAGCCAGAGAGCCAUU |
| 2016 | UUCAACACGCCAGCCUUGCA | 2493 | CGAUACAGCCAGAGAGCCAU |
| 2017 | UUUCAACACGCCAGCCUUGC | 2494 | UCGAUACAGCCAGAGAGCCA |
| 2018 | AUUUCAACACGCCAGCCUUG | 2495 | CUCGAUACAGCCAGAGAGCC |
| 2019 | CAUUUCAACACGCCAGCCUU | 2496 | CCUCGAUACAGCCAGAGAGC |
| 2020 | CCAUUUCAACACGCCAGCCU | 2497 | CCCUCGAUACAGCCAGAGAG |
| 2021 | UCCAUUUCAACACGCCAGCC | 2498 | GCCCUCGAUACAGCCAGAGA |
| 2022 | CUCCAUUUCAACACGCCAGC | 2499 | GGCCCUCGAUACAGCCAGAG |
| 2023 | CCUCCAUUUCAACACGCCAG | 2500 | GGGCCCUCGAUACAGCCAGA |
| 2024 | UCCUCCAUUUCAACACGCCA | 2501 | AGGGCCCUCGAUACAGCCAG |
| 2025 | AUCCUCCAUUUCAACACGCC | 2502 | AAGGGCCCUCGAUACAGCCA |
| 2026 | CAUCCUCCAUUUCAACACGC | 2503 | CAAGGGCCCUCGAUACAGCC |
| 2027 | GCAUCCUCCAUUUCAACACG | 2504 | CCAAGGGCCCUCGAUACAGC |
| 2028 | UGCAUCCUCCAUUUCAACAC | 2505 | CCCAAGGGCCCUCGAUACAG |
| 2029 | GUGCAUCCUCCAUUUCAACA | 2506 | CCCCAAGGGCCCUCGAUACA |
| 2030 | UGUGCAUCCUCCAUUUCAAC | 2507 | UCCCCAAGGGCCCUCGAUAC |
| 2031 | AUGUGCAUCCUCCAUUUCAA | 2508 | AUCCCCAAGGGCCCUCGAUA |
| 2032 | UAUGUGCAUCCUCCAUUUCA | 2509 | AAUCCCCAAGGGCCCUCGAU |
| 2033 | GUAUGUGCAUCCUCCAUUUC | 2510 | AAAUCCCCAAGGGCCCUCGA |
| 2034 | CGUAUGUGCAUCCUCCAUUU | 2511 | AAAAUCCCCAAGGGCCCUCG |
| 2035 | CCGUAUGUGCAUCCUCCAUU | 2512 | CAAAAUCCCCAAGGGCCCUC |
| 2036 | GCCGUAUGUGCAUCCUCCAU | 2513 | UCAAAAUCCCCAAGGGCCCU |
| 2037 | AGCCGUAUGUGCAUCCUCCA | 2514 | AUCAAAAUCCCCAAGGGCCC |
| 2038 | CAGCCGUAUGUGCAUCCUCC | 2515 | AAUCAAAAUCCCCAAGGGCC |
| 2039 | ACAGCCGUAUGUGCAUCCUC | 2516 | UAAUCAAAAUCCCCAAGGGC |
| 2040 | CACAGCCGUAUGUGCAUCCU | 2517 | GUAAUCAAAAUCCCCAAGGG |
| 2041 | UCACAGCCGUAUGUGCAUCC | 2518 | UGUAAUCAAAAUCCCCAAGG |
| 2042 | AUCACAGCCGUAUGUGCAUC | 2519 | UUGUAAUCAAAAUCCCCAAG |
| 2043 | GAUCACAGCCGUAUGUGCAU | 2520 | UUUGUAAUCAAAAUCCCCAA |
| 2044 | CGAUCACAGCCGUAUGUGCA | 2521 | AUUUGUAAUCAAAAUCCCCA |
| 2045 | CCGAUCACAGCCGUAUGUGC | 2522 | CAUUUGUAAUCAAAAUCCCC |
| 2046 | ACCGAUCACAGCCGUAUGUG | 2523 | ACAUUUGUAAUCAAAAUCCC |
| 2047 | AACCGAUCACAGCCGUAUGU | 2524 | CACAUUUGUAAUCAAAAUCC |
| 2048 | AAACCGAUCACAGCCGUAUG | 2525 | ACACAUUUGUAAUCAAAAUC |
| 2049 | CAAACCGAUCACAGCCGUAU | 2526 | GACACAUUUGUAAUCAAAAU |
| 2050 | GCAAACCGAUCACAGCCGUA | 2527 | GGACACAUUUGUAAUCAAAA |
| 2051 | GGCAAACCGAUCACAGCCGU | 2528 | UGGACACAUUUGUAAUCAAA |
| 2052 | UGGCAAACCGAUCACAGCCG | 2529 | AUGGACACAUUUGUAAUCAA |
| 2053 | UUGGCAAACCGAUCACAGCC | 2530 | CAUGGACACAUUUGUAAUCA |
| 2054 | CUUGGCAAACCGAUCACAGC | 2531 | CCAUGGACACAUUUGUAAUC |
| 2055 | ACUUGGCAAACCGAUCACAG | 2532 | UCCAUGGACACAUUUGUAAU |
| 2056 | CACUUGGCAAACCGAUCACA | 2533 | UUCCAUGGACACAUUUGUAA |
| 2057 | CCACUUGGCAAACCGAUCAC | 2534 | UUUCCAUGGACACAUUUGUA |
| 2058 | UCCACUUGGCAAACCGAUCA | 2535 | UUUUCCAUGGACACAUUUGU |
| 2059 | GUCCACUUGGCAAACCGAUC | 2536 | CUUUUCCAUGGACACAUUUG |
| 2060 | AGUCCACUUGGCAAACCGAU | 2537 | CCUUUUCCAUGGACACAUUU |
| 2061 | AAGUCCACUUGGCAAACCGA | 2538 | ACCUUUUCCAUGGACACAUU |
| 2062 | CAAGUCCACUUGGCAAACCG | 2539 | GACCUUUUCCAUGGACACAU |
| 2063 | UCAAGUCCACUUGGCAAACC | 2540 | GGACCUUUUCCAUGGACACA |
| 2064 | UUCAAGUCCACUUGGCAAAC | 2541 | AGGACCUUUUCCAUGGACAC |
| 2065 | AUUCAAGUCCACUUGGCAAA | 2542 | UAGGACCUUUUCCAUGGACA |
| 2066 | GAUUCAAGUCCACUUGGCAA | 2543 | GUAGGACCUUUUCCAUGGAC |
| 2067 | CGAUUCAAGUCCACUUGGCA | 2544 | AGUAGGACCUUUUCCAUGGA |
| 2068 | ACGAUUCAAGUCCACUUGGC | 2545 | CAGUAGGACCUUUUCCAUGG |
| 2069 | CACGAUUCAAGUCCACUUGG | 2546 | UCAGUAGGACCUUUUCCAUG |
| 2070 | CCACGAUUCAAGUCCACUUG | 2547 | CUCAGUAGGACCUUUUCCAU |
| 2071 | ACCACGAUUCAAGUCCACUU | 2548 | GCUCAGUAGGACCUUUUCCA |
| 2072 | GACCACGAUUCAAGUCCACU | 2549 | UGCUCAGUAGGACCUUUUCC |
| 2073 | UGACCACGAUUCAAGUCCAC | 2550 | CUGCUCAGUAGGACCUUUUC |
| 2074 | AUGACCACGAUUCAAGUCCA | 2551 | GCUGCUCAGUAGGACCUUUU |
| 2075 | AAUGACCACGAUUCAAGUCC | 2552 | AGCUGCUCAGUAGGACCUUU |
| 2076 | GAAUGACCACGAUUCAAGUC | 2553 | AAGCUGCUCAGUAGGACCUU |
| 2077 | AGAAUGACCACGAUUCAAGU | 2554 | CAAGCUGCUCAGUAGGACCU |
| 2078 | AAGAAUGACCACGAUUCAAG | 2555 | ACAAGCUGCUCAGUAGGACC |
| 2079 | AAAGAAUGACCACGAUUCAA | 2556 | GACAAGCUGCUCAGUAGGAC |
| 2080 | CAAAGAAUGACCACGAUUCA | 2557 | AGACAAGCUGCUCAGUAGGA |
| 2081 | GCAAAGAAUGACCACGAUUC | 2558 | GAGACAAGCUGCUCAGUAGG |
| 2082 | AGCAAAGAAUGACCACGAUU | 2559 | UGAGACAAGCUGCUCAGUAG |
| 2083 | CAGCAAAGAAUGACCACGAU | 2560 | GUGAGACAAGCUGCUCAGUA |
| 2084 | ACAGCAAAGAAUGACCACGA | 2561 | GGUGAGACAAGCUGCUCAGU |
| 2085 | CACAGCAAAGAAUGACCACG | 2562 | UGGUGAGACAAGCUGCUCAG |
| 2086 | ACACAGCAAAGAAUGACCAC | 2563 | CUGGUGAGACAAGCUGCUCA |
| 2087 | UACACAGCAAAGAAUGACCA | 2564 | UCUGGUGAGACAAGCUGCUC |
| 2088 | AUACACAGCAAAGAAUGACC | 2565 | CUCUGGUGAGACAAGCUGCU |
| 2089 | CAUACACAGCAAAGAAUGAC | 2566 | GCUCUGGUGAGACAAGCUGC |
| 2090 | UCAUACACAGCAAAGAAUGA | 2567 | GGCUCUGGUGAGACAAGCUG |
| 2091 | AUCAUACACAGCAAAGAAUG | 2568 | AGGCUCUGGUGAGACAAGCU |
| 2092 | CAUCAUACACAGCAAAGAAU | 2569 | CAGGCUCUGGUGAGACAAGC |
| 2093 | CCAUCAUACACAGCAAAGAA | 2570 | UCAGGCUCUGGUGAGACAAG |
| 2094 | CCCAUCAUACACAGCAAAGA | 2571 | UUCAGGCUCUGGUGAGACAA |
| 2095 | GCCCAUCAUACACAGCAAAG | 2572 | CUUCAGGCUCUGGUGAGACA |
| 2096 | UGCCCAUCAUACACAGCAAA | 2573 | ACUUCAGGCUCUGGUGAGAC |
| 2097 | AUGCCCAUCAUACACAGCAA | 2574 | GACUUCAGGCUCUGGUGAGA |
| 2098 | CAUGCCCAUCAUACACAGCA | 2575 | GGACUUCAGGCUCUGGUGAG |
| 2099 | GCAUGCCCAUCAUACACAGC | 2576 | UGGACUUCAGGCUCUGGUGA |
| 2100 | AGCAUGCCCAUCAUACACAG | 2577 | AUGGACUUCAGGCUCUGGUG |
| 2101 | CAGCAUGCCCAUCAUACACA | 2578 | CAUGGACUUCAGGCUCUGGU |
| 2102 | CCAGCAUGCCCAUCAUACAC | 2579 | UCAUGGACUUCAGGCUCUGG |
| 2103 | ACCAGCAUGCCCAUCAUACA | 2580 | AUCAUGGACUUCAGGCUCUG |
| 2104 | AACCAGCAUGCCCAUCAUAC | 2581 | UAUCAUGGACUUCAGGCUCU |
| 2105 | GAACCAGCAUGCCCAUCAUA | 2582 | AUAUCAUGGACUUCAGGCUC |
| 2106 | AGAACCAGCAUGCCCAUCAU | 2583 | AAUAUCAUGGACUUCAGGCU |
| 2107 | GAGAACCAGCAUGCCCAUCA | 2584 | CAAUAUCAUGGACUUCAGGC |
| 2108 | UGAGAACCAGCAUGCCCAUC | 2585 | UCAAUAUCAUGGACUUCAGG |
| 2109 | CUGAGAACCAGCAUGCCCAU | 2586 | UUCAAUAUCAUGGACUUCAG |
| 2110 | CCUGAGAACCAGCAUGCCCA | 2587 | UUUCAAUAUCAUGGACUUCA |
| 2111 | ACCUGAGAACCAGCAUGCCC | 2588 | CUUUCAAUAUCAUGGACUUC |
| 2112 | AACCUGAGAACCAGCAUGCC | 2589 | UCUUUCAAUAUCAUGGACUU |
| 2113 | CAACCUGAGAACCAGCAUGC | 2590 | AUCUUUCAAUAUCAUGGACU |
| 2114 | GCAACCUGAGAACCAGCAUG | 2591 | GAUCUUUCAAUAUCAUGGAC |
| 2115 | GGCAACCUGAGAACCAGCAU | 2592 | AGAUCUUUCAAUAUCAUGGA |
| 2116 | UGGCAACCUGAGAACCAGCA | 2593 | CAGAUCUUUCAAUAUCAUGG |
| 2117 | UUGGCAACCUGAGAACCAGC | 2594 | UCAGAUCUUUCAAUAUCAUG |
| 2118 | UUUGGCAACCUGAGAACCAG | 2595 | UUCAGAUCUUUCAAUAUCAU |
| 2119 | AUUUGGCAACCUGAGAACCA | 2596 | CUUCAGAUCUUUCAAUAUCA |
| 2120 | UAUUUGGCAACCUGAGAACC | 2597 | UCUUCAGAUCUUUCAAUAUC |
| 2121 | GUAUUUGGCAACCUGAGAAC | 2598 | UUCUUCAGAUCUUUCAAUAU |
| 2122 | AGUAUUUGGCAACCUGAGAA | 2599 | CUUCUUCAGAUCUUUCAAUA |
| 2123 | CAGUAUUUGGCAACCUGAGA | 2600 | UCUUCUUCAGAUCUUUCAAU |
| 2124 | GCAGUAUUUGGCAACCUGAG | 2601 | AUCUUCUUCAGAUCUUUCAA |
| 2125 | AGCAGUAUUUGGCAACCUGA | 2602 | CAUCUUCUUCAGAUCUUUCA |
| 2126 | CAGCAGUAUUUGGCAACCUG | 2603 | UCAUCUUCUUCAGAUCUUUC |
| 2127 | ACAGCAGUAUUUGGCAACCU | 2604 | AUCAUCUUCUUCAGAUCUUU |
| 2128 | CACAGCAGUAUUUGGCAACC | 2605 | GAUCAUCUUCUUCAGAUCUU |
| 2129 | UCACAGCAGUAUUUGGCAAC | 2606 | UGAUCAUCUUCUUCAGAUCU |
| 2130 | CUCACAGCAGUAUUUGGCAA | 2607 | CUGAUCAUCUUCUUCAGAUC |
| 2131 | GCUCACAGCAGUAUUUGGCA | 2608 | ACUGAUCAUCUUCUUCAGAU |
| 2132 | UGCUCACAGCAGUAUUUGGC | 2609 | AACUGAUCAUCUUCUUCAGA |
| 2133 | AUGCUCACAGCAGUAUUUGG | 2610 | GAACUGAUCAUCUUCUUCAG |
| 2134 | AAUGCUCACAGCAGUAUUUG | 2611 | UGAACUGAUCAUCUUCUUCA |
| 2135 | AAAUGCUCACAGCAGUAUUU | 2612 | AUGAACUGAUCAUCUUCUUC |
| 2136 | CAAAUGCUCACAGCAGUAUU | 2613 | AAUGAACUGAUCAUCUUCUU |
| 2137 | ACAAAUGCUCACAGCAGUAU | 2614 | UAAUGAACUGAUCAUCUUCU |
| 2138 | AACAAAUGCUCACAGCAGUA | 2615 | AUAAUGAACUGAUCAUCUUC |
| 2139 | UAACAAAUGCUCACAGCAGU | 2616 | GAUAAUGAACUGAUCAUCUU |
| 2140 | CUAACAAAUGCUCACAGCAG | 2617 | GGAUAAUGAACUGAUCAUCU |
| 2141 | UCUAACAAAUGCUCACAGCA | 2618 | AGGAUAAUGAACUGAUCAUC |
| 2142 | AUCUAACAAAUGCUCACAGC | 2619 | AAGGAUAAUGAACUGAUCAU |
| 2143 | GAUCUAACAAAUGCUCACAG | 2620 | CAAGGAUAAUGAACUGAUCA |
| 2144 | UGAUCUAACAAAUGCUCACA | 2621 | GCAAGGAUAAUGAACUGAUC |
| 2145 | GUGAUCUAACAAAUGCUCAC | 2622 | UGCAAGGAUAAUGAACUGAU |
| 2146 | UGUGAUCUAACAAAUGCUCA | 2623 | AUGCAAGGAUAAUGAACUGA |
| 2147 | AUGUGAUCUAACAAAUGCUC | 2624 | CAUGCAAGGAUAAUGAACUG |
| 2148 | GAUGUGAUCUAACAAAUGCU | 2625 | ACAUGCAAGGAUAAUGAACU |
| 2149 | UGAUGUGAUCUAACAAAUGC | 2626 | CACAUGCAAGGAUAAUGAAC |
| 2150 | GUGAUGUGAUCUAACAAAUG | 2627 | UCACAUGCAAGGAUAAUGAA |
| 2151 | GGUGAUGUGAUCUAACAAAU | 2628 | AUCACAUGCAAGGAUAAUGA |
| 2152 | UGGUGAUGUGAUCUAACAAA | 2629 | CAUCACAUGCAAGGAUAAUG |
| 2153 | UUGGUGAUGUGAUCUAACAA | 2630 | CCAUCACAUGCAAGGAUAAU |
| 2154 | AUUGGUGAUGUGAUCUAACA | 2631 | ACCAUCACAUGCAAGGAUAA |
| 2155 | UAUUGGUGAUGUGAUCUAAC | 2632 | UACCAUCACAUGCAAGGAUA |
| 2156 | UUAUUGGUGAUGUGAUCUAA | 2633 | AUACCAUCACAUGCAAGGAU |
| 2157 | GUUAUUGGUGAUGUGAUCUA | 2634 | GAUACCAUCACAUGCAAGGA |
| 2158 | GGUUAUUGGUGAUGUGAUCU | 2635 | AGAUACCAUCACAUGCAAGG |
| 2159 | UGGUUAUUGGUGAUGUGAUC | 2636 | CAGAUACCAUCACAUGCAAG |
| 2160 | CUGGUUAUUGGUGAUGUGAU | 2637 | CCAGAUACCAUCACAUGCAA |
| 2161 | CCUGGUUAUUGGUGAUGUGA | 2638 | CCCAGAUACCAUCACAUGCA |
| 2162 | UCCUGGUUAUUGGUGAUGUG | 2639 | UCCCAGAUACCAUCACAUGC |
| 2163 | AUCCUGGUUAUUGGUGAUGU | 2640 | AUCCCAGAUACCAUCACAUG |
| 2164 | AAUCCUGGUUAUUGGUGAUG | 2641 | CAUCCCAGAUACCAUCACAU |
| 2165 | AAAUCCUGGUUAUUGGUGAU | 2642 | ACAUCCCAGAUACCAUCACA |
| 2166 | AAAAUCCUGGUUAUUGGUGA | 2643 | AACAUCCCAGAUACCAUCAC |
| 2167 | UAAAAUCCUGGUUAUUGGUG | 2644 | UAACAUCCCAGAUACCAUCA |
| 2168 | UUAAAAUCCUGGUUAUUGGU | 2645 | AUAACAUCCCAGAUACCAUC |
| 2169 | UUUAAAAUCCUGGUUAUUGG | 2646 | CAUAACAUCCCAGAUACCAU |
| 2170 | CUUUAAAAUCCUGGUUAUUG | 2647 | CCAUAACAUCCCAGAUACCA |
| 2171 | CCUUUAAAAUCCUGGUUAUU | 2648 | CCCAUAACAUCCCAGAUACC |
| 2172 | CCCUUUAAAAUCCUGGUUAU | 2649 | UCCCAUAACAUCCCAGAUAC |
| 2173 | ACCCUUUAAAAUCCUGGUUA | 2650 | UUCCCAUAACAUCCCAGAUA |
| 2174 | GACCCUUUAAAAUCCUGGUU | 2651 | UUUCCCAUAACAUCCCAGAU |
| 2175 | AGACCCUUUAAAAUCCUGGU | 2652 | AUUUCCCAUAACAUCCCAGA |
| 2176 | CAGACCCUUUAAAAUCCUGG | 2653 | CAUUUCCCAUAACAUCCCAG |
| 2177 | GCAGACCCUUUAAAAUCCUG | 2654 | UCAUUUCCCAUAACAUCCCA |
| 2178 | UGCAGACCCUUUAAAAUCCU | 2655 | UUCAUUUCCCAUAACAUCCC |
| 2179 | CUGCAGACCCUUUAAAAUCC | 2656 | CUUCAUUUCCCAUAACAUCC |
| 2180 | CCUGCAGACCCUUUAAAAUC | 2657 | UCUUCAUUUCCCAUAACAUC |
| 2181 | UCCUGCAGACCCUUUAAAAU | 2658 | CUCUUCAUUUCCCAUAACAU |
| 2182 | CUCCUGCAGACCCUUUAAAA | 2659 | GCUCUUCAUUUCCCAUAACA |
| 2183 | GCUCCUGCAGACCCUUUAAA | 2660 | AGCUCUUCAUUUCCCAUAAC |
| 2184 | UGCUCCUGCAGACCCUUUAA | 2661 | GAGCUCUUCAUUUCCCAUAA |
| 2185 | GUGCUCCUGCAGACCCUUUA | 2662 | AGAGCUCUUCAUUUCCCAUA |
| 2186 | GGUGCUCCUGCAGACCCUUU | 2663 | CAGAGCUCUUCAUUUCCCAU |
| 2187 | AGGUGCUCCUGCAGACCCUU | 2664 | ACAGAGCUCUUCAUUUCCCA |
| 2188 | AAGGUGCUCCUGCAGACCCU | 2665 | CACAGAGCUCUUCAUUUCCC |
| 2189 | GAAGGUGCUCCUGCAGACCC | 2666 | UCACAGAGCUCUUCAUUUCC |
| 2190 | AGAAGGUGCUCCUGCAGACC | 2667 | AUCACAGAGCUCUUCAUUUC |
| 2191 | CAGAAGGUGCUCCUGCAGAC | 2668 | AAUCACAGAGCUCUUCAUUU |
| 2192 | ACAGAAGGUGCUCCUGCAGA | 2669 | AAAUCACAGAGCUCUUCAUU |
| 2193 | CACAGAAGGUGCUCCUGCAG | 2670 | AAAAUCACAGAGCUCUUCAU |
| 2194 | CCACAGAAGGUGCUCCUGCA | 2671 | CAAAAUCACAGAGCUCUUCA |
| 2195 | UCCACAGAAGGUGCUCCUGC | 2672 | ACAAAAUCACAGAGCUCUUC |
| 2196 | UUCCACAGAAGGUGCUCCUG | 2673 | UACAAAAUCACAGAGCUCUU |
| 2197 | UUUCCACAGAAGGUGCUCCU | 2674 | UUACAAAAUCACAGAGCUCU |
| 2198 | UUUUCCACAGAAGGUGCUCC | 2675 | CUUACAAAAUCACAGAGCUC |
| 2199 | AUUUUCCACAGAAGGUGCUC | 2676 | UCUUACAAAAUCACAGAGCU |
| 2200 | CAUUUUCCACAGAAGGUGCU | 2677 | AUCUUACAAAAUCACAGAGC |
| 2201 | ACAUUUUCCACAGAAGGUGC | 2678 | GAUCUUACAAAAUCACAGAG |
| 2202 | UACAUUUUCCACAGAAGGUG | 2679 | GGAUCUUACAAAAUCACAGA |
| 2203 | UUACAUUUUCCACAGAAGGU | 2680 | UGGAUCUUACAAAAUCACAG |
| 2204 | UUUACAUUUUCCACAGAAGG | 2681 | CUGGAUCUUACAAAAUCACA |
| 2205 | CUUUACAUUUUCCACAGAAG | 2682 | UCUGGAUCUUACAAAAUCAC |
| 2206 | UCUUUACAUUUUCCACAGAA | 2683 | GUCUGGAUCUUACAAAAUCA |
| 2207 | UUCUUUACAUUUUCCACAGA | 2684 | AGUCUGGAUCUUACAAAAUC |
| 2208 | AUUCUUUACAUUUUCCACAG | 2685 | AAGUCUGGAUCUUACAAAAU |
| 2209 | CAUUCUUUACAUUUUCCACA | 2686 | CAAGUCUGGAUCUUACAAAA |
| 2210 | CCAUUCUUUACAUUUUCCAC | 2687 | UCAAGUCUGGAUCUUACAAA |
| 2211 | UCCAUUCUUUACAUUUUCCA | 2688 | UUCAAGUCUGGAUCUUACAA |
| 2212 | UUCCAUUCUUUACAUUUUCC | 2689 | CUUCAAGUCUGGAUCUUACA |
| 2213 | AUUCCAUUCUUUACAUUUUC | 2690 | ACUUCAAGUCUGGAUCUUAC |
| 2214 | GAUUCCAUUCUUUACAUUUU | 2691 | GACUUCAAGUCUGGAUCUUA |
| 2215 | UGAUUCCAUUCUUUACAUUU | 2692 | UGACUUCAAGUCUGGAUCUU |
| 2216 | CUGAUUCCAUUCUUUACAUU | 2693 | GUGACUUCAAGUCUGGAUCU |
| 2217 | UCUGAUUCCAUUCUUUACAU | 2694 | AGUGACUUCAAGUCUGGAUC |
| 2218 | UUCUGAUUCCAUUCUUUACA | 2695 | CAGUGACUUCAAGUCUGGAU |
| 2219 | GUUCUGAUUCCAUUCUUUAC | 2696 | UCAGUGACUUCAAGUCUGGA |
| 2220 | UGUUCUGAUUCCAUUCUUUA | 2697 | AUCAGUGACUUCAAGUCUGG |
| 2221 | CUGUUCUGAUUCCAUUCUUU | 2698 | CAUCAGUGACUUCAAGUCUG |
| 2222 | CCUGUUCUGAUUCCAUUCUU | 2699 | UCAUCAGUGACUUCAAGUCU |
| 2223 | ACCUGUUCUGAUUCCAUUCU | 2700 | GUCAUCAGUGACUUCAAGUC |
| 2224 | AACCUGUUCUGAUUCCAUUC | 2701 | GGUCAUCAGUGACUUCAAGU |
| 2225 | AAACCUGUUCUGAUUCCAUU | 2702 | AGGUCAUCAGUGACUUCAAG |
| 2226 | AAAACCUGUUCUGAUUCCAU | 2703 | AAGGUCAUCAGUGACUUCAA |
| 2227 | GAAAACCUGUUCUGAUUCCA | 2704 | CAAGGUCAUCAGUGACUUCA |
| 2228 | AGAAAACCUGUUCUGAUUCC | 2705 | UCAAGGUCAUCAGUGACUUC |
| 2229 | CAGAAAACCUGUUCUGAUUC | 2706 | CUCAAGGUCAUCAGUGACUU |
| 2230 | CCAGAAAACCUGUUCUGAUU | 2707 | UCUCAAGGUCAUCAGUGACU |
| 2231 | UCCAGAAAACCUGUUCUGAU | 2708 | UUCUCAAGGUCAUCAGUGAC |
| 2232 | CUCCAGAAAACCUGUUCUGA | 2709 | UUUCUCAAGGUCAUCAGUGA |
| 2233 | UCUCCAGAAAACCUGUUCUG | 2710 | CUUUCUCAAGGUCAUCAGUG |
| 2234 | AUCUCCAGAAAACCUGUUCU | 2711 | ACUUUCUCAAGGUCAUCAGU |
| 2235 | AAUCUCCAGAAAACCUGUUC | 2712 | AACUUUCUCAAGGUCAUCAG |
| 2236 | CAAUCUCCAGAAAACCUGUU | 2713 | AAACUUUCUCAAGGUCAUCA |
| 2237 | UCAAUCUCCAGAAAACCUGU | 2714 | CAAACUUUCUCAAGGUCAUC |
| 2238 | AUCAAUCUCCAGAAAACCUG | 2715 | GCAAACUUUCUCAAGGUCAU |
| 2239 | CAUCAAUCUCCAGAAAACCU | 2716 | UGCAAACUUUCUCAAGGUCA |
| 2240 | UCAUCAAUCUCCAGAAAACC | 2717 | UUGCAAACUUUCUCAAGGUC |
| 2241 | UUCAUCAAUCUCCAGAAAAC | 2718 | AUUGCAAACUUUCUCAAGGU |
| 2242 | GUUCAUCAAUCUCCAGAAAA | 2719 | CAUUGCAAACUUUCUCAAGG |
| 2243 | UGUUCAUCAAUCUCCAGAAA | 2720 | UCAUUGCAAACUUUCUCAAG |
| 2244 | GUGUUCAUCAAUCUCCAGAA | 2721 | UUCAUUGCAAACUUUCUCAA |
| 2245 | UGUGUUCAUCAAUCUCCAGA | 2722 | CUUCAUUGCAAACUUUCUCA |
| 2246 | AUGUGUUCAUCAAUCUCCAG | 2723 | ACUUCAUUGCAAACUUUCUC |
| 2247 | CAUGUGUUCAUCAAUCUCCA | 2724 | UACUUCAUUGCAAACUUUCU |
| 2248 | UCAUGUGUUCAUCAAUCUCC | 2725 | CUACUUCAUUGCAAACUUUC |
| 2249 | CUCAUGUGUUCAUCAAUCUC | 2726 | ACUACUUCAUUGCAAACUUU |
| 2250 | UCUCAUGUGUUCAUCAAUCU | 2727 | GACUACUUCAUUGCAAACUU |
| 2251 | CUCUCAUGUGUUCAUCAAUC | 2728 | CGACUACUUCAUUGCAAACU |
| 2252 | ACUCUCAUGUGUUCAUCAAU | 2729 | UCGACUACUUCAUUGCAAAC |
| 2253 | AACUCUCAUGUGUUCAUCAA | 2730 | GUCGACUACUUCAUUGCAAA |
| 2254 | UAACUCUCAUGUGUUCAUCA | 2731 | UGUCGACUACUUCAUUGCAA |
| 2255 | AUAACUCUCAUGUGUUCAUC | 2732 | GUGUCGACUACUUCAUUGCA |
| 2256 | CAUAACUCUCAUGUGUUCAU | 2733 | GGUGUCGACUACUUCAUUGC |
| 2257 | ACAUAACUCUCAUGUGUUCA | 2734 | AGGUGUCGACUACUUCAUUG |
| 2258 | GACAUAACUCUCAUGUGUUC | 2735 | CAGGUGUCGACUACUUCAUU |
| 2259 | UGACAUAACUCUCAUGUGUU | 2736 | ACAGGUGUCGACUACUUCAU |
| 2260 | CUGACAUAACUCUCAUGUGU | 2737 | AACAGGUGUCGACUACUUCA |
| 2261 | UCUGACAUAACUCUCAUGUG | 2738 | AAACAGGUGUCGACUACUUC |
| 2262 | CUCUGACAUAACUCUCAUGU | 2739 | CAAACAGGUGUCGACUACUU |
| 2263 | UCUCUGACAUAACUCUCAUG | 2740 | ACAAACAGGUGUCGACUACU |
| 2264 | UUCUCUGACAUAACUCUCAU | 2741 | UACAAACAGGUGUCGACUAC |
| 2265 | CUUCUCUGACAUAACUCUCA | 2742 | AUACAAACAGGUGUCGACUA |
| 2266 | UCUUCUCUGACAUAACUCUC | 2743 | UAUACAAACAGGUGUCGACU |
| 2267 | UUCUUCUCUGACAUAACUCU | 2744 | UUAUACAAACAGGUGUCGAC |
| 2268 | UUUCUUCUCUGACAUAACUC | 2745 | CUUAUACAAACAGGUGUCGA |
| 2269 | GUUUCUUCUCUGACAUAACU | 2746 | CCUUAUACAAACAGGUGUCG |
| 2270 | UGUUUCUUCUCUGACAUAAC | 2747 | CCCUUAUACAAACAGGUGUC |
| 2271 | AUGUUUCUUCUCUGACAUAA | 2748 | UCCCUUAUACAAACAGGUGU |
| 2272 | CAUGUUUCUUCUCUGACAUA | 2749 | UUCCCUUAUACAAACAGGUG |
| 2273 | CCAUGUUUCUUCUCUGACAU | 2750 | CUUCCCUUAUACAAACAGGU |
| 2274 | ACCAUGUUUCUUCUCUGACA | 2751 | ACUUCCCUUAUACAAACAGG |
| 2275 | CACCAUGUUUCUUCUCUGAC | 2752 | GACUUCCCUUAUACAAACAG |
| 2276 | GCACCAUGUUUCUUCUCUGA | 2753 | CGACUUCCCUUAUACAAACA |
| 2277 | UGCACCAUGUUUCUUCUCUG | 2754 | UCGACUUCCCUUAUACAAAC |
| 2278 | CUGCACCAUGUUUCUUCUCU | 2755 | CUCGACUUCCCUUAUACAAA |
| 2279 | UCUGCACCAUGUUUCUUCUC | 2756 | UCUCGACUUCCCUUAUACAA |
| 2280 | AUCUGCACCAUGUUUCUUCU | 2757 | GUCUCGACUUCCCUUAUACA |
| 2281 | UAUCUGCACCAUGUUUCUUC | 2758 | UGUCUCGACUUCCCUUAUAC |
| 2282 | CUAUCUGCACCAUGUUUCUU | 2759 | UUGUCUCGACUUCCCUUAUA |
| 2283 | UCUAUCUGCACCAUGUUUCU | 2760 | GUUGUCUCGACUUCCCUUAU |
| 2284 | UUCUAUCUGCACCAUGUUUC | 2761 | UGUUGUCUCGACUUCCCUUA |
| 2285 | CUUCUAUCUGCACCAUGUUU | 2762 | AUGUUGUCUCGACUUCCCUU |
| 2286 | ACUUCUAUCUGCACCAUGUU | 2763 | CAUGUUGUCUCGACUUCCCU |
| 2287 | CACUUCUAUCUGCACCAUGU | 2764 | UCAUGUUGUCUCGACUUCCC |
| 2288 | CCACUUCUAUCUGCACCAUG | 2765 | CUCAUGUUGUCUCGACUUCC |
| 2289 | CCCACUUCUAUCUGCACCAU | 2766 | ACUCAUGUUGUCUCGACUUC |
| 2290 | ACCCACUUCUAUCUGCACCA | 2767 | CACUCAUGUUGUCUCGACUU |
| 2291 | GACCCACUUCUAUCUGCACC | 2768 | ACACUCAUGUUGUCUCGACU |
| 2292 | UGACCCACUUCUAUCUGCAC | 2769 | CACACUCAUGUUGUCUCGAC |
| 2293 | UUGACCCACUUCUAUCUGCA | 2770 | UCACACUCAUGUUGUCUCGA |
| 2294 | GUUGACCCACUUCUAUCUGC | 2771 | AUCACACUCAUGUUGUCUCG |
| 2295 | UGUUGACCCACUUCUAUCUG | 2772 | AAUCACACUCAUGUUGUCUC |
| 2296 | CUGUUGACCCACUUCUAUCU | 2773 | AAAUCACACUCAUGUUGUCU |
| 2297 | GCUGUUGACCCACUUCUAUC | 2774 | AAAAUCACACUCAUGUUGUC |
| 2298 | AGCUGUUGACCCACUUCUAU | 2775 | CAAAAUCACACUCAUGUUGU |
| 2299 | CAGCUGUUGACCCACUUCUA | 2776 | UCAAAAUCACACUCAUGUUG |
| 2300 | ACAGCUGUUGACCCACUUCU | 2777 | AUCAAAAUCACACUCAUGUU |
| 2301 | UACAGCUGUUGACCCACUUC | 2778 | GAUCAAAAUCACACUCAUGU |
| 2302 | CUACAGCUGUUGACCCACUU | 2779 | AGAUCAAAAUCACACUCAUG |
| 2303 | CCUACAGCUGUUGACCCACU | 2780 | CAGAUCAAAAUCACACUCAU |
| 2304 | ACCUACAGCUGUUGACCCAC | 2781 | ACAGAUCAAAAUCACACUCA |
| 2305 | CACCUACAGCUGUUGACCCA | 2782 | AACAGAUCAAAAUCACACUC |
| 2306 | ACACCUACAGCUGUUGACCC | 2783 | AAACAGAUCAAAAUCACACU |
| 2307 | GACACCUACAGCUGUUGACC | 2784 | AAAACAGAUCAAAAUCACAC |
| 2308 | AGACACCUACAGCUGUUGAC | 2785 | GAAAACAGAUCAAAAUCACA |
| 2309 | AAGACACCUACAGCUGUUGA | 2786 | GGAAAACAGAUCAAAAUCAC |
| 2310 | UAAGACACCUACAGCUGUUG | 2787 | UGGAAAACAGAUCAAAAUCA |
| 2311 | UUAAGACACCUACAGCUGUU | 2788 | UUGGAAAACAGAUCAAAAUC |
| 2312 | AUUAAGACACCUACAGCUGU | 2789 | UUUGGAAAACAGAUCAAAAU |
| 2313 | AAUUAAGACACCUACAGCUG | 2790 | AUUUGGAAAACAGAUCAAAA |
| 2314 | AAAUUAAGACACCUACAGCU | 2791 | CAUUUGGAAAACAGAUCAAA |
| 2315 | GAAAUUAAGACACCUACAGC | 2792 | GCAUUUGGAAAACAGAUCAA |
| 2316 | AGAAAUUAAGACACCUACAG | 2793 | UGCAUUUGGAAAACAGAUCA |
| 2317 | GAGAAAUUAAGACACCUACA | 2794 | GUGCAUUUGGAAAACAGAUC |
| 2318 | GGAGAAAUUAAGACACCUAC | 2795 | GGUGCAUUUGGAAAACAGAU |
| 2319 | GGGAGAAAUUAAGACACCUA | 2796 | GGGUGCAUUUGGAAAACAGA |
| 2320 | GGGGAGAAAUUAAGACACCU | 2797 | UGGGUGCAUUUGGAAAACAG |
| 2321 | UGGGGAGAAAUUAAGACACC | 2798 | UUGGGUGCAUUUGGAAAACA |
| 2322 | UUGGGGAGAAAUUAAGACAC | 2799 | UUUGGGUGCAUUUGGAAAAC |
| 2323 | GUUGGGGAGAAAUUAAGACA | 2800 | CUUUGGGUGCAUUUGGAAAA |
| 2324 | UGUUGGGGAGAAAUUAAGAC | 2801 | ACUUUGGGUGCAUUUGGAAA |
| 2325 | AUGUUGGGGAGAAAUUAAGA | 2802 | UACUUUGGGUGCAUUUGGAA |
| 2326 | UAUGUUGGGGAGAAAUUAAG | 2803 | AUACUUUGGGUGCAUUUGGA |
| 2327 | GUAUGUUGGGGAGAAAUUAA | 2804 | GAUACUUUGGGUGCAUUUGG |
| 2328 | AGUAUGUUGGGGAGAAAUUA | 2805 | CGAUACUUUGGGUGCAUUUG |
| 2329 | AAGUAUGUUGGGGAGAAAUU | 2806 | GCGAUACUUUGGGUGCAUUU |
| 2330 | UAAGUAUGUUGGGGAGAAAU | 2807 | GGCGAUACUUUGGGUGCAUU |
| 2331 | AUAAGUAUGUUGGGGAGAAA | 2808 | UGGCGAUACUUUGGGUGCAU |
| 2332 | AAUAAGUAUGUUGGGGAGAA | 2809 | CUGGCGAUACUUUGGGUGCA |
| 2333 | AAAUAAGUAUGUUGGGGAGA | 2810 | UCUGGCGAUACUUUGGGUGC |
| 2334 | GAAAUAAGUAUGUUGGGGAG | 2811 | UUCUGGCGAUACUUUGGGUG |
| 2335 | UGAAAUAAGUAUGUUGGGGA | 2812 | CUUCUGGCGAUACUUUGGGU |
| 2336 | AUGAAAUAAGUAUGUUGGGG | 2813 | GCUUCUGGCGAUACUUUGGG |
| 2337 | AAUGAAAUAAGUAUGUUGGG | 2814 | UGCUUCUGGCGAUACUUUGG |
| 2338 | UAAUGAAAUAAGUAUGUUGG | 2815 | CUGCUUCUGGCGAUACUUUG |
| 2339 | UUAAUGAAAUAAGUAUGUUG | 2816 | ACUGCUUCUGGCGAUACUUU |
| 2340 | GUUAAUGAAAUAAGUAUGUU | 2817 | CACUGCUUCUGGCGAUACUU |
| 2341 | AGUUAAUGAAAUAAGUAUGU | 2818 | UCACUGCUUCUGGCGAUACU |
| 2342 | CAGUUAAUGAAAUAAGUAUG | 2819 | UUCACUGCUUCUGGCGAUAC |
| 2343 | ACAGUUAAUGAAAUAAGUAU | 2820 | CUUCACUGCUUCUGGCGAUA |
| 2344 | CACAGUUAAUGAAAUAAGUA | 2821 | UCUUCACUGCUUCUGGCGAU |
| 2345 | CCACAGUUAAUGAAAUAAGU | 2822 | UUCUUCACUGCUUCUGGCGA |
| 2346 | UCCACAGUUAAUGAAAUAAG | 2823 | CUUCUUCACUGCUUCUGGCG |
| 2347 | CUCCACAGUUAAUGAAAUAA | 2824 | CCUUCUUCACUGCUUCUGGC |
| 2348 | UCUCCACAGUUAAUGAAAUA | 2825 | UCCUUCUUCACUGCUUCUGG |
| 2349 | GUCUCCACAGUUAAUGAAAU | 2826 | CUCCUUCUUCACUGCUUCUG |
| 2350 | AGUCUCCACAGUUAAUGAAA | 2827 | CCUCCUUCUUCACUGCUUCU |
| 2351 | GAGUCUCCACAGUUAAUGAA | 2828 | GCCUCCUUCUUCACUGCUUC |
| 2352 | UGAGUCUCCACAGUUAAUGA | 2829 | UGCCUCCUUCUUCACUGCUU |
| 2353 | UUGAGUCUCCACAGUUAAUG | 2830 | CUGCCUCCUUCUUCACUGCU |
| 2354 | CUUGAGUCUCCACAGUUAAU | 2831 | UCUGCCUCCUUCUUCACUGC |
| 2355 | UCUUGAGUCUCCACAGUUAA | 2832 | CUCUGCCUCCUUCUUCACUG |
| 2356 | CUCUUGAGUCUCCACAGUUA | 2833 | ACUCUGCCUCCUUCUUCACU |
| 2357 | CCUCUUGAGUCUCCACAGUU | 2834 | AACUCUGCCUCCUUCUUCAC |
| 2358 | ACCUCUUGAGUCUCCACAGU | 2835 | CAACUCUGCCUCCUUCUUCA |
| 2359 | AACCUCUUGAGUCUCCACAG | 2836 | CCAACUCUGCCUCCUUCUUC |
| 2360 | AAACCUCUUGAGUCUCCACA | 2837 | UCCAACUCUGCCUCCUUCUU |
| 2361 | UAAACCUCUUGAGUCUCCAC | 2838 | GUCCAACUCUGCCUCCUUCU |
| 2362 | GUAAACCUCUUGAGUCUCCA | 2839 | UGUCCAACUCUGCCUCCUUC |
| 2363 | AGUAAACCUCUUGAGUCUCC | 2840 | UUGUCCAACUCUGCCUCCUU |
| 2364 | AAGUAAACCUCUUGAGUCUC | 2841 | CUUGUCCAACUCUGCCUCCU |
| 2365 | AAAGUAAACCUCUUGAGUCU | 2842 | ACUUGUCCAACUCUGCCUCC |
| 2366 | CAAAGUAAACCUCUUGAGUC | 2843 | UACUUGUCCAACUCUGCCUC |
| 2367 | ACAAAGUAAACCUCUUGAGU | 2844 | GUACUUGUCCAACUCUGCCU |
| 2368 | UACAAAGUAAACCUCUUGAG | 2845 | GGUACUUGUCCAACUCUGCC |
| 2369 | CUACAAAGUAAACCUCUUGA | 2846 | AGGUACUUGUCCAACUCUGC |
| 2370 | CCUACAAAGUAAACCUCUUG | 2847 | CAGGUACUUGUCCAACUCUG |
| 2371 | UCCUACAAAGUAAACCUCUU | 2848 | CCAGGUACUUGUCCAACUCU |
| 2372 | UUCCUACAAAGUAAACCUCU | 2849 | UCCAGGUACUUGUCCAACUC |
| 2373 | GUUCCUACAAAGUAAACCUC | 2850 | UUCCAGGUACUUGUCCAACU |
| 2374 | UGUUCCUACAAAGUAAACCU | 2851 | AUUCCAGGUACUUGUCCAAC |
| 2375 | CUGUUCCUACAAAGUAAACC | 2852 | CAUUCCAGGUACUUGUCCAA |
| 2376 | CCUGUUCCUACAAAGUAAAC | 2853 | GCAUUCCAGGUACUUGUCCA |
| 2377 | UCCUGUUCCUACAAAGUAAA | 2854 | UGCAUUCCAGGUACUUGUCC |
| 2378 | UUCCUGUUCCUACAAAGUAA | 2855 | CUGCAUUCCAGGUACUUGUC |
| 2379 | UUUCCUGUUCCUACAAAGUA | 2856 | UCUGCAUUCCAGGUACUUGU |
| 2380 | CUUUCCUGUUCCUACAAAGU | 2857 | CUCUGCAUUCCAGGUACUUG |
| 2381 | ACUUUCCUGUUCCUACAAAG | 2858 | ACUCUGCAUUCCAGGUACUU |
| 2382 | AACUUUCCUGUUCCUACAAA | 2859 | UACUCUGCAUUCCAGGUACU |
| 2383 | GAACUUUCCUGUUCCUACAA | 2860 | CUACUCUGCAUUCCAGGUAC |
| 2384 | UGAACUUUCCUGUUCCUACA | 2861 | UCUACUCUGCAUUCCAGGUA |
| 2385 | AUGAACUUUCCUGUUCCUAC | 2862 | UUCUACUCUGCAUUCCAGGU |
| 2386 | AAUGAACUUUCCUGUUCCUA | 2863 | CUUCUACUCUGCAUUCCAGG |

*At least one nucleoside linkage of the oligonucleotide
is selected from a phosphorothioate linkage, an alkyl
phosphate linkage, an alkylphosphonate linkage, a
3-methoxypropyl phosphonate linkage, a
phosphorodithioate linkage, a phosphotriester linkage,
a methylphosphonate linkage, an aminoalkylphosphotriester
linkage, an alkylene phosphonate linkage, a phosphinate
linkage, a phosphoramidate linkage, a phosphoramidothioate
linkage, a phosphorodiamidate (e.g., comprising a
phosphorodiamidate morpholino (PMO), 3′ amino ribose,
or 5′ amino ribose) linkage, an aminoalkylphosphoramidate
linkage, a thiophosphoramidate linkage, a
thionoalkylphosphonate linkage, a thionoalkylphosphotriester
linkage, a thiophosphate linkage, a selenophosphate
linkage, and a boranophosphate linkage

Examples of particular PPM1A AONs, or pharmaceutically acceptable salts thereof, described herein include:
a PPM1A antisense oligonucleotide that includes the nucleotide sequence of SEQ ID NO: 87 (5′ GCTGCTTAGCCCATATCGCA 3′ (QPA-542)), or a pharmaceutically acceptable salt thereof,
a PPM1A antisense oligonucleotide that includes the nucleotide sequence of SEQ ID NO: 100 (5′ GCCAGCCTTGCATGCTGCTT 3′ (QPA-555)), or a pharmaceutically acceptable salt thereof,
a PPM1A antisense oligonucleotide that includes the nucleotide sequence of SEQ ID NO: 104 (5′ ACACGCCAGCCTTGCATGCT 3′ (QPA-559)), or a pharmaceutically acceptable salt thereof,
a PPM1A antisense oligonucleotide that includes the nucleotide sequence of SEQ ID NO: 144 (5′ TGGCAAACCGATCACAGCCG 3′ (QPA-599)), or a pharmaceutically acceptable salt thereof,
a PPM1A antisense oligonucleotide that includes the nucleotide sequence of SEQ ID NO: 147 (5′ ACTTGGCAAACCGATCACAG 3′ (QPA-602)), or a pharmaceutically acceptable salt thereof,
a PPM1A antisense oligonucleotide that includes the nucleotide sequence of SEQ ID NO: 148 (5′ CACTTGGCAAACCGATCACA 3′ (QPA-603)), or a pharmaceutically acceptable salt thereof,
a PPM1A antisense oligonucleotide that includes the nucleotide sequence of SEQ ID NO: 149 (5′ CCACTTGGCAAACCGATCAC 3′ (QPA-604)), or a pharmaceutically acceptable salt thereof,
a PPM1A antisense oligonucleotide that includes the nucleotide sequence of SEQ ID NO: 150 (5′ TCCACTTGGCAAACCGATCA 3′ (QPA-605)), or a pharmaceutically acceptable salt thereof,
a PPM1A antisense oligonucleotide that includes the nucleotide sequence of SEQ ID NO: 151 (5′ GTCCACTTGGCAAACCGATC 3′ (QPA-606)), or a pharmaceutically acceptable salt thereof,
a PPM1A antisense oligonucleotide that includes the nucleotide sequence of SEQ ID NO: 152 (5′ AGTCCACTTGGCAAACCGAT 3′ (QPA-607)), or a pharmaceutically acceptable salt thereof,
a PPM1A antisense oligonucleotide that includes the nucleotide sequence of SEQ ID NO: 153 (5′ AAGTCCACTTGGCAAACCGA 3′ (QPA-608)), or a pharmaceutically acceptable salt thereof,
a PPM1A antisense oligonucleotide that includes the nucleotide sequence of SEQ ID NO: 154 (5′ CAAGTCCACTTGGCAAACCG 3′ (QPA-609)), or a pharmaceutically acceptable salt thereof,
a PPM1A antisense oligonucleotide that includes the nucleotide sequence of SEQ ID NO: 170 (5′ AAGAATGACCACGATTCAAG 3′ (QPA-625)), or a pharmaceutically acceptable salt thereof,
a PPM1A antisense oligonucleotide that includes the nucleotide sequence of SEQ ID NO: 187 (5′ GCCCATCATACACAGCAAAG 3′ (QPA-642)), or a pharmaceutically acceptable salt thereof,
a PPM1A antisense oligonucleotide that includes the nucleotide sequence of SEQ ID NO: 189 (5′ ATGCCCATCATACACAGCAA 3′ (QPA-644)), or a pharmaceutically acceptable salt thereof,
a PPM1A antisense oligonucleotide that includes the nucleotide sequence of SEQ ID NO: 191 (5′ GCATGCCCATCATACACAGC 3′ (QPA-646)), or a pharmaceutically acceptable salt thereof,
a PPM1A antisense oligonucleotide that includes the nucleotide sequence of SEQ ID NO: 193 (5′ CAGCATGCCCATCATACACA 3′ (QPA-648)), or a pharmaceutically acceptable salt thereof,
a PPM1A antisense oligonucleotide that includes the nucleotide sequence of SEQ ID NO: 195 (5′ ACCAGCATGCCCATCATACA 3′ (QPA-650)), or a pharmaceutically acceptable salt thereof,
a PPM1A antisense oligonucleotide that includes the nucleotide sequence of SEQ ID NO: 197 (5′ GAACCAGCATGCCCATCATA 3′ (QPA-652)), or a pharmaceutically acceptable salt thereof,
a PPM1A antisense oligonucleotide that includes the nucleotide sequence of SEQ ID NO: 200 (5′ TGAGAACCAGCATGCCCATC 3′ (QPA-655)), or a pharmaceutically acceptable salt thereof,
a PPM1A antisense oligonucleotide that includes the nucleotide sequence of SEQ ID NO: 201 (5′ CTGAGAACCAGCATGCCCAT 3′ (QPA-656)), or a pharmaceutically acceptable salt thereof,
a PPM1A antisense oligonucleotide that includes the nucleotide sequence of SEQ ID NO: 253 (5′ CCTGGTTATTGGTGATGTGA 3′ (QPA-708)), or a pharmaceutically acceptable salt thereof,
a PPM1A antisense oligonucleotide that includes the nucleotide sequence of SEQ ID NO: 254 (5′ TCCTGGTTATTGGTGATGTG 3′ (QPA-709)), or a pharmaceutically acceptable salt thereof,
a PPM1A antisense oligonucleotide that includes the nucleotide sequence of SEQ ID NO: 339 (5′ CATGTGTTCATCAATCTCCA 3′ (QPA-794)), or a pharmaceutically acceptable salt thereof,
a PPM1A antisense oligonucleotide that includes the nucleotide sequence of SEQ ID NO: 340 (5′ TCATGTGTTCATCAATCTCC 3′ (QPA-795)), or a pharmaceutically acceptable salt thereof,
a PPM1A antisense oligonucleotide that includes the nucleotide sequence of SEQ ID NO: 440 (5′ TCTCCACAGTTAATGAAATA 3′ (QPA-895)), or a pharmaceutically acceptable salt thereof,
a PPM1A antisense oligonucleotide that includes the nucleotide sequence of SEQ ID NO: 445 (5′ TTGAGTCTCCACAGTTAATG 3′ (QPA-900)), or a pharmaceutically acceptable salt thereof,
a PPM1A antisense oligonucleotide that includes the nucleotide sequence of SEQ ID NO: 450 (5′ ACCTCTTGAGTCTCCACAGT 3′ (QPA-905)), or a pharmaceutically acceptable salt thereof,
a PPM1A antisense oligonucleotide that includes the nucleotide sequence of SEQ ID NO: 455 (5′ AGTAAACCTCTTGAGTCTCC 3′ (QPA-910)), or a pharmaceutically acceptable salt thereof,
a PPM1A antisense oligonucleotide that includes the nucleotide sequence of SEQ ID NO: 460 (5′ TACAAAGTAAACCTCTTGAG 3′ (QPA-915)), or a pharmaceutically acceptable salt thereof,
a PPM1A antisense oligonucleotide that includes the nucleotide sequence of SEQ ID NO: 507 (5′ ATTACTTGGTTTGTGATCTT 3′ (QPA-962)), or a pharmaceutically acceptable salt thereof,
a PPM1A antisense oligonucleotide that includes the nucleotide sequence of SEQ ID NO: 512 (5′ AGCGGATTACTTGGTTTGTG 3′ (QPA-967)), or a pharmaceutically acceptable salt thereof,
a PPM1A antisense oligonucleotide that includes the nucleotide sequence of SEQ ID NO: 517 (5′ TCTCCAGCGGATTACTTGGT 3′ (QPA-972)), or a pharmaceutically acceptable salt thereof,
a PPM1A antisense oligonucleotide that includes the nucleotide sequence of SEQ ID NO: 522 (5′ TTCTTTCTCCAGCGGATTAC 3′ (QPA-977)), or a pharmaceutically acceptable salt thereof,
a PPM1A antisense oligonucleotide that includes the nucleotide sequence of SEQ ID NO: 532 (5′ TCTGAATTCGTTCTTTCTCC 3′ (QPA-987)), or a pharmaceutically acceptable salt thereof,
a PPM1A antisense oligonucleotide that includes the nucleotide sequence of SEQ ID NO: 570 (5′ GCCATTCACACGCTGAATCA 3′ (QPA-1025)), or a pharmaceutically acceptable salt thereof,
a PPM1A antisense oligonucleotide that includes the nucleotide sequence of SEQ ID NO: 575 (5′ AGAGAGCCATTCACACGCTG 3′ (QPA-1030)), or a pharmaceutically acceptable salt thereof,
a PPM1A antisense oligonucleotide that includes the nucleotide sequence of SEQ ID NO: 579 (5′ AGCCAGAGAGCCATTCACAC 3′ (QPA-1034), or a pharmaceutically acceptable salt thereof,
a PPM1A antisense oligonucleotide that includes the nucleotide sequence of SEQ ID NO: 585 (5′ CGATACAGCCAGAGAGCCAT 3′ (QPA-1040)), or a pharmaceutically acceptable salt thereof,
a PPM1A antisense oligonucleotide that includes the nucleotide sequence of SEQ ID NO: 590 (5′ GCCCTCGATACAGCCAGAGA 3′ (QPA-1045)), or a pharmaceutically acceptable salt thereof,
a PPM1A antisense oligonucleotide that includes the nucleotide sequence of SEQ ID NO: 643 (5′ GCTGCTCAGTAGGACCTTTT 3′ (QPA-1098)), or a pharmaceutically acceptable salt thereof,
a PPM1A antisense oligonucleotide that includes the nucleotide sequence of SEQ ID NO: 906 (5′ TGCTTCTGGCGATACTTTGG 3′ (QPA-1361)), or a pharmaceutically acceptable salt thereof,
a PPM1A antisense oligonucleotide that includes the nucleotide sequence of SEQ ID NO: 911 (5′ TTCACTGCTTCTGGCGATAC 3′ (QPA-1366)), or a pharmaceutically acceptable salt thereof,
a PPM1A antisense oligonucleotide that includes the nucleotide sequence of SEQ ID NO: 916 (5′ CCTTCTTCACTGCTTCTGGC 3′ (QPA-1371)), or a pharmaceutically acceptable salt thereof,
a PPM1A antisense oligonucleotide that includes the nucleotide sequence of SEQ ID NO: 923 (5′ TCTGCCTCCTTCTTCACTGC 3′ (QPA-1378)), or a pharmaceutically acceptable salt thereof, and
a PPM1A antisense oligonucleotide that includes the nucleotide sequence of SEQ ID NO: 931 (5′ TGTCCAACTCTGCCTCCTTC 3′ (QPA-1386)), or a pharmaceutically acceptable salt thereof.

In various embodiments, a PPM1A AON includes linked nucleosides with a nucleobase sequence with a portion of at least 10 contiguous nucleobases that shares 100% identity with an equal length portion of any one of the AON sequences shown in Table 1 or Table 2 (e.g., SEQ ID NOs: 2-955 or SEQ ID NOs: 1910-2863). In various embodiments, a PPM1A AON includes linked nucleosides with a nucleobase sequence with a portion of at least 11, 12, 13, 14, 15, 16, 17, 18, 19, or 20 contiguous nucleobases that shares 100% identity with an equal length portion of any one of the AON sequences shown in Table 1 or Table 2 (e.g., SEQ ID NOs: 2-955 or SEQ ID NOs: 1910-2863).

Also described herein are PPM1A AONs that share less than 100% sequence identity with PPM1A AON sequences described herein. In various embodiments, a PPM1A AON includes linked nucleosides with a nucleobase sequence with a portion of at least 10 contiguous nucleobases that shares at least 90%, at least 91%, at least 92%, at least 93%, at least 94%, at least 95%, at least 96%, at least 97%, at least 98%, at least 99% identity with an equal length portion of any one of the AON sequences shown in Table 1 or Table 2 (e.g., SEQ ID NOs: 2-955 or SEQ ID NOs: 1910-2863). In various embodiments, a PPM1A AON includes linked nucleosides with a nucleobase sequence with a portion of at least 11, 12, 13, 14, 15, 16, 17, 18, 19, or 20 contiguous nucleobases that shares 100% identity with an equal length portion of any one of the AON sequences shown in Table 1 or Table 2 (e.g., SEQ ID NOs: 2-955 or SEQ ID NOs: 1910-2863).

PPM1A Gapmer AONs

In some embodiments, a PPM1A AON has a gapmer design or structure also referred herein merely as “gapmer.” In a gapmer structure the PPM1A AON comprises at least three distinct structural regions including a 5′-wing region, a central region, and a 3′-wing region, in ‘5→3’ orientation.

In various embodiments, the 5′ wing region includes one, two, three, four, five, six, seven, eight, nine, or ten linked nucleosides. In various embodiments, the 3′ wing region includes one, two, three, four, five, six, seven, eight, nine, or ten linked nucleosides. The 5′ and 3′ wing regions (also termed flanking regions) comprise at least one nucleoside that is adjacent to the central region, which comprises a stretch of contiguous nucleosides. The 5′ and 3′ wing regions may be symmetrical or asymmetrical with respect to the number of nucleosides they include.

In various embodiments, the 5′ wing region comprises one or more RNA nucleosides (e.g., ribonucleosides). In various embodiments, the 5′ wing region comprises one or more DNA nucleosides (e.g., deoxyribonucleosides). In various embodiments, the 5′ wing region comprises both RNA nucleosides and DNA nucleosides. In various embodiments, the 3′ wing region comprises one or more RNA nucleosides. In various embodiments, the 3′ wing region comprises one or more DNA nucleosides. In various embodiments, the 3′ wing region comprises both RNA nucleosides and DNA nucleosides.

In various embodiments, the central region includes one, two, three, four, five, six, seven, eight, nine, ten, eleven, twelve, thirteen, fourteen, fifteen, sixteen, seventeen, eighteen, nineteen, or twenty contiguous nucleosides. In some embodiments, the central region comprises a stretch of nucleosides that enable recruitment and activation of RNAseH. In some embodiments, the central region comprises one or more of linked DNA nucleosides, 2′-Fluoro Arabino Nucleic Acids (FANA), and Fluoro Cyclohexenyl nucleic acid (F-CeNA). In some embodiments, all nucleosides of the central region are DNA nucleosides. In some embodiments, the central region comprises a contiguous stretch of 5-16 DNA nucleosides. In some embodiments, the central region comprises a contiguous stretch of 6-15, 7-14, 8-13, or 9-11 DNA nucleosides. In various embodiments, the central region comprises a mix of DNA nucleosides and RNA nucleosides.

In some embodiments, all of the nucleosides of the central region are DNA nucleosides. In further embodiments the central region may consist of a mixture of DNA nucleosides and other nucleosides (2′-Fluoro Arabino Nucleic Acids (FANA), and Fluoro Cyclohexenyl nucleic acid (F-CeNA)) capable of mediating RNase H cleavage. In some embodiments, at least 50% of the nucleosides of the central region are DNA nucleosides, such as at least 60%, at least 65%, at least 70%, at least 75%, at least 80%, at least 85%, at least 90%, at least 95%, or 100% DNA nucleosides.

In particular embodiments, the PPM1A AON includes a 5′ wing region of 5 linked nucleosides, a central region of 10 linked nucleosides, and a 3′ wing region of 5 linked nucleosides, also referred to as a 5-10-5 gapmer. In particular embodiments, the PPM1A AON includes a 5′ wing region of 3 linked nucleosides, a central region of 8 linked nucleosides, and a 3′ wing region of 3 linked nucleosides, also referred to as a 3-8-3 gapmer. In particular embodiments, the PPM1A AON includes a 5′ wing region of 3 linked nucleosides, a central region of 10 linked nucleosides, and a 3′ wing region of 3 linked nucleosides, also referred to as a 3-10-3 gapmer. In particular embodiments, the PPM1A AON includes a 5′ wing region of 4 linked nucleosides, a central region of 10 linked nucleosides, and a 3′ wing region of 4 linked nucleosides, also referred to as a 4-10-4 gapmer. In particular embodiments, the PPM1A AON includes a 5′ wing region of 4 linked nucleosides, a central region of 8 linked nucleosides, and a 3′ wing region of 4 linked nucleosides, also referred to as a 4-8-4 gapmer.

Example PPM1A Gapmer AONs described herein include those identified below in Table 3:

TABLE 3
PPM1A Gapmer AONs. The five linked nucleosides at
the 5′ end and the five linked nucleosides at the
3′ end represent the wing regions and may include
a mixture of ribonucleosides and
deoxyribonucleosides (including modified
ribonucleosides and/or modified
deoxyribonucleosides) whereas the ten linked
nucleosides in the central region are
deoxyribonucleosides. Notation of AON sequences
in Table 3 are as follows: W is guanosine, X
is adenosine, Y is cytosine, and Z is thymidine.

| | Start Position of | PPM1A AON |
|---|---|---|
| SEQ ID NO: | SEQ ID NO: 2864 | Sequence (5′-3′) |
| 2868 | 542 | WYZWYTTAGCCCATAZYWYX |
| 2869 | 555 | WYYXWCCTTGCATGCZWYZZ |
| 2870 | 559 | XYXYWCCAGCCTTGCXZWYZ |
| 2871 | 599 | ZWWYXAACCGATCACXWYYW |
| 2872 | 602 | XYZZWGCAAACCGATYXYXW |
| 2873 | 603 | YXYZZGGCAAACCGAZYXYX |
| 2874 | 604 | YYXYZTGGCAAACCGXZYXY |
| 2875 | 605 | ZYYXYTTGGCAAACCWXZYX |
| 2876 | 606 | WZYYXCTTGGCAAACYWXZY |
| 2877 | 607 | XWZYYACTTGGCAAAYYWXZ |
| 2878 | 608 | XXWZYCACTTGGCAAXYYWX |
| 2879 | 609 | YXXWZCCACTTGGCAXXYYW |
| 2880 | 625 | XXWXXTGACCACGATZYXXW |
| 2881 | 642 | WYYYXTCATACACAGYXXXW |
| 2882 | 644 | XZWYYCATCATACACXWYXX |
| 2883 | 646 | WYXZWCCCATCATACXYXWY |
| 2884 | 648 | YXWYXTGCCCATCATXYXYX |
| 2885 | 650 | XYYXWCATGCCCATCXZXYX |
| 2886 | 652 | WXXYYAGCATGCCCAZYXZX |
| 2887 | 655 | ZWXWXACCAGCATGCYYXZY |
| 2888 | 656 | YZWXWAACCAGCATGYYYXZ |
| 2889 | 708 | YYZWWTTATTGGTGAZWZWX |
| 2890 | 709 | ZYYZWGTTATTGGTGXZWZW |
| 2891 | 794 | YXZWZGTTCATCAATYZYYX |
| 2892 | 795 | ZYXZWTGTTCATCAAZYZYY |
| 2893 | 895 | ZYZYYACAGTTAATGXXXZX |
| 2894 | 900 | ZZWXWTCTCCACAGTZXXZW |
| 2895 | 905 | XYYZYTTGAGTCTCCXYXWZ |
| 2896 | 910 | XWZXXACCTCTTGAGZYZYY |
| 2897 | 915 | ZXYXXAGTAAACCTCZZWXW |
| 2898 | 962 | XZZXYTTGGTTTGTGXZYZZ |
| 2899 | 967 | XWYWWATTACTTGGTZZWZW |
| 2900 | 972 | ZYZYYAGCGGATTACZZWWZ |
| 2901 | 977 | ZZYZZTCTCCAGCGGXZZXY |
| 2902 | 987 | ZYZWXATTCGTTCTTZYZYY |
| 2903 | 1025 | WYYXZTCACACGCTGXXZYX |
| 2904 | 1030 | XWXWXGCCATTCACAYWYZW |
| 2905 | 1034 | XWYYXGAGAGCCATTYXYXY |
| 2906 | 1040 | YWXZXCAGCCAGAGAWYYXZ |
| 2907 | 1045 | WYYYZCGATACAGCCXWXWX |
| 2908 | 1098 | WYZWYTCAGTAGGACYZZZZ |
| 2909 | 1361 | ZWYZZCTGGCGATACZZZWW |
| 2910 | 1366 | ZZYXYTGCTTCTGGCWXZXY |
| 2911 | 1371 | YYZZYTTCACTGCTTYZWWY |
| 2912 | 1378 | ZYZWYCTCCTTCTTCXYZWY |
| 2913 | 1386 | ZWZYYAACTCTGCCTYYZZY |

TABLE 4
PPM1A Gapmer AON sequences. The five linked
nucleosides at the 5′ end and the five linked
nucleosides at the 3′ end represent wing regions
and include a mixture of ribonucleosides and
deoxyribonucleosides (including modified
ribonucleosides and/or modified
deoxyribonucleosides) whereas the ten linked
nucleosides in the central region are
deoxyribonucleosides. Notation of AON sequences
in Table 4 are as follows: W is guanosine, X
is adenosine, Y is cytosine, and M is uridine.

| SEQ ID NO: | PPM1A AON Sequence (5′→3′) |
|---|---|
| 2914 | WYMWYTTAGCCCATAMYWYX |
| 2915 | WYYXWCCTTGCATGCMWYMM |
| 2916 | XYXYWCCAGCCTTGCXMWYM |
| 2917 | MWWYXAACCGATCACXWYYW |
| 2918 | XYMMWGCAAACCGATYXYXW |
| 2919 | YXYMMGGCAAACCGAMYXYX |
| 2920 | YYXYMTGGCAAACCGXMYXY |
| 2921 | MYYXYTTGGCAAACCWXMYX |
| 2922 | WMYYXCTTGGCAAACYWXMY |
| 2923 | XWMYYACTTGGCAAAYYWXM |
| 2924 | XXWMYCACTTGGCAAXYYWX |
| 2925 | YXXWMCCACTTGGCAXXYYW |
| 2926 | XXWXXTGACCACGATMYXXW |
| 2927 | WYYYXTCATACACAGYXXXW |
| 2928 | XMWYYCATCATACACXWYXX |
| 2929 | WYXMWCCCATCATACXYXWY |
| 2930 | YXWYXTGCCCATCATXYXYX |
| 2931 | XYYXWCATGCCCATCXMXYX |
| 2932 | WXXYYAGCATGCCCAMYXMX |
| 2933 | MWXWXACCAGCATGCYYXMY |
| 2934 | YMWXWAACCAGCATGYYYXM |
| 2935 | YYMWWTTATTGGTGAMWMWX |
| 2936 | MYYMWGTTATTGGTGXMWMW |
| 2937 | YXMWMGTTCATCAATYMYYX |
| 2938 | MYXMWTGTTCATCAAMYMYY |
| 2939 | MYMYYACAGTTAATGXXXMX |
| 2940 | MMWXWTCTCCACAGTMXXMW |
| 2941 | XYYMYTTGAGTCTCCXYXWM |
| 2942 | XWMXXACCTCTTGAGMYMYY |
| 2943 | MXYXXAGTAAACCTCMMWXW |
| 2944 | XMMXYTTGGTTTGTGXMYMM |
| 2945 | XWYWWATTACTTGGTMMWMW |
| 2946 | MYMYYAGCGGATTACMMWWM |
| 2947 | MMYMMTCTCCAGCGGXMMXY |
| 2948 | MYMWXATTCGTTCTTMYMYY |
| 2949 | WYYXMTCACACGCTGXXMYX |
| 2950 | XWXWXGCCATTCACAYWYMW |
| 2951 | XWYYXGAGAGCCATTYXYXY |
| 2952 | YWXMXCAGCCAGAGAWYYXM |
| 2953 | WYYYMCGATACAGCCXWXWX |
| 2954 | WYMWYTCAGTAGGACYMMMM |
| 2955 | MWYMMCTGGCGATACMMMWW |
| 2956 | MMYXWTGCTTCTGGCWXMXY |
| 2957 | YYMMYTTCACTGCTTYMWWY |
| 2958 | MYMWYCTCCTTCTTCXYMWY |
| 2959 | MWMYYAACTCTGCCTYYMMY |

Additional exemplary PPM1A Gapmer AONs described herein include:
a PPM1A AON that includes the nucleotide sequence of SEQ ID NO: 2868 (5′ WYZWYTTAGCCCATAZYWYX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2869 (5′ WYYXWCCTTGCATGCZWYZZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2870 (5′ XYXYWCCAGCCTTGCXZWYZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2871 (5′ ZWWYXAACCGATCACXWYYW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2872 (5′ XYZZWGCAAACCGATYXYXW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2873 (5′ YXYZZGGCAAACCGAZYXYX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2874 (5′ YYXYZTGGCAAACCGXZYXY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2875 (5′ ZYYXYTTGGCAAACCWXZYX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2876 (5′ WZYYXCTTGGCAAACYWXZY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2877 (5′ XWZYYACTTGGCAAAYYWXZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2878 (5′ XXWZYCACTTGGCAAXYYWX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2879 (5′ YXXWZCCACTTGGCAXXYYW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2880 (5′ XXWXXTGACCACGATZYXXW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2881 (5′ WYYYXTCATACACAGYXXXW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2882 (5′ XZWYYCATCATACACXWYXX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2883 (5′ WYXZWCCCATCATACXYXWY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2884 (5′ YXWYXTGCCCATCATXYXYX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2885 (5′ XYYXWCATGCCCATCXZXYX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2886 (5′ WXXYYAGCATGCCCAZYXZX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2887 (5′ ZWXWXACCAGCATGCYYXZY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2888 (5′ YZWXWAACCAGCATGYYYXZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2889 (5′ YYZWWTTATTGGTGAZWZWX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2890 (5′ ZYYZWGTTATTGGTGXZWZW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2891 (5′ YXZWZGTTCATCAATYZYYX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2892 (5′ ZYXZWTGTTCATCAAZYZYY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2893 (5′ ZYZYYACAGTTAATGXXXZX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2894 (5′ ZZWXWTCTCCACAGTZXXZW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2895 (5′ XYYZYTTGAGTCTCCXYXWZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2896 (5′ XWZXXACCTCTTGAGZYZYY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2897 (5′ ZXYXXAGTAAACCTCZZWXW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2898 (5′ XZZXYTTGGTTTGTGXZYZZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2899 (5′ XWYWWATTACTTGGTZZWZW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2900 (5′ ZYZYYAGCGGATTACZZWWZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2901 (5′ ZZYZZTCTCCAGCGGXZZXY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2902 (5′ ZYZWXATTCGTTCTTZYZYY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2903 (5′ WYYXZTCACACGCTGXXZYX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2904 (5′ XWXWXGCCATTCACAYWYZW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A antisense AON that includes the nucleotide sequence of SEQ ID NO: 2905 (5′ XWYYXGAGAGCCATTYXYXY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2906 (5′ YWXZXCAGCCAGAGAWYYXZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2907 (5′ WYYYZCGATACAGCCXWXWX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2908 (5′ WYZWYTCAGTAGGACYZZZZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2909 (5′ ZWYZZCTGGCGATACZZZWW 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine; and
a PPM1A AON that includes the nucleotide sequence of SEQ ID NO: 2910 (5′ ZZYXYTGCTTCTGGCWXZXY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine.
a PPM1A AON that includes the nucleotide sequence of SEQ ID NO: 2911 (5′ YYZZYTTCACTGCTTYZWWY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2912 (5′ ZYZWYCTCCTTCTTCXYZWY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;
a PPM1A AON that includes the nucleotide sequence of SEQ ID NO: 2913 (5′ ZWZYYAACTCTGCCTYYZZY 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)-5-methylcytosine, and Z is 2′-O-(2-methoxyethyl)thymidine;

In various embodiments, exemplary PPM1A gapmer AONs have one or more modified internucleoside linkages. For example, in various embodiments, all of the internucleoside linkages in a PPM1A Gapmer AON described above (e.g., SEQ ID NOs: 2868-2913) are phosphorothioate linkages.

Chemical Modifications to PPM1A AONs

As described herein, PPM1A AONs, such as PPM1A AONs with a sequence of any one of SEQ ID NOs: 2-955 or SEQ ID NOs: 1910-2863 or PPM1A Gapmer AONs with a sequence of any one of SEQ ID NOs: 2868-2959, may include one or more chemical modifications to one or more nucleosides and/or to one or more internucleoside linkages. A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to PPM1A AONs encompass substitutions or changes to internucleoside linkages and/or nucleosides (e.g., sugar moieties or nucleobases of nucleosides). Modified PPM1A AONs can be preferred over native forms because of desirable properties such as, for example, enhanced cellular uptake, enhanced affinity for nucleic acid target, increased stability in the presence of nucleases, or increased inhibitory activity. Chemically modified nucleosides, nucleobases, and internucleoside linkages are described in Agrawal and Gait, History and Development of Nucleotide Analogues in Nucleic Acids Drugs, in Drug Discovery Series No. 68, Advances in Nucleic Acid Therapeutics, 1-21 (Agrawal and Gait eds., 2019), the contents of which are incorporated by reference herein.

Modified Internucleoside Linkages

In various embodiments, PPM1A AONs, such as PPM1A AONs with a sequence of any one of SEQ ID NOs: 2-955 or SEQ ID NOs: 1910-2863 or PPM1A Gapmer AONs with a sequence of any one of SEQ ID NOs: 2868-2959, include one or more modified internucleoside linkages. The naturally occurring internucleoside linkage of RNA and DNA is a 3′ to 5′ phosphodiester linkage. PPM1A AONs having one or more modified, i.e., non-naturally occurring, internucleoside linkages can be selected over antisense compounds having naturally occurring internucleoside linkages because of desirable properties such as, for example, enhanced cellular uptake, enhanced affinity for target nucleic acids, and increased stability in the presence of nucleases.

In various embodiments, PPM1A AONs include linked nucleosides with one or more modified internucleoside linkages that link the individual nucleosides. In various embodiments, PPM1A AONs include one, two, three, four, five, six, seven, eight, nine, ten, eleven, twelve, thirteen, fourteen, fifteen, sixteen, seventeen, eighteen, or nineteen modified internucleoside linkages. Examples of modified internucleoside linkages include any one of a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, each modified internucleoside linkage of the PPM1A AON can be designed independent of other modified internucleoside linkages of the PPM1A AON. In other words, the modified internucleoside linkages of a PPM1A AON need not all be the same type of modified internucleoside linkage. In various embodiments, the modified internucleoside linkages are interspersed throughout the antisense compound.

In various embodiments, the PPM1A AON includes at least one phosphorothioate linkage. In various embodiments, the PPM1A AON includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or at least nineteen phosphorothioate linkages. In particular embodiments, the PPM1A AON includes thirteen, fifteen, seventeen, or nineteen phosphorothioate linkages. In particular embodiments, all internucleoside linkages of the PPM1A AON are phosphorothioate linkages.

In various embodiments, a PPM1A AON includes a mixture of modified internucleoside linkages and naturally occurring phosphodiester linkages. For example, a PPM1A AON includes at least one phosphodiester linkage and at least one phosphorothioate linkage. In various embodiments, a PPM1A AON includes between 6 and 10, between 6 and 9, between 6 and 8, between 7 and 10, between 7 and 9, or 6, 7, or 8 phosphorothioate linkages. In some embodiments, a PPM1A AON includes 6, 7, 8, 9, or 10 phosphorothioate linkages. In some embodiments, a PPM1A AON includes between 6 and 10, between 6 and 9, between 6 and 8, between 7 and 10, between 7 and 9, or 6, 7, or 8 phosphodiester linkages. In some embodiments, a PPM1A AON includes 6, 7, 8, 9, or 10 phosphodiester linkages.

In particular embodiments, a PPM1A AON includes 10 phosphorothioate linkages and 9 phosphodiester linkages. In particular embodiments, a PPM1A AON includes 6 phosphorothioate linkages and 7 phosphodiester linkages. In particular embodiments, a PPM1A AON includes 6 phosphorothioate linkages and 9 phosphodiester linkages. In particular embodiments, a PPM1A AON includes 8 phosphorothioate linkages and 9 phosphodiester linkages. In particular embodiments, a PPM1A AON includes 8 phosphorothioate linkages and 7 phosphodiester linkages.

In some embodiments, PPM1A AON includes internucleoside linkages that are designed according to the gapmer design of the PPM1A AON. In some embodiments, the 5′ wing region includes at least one modified internucleoside linkage (e.g., modified from the naturally occurring internucleoside linkage of a 3′ to 5′ phosphodiester linkage). In some embodiments, the 5′ wing region includes at least two, at least three, at least four, at least five, at least six, at least seven, at least eight, at least nine, or at least ten modified internucleoside linkages. In some embodiments, the 3′ wing region includes at least one modified internucleoside linkage. In some embodiments, the 3′ wing region includes at least two, at least three, at least four, at least five, at least six, at least seven, at least eight, at least nine, or at least ten modified internucleoside linkages. In some embodiments, the central region includes at least one modified internucleoside linkage. In some embodiments, the central region includes at least two, at least three, at least four, at least five, at least six, at least seven, at least eight, at least nine, or at least ten modified internucleoside linkages.

In particular embodiments all internucleoside linkages of the 5′ wing region are modified internucleoside linkages, such as phosphorothioate linkages. In particular embodiments all internucleoside linkages of the 3′ wing region are modified internucleoside linkages, such as phosphorothioate linkages. In particular embodiments all internucleoside linkages of the central region are modified internucleoside linkages, such as phosphorothioate linkages. In particular embodiments all internucleoside linkages of each of the 5′ wing region, 3′ wing region, and the central region are modified internucleoside linkages, such as phosphorothioate linkages.

In some embodiments, the one or more modified internucleoside linkages in the 5′ wing region, 3′ wing region, or the central region are phosphorothioate internucleoside linkages. In some embodiments, the phosphorothioate linkages are stereochemically pure phosphorothioate linkages. In some embodiments the phosphorothioate linkages are Sp phosphorothioate linkages. In other embodiments, the phosphorothioate linkages are Rp phosphorothioate linkages.

In some embodiments, the one or more modified internucleoside linkages in the 5′ wing region, 3′ wing region, or the central region can be any of an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In various embodiments, each modified internucleoside linkage of the 5′ wing region, 3′ wing region, or the central region can be designed independent of other modified internucleoside linkages. In other words, the modified internucleoside linkages of 5′ wing region, 3′ wing region, and the central region need not all be the same type of modified internucleoside linkage. In various embodiments, modified internucleoside linkages are interspersed throughout the antisense compound.

In various embodiments, one or more internucleoside linkages of the 5′ wing region, the 3′ wing region, or the central region are naturally occurring linkages (e.g., phosphodiester bonds). In various embodiments, all internucleoside linkages of the central region are unmodified internucleoside linkages (e.g., phosphodiester linkages).

In various embodiments, the internucleoside linkages of the one region (e.g., 5′ wing region, 3′ wing region, or the central region) may differ from the internucleoside linkages of another region. In particular embodiments, the 5′ wing region includes at least one modified internucleoside linkage, the 3′ wing region includes at least one modified internucleoside linkage, and all internucleoside linkages of the central region are unmodified internucleoside linkages (e.g., phosphodiester linkages). In some embodiments, the central region of the oligonucleotide comprises phosphodiester bonds and the 5′ wing region and 3′ wing region each comprises one or more phosphorothioate linkages. In particular embodiments, all internucleoside linkages of the 5′ wing region are modified internucleoside linkages, all internucleoside linkages of the 3′ wing region are modified internucleoside linkages, and all internucleoside linkages of the central region are unmodified internucleoside linkages (e.g., phosphodiester linkages).

In particular embodiments, the PPM1A gapmer AON is a 5-10-5 gapmer and the internucleoside linkages of the PPM1A gapmer AON are denoted as: sssssssssssssssssss (where “s” refers to a phosphorothioate bond) where all the phosphorothioate bonds are in the 5′ wing region or the 3′ wing region and all the phosphodiester bonds are in the central region of the PPM1A AON.

In particular embodiments, the PPM1A gapmer AON is a 5-10-5 gapmer and the internucleoside linkages of the PPM1A gapmer AON are denoted as any of: sssssooooooooosssss, ooooosssssssssooooo, oooooooooooooosssss, soosssssssssssssoos, soossssssssssssssss, ssssssssssssssssoos, and sssssoooooooooooooo (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In particular embodiments, the PPM1A gapmer AON is a 3-8-3 gapmer and the internucleoside linkages of the PPM1A gapmer AON are denoted as: sssssssssssss (where “s” refers to a phosphorothioate bond) where all the phosphorothioate bonds are in the 5′ wing region or the 3′ wing region and all the phosphodiester bonds are in the central region of the PPM1A AON.

In particular embodiments, the PPM1A gapmer AON is a 3-8-3 gapmer and the internucleoside linkages of the PPM1A gapmer AON are denoted as any of: sssooooooosss, ooosssssssooo, ssssssssssooo, sosssssssssos, sosssssssssss, sssssssssssos, and ooossssssssss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In particular embodiments, the PPM1A gapmer AON is a 3-10-3 gapmer and the internucleoside linkages of the PPM1A gapmer AON are denoted as: sssssssssssssss (where “s” refers to a phosphorothioate bond) where all the phosphorothioate bonds are in the 5′ wing region or the 3′ wing region and all the phosphodiester bonds are in the central region of the PPM1A AON.

In particular embodiments, the PPM1A gapmer AON is a 3-10-3 gapmer and the internucleoside linkages of the PPM1A gapmer AON are denoted as any of: sssooooooooosss, ooosssssssssooo, ssssssssssssooo, sosssssssssssos, sosssssssssssss, sssssssssssssos, and ooossssssssssss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In particular embodiments, the PPM1A gapmer AON is a 4-10-4 gapmer and the internucleoside linkages of the PPM1A gapmer AON are denoted as: sssssssssssssssss (where “s” refers to a phosphorothioate bond) where all the phosphorothioate bonds are in the 5′ wing region or the 3′ wing region and all the phosphodiester bonds are in the central region of the PPM1A AON.

In particular embodiments, the PPM1A gapmer AON is a 4-10-4 gapmer and the internucleoside linkages of the PPM1A gapmer AON are denoted as any of: ssssooooooooossss, oooosssssssssoooo, sssssssssssssoooo, soosssssssssssoos, soossssssssssssss, ssssssssssssssoos, and oooosssssssssssss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In particular embodiments, the PPM1A gapmer AON is a 4-8-4 gapmer and the internucleoside linkages of the PPM1A gapmer AON are denoted as: sssssssssssssss (where “s” refers to a phosphorothioate bond) where all the phosphorothioate bonds are in the 5′ wing region or the 3′ wing region and all the phosphodiester bonds are in the central region of the PPM1A AON.

In particular embodiments, the PPM1A gapmer AON is a 4-8-4 gapmer and the internucleoside linkages of the PPM1A gapmer AON are denoted as any of: ssssooooooossss, oooosssssssoooo, sssssssssssoooo, soosssssssssoos, soossssssssssss, ssssssssssssoos, and oooosssssssssss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON.

Modified Sugar Moieties

PPM1A AONs, such as PPM1A AONs with a sequence of any one of SEQ ID NOs: 2-955 or SEQ ID NOs: 1910-2863 or PPM1A Gapmer AONs with a sequence of any one of SEQ ID NOs: 2868-2959, can contain one or more nucleosides wherein the sugar group has been modified. Such sugar modified nucleosides may impart enhanced nuclease stability, increased binding affinity, or some other beneficial biological property to the antisense compounds.

In various embodiments, nucleosides with a modified sugar moiety include a ribose in which the 2′-OH group may be replaced by any one selected from the group consisting of OR, R, R′OR, SH, SR, NH₂, NR₂, N₃, CN, F, Cl, Br, and I (wherein R is an alkyl or aryl and R′ is an alkylene), a 2′-O-methyl (2′-OMe) nucleoside, 2′-O-(2-methoxyethyl) (2′MOE) nucleoside, peptide nucleic acid (PNA), bicyclic nucleic acid (BNA), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, morpholino oligomer, tcDNA, 2′-O, 4′-C-ethylene linked nucleic acid (ENA), hexitol nucleic acids (HNA), and tricyclic analog (e.g., tcDNA).

In certain embodiments, nucleosides comprise chemically modified ribofuranose ring moieties. Examples of chemically modified ribofuranose rings include without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S or CF₂ with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or 5), 4′-S, 2′-F, 2′-OCH₃, 2′-OCH₂CH₃, 2′-O CH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂S CH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ)—, where each Rₗ, Rₘ and Rₙ is, independently, H or substituted or unsubstituted C₁-C₁₀ alkyl.

Additional examples of modified sugar moieties include a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt) (4′-CH(CH₃)—O-2′), S-constrained ethyl (S-cEt) 2′-4′-bridged nucleic acid, 4′-CH₂—O—CH₂-2′, 4′-CH₂—N(R)-2′, 4′-CH(CH₂OCH₃)—O-2′ (“constrained MOE” or “cMOE”), hexitol nucleic acids (HNA), and tricyclic analog (e.g., tcDNA).

In some embodiments, a PPM1A AON comprises a 2′-O-methyl nucleoside (2′OMe) (e.g., a PPM1A AON comprising one or more 2′OMe modified sugar), 2′-O-(2-methoxyethyl) (2′-MOE) (e.g., a PPM1A AON comprising one or more 2′MOE modified sugar (e.g., 2′-MOE)), peptide nucleic acid (PNA) (e.g., a PPM1A AON comprising one or more N-(2-aminoethyl)-glycine units linked by amide bonds or carbonyl methylene linkage as repeating units in place of a sugar-phosphate backbone), locked nucleic acid (LNA) (e.g., a PPM1A AON comprising one or more locked ribose, and can be a mixture of 2′-deoxy nucleotides or 2′OMe nucleotides), constrained ethyl 2′-4′-bridged nucleic acid (c-ET) (e.g., a PPM1A AON comprising one or more cET sugar), cMOE (e.g., a PPM1A AON comprising one or more cMOE sugar), morpholino oligomer (e.g., a PPM1A AON comprising a backbone comprising one or more PMO), deoxy-2′-fluoro nucleoside (e.g., a PPM1A AON comprising one or more 2′-fluoro-β-D-arabinonucleoside), 2′-0,4′-C-ethylene linked nucleic acid (ENA) (e.g., a PPM1A AON comprising one or more ENA modified sugar), hexitol nucleic acid (HNA) (e.g., a PPM1A AON comprising one or more HNA modified sugar), or tricyclic analog (tcDNA) (e.g., a PPM1A AON comprising one or more tcDNA modified sugar).

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof (see U.S. Pat. No. 7,399,845, issued on Jul. 15, 2008)); 4′-C(CH₃)(CH₃)—O-2′ (and analogs thereof (see published International Application WO/2009/006478, published Jan. 8, 2009)); 4′-CH₂—N(OCH₃)-2′ (and analogs thereof (see published International Application WO/2008/150729, published Dec. 11, 2008)); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof (see published International Application WO 2008/154401, published on Dec. 8, 2008)).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. No. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—; wherein:

x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)—J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[—[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl, each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁).

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, α-L-methyleneoxy (4′-CH₂—O-2′) BNA, β-D-methyleneoxy (4′-CH₂—O-2′) BNA, ethyleneoxy (4′-(CH₂)₂—O-2) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA, methylene-thio (4′-CH₂—S-2′) BNA, methylene-amino (4′-CH₂—N(R)-2′) BNA, methyl carbocyclic (4′-CH₂—CH(CH₃)-2′) BNA, and propylene carbocyclic (4′-(CH₂)₃-2′) BNA.

As used herein, “locked nucleic acid” or “LNA” or “LNA nucleosides” refer to modified nucleosides having a bridge (e.g., methylene, ethylene, aminooxy, or oxyimino bridge) connecting two carbon atoms between the 4′ and 2′ position of the nucleoside sugar unit, thereby forming a bicyclic sugar. Examples of such bicyclic sugar include, but are not limited to (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA; wherein R is H, C₁-C₁₂ alkyl, or a protecting group (see U.S. Pat. No. 7,427,672, issued on Sep. 23, 2008).

As used herein, LNA nucleosides include, but are not limited to, nucleosides having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, — C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′- bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a bridge to form the bicyclic sugar moiety. The bridge can be a methylene (—CH₂—) group connecting the 2′ oxygen atom and the 4′ carbon atom, for which the term methyleneoxy (4′-CH₂—O-2′) LNA is used. Furthermore, in the case of the bicyclic sugar moiety having an ethylene bridging group in this position, the term ethyleneoxy (4′-CH₂CH₂—O-2′) LNA is used. α-L-methyleneoxy (4′-CH₂—O-2′), an isomer of methyleneoxy (4′-CH₂—O-2′) LNA is also encompassed within the definition of LNA, as used herein.

In some embodiments, PPM1A AON includes modified sugar moieties that are designed according to the gapmer design of the PPM1A gapmer AON. In various embodiments, PPM1A gapmer AONs include one or more modified sugar moieties. In various embodiments, the 5′ wing region includes at least one modified sugar moiety. In various embodiments, the 3′ wing region includes at least one modified sugar moiety. In various embodiments, the 5′ wing region includes at least two, at least three, at least four, at least five, at least six, at least seven, at least eight, at least nine, or at least ten modified sugar moieties. In various embodiments, the 3′ wing region includes at least two, at least three, at least four, at least five, at least six, at least seven, at least eight, at least nine, or at least ten modified sugar moieties. In some embodiments, each of the 5′ wing region and/or the 3′ wing region includes from 1 to 7 modified sugar moieties, such as from two to six modified sugar moieties, from two to five modified sugar moieties, from two to four modified sugar moieties, or from one to three modified sugar moieties. In particular embodiments, the 5′ wing region includes 3 modified sugar moieties and the 3′ wing region includes 3 modified sugar moieties. In particular embodiments, the 5′ wing region includes 4 modified sugar moieties and the 3′ wing region includes 4 modified sugar moieties. In particular embodiments, the 5′ wing region includes 5 modified sugar moieties and the 3′ wing region includes 5 modified sugar moieties.

In various embodiments, the nucleosides with a modified sugar moiety in the 5′ and 3′ wing regions are any one of a ribose in which the 2′-OH group may be replaced by any one selected from the group consisting of OR, R, R′OR, SH, SR, NH₂, NR₂, N₃, CN, F, Cl, Br, and I (wherein R is an alkyl or aryl and R′ is an alkylene), a 2′-O-methyl (2′-OMe) nucleoside, 2′-O-(2-methoxyethyl) (2′MOE) nucleoside, peptide nucleic acid (PNA), bicyclic nucleic acid (BNA), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, morpholino oligomer, tcDNA, 2′-0,4′-C-ethylene linked nucleic acid (ENA), hexitol nucleic acids (HNA), and tricyclic analog (e.g., tcDNA).

In some embodiments, the 5′ wing region and/or 3′ wing region comprises at least one 2′-MOE nucleoside. In some embodiments both the 5′ and 3′ wing regions comprise at least one 2′-MOE nucleoside. In some embodiments, each of the 5′ wing region and the 3′ wing region comprises two, three, four, five, six, seven, eight, nine, or ten 2′-MOE nucleosides. In some embodiments, all the nucleosides in each of the 5′ wing region and the 3′ wing region are 2′-MOE nucleosides.

In other embodiments, the wing regions may comprise both 2′-MOE nucleosides and other nucleosides (mixed wings), such as DNA nucleosides and/or non-MOE modified nucleosides, such as bicyclic nucleosides (BNAs) (e.g., locked nucleic acid (LNA) nucleosides or constrained ethyl 2′-4′-bridged nucleic acid (cEt) nucleosides), 2′-O-methyl nucleosides, tricycloDNA, S-cEt, morpholinos, or other 2′ substituted nucleosides.

In some embodiments, the 5′ wing region or the 3′ wing region comprises at least one BNA (e.g., at least one LNA nucleoside or cET nucleoside). In some embodiments each of the 5′ and 3′ wing regions comprises a BNA. In some embodiments all the nucleosides in the 5′ and 3′ wing regions are BNAs. In a further embodiment, the BNAs in the 5′ and/or 3′ wing regions are independently selected from the group comprising oxy-LNA, thio-LNA, amino-LNA, cET, and/or ENA, in either the beta-D or alpha-L configurations or combinations thereof.

In some embodiments, the 5′ and/or 3′ wing comprises at least one 2′-O-methyl nucleoside. In some embodiments, the 5′ wing comprises at least one 2′-O-methyl nucleoside. In some embodiments both the 5′ and 3′ wing regions comprise a 2′-O-methyl nucleoside. In some embodiments all the nucleosides in the wing regions are 2′-O-methyl nucleosides.

Modified Nucleobase

In various embodiments, PPM1A AONs, such as PPM1A AONs with a sequence of any one of SEQ ID NOs: 2-955 or SEQ ID NOs: 1910-2863 or PPM1A Gapmer AONs with a sequence of any one of SEQ ID NOs: 2868-2959, include one or more modified nucleobases.

Examples of modified nucleobases, including a 5-methylpyrimidine, for example, 5-methylcytosine or 5-methoxyuridine, a 5-methylpurine, for example, 5-methylguanine, or pseudouridine.

In various embodiments, a PPM1A AON includes at least one modified nucleobase. In various embodiments, a PPM1A AON includes two, three, four, five, six, seven, eight, nine, or ten modified nucleobases. In various embodiments, a PPM1A AON includes at least one 5-methylcytosine nucleobase. In various embodiments, a PPM1A AON includes two, three, four, five, six, seven, eight, nine, or ten 5-methylcytosine nucleobases.

In various embodiments, a PPM1A AON includes both modified and unmodified nucleobases. For example, a PPM1A AON may include both cytosines and 5-methyl cytosines. In some embodiments, a PPM1A AON may include one, two three, four, five, six, seven, eight, nine, or ten cytosines and further include one, two, three, four, five, six seven, eight, nine, or ten 5-methylcytosines.

In various embodiments, each of a particular type of nucleobase in the PPM1A AON is replaced with a corresponding modified nucleobase. For example, every guanine of the PPM1A AON is replaced with a 5-methyl guanine. As another example, every cytosine of the PPM1A AON is replaced with a 5-methylcytosine.

In some embodiments, a PPM1A AON includes modified nucleobases that are designed according to the gapmer design of the PPM1A gapmer AON. In various embodiments, the linked nucleosides of the 5′ wing region, the linked nucleosides of the 3′ wing region, or the linked nucleosides of the central region comprise one or more modified nucleobases. In some embodiments, the 5′ wing region and/or the 3′ wing region includes one to ten modified nucleobases, such as from two to eight modified nucleobases, from three to six modified nucleobases, or from four to five modified nucleobases. In some embodiments, the 5′ wing region and/or the 3′ wing region includes one, two, three, four, five, six, seven, eight, nine, or ten modified nucleobases. In some embodiments, the central region includes one to ten modified nucleobases, such as from two to eight modified nucleobases, from three to six modified nucleobases, or from four to five modified nucleobases. In some embodiments, the central region includes one, two, three, four, five, six, seven, eight, nine, or ten modified nucleobases. Examples of modified nucleobases include a 5-methylpyrimidine, for example, 5-methylcytosine or 5-methoxyuridine, a 5-methylpurine, for example, 5-methylguanine, or pseudouridine.

In various embodiments, at least one cytosine in the 5′ wing region and/or the 3′ wing region of the PPM1A AON is replaced with a modified nucleobase, such as a 5-methylcytosine. In various embodiments, at least one cytosine in the 5′ wing region is replaced with a modified nucleobase, such as a 5-methylcytosine. In various embodiments, at least one cytosine in the 3′ wing region is replaced with a modified nucleobase, such as a 5-methylcytosine. In various embodiments, at least one cytosine in the central region is replaced with a modified nucleobase, such as a 5-methylcytosine. In various embodiments, all cytosines in the 5′ wing region are replaced with modified nucleobases, such as 5-methylcytosines. In various embodiments, all cytosines in the 3′ wing region are replaced with modified nucleobases, such as 5-methylcytosines. In various embodiments, all cytosines in the central region are replaced with modified nucleobases, such as 5-methylcytosines.

In particular embodiments, all cytosines in the 5′ wing region, all cytosines in the 3′ wing region, and all cytosines in the central region are replaced with modified nucleobases, such as 5-methylcytosines. In particular embodiments, all cytosines in the 5′ wing region, all cytosines in the 3′ wing region are replaced with modified nucleobases, such as 5-methylcytosines; however, all cytosines in the central region are unmodified nucleobases.

Modified Oligonucleotides

Described herein are additional embodiments of modified oligonucleotides, which can include any of the modified internucleoside linkages and/or modified nucleosides (e.g., modified sugar moieties, and/or modified nucleobases) described above.

In some embodiments, a PPM1A AON, or a pharmaceutically acceptable salt thereof, includes the nucleotide sequence of any one of SEQ ID NOs: 2-955, SEQ ID NOs: 1910-2863, SEQ ID NOs: 2868-2913, and SEQ ID NOs: 2914-2959 where at least one nucleoside of the nucleoside sequence is substituted with a 2′-O-(2-methoxyethyl) nucleoside, a 2′-O-methyl nucleoside, a 2′-deoxy-2′-fluoro nucleoside, a 2′-fluoro-β-D-arabinonucleoside, a bicylic nucleic acid, a bridged nucleic acid, a locked nucleic acid (LNA), a constrained ethyl (cET) nucleic acid, a tricyclo-DNA (tcDNA), a 2′-0,4′-C-ethylene linked nucleic acid (ENA), or a peptide nucleic acid (PNA). In particular embodiments, at least one internucleoside linkage of the PPM1A AON is a phosphorothioate linkage. In some embodiments, all internucleoside linkages of the PPM1A AON are phosphorothioate linkages. Also described herein are pharmaceutical compositions that include any of the foregoing antisense oligonucleotides, or a pharmaceutically acceptable salt thereof, and a pharmaceutically acceptable excipient.

PPM1A AONs described herein, can include chemically modified nucleosides, including modified ribonucleosides and modified deoxyribonucleosides. Chemically modified nucleosides include 2′-substituted nucleosides in which the 2′ position of the sugar ring includes a moiety other than —H or —OH (for example, —F or an O-alkyl group). For example, chemically modified nucleosides include, but are not limited to 2′-O-(2-methoxyethyl) modifications, for example, 2′-O-(2-methoxyethyl)guanosine, 2′-O-(2-methoxyethyl)adenosine, 2′-O-(2-methoxyethyl)cytosine, and 2′-O-(2-methoxyethyl)thymidine.

In some embodiments, PPM1A AONs can include chemically modified nucleosides, for example, 2′ O-methyl ribonucleosides, for example, 2′ O-methyl cytidine, 2′ O-methyl guanosine, 2′ O-methyl uridine, and/or 2′ O-methyl adenosine. PPM1A AONs described herein, can also include one or more chemically modified bases, including a 5-methyl pyrimidine, for example, 5-methylcytosine, and/or a 5-methyl purine, for example, 5-methyl guanine. PPM1A AONs described herein, can also include any of the following chemically modified nucleosides: 5-methyl-2′-O-methylcytidine, 5-methyl-2′-O-methylthymidine, 5-methylcytidine, 5-methyluridine, and/or 5-methyl 2′-deoxycytidine.

It is contemplated that in some embodiments, a disclosed PPM1A AON may optionally have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

In certain embodiments, the disclosure provides mixed modalities of PPM1A AONs with combinations of modified nucleosides, e.g., a combination of a PPM1A peptide nucleic acid (PNA) and a PPM1A locked nucleic acid (LNA). Chemically modified nucleosides also include, but are not limited to, locked nucleic acids (LNAs), 2′-O-methyl, 2′-fluoro, and 2′-fluoro-β-D-arabinonucleotide (FANA) modifications. Chemically modified nucleosides that can be included in PPM1A AONs described herein are described in Johannes and Lucchino, (2018) “Current Challenges in Delivery and Cytosolic Translocation of Therapeutic RNAs” Nucleic Acid Ther. 28(3): 178-93; Rettig and Behlke, (2012) “Progress toward in vivo use of siRNAs-II” Mol Ther 20:483-512; and Khvorova and Watts, (2017) “The chemical evolution of oligonucleotide therapies of clinical utility” Nat Biotechnol., 35(3):238-48, the contents of each of which are incorporated by reference herein.

PPM1A AONs described herein can include chemical modifications that promote stabilization of an oligonucleotide's terminal 5′-phosphate and phosphatase-resistant analogs of 5′-phosphate. Chemical modifications that promote oligonucleotide terminal 5′-phosphate stabilization or which are phosphatase-resistant analogs of 5′-phosphate include, but are not limited to, 5′-methyl phosphonate, 5′-methylenephosphonate, 5′-methylenephosphonate analogs, 5′-E-vinyl phosphonate (5′-E-VP), 5′-phosphorothioate, and 5′-C-methyl analogs. Chemical modifications that promote AON terminal 5′-phosphate stabilization and phosphatase-resistant analogues of 5′-phosphate are described in Khvorova and Watts, (2017) “The chemical evolution of oligonucleotide therapies of clinical utility” Nat Biotechnol., 35(3):238-48, the contents of which are incorporated by reference herein.

In some embodiments described herein, a PPM1A AON, or a pharmaceutically acceptable salt thereof, is a modified oligonucleotide which includes the nucleotide sequence of any one of SEQ ID NOs: 2-955, SEQ ID NOs: 1910-2863, SEQ ID NOs: 2868-2913, and SEQ ID NOs: 2914-2959, wherein the PPM1A AON includes a modification of at least one nucleoside or at least one internucleoside linkage. For example, in some embodiments, a PPM1A AON, or a pharmaceutically acceptable salt thereof, includes the nucleotide sequence of any one of SEQ ID NOs: 2-955, SEQ ID NOs: 1910-2863, SEQ ID NOs: 2868-2913, and SEQ ID NOs: 2914-2959, and at least one nucleoside linkage of the nucleotide sequence is a a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In some embodiments of PPM1A AONs described herein, at least one internucleoside linkage of the nucleotide sequence is a phosphorothioate linkage. For example, in some embodiments of PPM1A AONs described herein, one, two, three, or more internucleoside linkages of the nucleotide sequence is a phosphorothioate linkage. In preferred embodiments of PPM1A AONs described herein, all internucleoside linkages of the nucleotide sequence are phosphorothioate linkages. Thus, in some embodiments, all of the nucleotide linkages of a PPM1A AON of any of SEQ ID NOs: 2-955, SEQ ID NOs: 1910-2863, SEQ ID NOs: 2868-2913, and SEQ ID NOs: 2914-2959 are phosphorothioate linkages. In some embodiments, one or more of the nucleotide linkages of a PPM1A AON of any of SEQ ID NOs: 2-955, SEQ ID NOs: 1910-2863, SEQ ID NOs: 2868-2913, and SEQ ID NOs: 2914-2959 are phosphorothioate linkages.

Contemplated PPM1A AONs may optionally include at least one modified sugar. For example, the sugar moiety of at least one nucleotide constituting the oligonucleotide is a ribose in which the 2′-OH group may be replaced by any one selected from the group consisting of OR, R, R′OR, SH, SR, NH₂, NR₂, N₃, CN, F, Cl, Br, and I (wherein R is an alkyl or aryl and R′ is an alkylene).

In particular embodiments, a PPM1A AON has a nucleoside sequence of eeeee-d10-eeeee (where “e” denotes a 2′-O-MOE modified nucleoside and where “d10” denotes a contiguous 10 DNA nucleobase sequence). In this embodiment, the 5′ wing region includes five 2′-O-MOE modified nucleosides, the gap region includes 10 contiguous DNA nucleobases, and the 3′ wing region includes five 2′-O-MOE modified nucleosides. The internucleoside linkages of the PPM1A AON can have the sequence of sssssooooooooosssss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eeeee-d10-eeeee (where “e” denotes a 2′-O-MOE modified nucleoside and where “d10” denotes a contiguous 10 DNA nucleobase sequence). In this embodiment, the 5′ wing region includes five 2′-O-MOE modified nucleosides, the gap region includes 10 contiguous DNA nucleobases, and the 3′ wing region includes five 2′-O-MOE modified nucleosides. The internucleoside linkages of the PPM1A AON can have the sequence of sssssssssssssssssss (where “s” refers to a phosphorothioate bond) where all internucleoside linkages of the PPM1A AON are phosphorothioate bonds. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eee-d8-eee (where “e” denotes a 2′-O-MOE modified nucleoside and where “d8” denotes a contiguous 8 DNA nucleobase sequence). In this embodiment, the 5′ wing region includes three 2′-O-MOE modified nucleosides, the gap region includes 8 contiguous DNA nucleobases, and the 3′ wing region includes three 2′-O-MOE modified nucleosides. The internucleoside linkages of the PPM1A AON can have the sequence of sssooooooosss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eee-d8-eee (where “e” denotes a 2′-O-MOE modified nucleoside and where “d8” denotes a contiguous 8 DNA nucleobase sequence). In this embodiment, the 5′ wing region includes three 2′-O-MOE modified nucleosides, the gap region includes 8 contiguous DNA nucleobases, and the 3′ wing region includes three 2′-O-MOE modified nucleosides. The internucleoside linkages of the PPM1A AON can have the sequence of sssssssssssss (where “s” refers to a phosphorothioate bond) where all internucleoside linkages of the PPM1A AON are phosphorothioate bonds. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eee-d10-eee (where “e” denotes a 2′-O-MOE modified nucleoside and where “d10” denotes a contiguous 10 DNA nucleobase sequence). In this embodiment, the 5′ wing region includes three 2′-O-MOE modified nucleosides, the gap region includes 10 contiguous DNA nucleobases, and the 3′ wing region includes three 2′-O-MOE modified nucleosides. The internucleoside linkages of the PPM1A AON can have the sequence of sssooooooooosss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eee-d10-eee (where “e” denotes a 2′-O-MOE modified nucleoside and where “d10” denotes a contiguous 10 DNA nucleobase sequence). In this embodiment, the 5′ wing region includes three 2′-O-MOE modified nucleosides, the gap region includes 10 contiguous DNA nucleobases, and the 3′ wing region includes three 2′-O-MOE modified nucleosides. The internucleoside linkages of the PPM1A AON can have the sequence of sssssssssssssss (where “s” refers to a phosphorothioate bond) where all internucleoside linkages of the PPM1A AON are phosphorothioate bonds. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eeee-d10-eeee (where “e” denotes a 2′-O-MOE modified nucleoside and where “d10” denotes a contiguous 10 DNA nucleobase sequence). In this embodiment, the 5′ wing region includes four 2′-O-MOE modified nucleosides, the gap region includes 10 contiguous DNA nucleobases, and the 3′ wing region includes four 2′-O-MOE modified nucleosides. The internucleoside linkages of the PPM1A AON can have the sequence of ssssooooooooossss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eeee-d10-eeee (where “e” denotes a 2′-O-MOE modified nucleoside and where “d10” denotes a contiguous 10 DNA nucleobase sequence). In this embodiment, the 5′ wing region includes four 2′-O-MOE modified nucleosides, the gap region includes 10 contiguous DNA nucleobases, and the 3′ wing region includes four 2′-O-MOE modified nucleosides. The internucleoside linkages of the PPM1A AON can have the sequence of sssssssssssssssss (where “s” refers to a phosphorothioate bond) where all internucleoside linkages of the PPM1A AON are phosphorothioate bonds. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eeee-d8-eeee (where “e” denotes a 2′-O-MOE modified nucleoside and where “d8” denotes a contiguous 8 DNA nucleobase sequence). In this embodiment, the 5′ wing region includes four 2′-O-MOE modified nucleosides, the gap region includes 8 contiguous DNA nucleobases, and the 3′ wing region includes four 2′-O-MOE modified nucleosides. The internucleoside linkages of the PPM1A AON can have the sequence of ssssooooooossss (where “s” refers to a phosphorothioate bond and “o” refers to a phosphodiester bond) where all the phosphorothioate bonds are in the 5′ wing region or the 3′ wing region and all the phosphodiester bonds are in the central region of the PPM1A AON. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

In particular embodiments, a PPM1A AON has a nucleoside sequence of eeee-d8-eeee (where “e” denotes a 2′-O-MOE modified nucleoside and where “d8” denotes a contiguous 8 DNA nucleobase sequence). In this embodiment, the 5′ wing region includes four 2′-O-MOE modified nucleosides, the gap region includes 8 contiguous DNA nucleobases, and the 3′ wing region includes four 2′-O-MOE modified nucleosides. The internucleoside linkages of the PPM1A AON can have the sequence of sssssssssssssss (where “s” refers to a phosphorothioate bond) where all internucleoside linkages of the PPM1A AON are phosphorothioate bonds. In various embodiments, the PPM1A AON includes unmodified cytosines. In various embodiments, the PPM1A AON includes modified cytosines (e.g., 5-methylcytosine). In various embodiments, all cytosines of the 5′ wing region and the 3′ wing region are modified cytosines (e.g., 5-methylcytosine).

PPM1A Gene Product

Generally, a PPM1A AON disclosed herein includes linked nucleosides with a nucleobase sequence that is at least 90%, at least 91%, at least 92%, at least 93%, at least 94%, at least 95%, at least 96%, at least 97%, at least 98%, at least 99%, or that is 100% complementary to a portion of a PPM1A gene product. In embodiments of the invention described herein, a PPM1A inhibitor can target PPM1A gene products of PPM1A genes of one or more species. For example, a PPM1A inhibitor can target a PPM1A gene product of a mammalian PPM1A gene, for example, a human (i.e., Homo sapiens) PPM1A gene, a rodent PPM1A gene (for example, a mouse (Mus musculus) PPM1A gene), and/or a primate PPM1A gene (for example, a Macaca fascicularis PPM1A gene or a Macaca mulatta PPM1A gene). In particular embodiments, the PPM1A inhibitor targets a human PPM1A gene product. A PPM1A gene product can be, for example, an RNA gene product, for example, an mRNA gene product, or a protein product of a PPM1A gene. In some embodiments, the PPM1A inhibitor includes a nucleotide sequence that is complementary to a nucleotide sequence of a PPM1A gene or a PPM1A RNA, for example a PPM1A mRNA, or a portion thereof. In some embodiments the PPM1A inhibitor includes a nucleobase sequence that is complementary to a portion of a nucleotide sequence that is shared between PPM1A genes or PPM1A RNAs (for example, PPM1A mRNAs) of multiple species. For example, in some embodiments, the PPM1A inhibitor is a PPM1A antisense therapeutic, for example, a PPM1A antisense oligonucleotide, that is complementary to a nucleotide sequence shared by a human, mouse, and/or primate PPM1A genes or PPM1A mRNAs.

In some embodiments of the disclosure, the PPM1A gene product is a PPM1A mRNA transcribed from nucleotide 41,932 to nucleotide 42,787 and from nucleotide 44,874 to nucleotide 44,990 of a PPM1A gene sequence (for example the PPM1A gene sequence of NCBI Reference Sequence NG_029698.1 (SEQ ID NO: 1) or a PPM1A coding sequence), or a portion thereof. In some embodiments of the disclosure, the PPM1A gene product is a is a nucleotide sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a PPM1A mRNA transcribed from nucleotide 41,932 to nucleotide 42,787 and from nucleotide 44,874 to nucleotide 44,990 of a PPM1A gene sequence (for example the PPM1A gene sequence of NCBI Reference Sequence NG_029698.1 (SEQ ID NO: 1) or a PPM1A coding sequence),), or a portion thereof.

In some embodiments of the disclosure, the PPM1A gene product is a PPM1A mRNA transcribed from any one of nucleotides 8470-8926, 41933-42787, 44874-45990, 49055-49164, 50647-50704, and 51703-58336 of a PPM1A gene sequence (for example the PPM1A gene sequence of NCBI Reference Sequence NG_029698.1 (SEQ ID NO: 1). In some embodiments of the disclosure, the PPM1A gene product is a PPM1A mRNA transcribed from the coding region of a PPM1A gene sequence, such as a coding region including nucleotides 8470-8926, 41933-42787, 44874-45990, 49055-49164, 50647-50704, and 51703-58336 of a PPM1A gene sequence (for example the PPM1A gene sequence of NCBI Reference Sequence NG_029698.1 (SEQ ID NO: 1). In various embodiments, the PPM1A mRNA is PPM1A mRNA transcript variant 1, corresponding to NCBI Reference Sequence NM_021003.5 (SEQ ID NO: 2864).

In some embodiments of the disclosure, the PPM1A gene product is a PPM1A mRNA transcribed from any one of nucleotides 8470-8926, 9629-9730, 41933-42787, and 44874-47804 of a PPM1A gene sequence (for example the PPM1A gene sequence of NCBI Reference Sequence NG_029698.1 (SEQ ID NO: 1). In some embodiments of the disclosure, the PPM1A gene product is a PPM1A mRNA transcribed from the coding region of a PPM1A gene sequence, such as a coding region including nucleotides 8470-8926, 9629-9730, 41933-42787, and 44874-47804 of a PPM1A gene sequence (for example the PPM1A gene sequence of NCBI Reference Sequence NG_029698.1 (SEQ ID NO: 1). In various embodiments, the PPM1A mRNA is PPM1A mRNA transcript variant 2, corresponding to NCBI Reference Sequence NM_177951.2 (SEQ ID NO: 2865)

In some embodiments of the disclosure, the PPM1A gene product is a PPM1A mRNA transcribed from any one of nucleotides 4999-5295, 41933-42787, 44874-44990, 49055-49164, 50647-50704, 51703-58336 of a PPM1A gene sequence (for example the PPM1A gene sequence of NCBI Reference Sequence NG_029698.1 (SEQ ID NO: 1). In some embodiments of the disclosure, the PPM1A gene product is a PPM1A mRNA transcribed from the coding region of a PPM1A gene sequence, such as a coding region including nucleotides 4999-5295, 41933-42787, 44874-44990, 49055-49164, 50647-50704, 51703-58336 of a PPM1A gene sequence (for example the PPM1A gene sequence of NCBI Reference Sequence NG_029698.1 (SEQ ID NO: 1). In various embodiments, the PPM1A mRNA is PPM1A mRNA transcript variant 3, corresponding to NCBI Reference Sequence NM_177952.2 (SEQ ID NO: 2866).

In some embodiments of the disclosure, the PPM1A gene product is a nucleotide sequence including nucleotides 457-1429 of PPM1A mRNA transcript variant 1 (i.e., nucleotides 457-1429 of, for example, PPM1A mRNA transcript variant 1, corresponding to NCBI Reference Sequence NM_021003.5), or a portion thereof. In some embodiments of the disclosure, the PPM1A gene product is a nucleotide sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with nucleotides 457-1429 of PPM1A mRNA transcript variant 1 (i.e., nucleotides 457-1429 of, for example, PPM1A mRNA transcript variant 1, corresponding to NCBI Reference Sequence NM_021003.5), or a portion thereof.

In some embodiments described herein, a PPM1A gene product is a PPM1A mRNA isoform transcript (for example, PPM1A mRNA transcript variant 1, corresponding to NCBI Reference Sequence NM_021003.5 (SEQ ID NO: 2864)), or a portion thereof. In some embodiments described herein, a PPM1A gene product is a nucleotide sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a PPM1A mRNA isoform transcript (for example, PPM1A mRNA transcript variant 1, corresponding to NCBI Reference Sequence NM_021003.5 (SEQ ID NO: 2864)), or a portion thereof.

PPM1A mRNA transcript variant 1, corresponding to NCBI Reference Sequence NM_021003.5 (SEQ ID NO: 2864)

| (SEQ ID NO: 2864) | |
|---|---|
| 1 | agaggcggcg gcggcggcgg tggcggcgct agggacggga gcgcgcgcgg gagctagaga |
| 61 | gcagtggtct cggcgctcgt ccggcccgca gcttcgggtc ctcaggcggc tgttgctccg |
| 121 | gaacgggtgg ttggggaggg gggggtgggg ggactctaga cagctgaggc gcgaaagcga |
| 181 | tgagtcctcg gctcttcctc ctccttctcc gggacccgct ctctgcctcc ctctccaacg |
| 241 | cccggatgat ctgagccgcg agggcgccga cagccggggg cccggacgca gcccggctcc |
| 301 | tcccctcctc cgccccttcc ccagcctgac ctggcccgcc gctgcagcgg tgacccctcc |
| 361 | cccggctgcc gccgtcgccg ccgcggtgac cccctccccg gctgccgccg ccgccgcctc |
| 421 | ggccgaccag ggacctgccc gcctgcggct gctccggacc tagaggatca agacataatg |
| 481 | ggagcatttt tagacaagcc aaagatggaa aagcataatg cccaggggca gggtaatggg |
| 541 | ttgcgatatg ggctaagcag catgcaaggc tggcgtgttg aaatggagga tgcacatacg |
| 601 | gctgtgatcg gtttgccaag tggacttgaa tcgtggtcat tctttgctgt gtatgatggg |
| 661 | catgctggtt ctcaggttgc caaatactgc tgtgagcatt tgttagatca catcaccaat |
| 721 | aaccaggatt ttaaagggtc tgcaggagca ccttctgtgg aaaatgtaaa gaatggaatc |
| 781 | agaacaggtt ttctggagat tgatgaacac atgagagtta tgtcagagaa gaaacatggt |
| 841 | gcagatagaa gtgggtcaac agctgtaggt gtcttaattt ctccccaaca tacttatttc |
| 901 | attaactgtg gagactcaag aggtttactt tgtaggaaca ggaaagttca tttcttcaca |
| 961 | caagatcaca aaccaagtaa tccgctggag aaagaacgaa ttcagaatgc aggtggctct |
| 1021 | gtaatgattc agcgtgtgaa tggctctctg gctgtatcga gggcccttgg ggattttgat |
| 1081 | tacaaatgtg tccatggaaa aggtcctact gagcagcttg tctcaccaga gcctgaagtc |
| 1141 | catgatattg aaagatctga agaagatgat cagttcatta tccttgcatg tgatggtatc |
| 1201 | tgggatgtta tgggaaatga agagctctgt gattttgtaa gatccagact tgaagtcact |
| 1261 | gatgaccttg agaaagtttg caatgaagta gtcgacacct gtttgtataa gggaagtcga |
| 1321 | gacaacatga gtgtgatttt gatctgtttt ccaaatgcac ccaaagtatc gccagaagca |
| 1381 | gtgaagaagg aggcagagtt ggacaagtac ctggaatgca gagtagaaga aatcataaag |
| 1441 | aagcaggggg aaggcgtccc cgacttagtc catgtgatgc gcacattagc gagtgagaac |
| 1501 | atccccagcc tcccaccagg gggtgaattg gcaagcaaga ggaatgttat tgaagccgtt |
| 1561 | tacaatagac tgaatcctta caaaaatgac gacactgact ctacatcaac agatgatatg |
| 1621 | tggtaaaact gctcatctag ccatggagtt taccttcacc tccaaaggag agtacagctc |
| 1681 | aactttgttg aaacttttaa catccatcct caactttaag gaaggggata tgacatgggt |
| 1741 | gagaatgatt acatcagaga acttcagcag tacaacagct agcccagaac tgattttttt |
| 1801 | tttttttttt gtaaatttga gacttatgta agcgtgattt caaaccataa ttcgtgttgt |
| 1861 | aaatcagact ccagcaattt ttgttgtatg attttgtttt tttgtaaagt gtaattgtcc |
| 1921 | ttgtacaaaa tgctcatatt taattatgaa ctgctttaaa tcactatcaa agttacaaga |
| 1981 | aatgtttggc ttattgtgtg atgcaacaga tatatagccc tttcaagtca tgttgtgttt |
| 2041 | ggacttgggg ttggaacagg gagagcagca gccatgtcag ctacacgctc aaatgtgcag |
| 2101 | atgattatgg aaaataacct caaaatctta caaagctgaa catccaagga gttattgaaa |
| 2161 | actatcttaa atgttcttgg taggggagtt ggcattgttg ataaagccag tcccttcatt |
| 2221 | taactgtctt tcaggatgtt ccttcgttgt ttccatgagt attgcaggta ataatacagt |
| 2281 | gtattcataa gaatctcaat cttggggcta aatgccttgt ttctttgcac ctcttttcaa |
| 2341 | gtccttacat ttaattacta attgataagc agcagcttcc tacatatagt aggaaactgc |
| 2401 | cacatttttg ctatcatgat tggctgggcc tgctgctgtt cctagtaaga tattctgaat |
| 2461 | tccattttat caataaagct tgatttaaca aacaagaaac ttaatcatgt atgtgtaatt |
| 2521 | cctcttttac cctggccttt taaaacactg tgccgttgta atgagacgtt tctcataggg |
| 2581 | aaagatgtta gtctctttta attggacaac actgtcactc aaggcataga tgaaactttc |
| 2641 | cttccattag aaagactaaa agatttaatt cttggttgta ccttaatcta ttttttaaat |
| 2701 | aggtttcttt caggctgctt atttttcatt aagatgtgta tcagcttgga tttgcctact |
| 2761 | gtttaattaa aatatttatt gtcaaagttt gacaatctaa cactctatgg taggggtgtg |
| 2821 | tgtgtgtgtg tttgtgagtg tgtgtttgcc tgtgattttt aattggccca tgtctttaga |
| 2881 | atccaagtgg ttaagatgta tttgtgattt gaaatatagc atgttgataa tatttagctg |
| 2941 | ttggccttta caaataactt tcaaagctta aggaattgta gatataaaaa taacctaatt |
| 3001 | taatttaggc ttaaattcct ctgattaagc atgtgaaagt aagttttaaa atctgtcgca |
| 3061 | ttgaaaagat tactgttccg tgcccttctg tatttttgtc tctttaggtt gaatattgta |
| 3121 | tttatcacca tgtaatcatt cagtaggcag attcccacta gaaaactgtt gaaatgtaag |
| 3181 | actaaaatac aacattgaat acaaaatcaa aattttgtgt ataaaaacca gtatagtcca |
| 3241 | ttttgttata tttgtttttt ccctaacttg gaaatataca tatttgtata tatagcctta |
| 3301 | aaattaatgt aaagttaggg gagtagtggg gaaagtaatg tgaaatgtct cagatttaag |
| 3361 | tagttaaata ccagcaaaat cttttcatta tccctcttat tttgtgaggt gattaaatgt |
| 3421 | aacttaattg tatttaattt atatcttatt ccagcatgaa tgaggaaaaa ctgaagtact |
| 3481 | atttatattt agaaattcat atcagttgaa attacagaac caattccata cttacaataa |
| 3541 | atacttaatg tctaaatctg tggtagagtg cgaagtatga taatgttcta agttatggct |
| 3601 | ttgcaagcat ctaaatgtgc atttaatgaa taccagtgct tctagtatag actaattacc |
| 3661 | agacatactg gtactgaaag ctaaatccct attataacaa accagttcct taatatttta |
| 3721 | agtagactga caactttagt tccagaaatt gcaaaacttt gaactggact gtgtaatctt |
| 3781 | ttgagatgca aaacttaagt cacaagtaga gtatgtgatg gaaagctgta tttcaaacca |
| 3841 | taacagcata tttagagcct tttttttttg agtctttaaa caagagaaaa ttaaaatatt |
| 3901 | cctgtcaaaa ttattagtat tgaaattagg cttggacacg agagagaacc gtatttgagt |
| 3961 | gatgtgagaa gactaaatct tttccacatg agtcagcact gccatactaa taattttttt |
| 4021 | actataaaaa tacaggaagg aagtatacat tataacagca gactgtgtgt gttcctgatt |
| 4081 | cctggaggta atagtggggg gaaaccaacc atacttttta aaggcacttt tgcacctcta |
| 4141 | ttgtgcactt cattcttgta ccacttaaat tcttcacccc catccccttt ttttgtgcta |
| 4201 | attagcatct cagggcaatg cctcaaaaat gtttgatgtg ttctgttctt tggagggaaa |
| 4261 | aagttcttat gtgatgataa tatagtactc aaaatatact tttatcattt aaatgtctta |
| 4321 | tttgctgcta tgaataggaa ataacatttt gtatagcagg ctctgtttta ccctaacatt |
| 4381 | aaaaaatttc actgatcttt ctttcattaa cagggtagaa tctcctaatt tccactttct |
| 4441 | tgggaatata cttttataga caatagaagc agttctcaat attagcatat actttaaaaa |
| 4501 | atcaaagtga taacttaatt cagctttgga agtatctcaa acatattttt actttatagt |
| 4561 | gcattaactt gcttctagag tacttaatgc aactgctcta gccacttaat tttttatact |
| 4621 | aatctcaaca ttaagaaatt tggattaagt aataaattag ttatgtaatt caagtaatct |
| 4681 | gaattacagc agtactttta gtgatcattc ataggactat atattaaccc agctaataac |
| 4741 | tcagtttttt tacaaaatgt ttcgagtatt attggtaaaa cactgttcta ggctaagcac |
| 4801 | attgggactg taaagaaatg agtagatcct tggcttcaag tttacatctg gacaatttat |
| 4861 | aatctagtgt atgttagtat tataactgga tcactcatca aaaaatatat atatatatct |
| 4921 | attgcccacc tgctatctac caggtactta gctgatcaag gcaggcccct gccctaaaga |
| 4981 | ccttgtttat acttccttta ctcacctgaa aactgttctc cagtttattt tcttctctct |
| 5041 | aaagttaaag agtaattcag aagaaaattt tgcttagcat aagaataaaa ttggactgaa |
| 5101 | gaggcttaag cccattcagt atccttgatt gcatttatcc aacggccttt attcttcctg |
| 4161 | ctgacagcag taactcagag gaataggtag tagatttctg aaaattatcc agccatggaa |
| 5221 | atgtaggtgg ggtttgagtt taaggcattt aaaaatgtaa atatctctag ctaaatttat |
| 5281 | cttaagtaga actctgtgtt tttgtaacac actgccagtg ttaatatcaa attttagcca |
| 5341 | aattattact atgtgtttta atattttaaa ataatttcac tgcccatctt tactggacaa |
| 5401 | actcatttgg agttcaactt gtgatttctg aaagaactga tgaaattggg tactgctttt |
| 5461 | tttctccatt tttcgttttg ttttaatttt gaatttcatg gtatatactt ttagttcaaa |
| 5521 | ctcagctgtt tgtacagtat tgtattagga tttggtatta gaaaagatgt gtaaatatct |
| 5581 | tagtatataa ttgtttctca tttgaggttt ttcttctaag ggaccttaaa gagttttata |
| 5641 | tacttttgct cacagaaact gctggtgaga ttaccatttt ttgagtatct agtcttctag |
| 5701 | tttttctttt aggcattagg aagccttctt tagagttcaa aattttagaa gcctaatttg |
| 5761 | ctcttacttc cttcaattat gtgccatgtg ttttggtttg tatatgtttt aaattgtata |
| 5821 | tttccttgga atatgcttga aatatttaag aatacatttt caaaatgtat aatactgtat |
| 5881 | tgttttgttg atcagaataa taagtctcag ttaaatgttt gttattactg atagtcaaaa |
| 5941 | tgctcaatag aaatgatgag aggcattggt tccaattcat tgtcaaatga acgttttcta |
| 6001 | attttgttca cagattcttt ccctttcgat tgttctgtat gttaagatag tggcttctgc |
| 6061 | tctcactgtt ttcctattta tattactagc aggtaggagt gctaattaga aaaacttaga |
| 6121 | tggtattgaa attacagttg acaacttata tttttatgag atggagaaaa aagattaagt |
| 6181 | tgatataaca acaaagtgga ctttttttct tccttatcct gcacgaaata ttgcccttgt |
| 6241 | ttcctctact ttcctcttgg tgttttctct ttttttcaaa cagaaacagg ccaattccat |
| 6301 | tttcttgagc aagaaagctt agtgtgttac ttcatcaagg ccagctaata ctgtgttaaa |
| 6361 | ccgggctgaa aatgagaaaa cttgggagat ggaggaatgg ggaaatggca gtgggatagg |
| 6421 | tagggaagga ttactcttaa ttgttttaaa agccatagga aagtcttcct tgtacgtggc |
| 6481 | tgtaaattta taagaactat tgtgtcacat aaaccaacaa gaatgaacct ttgctgcttc |
| 6541 | agataatttg atttttccag caaggaaatt aataagttac tgattcttca gcatagaaac |
| 6601 | aactgagaag aattaatgca atgtttcttc actagaaaac ccaacccttc atttcttttc |
| 6661 | attgctccaa aacccagttt tcaactaatg gttttctcat taaactaaat gtttagaaaa |
| 6721 | gttgtttaga gtttttcttt ttcttttaca tagtcctcct gatccagtat aagactattt |
| 6781 | agtaacgtgc atttgtatgg tactatctaa agtaagttag attgatgtaa gagatcgggt |
| 6841 | agctgcggaa caaaattagt tatatcctaa ttaggtacag tgaatgacac aaaatcattt |
| 6901 | tagcaatgct tcttaacctt ttggggtcac aggcgttttg agactgatga atcctaggga |
| 6961 | cttatttacc caggaaaatg cgtatataac atacatatct ccctaaagtt tacaatattg |
| 7021 | tagtggttca tgggccccct ggttaagagc ccattctaaa gtacaatagg gcatcatccc |
| 7081 | ttttcctgca aagcccaaaa gtatatttct agggcatgaa aataacttga gtctatttta |
| 7141 | aggaattgtt tcactctaga ggtagatagg ggacctggct agaatctgac attaaaatat |
| 7201 | actttttaaa aaatattata tttggggtgg ggaaagtgat taaaaggtga aaaaaaaaca |
| 7261 | tagtattcag aagttttgga ggttaatgtc tttctctaag atttgccact ttagaaattc |
| 7321 | aacagaaaag aggtaaaaca gaaatggaat gtatctggaa catttttggc ctccatagtg |
| 7381 | cagatatact atattaacaa gtaatacatt tatttacctg tcagatctcc aggttttaag |
| 7441 | attttgagct ttctagtatt aggattcatt aaatgttcaa ttcatttcat attctaagga |
| 7501 | attaggttat ttacttacta attcaggatg ttaaaataac atccaagtcg gacaaccacc |
| 7561 | accaatgcac acagttaatg agatttctaa aatataataa gtacaatgta acaaacgtat |
| 7621 | agaattttgc atttgttgcc aaaattagat gtttaatgac agcttattta attcccattt |
| 7681 | gtgggacttc tggaacatag aaaccattat cttacctggt tatcccttga ctaaatagca |
| 7741 | tatctgcagg aaaatatctt gtttgtagtg atatgcccca atagtgattg atttcactct |
| 7801 | tgaaatgagt tatatcactt aatttgtata aatgttatga gtggagagac atgtacatgt |
| 7861 | taaaagcatg ttgcattata tattcatttt ttaaactcta taaatgttaa gaataatata |
| 7921 | attgcagaaa tatttttctt aaatacaatg tgtaacaaaa ttctccgtag caactcaccc |
| 7981 | actttgcagt ttatgtgatc cacactttta aagaaattcc ataaatgtat attttgtatt |
| 8041 | atgtattatt tcctggtcca aagaaaatat gtgaattcag ttctaacttt aagaatgtac |
| 8101 | tgtttgtttt caagttcatt gaaaaattgc attcagcctg cgaatggttg cagattgtat |
| 8161 | gttagatgaa aagtagaaat aatttctagt ttggaaaact ggtgccacta aataaacagg |
| 8221 | caattacata a |

In some embodiments described herein, a PPM1A gene product is a PPM1A mRNA isoform transcript (for example, PPM1A mRNA transcript variant 2, corresponding to NCBI Reference Sequence NM_177951.2 (SEQ ID NO: 2865)), or a portion thereof. In some embodiments described herein, a PPM1A gene product is a nucleotide sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a PPM1A mRNA isoform transcript (for example, PPM1A mRNA transcript variant 2, corresponding to NCBI Reference Sequence NM_177951.2 (SEQ ID NO: 2865)), or a portion thereof.

In some embodiments described herein, a PPM1A gene product is a PPM1A mRNA isoform transcript (for example, PPM1A mRNA transcript variant 3, corresponding to NCBI Reference Sequence NM_177952.2 (SEQ ID NO: 2866)), or a portion thereof. In some embodiments described herein, a PPM1A gene product is a nucleotide sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with a PPM1A mRNA isoform transcript (for example, PPM1A mRNA transcript variant 3, corresponding to NCBI Reference Sequence NM_177952.2 (SEQ ID NO: 2866)), or a portion thereof.

In some embodiments described herein, a PPM1A gene product is a Mus musculus PPM1A mRNA isoform transcript (for example, Mus musculus PPM1A mRNA alpha isoform transcript, corresponding to NCBI Reference Sequence NM_008910.3 (SEQ ID NO: 2867)), or a portion thereof. In some embodiments described herein, a PPM1A gene product is a nucleotide sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99% identity, or 100% identity with a PPM1A mRNA isoform transcript (for example, Mus musculus PPM1A mRNA alpha isoform transcript, corresponding to NCBI Reference Sequence NM_008910.3 (SEQ ID NO: 2867)), or a portion thereof.

In some embodiments of the disclosure, the PPM1A gene product is a PPM1A mRNA transcript variant other than the PPM1A transcripts described above (e.g., PPM1A mRNA transcript variant 1, corresponding to NCBI Reference Sequence NM_021003.5 (SEQ ID NO: 2864), PPM1A mRNA transcript variant 2, corresponding to NCBI Reference Sequence NM_177951.2 (SEQ ID NO: 2865), PPM1A mRNA transcript variant 3, corresponding to NCBI Reference Sequence NM_177952.2 (SEQ ID NO: 2866), or Mus musculus PPM1A mRNA alpha isoform transcript, corresponding to NCBI Reference Sequence NM_008910.3 (SEQ ID NO: 2867)). In some embodiments, the PPM1A gene product is a nucleotide sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99% identity, or 100% identity with nucleotides homologous to nucleotides of PPM1A mRNA transcript variant 1, corresponding to NCBI Reference Sequence NM_021003.5 (SEQ ID NO: 2864), PPM1A mRNA transcript variant 2, corresponding to NCBI Reference Sequence NM_177951.2 (SEQ ID NO: 2865), PPM1A mRNA transcript variant 3, corresponding to NCBI Reference Sequence NM_177952.2 (SEQ ID NO: 2866), or Mus musculus PPM1A mRNA alpha isoform transcript, corresponding to NCBI Reference Sequence NM_008910.3 (SEQ ID NO: 2867). In some embodiments, the PPM1A gene product is a nucleotide sequence that shares at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity with nucleotides homologous to nucleotides 457-1429 of PPM1A mRNA transcript variant 1 (i.e., nucleotides 457-1429 of SEQ ID NO: 2864), or a portion thereof.

PPM1A AONs Targeting PPM1A Gene Product

In various embodiments, a PPM1A AON disclosed herein, such as PPM1A AONs with a sequence of any one of SEQ ID NOs: 2-955 or SEQ ID NOs: 1910-2863 or PPM1A Gapmer AONs with a sequence of any one of SEQ ID NOs: 2868-2959, target specific portions of a PPM1A gene product, such as a PPM1A mRNA transcript (e.g., any one of SEQ ID NO: 2864, SEQ ID NO: 2865, SEQ ID NO: 2866, or SEQ ID NO: 2867). In some embodiments, a PPM1A AON may be an oligonucleotide sequence at least 91%, at least 92%, at least 93%, at least 94%, at least 95%, at least 96%, at least 97%, at least 98%, at least 99%, or 100% complementary to a portion of a PPM1A gene product or to PPM1A gene sequence. In some embodiments described herein, a PPM1A AON targets a specific portion of a PPM1A gene product, such as a PPM1A mRNA transcript. Different embodiments of PPM1A mRNA transcripts targeted by PPM1A AONs are described in further detail below. For example, as described herein, a PPM1A AON includes linked nucleosides comprising a nucleobase sequence that is at least 91%, at least 92%, at least 93%, at least 94%, at least 95%, at least 96%, at least 97%, at least 98%, or at least 99% complementary to a PPM1A gene product, for example, a PPM1A mRNA transcript. In some embodiments, a PPM1A AON includes linked nucleosides comprising a nucleobase sequence that is 100% complementary to a PPM1A gene product, for example, a PPM1A mRNA transcript. In some embodiments, a PPM1A AON includes linked nucleosides comprising a nucleobase sequence that is at least 91%, at least 92%, at least 93%, at least 94%, at least 95%, at least 96%, at least 97%, at least 98%, or at least 99% complementary to a nucleotide sequence of an exon of a PPM1A gene sequence or a PPM1A mRNA sequence. In some embodiments, a PPM1A AON includes linked nucleosides comprising a nucleobase sequence that is 100% complementary to a nucleotide sequence of an exon of a PPM1A gene sequence or a PPM1A mRNA sequence. In some embodiments, a PPM1A AON includes linked nucleosides comprising a nucleobase sequence that is at least 91%, at least 92%, at least 93%, at least 94%, at least 95%, at least 96%, at least 97%, at least 98%, or at least 99% complementary to a nucleotide sequence of an untranslated region (UTR) of a PPM1A mRNA sequence, for example a 5′ UTR or a 3′ UTR of a PPM1A mRNA sequence. In some embodiments, a PPM1A AON includes linked nucleosides comprising a nucleobase sequence that is 100% complementary to a nucleotide sequence of an untranslated region (UTR) of a PPM1A mRNA sequence, for example a 5′ UTR or a 3′ UTR of a PPM1A mRNA sequence.

In some embodiments, a PPM1A AON targets a specific portion of the PPM1A gene product, the specific portion of the PPM1A gene product having a length of 10 nucleobases. In some embodiments, a PPM1A AON targets a specific portion of the PPM1A gene product, the specific portion of the PPM1A gene product having a length of 11, 12, 13, 14, 15, 16, 17, 18, 19, 20, 21, 22, 23, 24, or 25 nucleobases in length.

In some embodiments, a PPM1A AON disclosed herein target a contiguous nucleobase portion of a PPM1A gene product, such as a PPM1A mRNA transcript (e.g., any one of SEQ ID NO: 2864, SEQ ID NO: 2865, SEQ ID NO: 2866, or SEQ ID NO: 2867). In various embodiments, a PPM1A AON is at least 90% complementary to a contiguous 15 to 50 nucleobase portion of a PPM1A mRNA transcript (e.g., any one of SEQ ID NO: 2864, SEQ ID NO: 2865, SEQ ID NO: 2866, or SEQ ID NO: 2867).). In various embodiments, a PPM1A AON is at least 91%, at least 92%, at least 93%, at least 94%, at least 95%, at least 96%, at least 97%, at least 98%, at least 99%, or is 100% complementary to a contiguous 15 to 50 nucleobase portion of a PPM1A mRNA transcript (e.g., any one of SEQ ID NO: 2864, SEQ ID NO: 2865, SEQ ID NO: 2866, or SEQ ID NO: 2867). In various embodiments, a PPM1A AON is at least 91%, at least 92%, at least 93%, at least 94%, at least 95%, at least 96%, at least 97%, at least 98%, at least 99%, or is 100% complementary to a contiguous 16 to 45 nucleobase portion, 17 to 35 nucleobase portion, 18 to 30 nucleobase portion, 19 to 28 nucleobase portion, or 20 to 25 nucleobase portion of a PPM1A mRNA transcript (e.g., any one of SEQ ID NO: 2864, SEQ ID NO: 2865, SEQ ID NO: 2866, or SEQ ID NO: 2867).

In some embodiments, a PPM1A AON targets a specific portion of the PPM1A gene product, the specific portion of the PPM1A gene product comprising nucleotides 457-1429 of PPM1A mRNA transcript variant 1 (SEQ ID NO: 2864). In some embodiments, a PPM1A AON targets a specific portion of nucleotides 457-1429 of PPM1A mRNA transcript variant 1 (SEQ ID NO: 2864). In one embodiment, a PPM1A AON includes linked nucleosides with a nucleobase sequence having a portion of at least 10 contiguous nucleobases that is at least 90% complementary to an equal length portion of nucleobases in nucleotides 457-1429 of PPM1A mRNA transcript variant 1 (SEQ ID NO: 2864). In one embodiment, a PPM1A AON includes linked nucleosides with a nucleobase sequence having a portion of at least 10, at least 11, at least 12, at least 13, at least 14, at least 15, at least 16, at least 17, at least 18, or at least 19 contiguous nucleobases that is at least 91%, at least 92%, at least 93%, at least 94%, at least 95%, at least 96%, at least 97%, at least 98%, at least 99%, or is 100% complementary to an equal length portion of nucleobases in nucleotides 457-1429 of PPM1A mRNA transcript variant 1 (SEQ ID NO: 2864).

In various embodiments, a PPM1A AON targets any one of positions 542-814, 895-1006, 1025-1117, or 1361-1407 of SEQ ID NO: 2864. In one embodiment, a PPM1A AON includes linked nucleosides with a nucleobase sequence having a portion of at least 10 contiguous nucleobases that is at least 90% complementary to an equal length portion of nucleobases in positions 542-814, 895-1006, 1025-1117, or 1361-1407 of SEQ ID NO: 2864. In one embodiment, a PPM1A AON includes linked nucleosides with a nucleobase sequence having a portion of at least 10, at least 11, at least 12, at least 13, at least 14, at least 15, at least 16, at least 17, at least 18, or at least 19 contiguous nucleobases that is at least 91%, at least 92%, at least 93%, at least 94%, at least 95%, at least 96%, at least 97%, at least 98%, at least 99%, or is 100% complementary to an equal length portion of nucleobases in positions 542-814, 895-1006, 1025-1117, or 1361-1407 of SEQ ID NO: 2864.

In various embodiments, a PPM1A AON targets any one of positions 542-561, 555-574, 559-578, 599-618, 602-621, 603-622, 604-623, 605-624, 606-625, 607-626, 608-627, 609-628, 625-644, 642-661, 644-663, 646-665, 648-667, 650-669, 652-671, 655-674, 656-675, 708-727, 709-728, 794-813, 795-814, 895-914, 900-919, 905-924, 910-929, 915-934, 962-981, 967-986, 972-991, 977-996, 987-1006, 1025-1044, 1030-1049, 1034-1053, 1040-1059, 1045-1064, 1098-1117, 1361-1380, 1366-1385, 1371-1390, 1378-1397, and 1386-1405 of SEQ ID NO: 2864. In one embodiment, a PPM1A AON includes linked nucleosides with a nucleobase sequence having a portion of at least 10 contiguous nucleobases that is at least 90% complementary to an equal length portion of nucleobases in positions 542-561, 555-574, 559-578, 599-618, 602-621, 603-622, 604-623, 605-624, 606-625, 607-626, 608-627, 609-628, 625-644, 642-661, 644-663, 646-665, 648-667, 650-669, 652-671, 655-674, 656-675, 708-727, 709-728, 794-813, 795-814, 895-914, 900-919, 905-924, 910-929, 915-934, 962-981, 967-986, 972-991, 977-996, 987-1006, 1025-1044, 1030-1049, 1034-1053, 1040-1059, 1045-1064, 1098-1117, 1361-1380, 1366-1385, 1371-1390, 1378-1397, and 1386-1405 of SEQ ID NO: 2864. In one embodiment, a PPM1A AON includes linked nucleosides with a nucleobase sequence having a portion of at least 10, at least 11, at least 12, at least 13, at least 14, at least 15, at least 16, at least 17, at least 18, or at least 19 contiguous nucleobases that is at least 91%, at least 92%, at least 93%, at least 94%, at least 95%, at least 96%, at least 97%, at least 98%, at least 99%, or is 100% complementary to an equal length portion of nucleobases in positions 542-561, 555-574, 559-578, 599-618, 602-621, 603-622, 604-623, 605-624, 606-625, 607-626, 608-627, 609-628, 625-644, 642-661, 644-663, 646-665, 648-667, 650-669, 652-671, 655-674, 656-675, 708-727, 709-728, 794-813, 795-814, 895-914, 900-919, 905-924, 910-929, 915-934, 962-981, 967-986, 972-991, 977-996, 987-1006, 1025-1044, 1030-1049, 1034-1053, 1040-1059, 1045-1064, 1098-1117, 1361-1380, 1366-1385, 1371-1390, 1378-1397, and 1386-1405 of SEQ ID NO: 2864.

Nuclease-Mediated PPM1A Inhibition

In one aspect, the present disclosure provides a nuclease to reduce PPM1A expression. In some embodiments, the nuclease can be a Zinc Finger nuclease (ZFN), a meganuclease, a transcription activator-like effector nuclease (TALEN), or a clustered regularly interspaced short palindromic repeats (CRISPR) associated protein.

In certain embodiments, PPM1A inhibition is achieved using zinc finger nucleases (ZFNs). Synthetic ZFNs are composed of a zinc finger binding domain fused with, e.g., a FokI DNA cleavage domain. ZFNs can be designed/engineered for editing the genome of a cell, including, but not limited to, knock-out or knock-in gene expression, in a wide range of organisms. A meganuclease, a TALEN, or a CRISPR associated protein can be used for genome engineering in cells of a patient suffering from or at risk of a neurological disease, including neurons, for example, motor neurons, and other cells of the nervous system. The described reagents can be used to target promoters, protein-encoding regions (exons), introns, 5′ and 3′ UTRs, and more.

CRISPR genome editing typically comprises two distinct components: (1) a guide RNA and (2) an endonuclease, specifically a CRISPR associated (Cas) nuclease (e.g., Cas9). The guide RNA is a combination of the endogenous bacterial crRNA and tracrRNA into a single chimeric guide RNA (gRNA) transcript. Without being bound by theory, it is believed that when gRNA and the Cas are expressed in the cell, the genomic target sequence can be modified or permanently disrupted.

A gRNA/Cas complex can be recruited to a target sequence, for example, the PPM1A gene, by base-pairing between the gRNA sequence and the complement to the target DNA sequence in the PPM1A gene. An appropriate genomic target sequence contains a Protospacer Adjacent Motif (PAM) sequence immediately following the target sequence. The binding of the gRNA/Cas complex localizes the Cas to the PPM1A target sequence, allowing wild-type Cas to cut both strands of DNA, causing a double strand break. The double strand break is repaired through one of two general repair pathways: (1) the non-homologous end joining DNA repair pathway or (2) the homology directed repair pathway. The non-homologous repair pathway can result in insertions/deletions at the double strand break that can lead to frameshifts and/or premature stop codons, effectively disrupting the open reading frame of the target gene. The homology directed repair pathway requires the presence of a repair template, which is used to fix the double strand break.

In certain embodiments, PPM1A expression is reduced using CRISPR genome editing. In some embodiments, a gRNA pair is used to target a PPM1A gene to reduce and/or eliminate expression of PPM1A. In certain embodiments, one gRNA pair is used to reduce expression of PPM1A. In certain other embodiments, multiple gRNA pairs are used to reduce expression of PPM1A. gRNA pairs can be designed using known techniques and based on the PPM1A gene sequence. In certain embodiments, gRNA sequences may include modifications such as 2′ O-methyl analogs and 3′ phosphorothioate internucleotide linkages in the terminal three nucleotides on both 5′ and 3′ ends of the gRNA.

Neurological Diseases

Methods described herein may be used to treat neurological diseases including, but not limited to, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease.

Motor neuron diseases are a group of diseases characterized by loss of function of motor neurons that coordinate voluntary movement of muscles by the brain. Motor neuron diseases may affect upper and/or lower motor neurons, and may have sporadic or familial origins. Motor neuron diseases include amyotrophic lateral sclerosis (ALS or Lou Gehrig's disease), progressive bulbar palsy, pseudobulbar palsy, progressive muscular atrophy, primary lateral sclerosis, spinal muscular atrophy, post-polio syndrome, and ALS with frontotemporal dementia.

Symptoms of motor neuron diseases include muscle decay or weakening, muscle pain, spasms, slurred speech, difficulty swallowing, loss of muscle control, joint pain, stiff limbs, difficulty breathing, drooling, and complete loss of muscle control, including over basic functions such as breathing, swallowing, eating, speaking, and limb movement. These symptoms are also sometimes accompanied by depression, loss of memory, difficulty with planning, language deficits, altered behavior, and difficulty assessing spatial relationships and/or changes in personality.

Motor neuron diseases can be assessed and diagnosed by a clinician of skill, for example, a neurologist, using various tools and tests. For example, the presence or risk of developing a motor neuron disease can be assessed or diagnosed using blood and urine tests (for example, tests that assay for the presence of creatinine kinase), magnetic resonance imaging (MRI), electromyography (EMG), nerve conduction study (NCS), spinal tap, lumbar puncture, and/or muscle biopsy. Motor neuron diseases can be diagnosed with the aid of a physical exam and/or a neurological exam to assess motor and sensory skills, nerve function, hearing and speech, vision, coordination and balance, mental status, and changes in mood or behavior.

Amyotrophic Lateral Sclerosis

ALS is a progressive motor neuron disease that disrupts signals to all voluntary muscles. ALS results in atrophy of both upper and lower motor neurons. Symptoms of ALS include weakening and wasting of the bulbar muscles, general and bilateral loss of strength, spasticity, muscle spasms, muscle cramps, fasciculations, slurred speech, and difficulty breathing or loss of ability to breathe. Some individuals with ALS also suffer from cognitive decline. At the molecular level, ALS is characterized by protein and RNA aggregates in the cytoplasm of motor neurons, including aggregates of the RNA-binding protein TDP43.

ALS is most common in males above 40 years of age, although it can also occur in women and children. Risk of ALS is also heightened in individuals who smoke, are exposed to chemicals such as lead, or who have served in the military. Most instances of ALS are sporadic, while only about 10% of cases are familial. Causes of ALS include sporadic or inherited genetic mutations, high levels of glutamate, protein mishandling. Genetic mutations associated with ALS include mutations in the genes SOD1, C9orf72, TARDP, FUS, ANG, ATXN2, CHCHD10, CHMP2B, DCTN1, ERBB4, FIG4, HNRPA1, MATR3, NEFH, OPTN, PFN1, PRPH, SETX, SIGMAR1, SMN1, SPG11, SQSTM1, TBK1, TRPM7, TUBA4A, UBQLN2, VAPB, and VCP.

Frontotemporal Dementia

Frontotemporal dementia (FTD) is a form of dementia that affects the frontal and temporal lobes of the brain. It has an earlier average age of onset than Alzheimer's disease—40 years of age. Symptoms of FTD include extreme changes in behavior and personality, speech and language problems, and movement-related symptoms such as tremor, rigidity, muscle spasm, weakness, and difficulty swallowing. Subtypes of FTD include behavior variant frontotemporal dementia (bvFTD), characterized by changes in personality and behavior) and primary progressive aphasia (PPA), which affects language skills, speaking, writing and comprehension. FTD is associated with tau protein accumulation (Pick bodies) and function of altered TDP43 function. About 30% of cases of FTD are familial, and no other risk factors other than family history of the disease are known. Genetic mutations associated with FTD include mutations in the genes C9orf72, Progranulin (GRN), microtubule-associated protein tau (MAPT), UBQLN2, VPC, CHMP2B, TARDP, FUS, ITM2B, CHCHD10, SQSTM1, PSEN1, PSEN2, CTSF, CYP27A1, TBK1 and TBP.

Amyotrophic lateral sclerosis with frontotemporal dementia (ALS with FTD) is a clinical syndrome in which FTD and ALS occur in the same individual. Interestingly, mutations in C9orf72 are the most common cause of familial forms of ALS and FTD. Additionally, mutations in TBK1, VCP, SQSTMI, UBQLN2 and CHMP2B are also associated with ALS with FTD. Symptoms of ALS with FTD include dramatic changes in personality, as well as muscle weakness, muscle atrophy, fasciculations, spasticity, dysarthria, dysphagia, and degeneration of the spinal cord, motor neurons, and frontal and temporal lobes of the brain. At the molecular level, ALS with FTD is characterized by the accumulation of TDP-43 and/or FUS proteins. TBK1 mutations are associated with ALS, FTD, and ALS with FTD.

TBK1 and RIPK1 Function

In one aspect, methods described herein include exposing a cell to a PPM1A inhibitor to modify the activity, function, or other characteristics of a gene or a gene product, for example, an mRNA or protein. For example, methods described herein include a method of increasing or decreasing or inhibiting the activity, function, or other characteristics of a gene or a gene product. For example, described herein is a method of increasing phosphorylation of a residue of TANK-binding kinase 1 (also known as Serine/threonine-protein kinase TBK1; “TBK1”). For example, described herein is a method of increasing TBK1 serine residue 172 (ser172) phosphorylation in a cell, where the method includes exposing the cell to a PPM1A inhibitor. In some embodiments, TBK1 ser172 phosphorylation is increased in a cell of a patient suffering from ALS, FTD, or ALS with FTD. In some embodiments, the method of increasing TBK1 ser172 phosphorylation includes exposing a cell to a PPM1A antisense oligonucleotide of any one of SEQ ID NOs: 2-955, SEQ ID NOs: 1910-2863, SEQ ID NOs: 2868-2913, and SEQ ID NOs: 2914-2959.

Also described herein is a method of increasing TBK1 function in a cell, where the method includes exposing the cell to a PPM1A inhibitor. For example, described herein is a method of increasing TBK1 function in a cell, where the method includes exposing the cell to a PPM1A inhibitor. In some embodiments, TBK1 function is increased in a cell of a patient suffering from ALS, FTD, or ALS with FTD. In some embodiments, the method of increasing TBK1 function includes exposing a cell to a PPM1A antisense oligonucleotide of any one of SEQ ID NOs: 2-955, SEQ ID NOs: 1910-2863, SEQ ID NOs: 2868-2913, and SEQ ID NOs: 2914-2959.

Tank-binding kinase 1 (TBK1) is an IKK family of kinases that induces type-1 interferon activity and plays a major role in the phosphorylation of autophagy adaptors. Mutations in TBK1 are thought to result in impaired autophagy and contribute to the accumulation of protein aggregates and ALS pathology. At least 92 mutations in TBK1 have been identified in patients with ALS, FTD, or ALS with FTD (see Oakes et al., (2017) “TBK1: a new player in ALS linking autophagy and neuroinflammation” Molecular Brain 10:5, pg. 1-10). Furthermore, along with mutations in C9orf72, OPTN, SQSTM1/p62, UBQLN2, and TDP43, mutations in TBK1 account for approximately 15% of ALS and FTD patients. Furthermore, TBK1 haploinsufficiency associated with loss of function mutations has been identified as a major driver of familial ALS (see Freischmidt et al., (2015) “Haploinsufficiency of TBK1 causes familial ALS and fronto-temporal dementia” Nature Neuroscience, 18(5):631-6).

Autophagy is a process by which ubiquitinated proteins and damaged organelles are degraded and recycled. Abnormal protein aggregates are a hallmark of ALS pathology, and mutations in several genes involved in regulating autophagy are associated with ALS (for example, SQSTM1, SOD1, OPTN, VCP, UBQLN2, and TBK1). Thus, disruption of autophagy appears to contribute to ALS pathology.

Phosphorylation of residue Ser172 of TBK1 results in conformational changes in TBK1, that allow substrate binding by the protein's kinase domain. TBK1 phosphorylates a number of autophagy adaptors, and several TBK1 mutations identified in ALS patients inhibit the ability of TBK1 to phosphorylate these adaptors. Other TBK1 mutations result in decreased mRNA and protein levels. Additionally, individuals carrying mutations in TBK1 also display TDP43-positive aggregates in various brain regions. Thus, TBK1 mutations may result in decreased autophagy and accumulation of protein aggregates in motor neurons.

PPM1A is a member of the PP2C family of Ser/Thr protein phosphatases. PP2C family members are negative regulators of cellular stress-response pathways and are involved in regulating the cell-cycle and NF-κB pathways. PPM1A also dephosphorylates and inactivates TBK1. In particular, PPM1A dephosphorylates Ser172 of TBK1. Activated TBK1 can phosphorylate RIPK1 in such a manner that RIPK1 is deactivated. Thus PPM1A indirectly inactivates RIPK1

The present disclosure is based in part on the finding that increasing TBK1 activity, for example, increasing TBK1 activity in an individual or the cell of an individual that suffering from TBK1 haploinsufficiency, can be used as a mechanism to treat neurological diseases, for example, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

The disclosure is also based in part on the finding that increasing TBK1 activity, for example, increasing residual TBK1 activity in an individual and/or a cell of an individual suffering from TBK1 haploinsufficiency, can be achieved by increasing the amount of phosphorylated TBK1, for example, by increasing the amount of phosphorylated Ser172 TBK1, for example, an individual and/or a cell of an individual suffering from TBK1 haploinsufficiency. The disclosure is also based in part on the finding that increasing TBK1 activity, for example, increasing residual TBK1 activity in an individual and/or a cell of an individual suffering from TBK1 haploinsufficiency, can be achieved by increasing the ratio of phosphorylated TBK1 to total TBK1, for example, increasing the ratio of phosphorylated Ser172 TBK1 to unphosphorylated Ser172 TBK1, for example, in an individual and/or a cell of an individual suffering from TBK1 haploinsufficiency.

The disclosure is further based in part on the finding that increasing TBK1 activity (for example, increasing residual TBK1 activity in an individual and/or a cell of an individual suffering from TBK1 haploinsufficiency), increasing the amount of phosphorylated TBK1 (for example, increasing the amount of phosphorylated Ser172 TBK1, for example, in an individual and/or a cell of an individual suffering from TBK1 haploinsufficiency), and/or increasing the ratio of phosphorylated TBK1 to unphosphorylated TBK1 (for example, increasing the ratio of phosphorylated Ser172 TBK1 to unphosphorylated Ser172 TBK1, for example, in an individual and/or a cell of an individual suffering from TBK1 haploinsufficiency) can be achieved by inhibiting PPM1A activity and/or decreasing PPM1A protein levels, for example, in an individual and/or a cell of an individual suffering from a TBK1 haploinsufficiency. Without being bound by theory, it is believed that inhibiting PPM1A activity and/or decreasing PPM1A protein levels can be achieved by administering to a patient or a cell of a patient, a PPM1A inhibitor, for example, a PPM1A inhibitor described herein. In particular embodiments, the disclosure provides methods of inhibiting PPM1A activity and/or decreasing PPM1A protein amounts by administering to a patient or a cell of a patient (for example, a patient suffering from a neurological disease or a cell of a patient suffering from a neurological disease, for example, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease) a PPM1A AON, for example, a PPM1A AON comprising the nucleotide sequence of any one of SEQ ID NOs: 2-955, SEQ ID NOs: 1910-2863, SEQ ID NOs: 2868-2913, and SEQ ID NOs: 2914-2959.

Additionally disclosed herein is a method of modulating activity of Receptor Interacting Serine/Threonine Kinase 1 (also known as “RIPK1”). For example, described herein is a method of modulating RIPK1 activity in a cell, where the method includes exposing the cell to a PPM1A inhibitor. In various embodiments, modulating activity of RIPK1 can be useful for treating various diseases, including acute neuronal injury, multiple sclerosis, ALS, Alzheimer's Disease, Lysosomal Storage Diseases, Parkinson's Disease, and other human central nervous system diseases. In some embodiments, RIPK1 activity is modulated in a cell of a patient suffering from ALS, FTD, or ALS with FTD. In some embodiments, the method of modulating RIPK1 activity includes exposing a cell to a PPM1A antisense oligonucleotide of any one of SEQ ID NOs: 2-955, SEQ ID NOs: 1910-2863, SEQ ID NOs: 2868-2913, and SEQ ID NOs: 2914-2959.

TBK1 regulates RIPK1 through direct phosphorylation on multiple sites including Thr189 to suppress RIPK1 kinase activity by blocking the interaction with its substrates. Degterev, A. et al Targeting RIPK1 for the Treatment of Human Diseases, PNAS (2019), 116(20) 9714-9722 Therefore, increasing TBK1 function by increasing phosphorylation of a residue of TANK-binding kinase 1 can result in suppression of RIPK1 activity.

Methods of Treatment

The disclosure contemplates, in part, treating neurological diseases (for example, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease) in a patient in need thereof comprising administering a disclosed PPM1A inhibitor, for example, a PPM1A AON. In some embodiments, provided herein are methods for treatment of a neurological disease in a patient in need thereof, comprising administering a disclosed PPM1A inhibitor. In some embodiments of the disclosure, an effective amount of a disclosed PPM1A inhibitor may be administered to a patient in need thereof to treat a neurological disease, for example, to restore autophagy in cells of a patient suffering from a neurological disease, and/or to reduce or inhibit PPM1A. In some embodiments of the disclosure, an effective amount of a disclosed PPM1A inhibitor may be administered to a patient in need thereof to increase TBK1 phosphorylation (for example TBK1 ser172 phosphorylation) in a cell and/or to increase TBK1 function (for example, TBK1 kinase function) in a cell.

In some embodiments, methods of treating a neurological disease associated with impaired autophagy and/or protein aggregation (for example, TDP-43 protein aggregation, for example, in motor neurons) in a patient in need thereof are provided comprising administering a disclosed compound. In some embodiments, treating a neurological disease comprises at least ameliorating or reducing one symptom associated with the neurological disease (for example, reducing muscle weakness in a patient with ALS). Methods of treating a neurological disease (for example, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease) in a patient suffering therefrom are provided, that include administering a disclosed PPM1A inhibitor, for example, a PPM1A AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed PPM1A inhibitor, for example, a PPM1A AON. The methods include for example, treating a subject at risk of developing a neurological disease; e.g., administering to the subject an effective amount of a disclosed PPM1A AON. Neurological diseases that can be treated in this manner include amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

Methods of preventing or treating neurological diseases (for example, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease) form part of this disclosure. Such methods may comprise administering to a patient in need thereof or a patient at risk, a pharmaceutical preparation comprising an PPM1A AON such as a PPM1A AON disclosed herein. For example, a method of preventing or treating a neurological disease is provided comprising administering to a patient in need thereof a PPM1A AON disclosed herein.

Patients treated using an above method may experience a reduction of at least about 5%, 10%, 20%, 30%, 40%, 50%, 60%, 70%, 80%, 90%, or even 95% in the amount of PPM1A in a target cell (for example, a motor neuron) after administering PPM1A inhibitor, after e.g. 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. Administering such PPM1A inhibitor may be on, e.g., at least a daily basis. The PPM1A inhibitor may be administered orally. In some embodiments, the PPM1A inhibitor is administered intrathecally or intracisternally. For example, in an embodiment described herein, a PPM1A inhibitor is administered intrathecally or intracisternally about every 3 months. The delay or worsening of clinical manifestation of a neurological disease in a patient as a consequence of administering a PPM1A inhibitor disclosed here may be at least e.g., 6 months, 1 year, 18 months or even 2 years or more as compared to a patient who is not administered a PPM1A inhibitor such as one disclosed herein.

In another aspect, the disclosure provides methods of preventing, ameliorating, and/or treating a neurological disease, for example, a motor neuron disease. For example described herein are methods of preventing, ameliorating, and/or treating ALS, FTD, and ALS with FTD. In some embodiments, the disclosure provides a method of treating a neurological disease in a patient, for example, a patient in need of treatment of a neurological disease, where the method comprises administering to the patient a PPM1A inhibitor. In some embodiments, the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease.

In some embodiments, the patient is a mammal, for example, a human, a primate, a dog, a cat, a horse, a cow, a goat, a sheep, a mouse, or a rat. In particular embodiments, the patient is a human patient, for example, a human patient in need of treatment of a neurological disease, for example, ALS, FTD, or ALS with FTD. In some embodiments, the patient is a patient at risk of developing a neurological disease, for example, ALS, FTD, or ALS with FTD. In some embodiments, the patient is a patient suffering from a neurological disease, for example, ALS, FTD, or ALS with FTD. In some embodiments, the patient is a patient exhibiting symptoms associated with a neurological disease, for example, ALS, FTD, or ALS with FTD.

In another aspect, described herein are methods of modifying or restoring cellular function or activity, for example, cellular function or activity of a motor neuron. For example, described herein is a method of modifying or restoring cellular function or activity of a motor neuron of a patient at risk of or suffering from a neurological disease, for example, amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease. In some embodiments, the method includes exposing a cell to a PPM1A inhibitor, for example, a PPM1A antisense oligonucleotide. In some embodiments, the method includes exposing the cell to a PPM1A inhibitor in vivo or ex vivo.

In an embodiment described herein, the disclosure provides a method of increasing or restoring autophagy in a cell, where the method includes exposing the cell to a PPM1A inhibitor or contacting the cell with a PPM1A inhibitor. In some embodiments, the cell is a cell of a patient in need of treatment of a neurological disease. In some embodiments, the neurological disease is any one of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease. In some embodiments, the exposing or contacting is performed in vivo or ex vivo. For example, in an embodiment described herein, a cell of a patient suffering from ALS, FTD, or ALS with FTD is exposed to or contacted with a PPM1A inhibitor, for example, a PPM1A antisense therapeutic, for example, a PPM1A antisense oligonucleotide of any one of SEQ ID NOs: 2-955, SEQ ID NOs: 1910-2863, SEQ ID NOs: 2868-2913, and SEQ ID NOs: 2914-2959.

The PPM1A inhibitors, for example PPM1A AONs, of the invention can be used alone or in combination with each other where by at least two PPM1A inhibitors of the invention are used together in a single composition or as part of a treatment regimen. The PPM1A inhibitors of the invention may also be used in combination with other drugs for treating neurological diseases or conditions.

In various embodiments, methods of treating a neurological disease comprises selecting a patient for treatment using a PPM1A inhibitor disclosed herein. Selecting a patient for treatment can include measuring the presence or level of expression of certain markers of neurological disease. Examples of markers include neurofilament light (NEFL), neurofilament heavy (NEFH), phosphorylated neurofilament heavy chain (pNFH), TDP-43, or p75^{ECD}. Such markers can be measured from the plasma, the spinal cord fluid, the cerebrospinal fluid, the extracellular vesicles (for example, CSF exosomes), the blood, the urine, the lymphatic fluid, fecal matter, or a tissue of the patient.

In particular embodiments, the patient for treatment is selected by measuring phosphorylated neurofilament heavy chain (pNFH) in cerebrospinal fluid (CSF). In particular embodiments, the the pNFH in the CSF of the patient is used to predict disease status and survival in C9ORF72-associated amyotrophic lateral sclerosis (c9ALS) patients after initial administration and/or during on-going treatment.

In some embodiments, selecting a patient for treatment can include determining whether the patient expresses a mutation of a disease-associated gene. For example, a disease-associated gene can be an ALS-associated gene selected from any of TBK1, TARDBP, SQSTM1, VCP, C9orf72, FUS, and CHCHD10. For example, the patient can be identified as a candidate patient for treatment according to the determination that the patient includes one or more mutations in the disease-associated genes.

In various embodiments, a patient selected for treatment can be administered a PPM1A inhibitor disclosed herein and/or or a pharmaceutical composition thereof.

Treatment and Evaluation

In another aspect, the methods described herein include exposing a cell to a PPM1A inhibitor to inhibit or decrease activity or function of a gene or gene product, for example, an mRNA or protein. For example, described herein is a method of inhibiting PPM1A expression, activity, and/or function in a cell. For example, described herein is a method of inhibiting PPM1A in a cell, where the method includes exposing the cell to a PPM1A inhibitor. In some embodiments, PPM1A expression, activity, and/or function is inhibited in a cell of a patient suffering from ALS, FTD, or ALS with FTD. In some embodiments, the method of inhibiting PPM1A includes exposing a cell to a PPM1A antisense oligonucleotide of any one of SEQ ID NOs: 2-955, SEQ ID NOs: 1910-2863, SEQ ID NOs: 2868-2913, and SEQ ID NOs: 2914-2959.

In methods described herein, exposing a cell to a PPM1A inhibitor can include administering the PPM1A inhibitor, or a pharmaceutical composition that includes the PPM1A inhibitor, to a patient, for example, a patient suffering from or at risk of developing a neurological disease such as amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease. Thus, embodiments described herein can include administering a PPM1A inhibitor, or a pharmaceutical composition that includes a PPM1A inhibitor, to a patient in need of treatment, for example, a patient suffering from or at risk of developing a neurological disease such as amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease. Methods described herein embrace methods of administering a PPM1A inhibitor that allow administration of a therapeutically effective amount of the PPM1A inhibitor to a patient, for example, to a cell of a patient and/or to a site for treatment of a patient. For example, methods described herein include, but are not limited to, methods where a PPM1A inhibitor, or a pharmaceutical composition that includes a PPM1A inhibitor, is administered topically, parenterally, orally, buccally, sublingually, pulmonarily, intrathecally, intracisternally, intratracheally, intranasally, transdermally, rectally, vaginally, or intraduodenally. In particular embodiments, the PPM1A inhibitor is administered orally. In some embodiments, the PPM1A inhibitor is administered intrathecally or intracisternally. In embodiments described herein, the methods include administering a therapeutically effective amount of a PPM1A inhibitor, for example, a therapeutically effective amount of a PPM1A antisense oligonucleotide.

The methods described herein include methods of administering to a patient and/or exposing a cell to a PPM1A inhibitor, where the PPM1A inhibitor includes a PPM1A antisense oligonucleotide, for example, a PPM1A antisense oligonucleotide of any one of SEQ ID NOs: 2-955, SEQ ID NOs: 1910-2863, SEQ ID NOs: 2868-2913, and SEQ ID NOs: 2914-2959, or a pharmaceutically acceptable salt thereof. In some embodiments, the PPM1A inhibitor is formulated as a pharmaceutical formulation that includes a PPM1A antisense oligonucleotide, for example, a PPM1A antisense oligonucleotide of any one of SEQ ID NOs: 2-955, SEQ ID NOs: 1910-2863, SEQ ID NOs: 2868-2913, and SEQ ID NOs: 2914-2959, or a pharmaceutically acceptable salt thereof.

The methods described herein also include methods of administering to a patient and/or exposing a cell to a PPM1A inhibitor, where the PPM1A inhibitor is selected from the group consisting of a PPM1A small hairpin RNA (shRNA), a PPM1A small interfering RNA (siRNA), a PPM1A peptide nucleic acid (PNA), a PPM1A locked nucleic acid (LNA), and a PPM1A morpholino oligomer. In some embodiments, the PPM1A inhibitor is formulated as a pharmaceutical formulation that includes a PPM1A shRNA, a PPM1A siRNA, a PPM1A PNA, a PPM1A LNA, or a PPM1A morpholino oligomer, or a pharmaceutically acceptable salt of any of a PPM1A shRNA, a PPM1A siRNA, a PPM1A PNA, a PPM1A LNA, or a PPM1A morpholino oligomer.

In a further aspect, described herein is a use of a PPM1A inhibitor in the manufacture of a medicament for the treatment of neurological disease. For example, described herein is a use of a PPM1A inhibitor in the manufacture of a medicament for the treatment of ALS, FTD, or ALS with FTD. In some embodiments, the PPM1A inhibitor for use in the manufacture of a medicament for treatment is a PPM1A antisense oligonucleotide, or a pharmaceutically acceptable salt thereof, for example, a PPM1A antisense oligonucleotide of any one of SEQ ID NOs: 2-955, SEQ ID NOs: 1910-2863, SEQ ID NOs: 2868-2913, and SEQ ID NOs: 2914-2959, or a pharmaceutically acceptable salt thereof.

In a further aspect, described herein is a method of treating a neurological disease in a patient in need thereof, where the method includes administering to the patient in need thereof a pharmaceutical composition comprising a therapeutically effective amount of a PPM1A inhibitor, and a pharmaceutically acceptable excipient. In some embodiments, the neurological disease is amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease. In some embodiments, the PPM1A inhibitor is a PPM1A antisense oligonucleotide, or a pharmaceutically acceptable salt thereof, for example, a PPM1A antisense oligonucleotide of any one of SEQ ID NOs: 2-955, SEQ ID NOs: 1910-2863, SEQ ID NOs: 2868-2913, and SEQ ID NOs: 2914-2959, or a pharmaceutically acceptable salt thereof. In some embodiments, the PPM1A inhibitor is a PPM1A shRNA, a PPM1A siRNA, a PPM1A PNA, a PPM1A LNA, or a PPM1A morpholino oligomer. In some embodiments, the PPM1A inhibitor is a pharmaceutically acceptable salt of any of a PPM1A shRNA, a PPM1A siRNA, a PPM1A PNA, a PPM1A LNA, or a PPM1A morpholino oligomer.

In embodiments described herein, the pharmaceutical composition comprising a therapeutically effective amount of a PPM1A inhibitor, and a pharmaceutically acceptable excipient can be administered in any number of ways to achieve therapeutic delivery to a cell of a patient and/or to a site for treatment of a patient in need thereof. For example, in embodiments described herein, a pharmaceutical composition comprising a therapeutically effective amount PPM1A inhibitor, and a pharmaceutically acceptable excipient can be administered topically, parenterally, intrathecally, orally, pulmonarily, intratracheally, intranasally, transdermally, buccally, sublingually, rectally, vaginally, or intraduodenally. In particular embodiments, the pharmaceutical composition is administered orally. In some embodiments, the pharmaceutical composition is administered intrathecally or intracisternally. In embodiments described herein, the patient is a mammal, for example, a human patient.

In some embodiments, a PPM1A inhibitor described herein is for use as a medicament. For example, described herein is a PPM1A antisense oligonucleotide of any one of SEQ ID NOs: 2-955, SEQ ID NOs: 1910-2863, SEQ ID NOs: 2868-2913, and SEQ ID NOs: 2914-2959, or a pharmaceutically acceptable salt thereof, for use as a medicament.

In some embodiments, a PPM1A inhibitor, for example, a PPM1A antisense oligonucleotide described herein, is for use in the treatment of a neurological disease. For example, described herein is a PPM1A antisense oligonucleotide of any one of SEQ ID NOs: 2-955, SEQ ID NOs: 1910-2863, SEQ ID NOs: 2868-2913, and SEQ ID NOs: 2914-2959, or a pharmaceutically acceptable salt thereof, for use in the treatment of a neurological disease. In some embodiments, the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), and Gaucher's disease.

A patient, as described herein, refers to any animal at risk for, suffering from or diagnosed with a neurological disease, including, but not limited to, mammals, primates, and humans. In certain embodiments, the patient may be a non-human mammal such as, for example, a cat, a dog, or a horse. A patient may be an individual diagnosed with a high risk of developing a neurological disease, someone who has been diagnosed with a neurological disease, someone who previously suffered from a neurological disease, or an individual evaluated for symptoms or indications of a neurological disease, for example, decreased TBK1 expression signal or activity, impaired autophagy, TDP43 aggregation, or any of the signs or symptoms associated with neurological diseases such as amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

“A patient in need,” as used herein, refers to a patient suffering from any of the symptoms or manifestations of a neurological disease, a patient who may suffer from any of the symptoms or manifestations of a neurological disease, or any patient who might benefit from a method of the disclosure for treating a neurological disease. A patient in need may include a patient who is diagnosed with a risk of developing a neurological disease, a patient who has suffered from a neurological disease in the past, or a patient who has previously been treated for a neurological disease. Of particular relevance are individuals that suffer from a neurological disease associated with impaired TBK1 expression or activity or deleterious PPM1A expression or activity.

“Effective amount,” as used herein, refers to the amount of an agent that is sufficient to at least partially treat a condition when administered to a patient. The therapeutically effective amount will vary depending on the severity of the condition, the route of administration of the component, and the age, weight, etc. of the patient being treated. Accordingly, an effective amount of a disclosed PPM1A inhibitor is the amount of the PPM1A inhibitor necessary to treat a neurological disease in a patient such that administration of the agent prevents a neurological disease from occurring in a subject, prevents neurological disease progression (e.g., prevents the onset or increased severity of symptoms of the neurological disease such as muscle weakening, spasms, or fasciculation), or relieves or completely ameliorates all associated symptoms of a neurological disease, e.g., causes regression of the disease.

Efficacy of treatment may be evaluated by means of evaluation of gross symptoms associated with a neurological disease, analysis of tissue histology, biochemical assay, imaging methods such as, for example, magnetic resonance imaging, or other known methods. For instance, efficacy of treatment may be evaluated by analyzing gross symptoms of the disease such as changes in muscle strength and control or other aspects of gross pathology associated with a neurological disease following administration of a disclosed PPM1A inhibitor to a patient suffering from a neurological disease.

Efficacy of treatment may also be evaluated at the tissue or cellular level, for example, by means of obtaining a tissue biopsy (e.g., a brain, spinal, muscle, or motor neuron tissue biopsy) and evaluating gross tissue or cell morphology or staining properties, or by obtaining a biofluid (e.g., cerebrospinal fluid, exosomes, plasma, or urine) and examining PPM1A expression in the fluid using a biochemical assay that examines protein or RNA expression. Such biochemical assays can include ddPCR, qRT-PCR, western blot, ELISA, and/or SIMOA. For instance, one may evaluate levels of a protein (e.g., TBK1 or levels of another protein or gene product) indicative of a disease or a neurological disease, in dissociated cells or non-dissociated tissue via immunocytochemical, immunohistochemical, Western blotting, or Northern blotting methods, or methods useful for evaluating RNA levels such as quantitative or semi-quantitative polymerase chain (e.g., digital PCR (DigitalPCR, dPCR, or dePCR), qPCR etc.) reaction. One may also evaluate the presence or level of expression of useful biomarkers (e.g., neurofilament light (NEFL), neurofilament heavy (NEFH), TDP-43 or p75 extracellular domain (p75^{ECD})) found in spinal cord fluid, cerebrospinal fluid, plasma, extracellular vesicles (for example, exosome-like cerebrospinal fluid extracellular vesicles (“CSF exosomes”), such as those described in Welton et al., (2017) “Cerebrospinal fluid extracellular vesicle enrichment for protein biomarker discovery in neurological disease; multiple sclerosis” J Extracell Vesicles., 6(1):1-10; and Street et al., (2012) “Identification and proteomic profiling of exosomes in human cerebrospinal fluid” J Transl. Med., 10:5), urine, fecal matter, lymphatic fluid, blood, plasma, or serum to evaluate disease state and efficacy of treatment. Additional measurements of efficacy may include strength duration time constant (SDTC), short interval cortical inhibition (SICI), dynamometry, accurate test of limb isometric strength (ATLIS), compound muscle action potential (CMAP), and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilanent heavy chain (pNFH) in cerebrospinal fluid (CSF) predict disease status and survival in C9ORF72-associated amyotrophic lateral sclerosis (c9ALS) patients. CSF pNFH can serve as a prognostic biomarker for clinical trials, which will increase the likelihood of successfully developing a treatment for c9ALS.

In some embodiments, in evaluating the efficacy of a treatment against Alzheimer's disease, mental performance can be used for measuring efficacy such as with the Mini-Mental State Examination (MMSE). For measuring efficacy, the Functional Assessment Staging Test (FAST), the Motor Screening Task, Paired Associates Learning, Spatial Working Memory, Reaction time, Rapid Visual Information Processing, Delayed Matching to Sample, Pattern Recognition Memory can be used.

In some embodiments, in evaluating the efficacy of a treatment against Parkinson's disease, the Unified Parkinson's Disease Rating Scale (UPDRS) can be implemented as the performance measure. Other measures for quantifying aspects of functional performance not measured by the UPDRS can include the Berg Balance Scale (BBS), Forward Functional Reach Test (FFR), Backward Functional Reach Test (BFR), Timed “Up & Go” Test (TUG), and gait speed.

In evaluating efficacy of treatment, suitable controls may be chosen to ensure a valid assessment. For instance, one can compare symptoms evaluated in a patient with a neurological disease following administration of a disclosed PPM1A inhibitor to those symptoms in the same patient prior to treatment or at an earlier point in the course of treatment or in another patient not diagnosed with the neurological disease. Alternatively, one may compare the results of biochemical or histological analysis of tissue following administration of a disclosed PPM1A inhibitor with those of tissue from the same patient or from an individual not diagnosed with the neurological disease or from the same patient prior to administration of the PPM1A inhibitor. Additionally, one may compare blood, plasma, serum, cell, urine, lymphatic fluid, spinal cord fluid, cerebrospinal fluid, or fecal samples following administration of the PPM1A inhibitor with comparable samples from an individual not diagnosed with the neurological disease or from the same patient prior to administration of the PPM1A inhibitor. In some embodiments one may compare extracellular vesicles (for example CSF exosomes), following administration of the PPM1A inhibitor with extracellular vesicles from an individual not diagnosed with the neurological disease or from the same patient prior to administration of the PPM1A inhibitor.

Validation of PPM1A inhibition may be determined by direct or indirect assessment of PPM1A expression levels or activity. For instance, biochemical assays that measure PPM1A protein or RNA expression may be used to evaluate overall PPM1A inhibition. For instance, one may measure PPM1A protein levels in cells or tissue by Western blot to evaluate overall PPM1A levels. One may also measure PPM1A mRNA levels by means of Northern blot or quantitative polymerase chain reaction to determine overall PPM1A inhibition. One may also evaluate PPM1A protein levels or levels of another protein indicative of PPM1A signaling activity in dissociated cells, non-dissociated tissue, extracellular vesicles (for example, CSF exosomes), blood, serum, or fecal matter via immunocytochemical or immunohistochemical methods. PPM1A inhibition may also be evaluated indirectly by measuring parameters such as autophagy, endocytosis, protein aggregation, TBK1 expression, TBK1 kinase activity, changes in patient strength, muscle tone, presence of muscle spasms, enhanced speech, walking, breathing, or memory, or other parameters correlated with changes in PPM1A activity, including TBK1 target phosphorylation and other indicators of signaling activation of TBK1. For instance, one may measure levels of active TBK1 phosphorylation or the ratio of active (phosphorylated) to inactive TBK1 in cells of a patient treated with a disclosed PPM1A inhibitor as an indication of PPM1A activity in said cells. One may also evaluate the presence or level of expression of useful biomarkers (e.g., neurofilament light (NEFL), neurofilament heavy (NEFH), TDP-43, or p75^{ECD} found in plasma, spinal cord fluid, cerebrospinal fluid, extracellular vesicles (for example, CSF exosomes), blood, urine, lymphatic fluid, fecal matter, or tissue to evaluate efficacy of PPM1A inhibition. Additional measurements may include strength duration time constant (SDTC), short interval cortical inhibition (SICI), dynamometry, accurate test of limb isometric strength (ATLIS), compound muscle action potential, and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in c9ALS patients. CSF pNFH can serve as a prognostic biomarker for clinical trials, which will increase the likelihood of successfully developing a treatment for c9ALS.

Methods of treatment disclosed herein include methods of increasing or restoring autophagy in a cell. “Autophagy” refers to the natural, regulated mechanism of the cell that disassembles unnecessary or dysfunctional components, allowing orderly degradation and recycling of cellular components. Autophagy is generally responsible for degrading relatively long-lived, cytoplasmic proteins, soluble and insoluble misfolded proteins, and also entire organelles. Failure in autophagy machinery is thought to contribute to the formation of toxic protein aggregates in motor neurons (See Ramesh and Pandley, (2017) “Autophagy Dysregulation in ALS: When Protein Aggregates Get Out of Hand” Front Mol Neurosci. 10 (Article 263)). Dysregulation of autophagy and protein aggregation and mislocalization is implicated in neurological diseases, including ALS. Methods of increasing or restoring autophagy include methods that reduce expression levels of PPM1A in a patient suffering from a neurological disease. Methods of increasing or restoring autophagy also include methods that increase TBK1 activity or expression or TBK1 phosphorylation (for example, TBK1 ser172 phosphorylation) in cells of a patient suffering from a neurological disease.

The disclosure also provides methods of inhibiting PPM1A in cells of a patient suffering from a neurological disease. PPM1A may be inhibited in any cell in which PPM1A expression or activity occurs, including cells of the nervous system (including the central nervous system, the peripheral nervous system, motor neurons, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

Pharmaceutical Compositions and Routes of Administration

The present disclosure also provides methods for treating a neurological disease via administration of a pharmaceutical composition comprising a disclosed PPM1A inhibitor. In another aspect, the disclosure provides a pharmaceutical composition for use in treating a neurological disease. The pharmaceutical composition may be comprised of a disclosed antisense oligonucleotide that targets PPM1A and a pharmaceutically acceptable carrier. As used herein the term “pharmaceutical composition” means, for example, a mixture containing a specified amount of a therapeutic compound, e.g., a therapeutically effective amount, of a therapeutic compound in a pharmaceutically acceptable carrier to be administered to a mammal, e.g., a human, in order to treat a neurological disease. In some embodiments, contemplated herein are pharmaceutical compositions comprising a disclosed PPM1A inhibitor and a pharmaceutically acceptable carrier. In another aspect, the disclosure provides use of a disclosed PPM1A inhibitor in the manufacture of a medicament for treating a neurological disease. “Medicament,” as used herein, has essentially the same meaning as the term “pharmaceutical composition.”

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating suitable for regulating the site of absorption of the encapsulated substances within the digestive system or gut. For example, an enteric coating can include an ethylacrylate-methacrylic acid copolymer.

In some embodiments, a PPM1A inhibitor of the disclosure, for example, a PPM1A antisense oligonucleotide, is in the form of a pharmaceutically acceptable salt. PPM1A inhibitors described herein that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Pharmaceutically acceptable salts of the disclosure include, for example, pharmaceutically acceptable salts of a PPM1A antisense oligonucleotide of any of SEQ ID NOs: 2-955, SEQ ID NOs: 1910-2863, SEQ ID NOs: 2868-2913, and SEQ ID NOs: 2914-2959.

Also described herein are pharmaceutical compositions comprising a PPM1A inhibitor and a pharmaceutically acceptable excipient. For example, a pharmaceutical composition described herein can include a PPM1A antisense oligonucleotide, for example, a PPM1A antisense oligonucleotide of any one of SEQ ID NOs: 2-955, SEQ ID NOs: 1910-2863, SEQ ID NOs: 2868-2913, and SEQ ID NOs: 2914-2959, and a pharmaceutically acceptable excipient.

In some embodiments, a PPM1A inhibitor, for example a PPM1A AON, can be encapsulated in a nanoparticle coating. It is believed that nanoparticle encapsulation prevents AON degradation and enhances cellular uptake. For example, in some embodiments a PPM1A inhibitor is encapsulated in a coating of a cationic polymer, for example, a synthetic polymer (e.g., poly-L-lysine, polyamidoamine, a poly(O-amino ester), and polyethyleneimine) or a naturally occurring polymer (e.g., chitosan and a protamine). In some embodiments, a PPM1A inhibitor is encapsulated in a lipid or lipid-like material, for example, a cationic lipid, a cationic lipid-like material, or an ionizable lipid that is positively charged only at an acidic pH. For example, in some embodiments, a PPM1A inhibitor is encapsulated in a lipid nanoparticle that includes hydrophobic moieties, e.g., cholesterol and/or a polyethylene glycol (PEG) lipid.

In some embodiments, a PPM1A inhibitor, for example, a PPM1A AON, is conjugated to a bioactive ligand. For example, in some embodiments described herein, a PPM1A inhibitor such as a PPM1A AON is conjugated to a peptide, a lipid, N-acetylgalactosamine (GalNAc), cholesterol, vitamin E, an antibody, or a cell-penetrating peptide (for example, transactivator of transcription (TAT) and penetratine).

Pharmaceutical compositions containing a disclosed PPM1A inhibitor, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, for example, are sterile. Sterilization can be accomplished, for example, by filtration through sterile filtration membranes. Where the composition is lyophilized, filter sterilization can be conducted prior to or following lyophilization and reconstitution.

In one embodiment, a disclosed PPM1A inhibitor and any pharmaceutical composition thereof may be administered by one or several routes, including topically, intrathecally, parenterally, orally, rectally, buccally, sublingally, vaginally, pulmonarily, intratracheally, intracisternally, intranasally, transdermally, or intraduodenally. The term parenteral as used herein includes subcutaneous injections, intrapancreatic administration, intravenous, intracisternal, intrathecal, intramuscular, intraperitoneal, intrasternal injection or infusion techniques. For example, a disclosed PPM1A inhibitor may be administered subcutaneously to a subject. In another example, a disclosed PPM1A inhibitor may be administered orally to a subject. In another example, a disclosed PPM1A inhibitor may be administered directly to the nervous system, or specific regions or cells of the nervous system (e.g., the brain, brain stem, lower motor neurons, spinal cord, upper motor neurons) via parenteral administration, for example, a disclosed PPM1A inhibitor may be administered intrathecally or intracisternally.

It will be appreciated that the PPM1A inhibitor, for example, the PPM1A antisense oligonucleotide administered to the patient having or at risk of a neurological disease in methods described herein, can be administered by various administration routes. In various embodiments, the PPM1A inhibitor can be administered by one or several routes, including orally (e.g., by inhalation spray), topically, vaginally, rectally, intrathecally, intracisternally, buccally, sublingually, parenterally, e.g., by subcutaneous injection. The term parenteral as used herein includes subcutaneous injections, intrapancreatic administration, and intravenous, intrathecal, intracisternal, intramuscular, intraperitoneal, and intrasternal injection or infusion techniques.

Parenteral Administration

The pharmaceutical compositions of the disclosure can be formulated for parenteral administration, e.g., formulated for injection via the intravenous, intracisternal, intramuscular, subcutaneous, intrathecal, intralesional, or intraperitoneal routes. The preparation of an aqueous composition, such as an aqueous pharmaceutical composition containing a disclosed PPM1A inhibitor, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, a disclosed PPM1A antisense oligonucleotide may be suspended in a carrier fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a disclosed PPM1A antisense oligonucleotide in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the disclosed PPM1A inhibitor to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

Intrathecal Administration

In some embodiments, a PPM1A inhibitor, or a pharmaceutical composition of the disclosure that includes a PPM1A inhibitor, is delivered to the CNS through intrathecal administration, thereby ensuring delivery into the cerebrospinal fluid (CSF) of a patient in need of treatment. In various embodiments, intrathecal administration (also referred to as intrathecal injection) refers to an injection into the spinal canal (intrathecal space surrounding the spinal cord). Various techniques may be used including, without limitation, lateral cerebroventricular injection through a burrhole or cisternal or lumbar puncture or the like. In some embodiments, “intrathecal administration” or “intrathecal delivery” according to the present invention refers to IT administration or delivery via the lumbar area or region, e.g., lumbar IT administration or delivery. As used herein, the term “lumbar region” or “lumbar area” refers to the area between the third and fourth lumbar (lower back) vertebrae and, more inclusively, the L2-S1 region of the spine.

In various embodiments, compositions comprising a disclosed PPM1A inhibitor can be suitable for intrathecal delivery. For example, a composition suitable for intrathecal delivery can comprise the PPM1A inhibitor and any of cerebrospinal fluid, artificial cerebrospinal fluid, phosphate buffered saline (PBS), or salt buffer.

Oral Administration

In some embodiments, contemplated herein are compositions suitable for oral delivery of a disclosed PPM1A inhibitor, e.g., tablets that include an enteric coating, e.g., a gastro-resistant coating, such that the compositions may deliver a PPM1A inhibitor to, e.g., the gastrointestinal tract of a patient.

For example, a tablet for oral administration is provided that comprises granules (e.g., is at least partially formed from granules) that include a disclosed PPM1A inhibitor, e.g., an PPM1A antisense oligonucleotide, e.g., a PPM1A antisense oligonucleotide represented by any of SEQ ID NOs: 2-955, SEQ ID NOs: 1910-2863, SEQ ID NOs: 2868-2913, and SEQ ID NOs: 2914-2959, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed PPM1A inhibitor, e.g. a PPM1A antisense oligonucleotide, e.g., a PPM1A antisense oligonucleotide represented by any of SEQ ID NOs: 2-955, SEQ ID NOs: 1910-2863, SEQ ID NOs: 2868-2913, and SEQ ID NOs: 2914-2959, and a pharmaceutically acceptable salt, e.g. a PPM1A antisense oligonucleotide, e.g., an antisense oligonucleotide represented by any of SEQ ID NOs: 2-955, SEQ ID NOs: 1910-2863, SEQ ID NOs: 2868-2913, and SEQ ID NOs: 2914-2959, and a pharmaceutically acceptable filler. For example, a disclosed PPM1A inhibitor and a filler may be blended together, optionally, with other excipients, and formed into granules. In some embodiments, the intragranular phase may be formed using wet granulation, e.g. a liquid (e.g., water) is added to the blended PPM1A inhibitor compound and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or an extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as but are not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed PPM1A inhibitor and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8 to about 15%, about 8% to about 20%, about 10% to about 20%, or about 12 to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g. about 0.5% to about 10%, or about 1% to about 20%, by weight of a disclosed PPM1A antisense oligonucleotide or a pharmaceutically acceptable salt thereof. Such a tablet may include for example, about 0.5% to about 60% by weight of mannitol, e.g. about 30% to about 50% by weight mannitol, e.g. about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a disclosed PPM1A antisense oligonucleotide, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g. about 2% to about 4% sodium starch glycolate by weight.

In another contemplated embodiment, a pharmaceutical tablet formulation for oral administration of a disclosed PPM1A inhibitor comprises an intra-granular phase, wherein the intra-granular phase includes a disclosed PPM1A AON or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may comprise about 0.5% to 10% by weight of a disclosed PPM1A AON, e.g., a disclosed PPM1A AON or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a disclosed PPM1A AON, e.g., a disclosed PPM1A AON or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., PCT Publication No. WO 2010/054826, which is hereby incorporated by reference in its entirety).

The rate at which the coating dissolves and the active ingredient is released is its dissolution rate. In an embodiment, a contemplated tablet may have a dissolution profile, e.g. when tested in a USP/EP Type 2 apparatus (paddle) at 100 rpm and 37° C. in a phosphate buffer with a pH of 7.2, of about 50% to about 100% of the PPM1A inhibitor releasing after about 120 minutes to about 240 minutes, for example after 180 minutes. In another embodiment, a contemplated tablet may have a dissolution profile, e.g. when tested in a USP/EP Type 2 apparatus (paddle) at 100 rpm and 3TC in diluted HCl with a pH of 1.0, where substantially none of the PPM1A inhibitor is released after 120 minutes. A contemplated tablet, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the PPM1A inhibitor releasing after 30 minutes.

In some embodiments, methods provided herein may further include administering at least one other agent that is directed to treatment of diseases and disorders disclosed herein. In one embodiment, contemplated other agents may be co-administered (e.g., sequentially or simultaneously).

Dosage and Frequency of Administration

Exemplary formulations include dosage forms that include or consist essentially of about 35 mg to about 500 mg of a disclosed PPM1A inhibitor, for example, a PPM1A AON. For example, formulations that include about 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, 1 g, 1.5 g, 2.0 g, 2.5 g, 3.0 g, 3.5 g, 4.0 g, 4.5 g, or 5.0 g of a disclosed PPM1A inhibitor are contemplated herein. In one embodiment, a formulation may include about 40 mg, 80 mg, or 160 mg of a disclosed PPM1A inhibitor. In some embodiments, a formulation may include at least 100 μg of a disclosed PPM1A inhibitor. For example, formulations may include about 0.1 mg, 0.2 mg, 0.3 mg, 0.4 mg, 0.5 mg, 1 mg, 5 mg, 10 mg, 15 mg, 20 mg, 25 mg, or 30 mg of a disclosed PPM1A inhibitor.

In some embodiments, methods described herein include administering at least 1 μg, at least 5 μg, at least 10 μg, at least 20 μg, at least 30 μg, at least 40 μg, at least 50 μg, at least 60 μg, at least 70 μg, at least 80 μg, at least 90 μg, or at least 100 μg of a PPM1A inhibitor, for example a PPM1A inhibitor. In some embodiments, methods of the invention include administering from 35 mg to 500 mg, from 1 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of a PPM1A inhibitor.

The amount administered will depend on variables such as the type and extent of disease or indication to be treated, the overall health and size of the patient, the in vivo potency of the PPM1A inhibitor, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study. Dosing frequency can vary, depending on factors such as route of administration, dosage amount and the disease being treated. Exemplary dosing frequencies are once per day, once per week and once every two weeks. In some embodiments, dosing is once per day for 7 days. In some embodiments, dosing is once per month. In some embodiments, dosing is once every 3 months.

Combination Therapies

In various embodiments, a PPM1A AON as disclosed herein can be administered in combination with one or more additional therapies. The combination therapy of the disclosed oligonucleotide and the one or more additional therapies can, in some embodiments, be synergistic in treating any of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

Example additional therapies for treating amyotrophic lateral sclerosis (ALS), frontotemporal dementia (FTD), or ALS with FTD include any of Riluzole (Rilutek), troriluzole, Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO®), ZILUCOPLAN (RA101495), dual AON intrathecal administration (e.g., BIIB067, BIIB078), BIIB100, levodopa/carbidopa, dopaminergic agents (e.g., ropinirole, pramipexole, rotigotine), medroxyprogesterone, KCNQ2/KCNQ3 openers, Pridopidine, PrimeC (combination of ciprofloxacin and Celebrex), lithium, or anticonvulsants and psychostimulant agents. Additional therapies can further include breathing care, physical therapy, occupational therapy, speech therapy, and nutritional support. In various embodiments, an additional therapy can be a second antisense oligonucleotide. As an example, the second antisense oligonucleotide may be a second PPM1A AON that targets a PPM1A transcript.

A combination therapy (e.g., in combination with a PPM1A AON) may be selected according to the disease that is to be treated. For example, for treating Alzheimer's Disease, any of Memantine, Rivastigmine, Galantamine, Donepezil, Aricept®, Exelon® (Rivastigmine), Razadyne®, Aducanumab, BAN2401, BIIB091 (gosuranemab), BIIB076, BIIB080 (IONIS-MAPTRx), Elayta (CT1812), MK1942, allogenic hMSC, nilotinib, A1BT-957, acitretin, ABT-354, GV1001, Riluzole, CAD106, CNP520, AD-35, Rilapladib, DHP1401, T-817 MA, TC-5619, TPI-287, RVT-101, LY450139, JNJ-54861911, Dapagliflozin, GSK239512, PF-04360365, ASP0777, SB-742457 (a 5-HT6 receptor antagonist), PF-03654746 (an H₃ receptor antagonist), GSK933776 (an Fc-inactivated anti-(3 amyloid (AD) monoclonal antibody (mAb)), Posiphen ((+)-phenserine tartrate), AMX0035 (ELYBRIO®), coenzyme Q10 or any combination thereof can be selected as an additional therapy.

For example for treating Parkinson's Disease, any of Levodopa, Carbidopa-levidopa, pramipexole, ropinirole, rotigotine, apomorphine, selegiline, rasagiline, entacapone, tolcapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stein stimulation, nicotine, nabilone, PF-06649751, DNL201, LRRK2 inhibitors. CK1 inhibitors, isradipine, CLR4001, IRX4204, Yohimbine, coenzyme Q10, OXB-10, duloxetine, pioglitazone, preladenant, or any combination thereof can be selected as an additional therapy.

For example, for treating progressive supranuclear palsy (PSP), any of UCB0107, ABBV-8E12, F-18 AV1451, BIIB092, C-2N-8E12, tideglusib, deep transcranial magnetic stimulation, lipoic acid, tolfenamica acid, lithium, AZP2006, Glial Cell Line-Derived Neurotrophic Factor, NBMI, suvorxant, zolpidem, TPI 287, davunetide, pirnavanserin, Levodopa, Carbidopa-levidopa, pramipexole, ropinirole, rotigotine, apomorphine, selegiline, rasagiline, entacapone, toicapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, or any combination thereof can be selected as an additional therapy.

For example for treating Huntington's Disease, any of Tetrabenazine, deutetrabenazine, physical therapy, risperidone, haloperidol, chlorpromazine, clonazepam, diazepan, benzodiazepines, selective serotonin reuptake inhibitors, quetiapine, carbatrol, valproate, lamotrigine, pridopidine, delta-9-tetrahydrocannabinol, cannabidiol, stem-cell therapy, ISIS-443139, nilotinib, resveratrol, neflanapimod, fenofibrate, creatine, RO7234292, SAGE-718, WVE-120102, WVE-120101, dime bon, minocycline, deep brain stimulation, ursodiol, coenzyme Q10, OMS643762, VX15/2503, PF-02545920, BN82451B, SEN0014196, olanzapine, tiapridal (tiapride), or any combination thereof, can be selected as an additional therapy.

For example, for treating brain trauma, any of anticoagulants, antidepressants, muscle relaxants, stimulants, anticonvulsants, anti-anxiety medication, erythropoietin, hyperbaric treatment, rehabilitation therapies (e.g., physical, occupational, speech, psychological, or vocational counseling), or any combination thereof can be selected as an additional therapy.

For example, for treating spinal cord injury, any of AXER-204, glyburide, 5-hydroxytryptophan (5-HTP), L-3,4-dihydroxyphenylalanine (L-DOPA), or rehabilitation therapies (e.g., physical therapy, occupational therapy, recreational therapy, use of assistive devices, improved strategies for exercise and healthy diets), or any combination thereof can be selected as an additional therapy.

For example, for treating corticobasal degeneration, any of TPI-287, lithium, occupational, physical, and speech therapy, or any combination thereof can be selected as an additional therapy.

For example, for treating neuropathies, such as a chemotherapy induced neuropathy, any of gabapentin, pregabalin, lamotrigine, carbamazepine, duloxetine, gabapentinoids, tricyclic antidepressants, serotonin-norepinephrine reuptake inhibitors, opioids, neurotoxin, dextromethorphan, nicotinamide riboside, auto-antibodies targeting neuronal antigens (TS-HDS and FGFR3), or any combination thereof can be selected as an additional therapy.

For example, for treating spinocerebellar ataxia, any of troriluzole, BHV-4157, or a combination thereof can be selected as an additional therapy.

For example, for treating Niemann-Pick disease type C, any of anti-seizure medications, speech therapy, physical therapy, occupational therapy, Adrabetadex, Arimoclomol, N-Acetyl-L-Leucine, or any combination thereof can be selected as an additional therapy.

For example, for treating Charcot-Marie-Tooth Disease (CMT), any of physical and occupational therapies, orthopedic surgery, orthopedic devices, PXT3003, or any combination thereof can be selected as an additional therapy.

For example, for treating Mucopolysaccharidosis type II (MPSIIA), any of enzyme replacement therapy: idursulfase (Elaprase), surgical intervention (tonsillectomy and/or adenoidectomy), RGX-121 gene therapy, adalimumab, MT2013-31, or any combination thereof can be selected as an additional therapy.

For example, for treating Mucolipidosis IV, any of physical, occupational, and speech therapies, contact lenses and artificial tears, genetic counseling, or any combination thereof can be selected as an additional therapy.

For example, for treating GM1 gangliosidosis, any of anticonvulsants, physical and occupational therapies, galactosidase, gene delivery of galactosidase, LYS-GM101 gene therapy, or any combination thereof can be selected as an additional therapy.

For example, for treating Sporadic inclusion body myositis (sIBM), any of physical and occupational therapies, use of devices such as braces, walkers, wheelchairs, immunosuppressants, BYM338, or any combination thereof can be selected as an additional therapy.

For example, for treating Henoch-Schonlein purpura (HSP), any of corticosteroids, colchicine, dapsone, azathioprine, or any combination thereof can be selected as an additional therapy.

For example, for treating Gaucher's disease, any of enzyme replacement therapy, substrate reduction therapy, N-acetylcysteine, GZ/SAR402671, cerezyme, or any combination thereof can be selected as an additional therapy.

In various embodiments, the disclosed oligonucleotide and the one or more additional therapies can be conjugated to one another and provided in a conjugated form. Further description regarding conjugates involving the disclosed oligonucleotide is described below.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. In various embodiments, the disclosed oligonucleotide and one or more additional therapies are provided concurrently. In various embodiments, the disclosed oligonucleotide and one or more additional therapies are provided simultaneously. In various embodiments, the disclosed oligonucleotide and one or more additional therapies are provided sequentially.

Conjugates

In certain embodiments, provided herein are oligomeric compounds, which comprise an oligonucleotide (e.g., PPM1A AON) and optionally one or more conjugate groups and/or terminal groups. Conjugate groups include one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

Conjugate Groups

In certain embodiments, a PPM1A AON is covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In particular embodiments, conjugate groups modify the circulation time (e.g., increase) of the oligonucleotides in the bloodstream such that increased concentrations of the oligonucleotides are delivered to the brain. In particular embodiments, conjugate groups modify the residence time (e.g., increase residence time) of the oligonucleotides in a target organ (e.g., brain) such that increased residence time of the oligonucleotides improves their performance (e.g., efficacy). In particular embodiments, conjugate groups increase the delivery of the oligonucleotide to the brain through the blood brain barrier and/or brain parenchyma (e.g., through receptor mediated transcytosis). In particular embodiments, conjugate groups enable the oligonucleotide to target a specific organ (e.g., the brain). In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. NY. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes. In particular embodiments, conjugate moieties are selected from a peptide, a lipid, N-acetylgalactosamine (GalNAc), cholesterol, vitamin E, lipoic acid, panthothenic acid, polyethylene glycol, an antibody (e.g., an antibody for crossing the blood brain barrier such as anti-transferrin receptor antibody), or a cell-penetrating peptide (e.g., transactivator of transcription (TAT) and penetratine).

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

Conjugate Linkers

Conjugate moieties are attached to a PPM1A AON through conjugate linkers. In certain oligomeric compounds, the conjugate linker is a single chemical bond (e.g., the conjugate moiety is attached directly to an oligonucleotide through a single bond). In certain embodiments, the conjugate linker comprises a chain structure, an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise 3 linker-nucleosides.

In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In certain embodiments, it is desirable for a conjugate group to be cleaved from the PPM1A AON. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxy adenosine.

Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

Diagnostic Methods

The disclosure also provides a method of diagnosing a patient with a neurological disease that relies upon detecting levels of PPM1A expression signal in one or more biological samples of a patient. As used herein, the term “PPM1A expression signal” can refer to any indication of PPM1A gene expression, or gene or gene product activity. PPM1A gene products include RNA (e.g., mRNA), peptides, and proteins. Indices of PPM1A gene expression that can be assessed include, but are not limited to, PPM1A gene or chromatin state, PPM1A gene interaction with cellular components that regulate gene expression, PPM1A gene product expression levels (e.g., PPM1A RNA expression levels, PPM1A protein expression levels), or interaction of PPM1A RNA or protein with transcriptional, translational, or post-translational processing machinery. Indices of PPM1A gene product activity include, but are not limited to, assessment of PPM1A signaling activity (e.g., assessment of TBK1 activation or phosphorylation).

Detection of PPM1A expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, extracellular vesicles (for example, CSF exosomes), or cells of a patient. Detection may be achieved by measuring PPM1A expression signal in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, or serum. Biochemical assays that examine protein or RNA expression may also be used for detection. For instance, one may evaluate levels of a protein (e.g., TBK1 or levels of another protein or gene product) indicative of a neurological disease, in dissociated cells or non-dissociated tissue via immunocytochemical, immunohistochemical, Western blotting, or Northern blotting methods, or methods useful for evaluating RNA levels such as quantitative or semi-quantitative polymerase chain (e.g., digital PCR (DigitalPCR, dPCR, or dePCR), qPCR etc.) reaction.

One may also evaluate the presence or level of expression of useful biomarkers (e.g., neurofilament light (NEFL), neurofilament heavy (NEFH), TDP-43 or p75 extracellular domain (p75^{ECD})) found in spinal cord fluid, cerebrospinal fluid, plasma, extracellular vesicles (for example, exosome-like cerebrospinal fluid extracellular vesicles (“CSF exosomes”), such as those described in Welton et al., (2017) “Cerebrospinal fluid extracellular vesicle enrichment for protein biomarker discovery in neurological disease; multiple sclerosis” J Extracell Vesicles., 6(1):1-10; and Street et al., (2012) “Identification and proteomic profiling of exosomes in human cerebrospinal fluid” J Transl. Med., 10:5), urine, fecal matter, lymphatic fluid, blood, plasma, or serum to evaluate disease state. Additional measurements may include strength duration time constant (SDTC), short interval cortical inhibition (SICI), dynamometry, accurate test of limb isometric strength (ATLIS), compound muscle action potential (bio), and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in C9ORF72-associated amyotrophic lateral sclerosis (c9ALS) patients. CSF pNFH can serve as a prognostic biomarker for clinical trials, which will increase the likelihood of successfully developing a treatment for c9ALS.

In some embodiments, diagnosing a patient with a neurological disease such as Alzheimer's disease can involve evaluating mental performance of the patient. Evaluation of mental performance can involve a Mini-Mental State Examination (MMSE). Additional examples for measuring mental performance include the Functional Assessment Staging Test (FAST), the Motor Screening Task, Paired Associates Learning, Spatial Working Memory, Reaction time, Rapid Visual Information Processing, Delayed Matching to Sample, and Pattern Recognition Memory In some embodiments, diagnosing a patient with a neurological disease such as Parkinson's disease involves implementing the Unified Parkinson's Disease Rating Scale (UPDRS) as the performance measure. Other measures for quantifying aspects of functional performance not measured by the UPDRS can include the Berg Balance Scale (BBS), Forward Functional Reach Test (FFR), Backward Functional Reach Test (BFR), Timed “Up & Go” Test (TUG), and gait speed.

### Additional Embodiments

Disclosed herein is a Protein Phosphatase 1A (PPM1A) antisense oligonucleotide comprising a nucleotide sequence complementary to a nucleotide sequence of nucleotide 41,932 to nucleotide 42,787 and from nucleotide 44,871 to nucleotide 44,990 of a PPM1A gene sequence (SEQ ID NO: 1), or a portion thereof. Additionally disclosed herein is a Protein Phosphatase 1A (PPM1A) antisense oligonucleotide comprising the nucleotide sequence of SEQ ID NO: 2895 (5′ XYYZYTTGAGTCTCCXYXWZ 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)cytosine, and Z is 2′-O-(2-methoxyethyl)thymidine. Additionally disclosed herein is a Protein Phosphatase 1A (PPM1A) antisense oligonucleotide comprising the nucleotide sequence of SEQ ID NO: 2900 (5′ ZYZYYAGCGGATTACZZWWZ 3′), or a pharmaceutically acceptable salt thereof, wherein W is 2′-O-(2-methoxyethyl)guanosine, Y is 2′-O-(2-methoxyethyl)cytosine, and Z is 2′-O-(2-methoxyethyl)thymidine. Additionally disclosed herein is a Protein Phosphatase 1A (PPM1A) antisense oligonucleotide comprising the nucleotide sequence of SEQ ID NO: 2905 (5′ XWYYXGAGAGCCATTYXYXY 3′), or a pharmaceutically acceptable salt thereof, wherein W is 2′-O-(2-methoxyethyl)guanosine, X is 2′-O-(2-methoxyethyl)adenosine, and Y is 2′-O-(2-methoxyethyl)cytosine. Additionally disclosed herein is a Protein Phosphatase 1A (PPM1A) antisense oligonucleotide comprising the nucleotide sequence of SEQ ID NO: 2907 (5′ WYYYZCGATACAGCCXWXWX 3′), or a pharmaceutically acceptable salt thereof, wherein W is 2′-O-(2-methoxyethyl)guanosine, X is 2′-O-(2-methoxyethyl)adenosine, Y is 2′-O-(2-methoxyethyl)cytosine, and Z is 2′-O-(2-methoxyethyl)thymidine. Additionally disclosed herein is a Protein Phosphatase 1A (PPM1A) antisense oligonucleotide comprising the nucleotide sequence of SEQ ID NO: 2911 (5′ YYZZYTTCACTGCTTYZWWY 3′), or a pharmaceutically acceptable salt thereof, wherein W is 2′-O-(2-methoxyethyl)guanosine, Y is 2′-O-(2-methoxyethyl)cytosine, and Z is 2′-O-(2-methoxyethyl)thymidine. Additionally disclosed herein is a Protein Phosphatase 1A (PPM1A) antisense oligonucleotide comprising the nucleotide sequence of SEQ ID NO: 2893 (5′ ZYZYYACAGTTAATGXXXZX 3′), or a pharmaceutically acceptable salt thereof, wherein Y is 2′-O-(2-methoxyethyl)cytosine, X is 2′-O-(2-methoxyethyl)adenosine, and Z is 2′-O-(2-methoxyethyl)thymidine.

In some embodiments, at least one nucleoside linkage of the nucleotide sequence is selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments, at least one internucleoside linkage of the nucleotide sequence is a phosphorothioate linkage. In some embodiments, all internucleoside linkages of the nucleotide sequence are phosphorothioate linkages.

Additionally disclosed herein is a pharmaceutical composition comprising the antisense oligonucleotide described above, or a pharmaceutically acceptable salt thereof, and a pharmaceutically acceptable excipient.

Additionally disclosed herein is a method of treating a neurological disease in a patient in need thereof, the method comprising administering to the patient a PPM1A inhibitor. In various embodiments, the neurological disease is selected from the group consisting of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, and corticobasal degeneration (CBD).

Additionally disclosed herein is a method of restoring autophagy in a cell, the method comprising exposing the cell to a PPM1A inhibitor. Additionally disclosed herein is a method of increasing TBK1 ser172 phosphorylation in a cell, the method comprising exposing the cell to a PPM1A inhibitor. Additionally disclosed herein is a method of increasing TBK1 function in a cell, the method comprising exposing the cell to a PPM1A inhibitor. Additionally disclosed herein is a method of inhibiting PPM1A in a cell, the method comprising exposing the cell to a PPM1A inhibitor.

In various embodiments, the cell is a cell of a patient in need of treatment of a neurological disease. In various embodiments, the neurological disease is selected from the group consisting of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, and corticobasal degeneration (CBD).

In various embodiments, the exposing is performed in vivo or ex vivo. In various embodiments, the exposing comprises administering the PPM1A inhibitor to a patient in need thereof. In various embodiments, the PPM1A inhibitor is administered topically, parenterally, intrathecally, intracisternally, orally, rectally, buccally, sublingually, vaginally, pulmonarily, intratracheally, intranasally, transdermally, or intraduodenally. In various embodiments, the PPM1A inhibitor is administered orally.

In various embodiments, a therapeutically effective amount of the PPM1A inhibitor is administered. In various embodiments, the patient is a human. In various embodiments, the PPM1A inhibitor comprises the PPM1A antisense oligonucleotide described above, or a pharmaceutically acceptable salt thereof.

In various embodiments, the PPM1A inhibitor is selected from the group consisting of a PPM1A small hairpin RNA (shRNA), a PPM1A small interfering RNA (siRNA), a PPM1A peptide nucleic acid (PNA), a PPM1A locked nucleic acid (LNA), and a PPM1A morpholino oligomer. In various embodiments, the pharmaceutical composition is suitable for topical, intrathecal, parenteral, oral, pulmonary, intratracheal, intranasal, transdermal, rectal, buccal, sublingual, vaginal, or intraduodenal administration.

Additionally disclosed herein is a use of a PPM1A inhibitor in the manufacture of a medicament for the treatment of neurological disease. In various embodiments, the neurological disease is selected from the group consisting of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, and corticobasal degeneration (CBD). In various embodiments, the PPM1A inhibitor is the PPM1A antisense oligonucleotide described above.

Additionally disclosed herein is a method of treating a neurological disease in a patient in need thereof, the method comprising administering to a patient in need thereof a therapeutically effective amount of a pharmaceutical composition comprising a PPM1A inhibitor, and a pharmaceutically acceptable excipient. In various embodiments, the neurological disease is selected from the group consisting of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, and corticobasal degeneration (CBD). In various embodiments, the PPM1A inhibitor is the PPM1A antisense oligonucleotide of any one of claims 1-10, or a pharmaceutically acceptable salt thereof. In various embodiments, the PPM1A inhibitor is selected from the group consisting of a PPM1A small hairpin RNA (shRNA), a PPM1A small interfering RNA (siRNA), a PPM1A peptide nucleic acid (PNA), a PPM1A locked nucleic acid (LNA), and a PPM1A morpholino oligomer.

In various embodiments, the pharmaceutical composition is administered topically, parenterally, orally, pulmonarily, rectally, buccally, sublingually, vaginally, intratracheally, intranasally, intrathecally, intracisternally, transdermally, or intraduodenally. In various embodiments, the pharmaceutical composition is administered orally. In various embodiments, the patient is human.

Additionally disclosed herein is a PPM1A antisense oligonucleotide described above, or a pharmaceutically acceptable salt thereof, for use as a medicament. Additionally disclosed herein is a PPM1A antisense oligonucleotide described above, or a pharmaceutically acceptable salt thereof, for use in the treatment of a neurological disease. In various embodiments, said neurological disease is selected from the group consisting of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, and corticobasal degeneration (CBD).

Additionally disclosed herein is a Protein Phosphatase 1A (PPM1A) antisense oligonucleotide selected from the group consisting of a PPM1A antisense oligonucleotide comprising the nucleotide sequence of SEQ ID NO: 450 (5′ ACCTCTTGAGTCTCCACAGT 3′), a PPM1A antisense oligonucleotide comprising the nucleotide sequence of SEQ ID NO: 517 (5′ TCTCCAGCGGATTACTTGGT 3′), a PPM1A antisense oligonucleotide comprising the nucleotide sequence of SEQ ID NO: 579 (5′ AGCCAGAGAGCCATTCACAC 3′), a PPM1A antisense oligonucleotide comprising the nucleotide sequence of SEQ ID NO: 590 (5′ GCCCTCGATACAGCCAGAGA 3′), a PPM1A antisense oligonucleotide comprising the nucleotide sequence of SEQ ID NO: 916 (5′ CCTTCTTCACTGCTTCTGGC 3′), or a pharmaceutically acceptable salt thereof, and a PPM1A antisense oligonucleotide comprising the nucleotide sequence of SEQ ID NO: 440 (5′ TCTCCACAGTTAATGAAATA 3′), or a pharmaceutically acceptable salt thereof; wherein at least one nucleoside linkage of the nucleotide sequence is selected from the group consisting of: a phosphorothioate linkage, a phosphorodithioate linkage, a phosphotriester linkage, an alkylphosphonate linkage, a methylphosphonate linkage, a dimethylphosphonate linkage, an aminoalkylphosphotriester linkage, an alkylene phosphonate linkage, a phosphinate linkage, a phosphoramidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage; and/or wherein at least one nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), and a peptide nucleic acid (PNA).

In various embodiments, at least one internucleoside linkage of the nucleotide sequence is a phosphorothioate linkage. In various embodiments, all internucleoside linkages of the nucleotide sequence are phosphorothioate linkages.

Additionally disclosed herein is a pharmaceutical composition comprising the antisense oligonucleotide described above, or a pharmaceutically acceptable salt thereof, and a pharmaceutically acceptable excipient. Additionally disclosed herein is a Protein Phosphatase 1A (PPM1A) antisense oligonucleotide comprising a nucleic acid sequence that shares at least 90% identity with a continuous 10 nucleobase sequence of SEQ ID NOs: 2-955 or SEQ ID NOs: 1910-2863. In various embodiments, the nucleic acid sequence shares at least 90% identity with a continuous 11, 12, 13, 14, 15, 16, or 17 nucleobase sequence of SEQ ID NOs: 2-955 or SEQ ID NOs: 1910-2863.

Additionally disclosed herein is a PPM1A antisense oligonucleotide of any one of SEQ ID NOs: 2-955 or SEQ ID NOs: 1910-2863. Additionally disclosed herein is a pharmaceutical composition comprising a PPM1A antisense oligonucleotide of any one of SEQ ID NOs: 2-955 or SEQ ID NOs: 1910-2863, and a pharmaceutically acceptable excipient.

In various embodiments, at least one nucleoside linkage of the antisense oligonucleotide sequence is selected from the group consisting of: a phosphorothioate linkage, a phosphorodithioate linkage, a phosphotriester linkage, an alkylphosphonate linkage, a methylphosphonate linkage, a dimethylphosphonate linkage, an aminoalkylphosphotriester linkage, an alkylene phosphonate linkage, a phosphinate linkage, a phosphoramidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage; and/or wherein at least one nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), and a peptide nucleic acid (PNA). In various embodiments, at least one internucleoside linkage of the nucleotide sequence is a phosphorothioate linkage. In various embodiments, all internucleoside linkages of the nucleotide sequence are phosphorothioate linkages.

Additionally disclosed herein is a PPM1A antisense oligonucleotide or a pharmaceutical composition for use in the treatment of a neurological disease. In various embodiments, said neurological disease is selected from the group consisting of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, and corticobasal degeneration (CBD).

### EXAMPLES

The disclosure is further illustrated by the following examples. The examples are provided for illustrative purposes only, and are not to be construed as limiting the scope or content of the disclosure in any way.

### Example 1: Design and Selection of PPM1A Antisense Oligonucleotides

Analysis of a human PPM1A mRNA sequence (NCBI Reference Sequence: NM_021003.5; SEQ ID NO: 2864) revealed 7,776 potential PPM1A AON candidate sequences. However, the majority of candidates did not meet the candidate filtering thresholds due to variability in the 5′UTR and 3′UTR sequences of the different PPM1A splice variants. A region spanning nucleotides 457 to 1410 of NM_021003.5 was identified as common to all known PPM1A splice variants. PPM1A AON candidates were identified that met the aforementioned filtering criteria and that target this region.

As used in the subsequent Examples, descriptions, and corresponding Figures, each PPM1A AON is identified using a “Legacy ID.” The Legacy ID of a PPM1A AON includes the notation of “QPA-” appended with the start position of the PPM1A transcript (specifically PPM1A transcript of SEQ ID NO: 2864) that the PPM1A AON is complementary to. For example, the PPM1A AON of SEQ ID NO: 2868 (5′ WYZWYTTAGCCCATAZYWYX 3′) is complementary to positions 542-561 of the PPM1A transcript of SEQ ID NO: 2864, where position 542 is the start position. Thus, the PPM1A AON of SEQ ID NO: 2868 is referred to below as QPA-542.

Table 5 below documents the PPM1A AON candidates that were designed and subsequently evaluated for ability to knockdown PPM1A expression. Additional development involved generating PPM1A AON candidates with a cholesterol conjugate group located on the 3′ end of the PPM1A AON. The PPM1A AON candidates with a cholesterol conjugate group are shown below in Table 6.

TABLE 5
Evaluated PPM1A AONs

| SEQ | | Oligonucleotide |
|---|---|---|
| ID NO: | Legacy ID | Sequence (5′→3′)*# |
| 2868 | QPA-542 | WYZWYTTAGCCCATAZYWYX |
| 2869 | QPA-555 | WYYXWCCTTGCATGCZWYZZ |
| 2870 | QPA-559 | XYXYWCCAGCCTTGCXZWYZ |
| 2871 | QPA-599 | ZWWYXAACCGATCACXWYYW |
| 2872 | QPA-602 | XYZZWGCAAACCGATYXYXW |
| 2873 | QPA-603 | YXYZZGGCAAACCGAZYXYX |
| 2874 | QPA-604 | YYXYZTGGCAAACCGXZYXY |
| 2875 | QPA-605 | ZYYXYTTGGCAAACCWXZYX |
| 2876 | QPA-606 | WZYYXCTTGGCAAACYWXZY |
| 2877 | QPA-607 | XWZYYACTTGGCAAAYYWXZ |
| 2878 | QPA-608 | XXWZYCACTTGGCAAXYYWX |
| 2879 | QPA-609 | YXXWZCCACTTGGCAXXYYW |
| 2880 | QPA-625 | XXWXXTGACCACGATZYXXW |
| 2881 | QPA-642 | WYYYXTCATACACAGYXXXW |
| 2882 | QPA-644 | XZWYYCATCATACACXWYXX |
| 2883 | QPA-646 | WYXZWCCCATCATACXYXWY |
| 2884 | QPA-648 | YXWYXTGCCCATCATXYXYX |
| 2885 | QPA-650 | XYYXWCATGCCCATCXZXYX |
| 2886 | QPA-652 | WXXYYAGCATGCCCAZYXZX |
| 2887 | QPA-655 | ZWXWXACCAGCATGCYYXZY |
| 2888 | QPA-656 | YZWXWAACCAGCATGYYYXZ |
| 2889 | QPA-708 | YYZWWTTATTGGTGAZWZWX |
| 2890 | QPA-709 | ZYYZWGTTATTGGTGXZWZW |
| 2891 | QPA-794 | YXZWZGTTCATCAATYZYYX |
| 2892 | QPA-795 | ZYXZWTGTTCATCAAZYZYY |
| 2893 | QPA-895 | ZYZYYACAGTTAATGXXXZX |
| 2894 | QPA-900 | ZZWXWTCTCCACAGTZXXZW |
| 2895 | QPA-905 | XYYZYTTGAGTCTCCXYXWZ |
| 2896 | QPA-910 | XWZXXACCTCTTGAGZYZYY |
| 2897 | QPA-915 | ZXYXXAGTAAACCTCZZWXW |
| 2898 | QPA-962 | XZZXYTTGGTTTGTGXZYZZ |
| 2899 | QPA-967 | XWYWWATTACTTGGTZZWZW |
| 2900 | QPA-972 | ZYZYYAGCGGATTACZZWWZ |
| 2901 | QPA-977 | ZZYZZTCTCCAGCGGXZZXY |
| 2902 | QPA-987 | ZYZWXATTCGTTCTTZYZYY |
| 2903 | QPA-1025 | WYYXZTCACACGCTGXXZYX |
| 2904 | QPA-1030 | XWXWXGCCATTCACAYWYZW |
| 2905 | QPA-1034 | XWYYXGAGAGCCATTYXYXY |
| 2906 | QPA-1040 | YWXZXCAGCCAGAGAWYYXZ |
| 2907 | QPA-1045 | WYYYZCGATACAGCCXWXWX |
| 2908 | QPA-1098 | WYZWYTCAGTAGGACYZZZZ |
| 2909 | QPA-1361 | ZWYZZCTGGCGATACZZZWW |
| 2910 | QPA-1366 | ZZYXYTGCTTCTGGCWXZXY |
| 2911 | QPA-1371 | YYZZYTTCACTGCTTYZWWY |
| 2912 | QPA-1378 | ZYZWYCTCCTTCTTCXYZWY |
| 2913 | QPA-1386 | ZWZYYAACTCTGCCTYYZZY |

*In each of the oligonucleotide sequences included
in Table 5, the individual nucleosides are as
follows: A is adenosine, G is guanosine, C is
cytosine, T is thymidine, W is
2′-O-(2-methoxyethyl)guanosine, X is
2′-O-(2-methoxyethyl)adenosine, Y is
2′-O-(2-methoxyethyl)-5-methylcytosine, and Z is
2′-O-(2-methoxyethyl)thymidine.
#In each of the oligonucleotide sequences included
in Table 5, the individual nucleosides are each
linked by a phosphorothioate bond.

TABLE 6
Additional Evaluated PPM1A AONs with a
cholesterol conjugate group on 3′ end of the AON

| SEQ | | Oligonucleotide |
|---|---|---|
| ID NO: | Legacy ID | Sequence (5′→3′)*#^ |
| 2876 | QPA-606-C | WZYYXCTTGGCAAACYWXZY |
| 2881 | QPA-642-C | WYYYXTCATACACAGYXXXW |
| 2882 | QPA-644-C | XZWYYCATCATACACXWYXX |

*In each of the oligonucleotide sequences included
in Table 6, the individual nucleosides are as follows:
A is adenosine, G is guanosine, C is cytosine, T
is thymidine, W is 2′-O-(2-methoxyethyl)guanosine,
is 2′-O-(2-methoxyethyl)adenosine, Y is
2′-O-(2-methoxyethyl)-5-methylcytosine, and Z is
2′-O-(2-methoxyethyl)thymidine.
#In each of the oligonucleotide sequences included
in Table 6, the individual nucleosides are each
linked by a phosphorothioate bond.
^In each of the oligonucleotide sequences in Table 6,
a cholesterol conjugate group is located on the 3′
end of the oligonucleotide.

### Example 2: Analysis of PPM1A AON Knockdown Efficacy

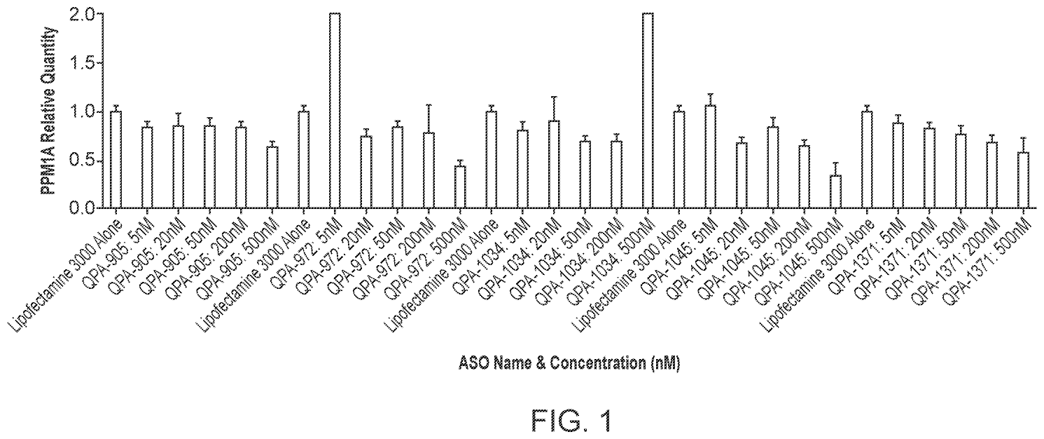

A subset of the PPM1A AONs shown above in Tables 5 and 6 (specifically QPA-905, QPA-972, QPA-1034, QPA-1045, and QPA-1371) were evaluated by screening for PPM1A mRNA knockdown using reverse transcription quantitative polymerase chain reaction (RT-qPCR) analysis. To analyze the knockdown efficacy of PPM1A AONs, cells from the lymphoblastoid cell line BP6074 were transfected with either Lipofectamine 3000 transfection reagent (Thermo Fisher Scientific, Waltham, Mass., USA) alone or with Lipofectamine 3000 and varying amounts (5 nM, 20 nM, 50 nM, 200 nM, or 500 nM) of one of the PPM1A AON's listed in Tables 5 or 6. The BP6074 cell line is derived from a 48 year-old male ALS patient, and harbors a TBK1 protein-truncating mutation (C992+1 G>A) that results in a frameshift and decreased TBK1 protein expression (see van der Zee et al. (2017) “TBK1 Mutation Spectrum in an Extended European Patient Cohort with Frontotemporal Dementia and Amyotrophic Lateral Sclerosis” Hum Mutat. 38(3): 297-309). Cells were transfected or exposed to transfection reagent alone, and levels of PPM1A expression were evaluated by qPCR 72 hours later. All experiments were performed in triplicate (FIG. 1). As shown in FIG. 1, all candidate PPM1A AONs showed efficacy in knocking down levels of PPM1A mRNA transcript expression, especially at the higher concentrations tested. These results demonstrate that QPA-905, QPA-972, QPA-1034, QPA-1045, and QPA-1371 were each able to knock down levels of PPM1A mRNA relative to control levels in an ALS patient cell line 72 hours after transfection. Results of the knockdown of PPM1A mRNA transcript expression is shown in Table 7.

TABLE 7
Knockdown of PPM1A transcript relative to control (lipofectamine
3000 alone). Mean +/− standard deviation

| | Control | 5 nM | 20 nM | 50 nM | 200 nM | 500 nM |
|---|---|---|---|---|---|---|
| Lipofectamine | 1.00 ± 0.05 | | | | | |
| 3000 Alone | | | | | | |
| QPA-905 | | 0.85 ± 0.04 | 0.86 ± 0.11 | 0.85 ± 0.07 | 0.83 ± 0.06 | 0.64 ± 0.04 |
| QPA-972 | | 4.58 ± 4.30 | 0.74 ± 0.07 | 0.84 ± 0.05 | 0.79 ± 0.28 | 0.44 ± 0.06 |
| QPA-1034 | | 0.81 ± 0.08 | 0.91 ± 0.23 | 0.69 ± 0.04 | 0.69 ± 0.06 | 6.4 ± 3.5 |
| QPA-1045 | | 1.06 ± 0.12 | 0.68 ± 0.03 | 0.84 ± 0.09 | 0.65 ± 0.05 | 0.34 ± 0.12 |
| QPA-1371 | | 0.88 ± 0.07 | 0.82 ± 0.07 | 0.76 ± 0.09 | 0.68 ± 0.07 | 0.57 ± 0.15 |

Knockdown efficacy of PPM1A AON candidates was also evaluated in the human neuroblastoma cell line SY5Y. SY5Y cells were plated in 96-well plates at a concentration of 5,000 cells/well and grown in media containing: Minimum essential medium eagle (Cat. No. M2279, Sigma, St. Louis, Mo., USA), nutrient mixture F-12 Ham (Cat. No. N4888, Sigma, St. Louis, Mo., USA), 100% Fetal Bovine Serum (Cat. No. 16140071, Life technologies, Carlsbad, Calif., USA), Glutamax 100× (Cat. No. 35050-061, Gibco), NEAA (Cat. No. 11140-050, Gibco), and penicillin-streptomycin (Cat. No. 30-001-C1, Corning). Cells were left untreated, treated with Lipofectamine 3000 alone, or transfected with PPM1A AON at various concentrations (5 nM, 20 nM, 50 nM, 200 nM, or 500 nM) using Lipofectamine 3000. Cells were separately transfected with 50 nM control siRNA (siControl, ON-TARGETplus Non-targeting Pool human, Dharmacon D-001810-10) or PPM1A siRNA (siPPM1A, ON-TARGETplus PPM1A, Dharmacon L-009574-00-0005) to provide an additional negative and positive control, respectively. 48 hours after transfection, RNA was isolated, cDNA generated and multiplexed RT-qPCR assay performed with tagman probes for PPM1A (Hs06637123_g1, Thermofisher 4351370) and reference GAPDH (Hs03929097_g1, Thermofisher 4448490) quantification.

PPM1A signal (Ct) was normalized to GAPDH (deltaCt). To visualize the quantitative changes (e.g., % decrease PPM1A transcripts), the normalized PPM1A signal was further normalized to the vehicle (treated with transfection agent alone, deltadeltaCt). Relative quantity of transcript level was calculated using the equation RQ=2^{-deltadeltaCt} and is used to describe the treatment condition comparison to normal, healthy levels (1.0).

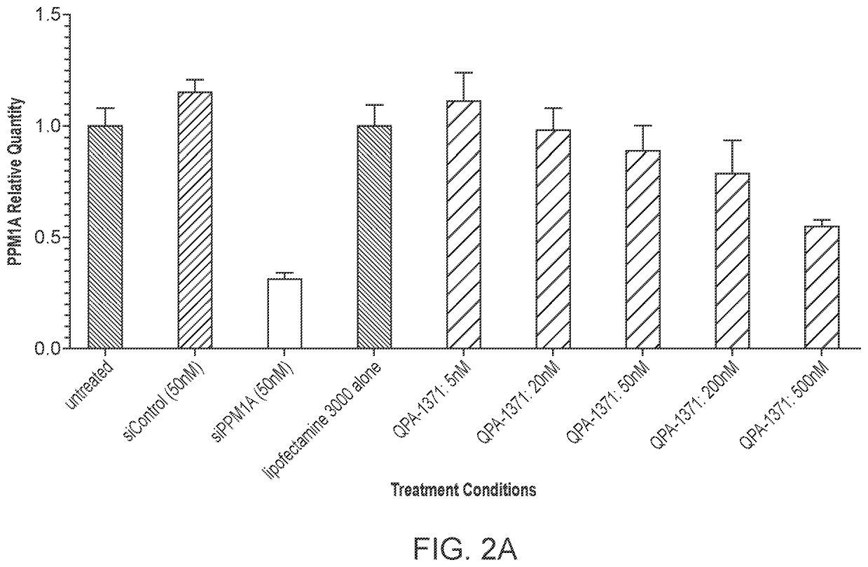

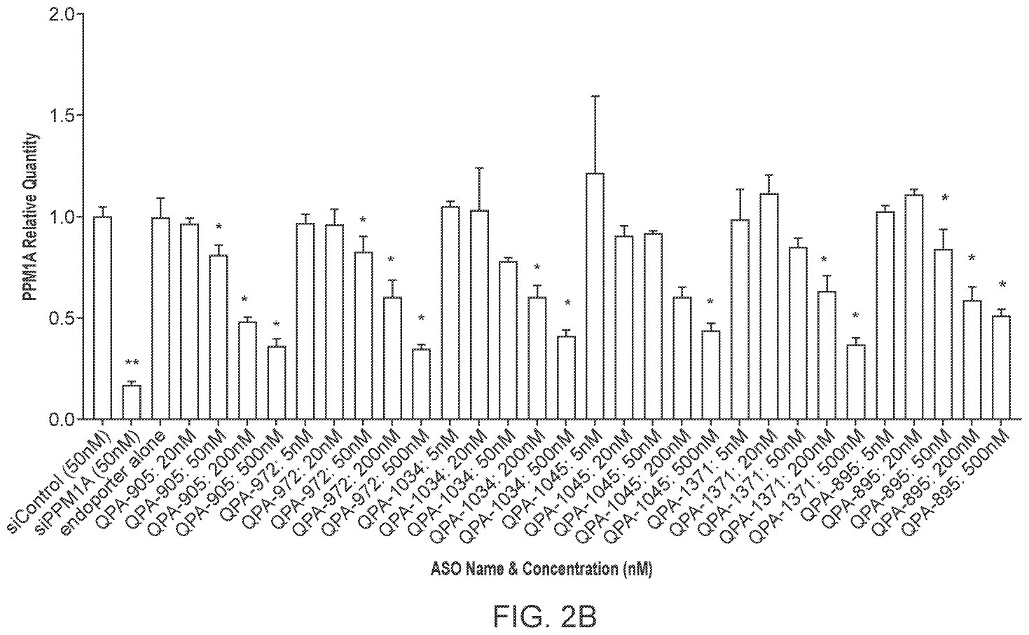

Transfection of SY5Y cells with the PPM1A AON QPA-1371 resulted in a dose-dependent decrease in PPM1A expression that changed inversely with increasing amounts of transfected PPM1A AON (FIG. 2A, Table 8).

TABLE 8
Knockdown of PPM1A expression in response to QPA-1371. Mean +/− standard deviation

| | Control | 5 nM | 20 nM | 50 nM | 200 nM | 500 nM |
|---|---|---|---|---|---|---|
| Untreated | 1.00 ± 0.08 | | | | | |
| siControl | 1.16 ± 0.05 | | | | | |
| (50 nM) | | | | | | |
| siPPM1A (50 | 0.32 ± 0.02 | | | | | |
| nM) | | | | | | |
| Lipofectamine | 1.00 ± 0.09 | | | | | |
| 3000 alone | | | | | | |
| QPA-1371 | | 1.12 ± 0.12 | 0.99 ± 0.09 | 0.89 ± 0.11 | 0.79 ± 0.14 | 0.56 ± 0.02 |

Similarly, in a second experiment, transfection of SY5Y cells (using Endo-Porter delivery reagent as the transfection agent, Gene Tools, Inc., Oregon, USA) with the PPM1A AON QPA-905, QPA-1371, QPA-972, QPA-1034, QPA-1045, or QPA-895 resulted in a dose-dependent decrease in PPM1A expression that changed inversely with increasing amounts of transfected PPM1A AON (FIG. 2B, Table 9). Asterisk indicates p<0.05 vs endoporter alone in one-way ANOVA. Double asterisk indicates p<0.05 vs. siControl in a t-test. These results demonstrate that PPM1A AONs were able to inhibit PPM1A transcript expression in multiple cell lines.

TABLE 9
Knockdown of PPM1A expression in response to various
PPM1A AONs. Mean ±/− standard deviation

| | Control | 5 nM | 20 nM | 50 nM | 200 nM | 500 nM |
|---|---|---|---|---|---|---|
| siControl | 1.16 ± 0.05 | | | | | |
| (50nM) | | | | | | |
| siPPMIA | 0.32 ± 0.02 | | | | | |
| (50 nM) | | | | | | |
| Endoporter | 1.00 ± 0.09 | | | | | |
| QPA-905 | | | 0.97 ± 0.02 | 0.82 ± 0.05 | 0.49 ± 0.02 | 0.37 ± 0.03 |
| QPA-972 | | 0.97 ± 0.04 | 0.97 ± 0.07 | 0.83 ± 0.07 | 0.61 ± 0.08 | 0.35 ± 0.2 |
| QPA-1034 | | 1.06 ± 0.02 | 1.04 ± 0.20 | 0.79 ± 0.01 | 0.61 ± 0.05 | 0.42 ± 0.02 |
| QPA-1045 | | 1.22 ± 0.37 | 0.91 ± 0.04 | 0.92 ± 0.01 | 0.61 ± 0.4 | 0.44 ± 0.03 |
| QPA-1371 | | 0.99 ± 0.14 | 1.12 ± 0.09 | 0.86 ± 0.04 | 0.64 ± 0.07 | 0.37 ± 0.03 |
| QPA-895 | | 1.03 ± 0.02 | 1.11 ± 0.02 | 0.84 ± 0.09 | 0.59 ± 0.06 | 0.51 ± 0.03 |

To further evaluate the ability of PPM1A AON candidates to inhibit PPM1A expression, Western blotting experiments were performed. Specifically, 2 PPM1A AON candidates, QPA-1045 and QPA-1371, were selected to evaluate the effect of PPM1A AON transfection on PPM1A protein levels and the ratio of active to total TBK1. Lymphoblastoid cells from a healthy individual (“healthy cells”) or an ALS patient harboring a TBK1 mutation (“patient cells”) were transfected with RNAiMax transfection reagent (Thermo Fisher Scientific, Waltham, Mass., USA) alone or PPM1A AON at 5 μM using RNAiMax transfection reagent. 24 hours after transfection, cell media was changed to remove transfection reagent. Cells were then incubated for a further 48 hours, after which protein was extracted from cells for analysis. Protein extracts were probed by Western blot analysis using antibodies able to detect GAPDH (Cat. No. ab181602; Abcam, Cambridge, Mass., USA), total TBK1 (Cat. No. ab40676; Abcam, Cambridge, Mass., USA), phosphorylated TBK1 (Cat. No. 5483s; Cell Signaling Technologies, Danvers, Mass., USA), and PPM1A (Cat. No. ab14824; Abcam, Cambridge, Mass., USA). Secondary antibodies used included anti-rabbit IgG, HRP-linked (Cat. No. 7074; Cell Signaling Technologies, Danvers, Mass., USA) and anti-mouse IgG, HRP-linked (Cat. No. 7076; Cell Signaling Technologies, Danvers, Mass., USA). All experiments were performed in triplicate.

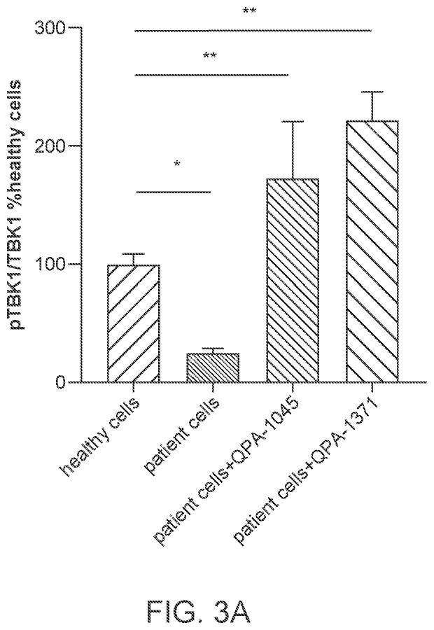

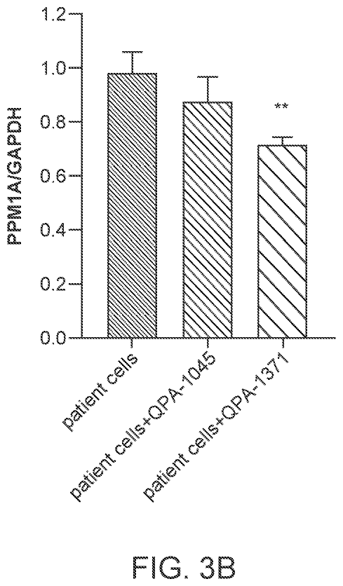

The ratio of phosphorylated TBK1 to total TBK1 was evaluated, using GAPDH as a control to normalize levels of phosphorylated TBK1 and total TBK1. Compared to lymphoblastoid cells not harboring the BP6074 cell line TBK1 protein-truncating mutation (“healthy cells”), BP6074 cells (“patient cells”) showed a significantly lower ratio of phosphorylated TBK1 to total TBK1 (FIG. 3A, healthy cells v patient cells, p<0.05). Furthermore, transfection of BP6074 cells with PPM1A AONs QPA-1045 and QPA-1371 resulted in a significant increase in the ratio of phosphorylated TBK1 to total TBK1, over that of even healthy cells (FIG. 3A, healthy cells v patient cells+QPA-1045, healthy cells v patient cells+QPA-1371, p<0.01; an approximately 8.5-fold increase over untransfected patient cells). Results are shown below in Table 10.

TABLE 10
Effects of PPM1A AONs on TBK1 levels. Mean
+/− standard deviation

| | PTBK1/TBK1 | |
|---|---|---|
| | % healthy cells | PPM1A/GAPDH |
| patient cells | 25.98 ± 2.94 | 0.99 ± 0.07 |
| patient cells + QPA-1045 | 173.6 ± 46.18 | 0.88 ± 0.08 |
| patient cells + QPA-1371 | 223.4 ± 22.99 | 0.72 ± 0.02 |
| healthy cells | 100 ± 8.59 | |

Additionally, PPM1A levels were evaluated in BP6074 cells exposed to transfection reagent alone or transfected with PPM1A AONs QPA-1045 and QPA-1371, using the same transfection protocol described above. PPM1A levels were normalized to GAPDH protein levels. Compared to BP6074 cells exposed to transfection reagent alone, BP6074 cells transfected with PPM1A AON QPA-1045 or QPA-1371 showed a decrease in PPM1A protein levels of about 10-25%. Transfection with QPA-1371 showed a statistically significant decrease in PPM1A levels (FIG. 3B, patient cells v patient cells+QPA-1371, p<0.01).

These results demonstrate that PPM1A AONs were able to decrease levels of PPM1A in an ALS patient cell line. These results also demonstrate that transfection of PPM1A AONs in an ALS patient cell line significantly increased the ratio of active (phosphorylated) TBK1 to total TBK1 in the patient cell line, even surpassing the ratio of active (phosphorylated) TBK1 to total TBK1 found in healthy cells. Thus, these results demonstrate that PPM1A AONs identified herein were capable of inhibiting PPM1A expression and increasing the ratio of active TBK1 in ALS patient cells.

RNA-knockdown potency was evaluated in SY5Y cells by several exemplary PPM1A AONs transfected with endoporter and tested for knockdown at 48 hours. FIG. 4A-FIG. 4Y are line graphs of RNA-knockdown potency of various candidate antisense oligonucleotides quantifying the decrease in PPM1A RNA with increasing AON concentration. Non-linear regression four parameter curves were fit and plotted using Graphpad Prism software (San Diego, Calif.), with the bottom of the curve fixed at 0. FIG. 4A represents RNA-knockdown potency of SEQ ID NO: 2898 (QPA-962); FIG. 4B represents RNA-knockdown potency of SEQ ID NO: 2899 (QPA-967); FIG. 4C represents RNA-knockdown potency of SEQ ID NO:2900 (QPA-972); FIG. 4D represents RNA-knockdown potency of SEQ ID NO: 2901 (QPA-977); FIG. 4E represents RNA-knockdown potency of SEQ ID NO: 2902 (QPA-987); FIG. 4F represents RNA-knockdown potency of SEQ ID NO: 2903 (QPA-1025); FIG. 4G represents RNA-knockdown potency of SEQ ID NO: 2904 (QPA-1030); FIG. 4H represents RNA-knockdown potency of SEQ ID NO: 2905 (QPA-1034); FIG. 4I represents RNA-knockdown potency of SEQ ID NO: 2906 (QPA-1040); FIG. 4J represents RNA-knockdown potency of SEQ ID NO: 2907 (QPA-1045); FIG. 4K represents RNA-knockdown potency of SEQ ID NO: 2909 (QPA-1361); FIG. 4L represents RNA-knockdown potency of SEQ ID NO: 2910 (QPA-1366); FIG. 4M represents RNA-knockdown potency of SEQ ID NO: 2911 (QPA-1371); FIG. 4N represents RNA-knockdown potency of SEQ ID NO: 2912 (QPA-1378); FIG. 4O represents RNA-knockdown potency of SEQ ID NO: 2913 (QPA-1386); FIG. 4P represents RNA-knockdown potency of SEQ ID NO: 2868 (QPA-542); FIG. 4Q represents RNA-knockdown potency of SEQ ID NO: 2869 (QPA-555); FIG. 4R represents RNA-knockdown potency of SEQ ID NO: 2883 (QPA-646); FIG. 4S represents RNA-knockdown potency of SEQ ID NO: 2870 (QPA-559); FIG. 4T represents RNA-knockdown potency of SEQ ID NO: 2908 (QPA-1098); FIG. 4U represents RNA-knockdown potency of SEQ ID NO: 2893 (QPA-895); FIG. 4V represents RNA-knockdown potency of SEQ ID NO: 2894 (QPA-900); FIG. 4W represents RNA-knockdown potency of SEQ ID NO: 2895 (QPA-905); FIG. 4X represents RNA-knockdown potency of SEQ ID NO: 2896 (QPA-910); and FIG. 4Y represents RNA-knockdown potency of SEQ ID NO: 2897 (QPA-915). IC50 calculated from the fitted non-linear regression curves are listed in Table 11.

TABLE 11
IC50 values for PPM1A AONs shown in FIGS. 4A-4Y.

| AON | IC50 (nM) |
|---|---|
| QPA-915 | 387.1 |
| QPA-1040 | 568.5 |
| QPA-977 | 291.9 |
| QPA-555 | 345.2 |
| QPA-1025 | 370.3 |
| QPA-1030 | 405.8 |
| QPA-967 | 419 |
| QPA-910 | 169.7 |
| QPA-1098 | 610.3 |
| QPA-962 | 362.5 |
| QPA-1386 | 667.5 |
| QPA-900 | 395.1 |
| QPA-1366 | 615.4 |
| QPA-1378 | 460.5 |
| QPA-987 | 280 |
| QPA-646 | 120.8 |
| QPA-542 | 411.4 |
| QPA-559 | 338.5 |
| QPA-1361 | 662.1 |
| QPA-905 | 207.9 |
| QPA-972 | 281.1 |
| QPA-1034 | 272 |
| QPA-1045 | 275.8 |
| QPA-1371 | 295.1 |
| QPA-895 | 349.8 |

PPM1A AON were also tested for potency to reduce PPM1A transcripts in human motor neurons. iCELL MN (Cellular Dynamics Internation Fujifilm C1050) were seeded onto 96 well plate (0.32 cm²/well) at a density of 10,000 cells/well. Cell were maintained following CDI guide instructions with a few modifications. Cells were thawed and plated in complete iCELL neuron media (CDI R1051) supplemented with 10 uM of Y-27632 dihydrochloride (Tocris 1254) overnight. The cells received a full media exchange the day after. Three days post plating the cells received a media exchange composed of 50% iCELL MN neuron media and 50% complete neuronal maturation media (Neurobasal-Thermofisher 21103049, lx Glutamax-Thermofisher 35050061, lx NEAA-Thermofisher 11140050, lx B-27 plus supplement-Thermofisher A3582801, 1×N2 supplement-Thermofisher 17502048, 0.2 ug/mL ascorbic acid-Sigma A4403 supplemented with growth factors BDNF, CNTF and GDNF (10 /mL BDNF-R&D Systems 248-BDB, 10 ng/mL CNTF R&D 257-N and 10 ng/mL GDNF-R&D Systems 212-GD). The cells were transfected 5 days post-plating in complete neuronal maturation media. The transfection of AONs were done using 6 uM Endoporter (Gene Tool Endo-Porter-PEG-1 mL). The transfection for control conditions used Lipofectamine RNAiMAX (Thermofisher 13778150). Negative control (siCtrol) consisted of 50 nM of ON-TARGETplus Non-targeting Pool human (Dharmacon D-001810-10) and positive control (siPPM1A) consisted of 50 nM ON-TARGETplus PPM1A (Dharmacon L-009574-00-0005). 48 hours post transfection the cells with siRNA were washout out to remove the RNAimax. 72 hours post-transfection, RNA was isolated from all treatment conditions, cDNA generated and multiplexed RT-qPCR assay performed with taqman probes for PPM1A (Hs06637123_g1, Thermofisher 4351370) and reference GAPDH (Hs03929097_g1, Thermofisher 4448490). RT-qPCR was performed using the TaqMan Fast Advanced Cells-to-CT Kit (Thermofisher A35378) and TaqMan Fast Advanced Master Mix (Thermofisher 4444557) following manufacturer's protocol and run on the Applied Biosystems QuantStudio 6 pro/7pro real time PCR system. One cycle of reverse transcription was performed at a temperature of 50° C. for 5 min. One cycle of RT inactivation/initial denaturation was performed at a temperature of 95° C. for 20 seconds. Forty cycles of amplification were performed at a temperature of 95° C. for 1 second followed by 60° C. for 20 seconds. Relative quantity was calculated as described for SY5Y.

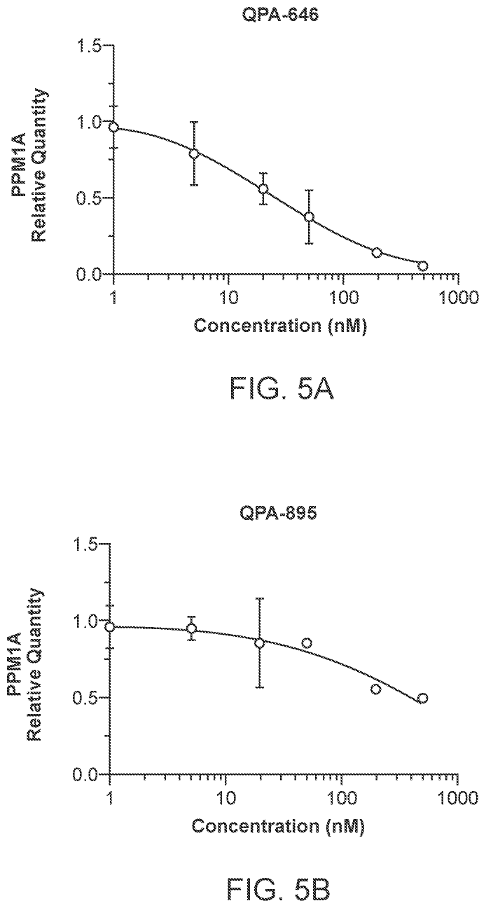

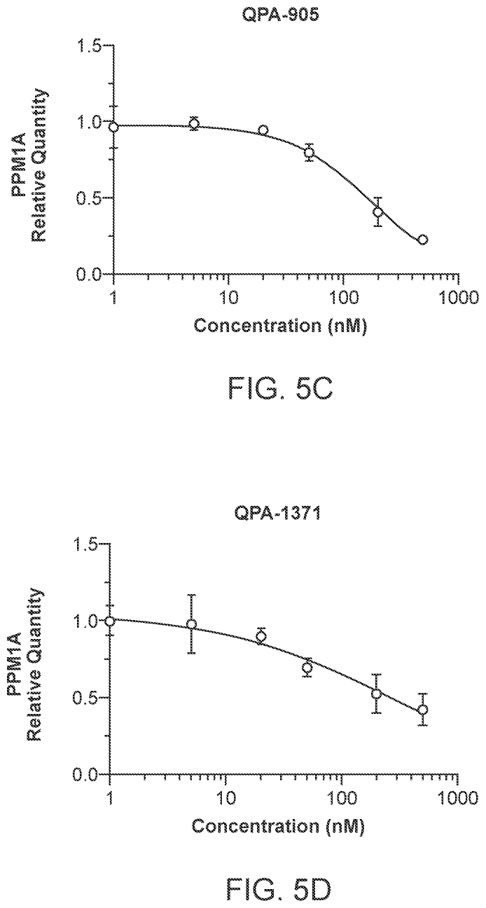

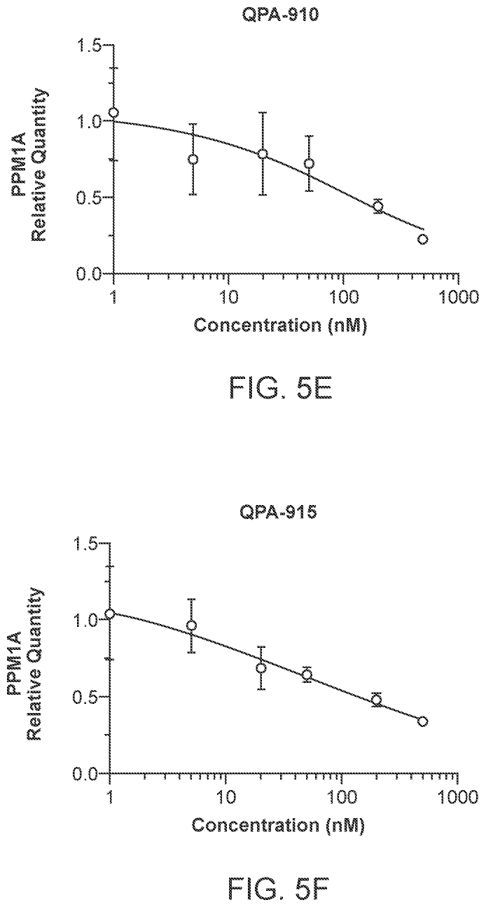

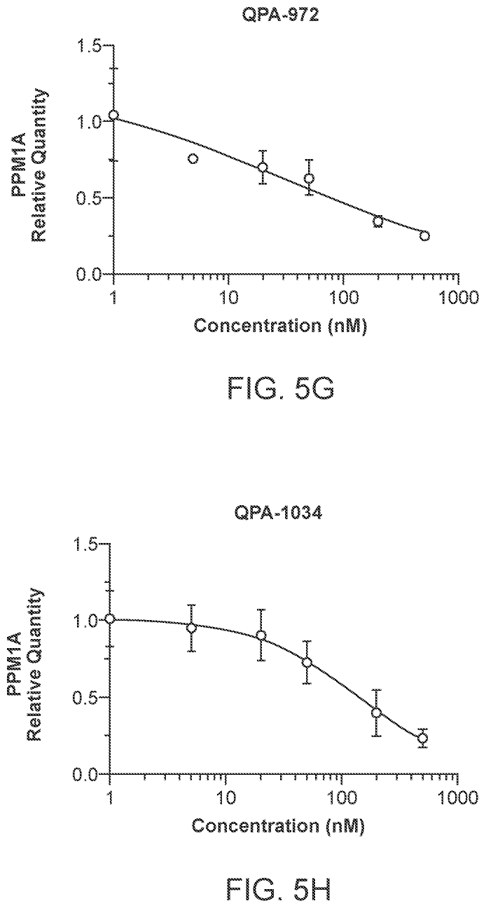

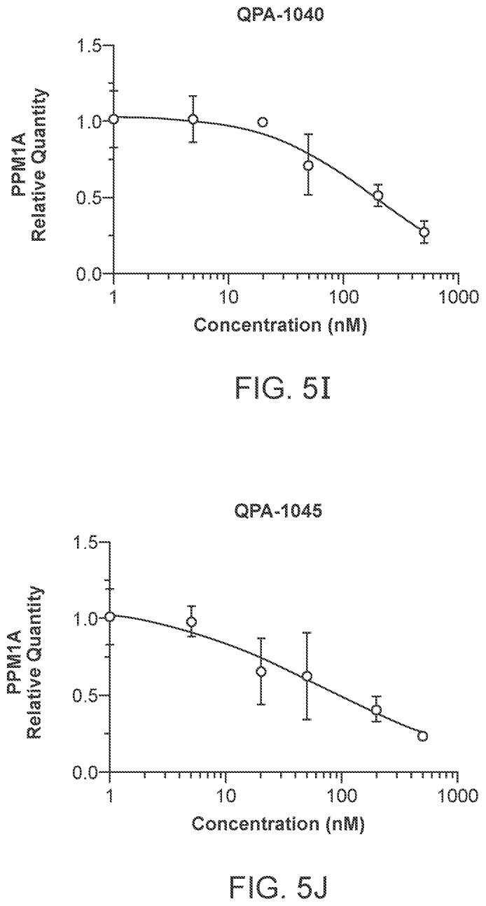

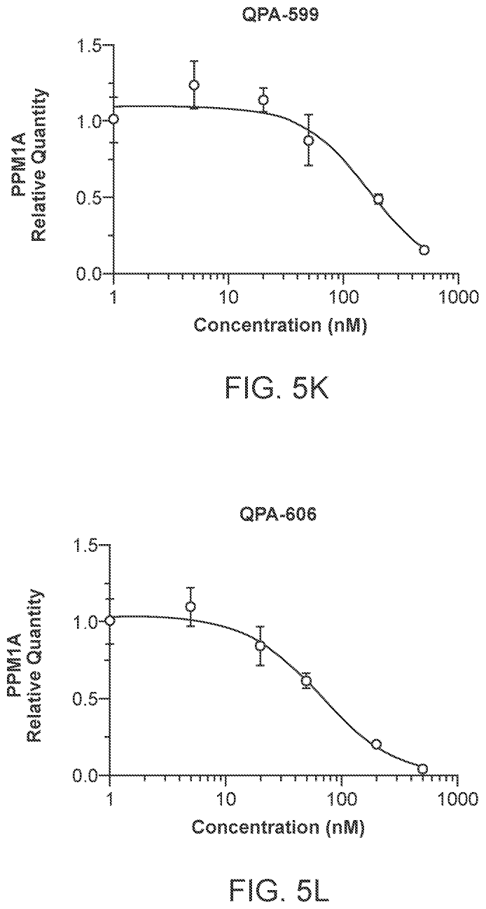

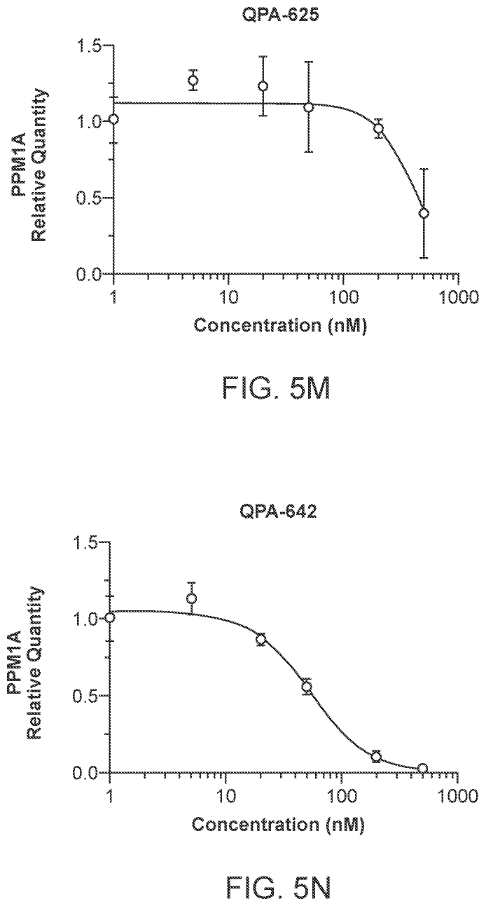

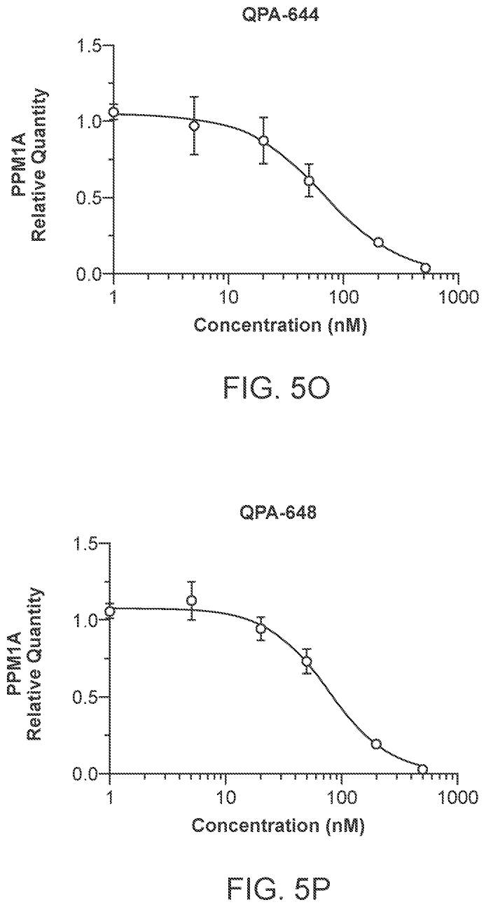

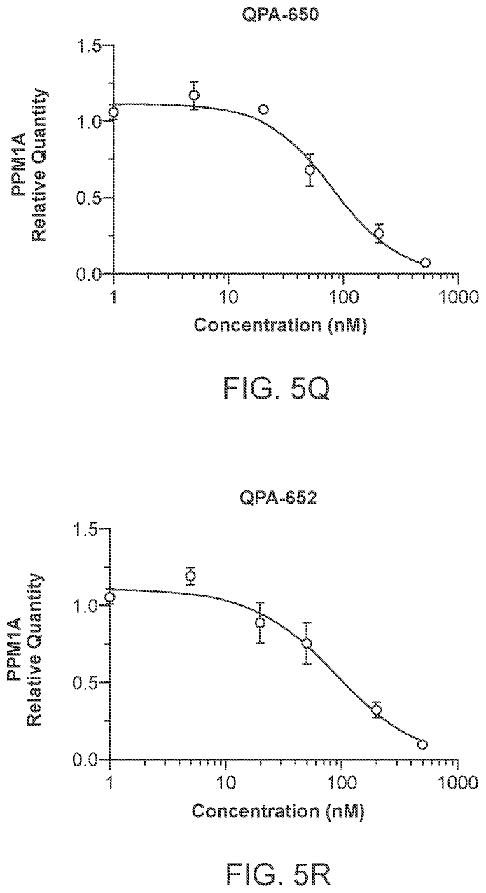

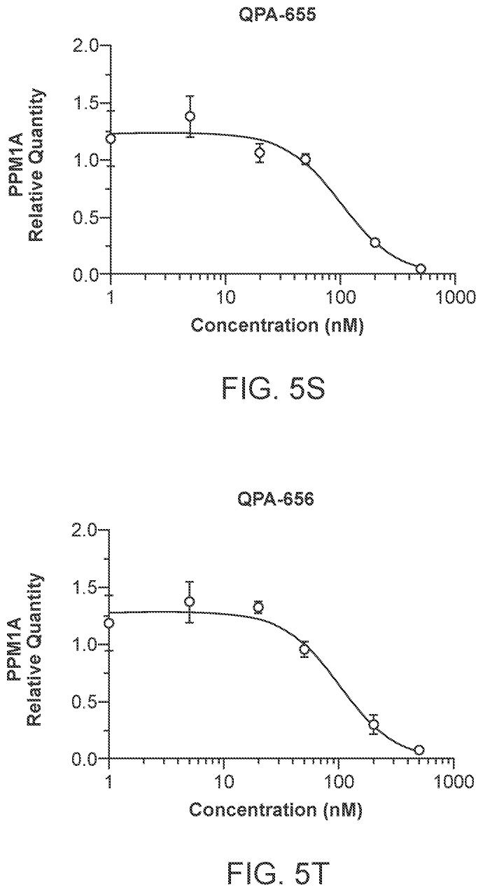

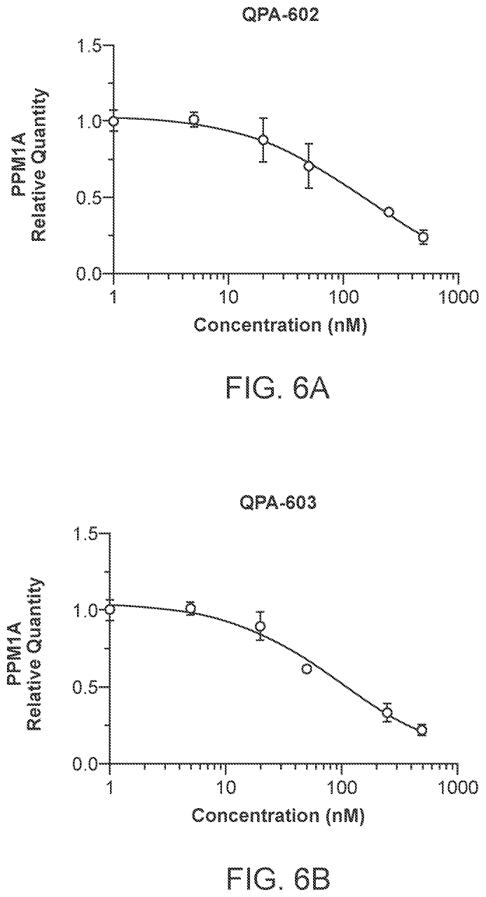

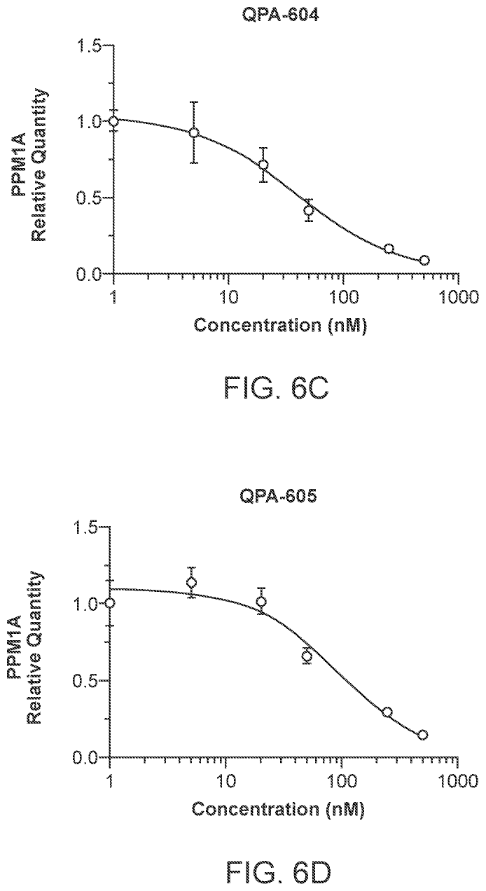

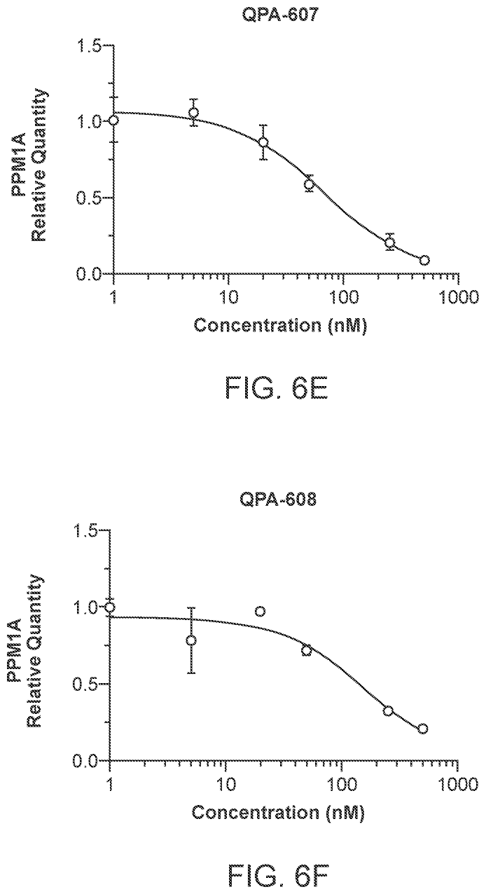

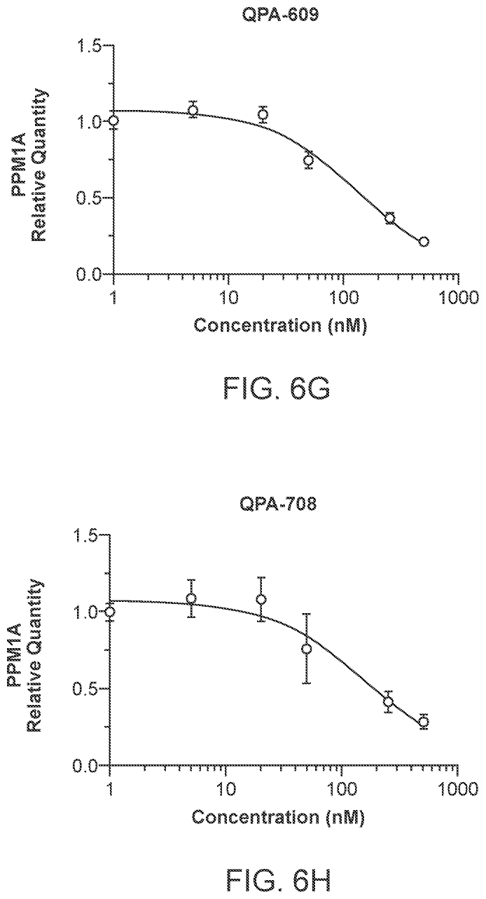

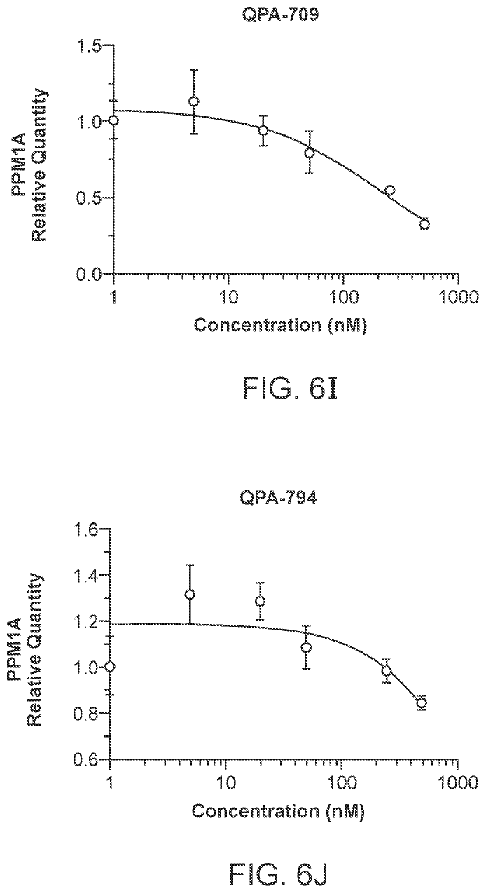

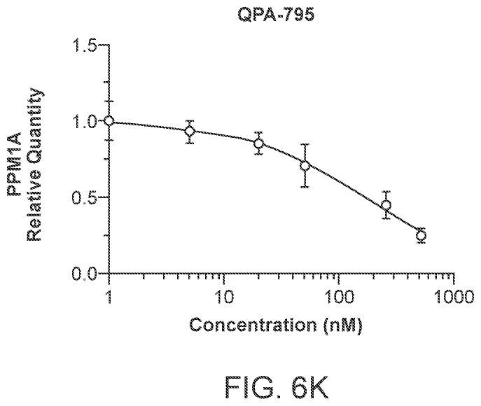

Knockdown potency of example PPM1A AON are represented in FIGS. 5A-5T and FIGS. 6A-6K, which are line graphs of RNA-knockdown potency of various candidate antisense oligonucleotides quantifying the decrease in PPM1A RNA with increasing AON concentration. Non-linear regression four parameter curves were fit and plotted using Graphpad Prism software (San Diego, Calif.), with the bottom of the curve fixed at 0. FIG. 5A represents RNA-knockdown potency of SEQ ID NO: 2883 (QPA-646); FIG. 5B represents RNA-knockdown potency of SEQ ID NO: 2893 (QPA-895); FIG. 5C represents RNA-knockdown potency of SEQ ID NO: 2895 (QPA-905); FIG. 5D represents RNA-knockdown potency of SEQ ID NO: 2911 (QPA-1371); FIG. 5E represents RNA-knockdown potency of SEQ ID NO: 2896 (QPA-910); FIG. 5F represents RNA-knockdown potency of SEQ ID NO: 2897 (QPA-915); FIG. 5G represents RNA-knockdown potency of SEQ ID NO: 2900 (QPA-972); FIG. 5H represents RNA-knockdown potency of SEQ ID NO: 2905 (QPA-1034); FIG. 5I represents RNA-knockdown potency of SEQ ID NO: 2906 (QPA-1040); FIG. 5J represents RNA-knockdown potency of SEQ ID NO: 2907 (QPA-1045); FIG. 5K represents RNA-knockdown potency of SEQ ID NO: 2871 (QPA-599); FIG. 5L represents RNA-knockdown potency of SEQ ID NO: 2876 (QPA-606); FIG. 5M represents RNA-knockdown potency of SEQ ID NO: 2880 (QPA-625); FIG. 5N represents RNA-knockdown potency of SEQ ID NO: 2881 (QPA-642); FIG. 5O represents RNA-knockdown potency of SEQ ID NO: 2882 (QPA-644); FIG. 5P represents RNA-knockdown potency of SEQ ID NO: 2884 (QPA-648); FIG. 5Q represents RNA-knockdown potency of SEQ ID NO: 2885 (QPA-650); FIG. 5R represents RNA-knockdown potency of SEQ ID NO: 2886 (QPA-652); FIG. 5S represents RNA-knockdown potency of SEQ ID NO: 2887 (QPA-655); FIG. 5T represents RNA-knockdown potency of SEQ ID NO: 2888 (QPA-656); FIG. 6A represents RNA-knockdown potency of SEQ ID NO: 2872 (QPA-602); FIG. 6B represents RNA-knockdown potency of SEQ ID NO: 2873 (QPA-603); FIG. 6C represents RNA-knockdown potency of SEQ ID NO: 2874 (QPA-604); FIG. 6D represents RNA-knockdown potency of SEQ ID NO: 2875 (QPA-605); FIG. 6E represents RNA-knockdown potency of SEQ ID NO: 2877 (QPA-607); FIG. 6F represents RNA-knockdown potency of SEQ ID NO: 2878 (QPA-608); FIG. 6G represents RNA-knockdown potency of SEQ ID NO: 2879 (QPA-609); FIG. 6H represents RNA-knockdown potency of SEQ ID NO: 2889 (QPA-708); FIG. 6I represents RNA-knockdown potency of SEQ ID NO: 2890 (QPA-709); FIG. 6J represents RNA-knockdown potency of SEQ ID NO: 2891 (QPA-794); and FIG. 6K represents RNA-knockdown potency of SEQ ID NO: 2892 (QPA-795). IC50 calculated from the fitted non-linear regression curves are listed in Table 12.

TABLE 12
IC50 values for PPM1A AONs shown
in FIGS. 5A-5T and FIGS. 6A-6K.

| AON | IC50 (nM) |
|---|---|
| QPA-646 | 23.99 |
| QPA-895 | 436.9 |
| QPA-905 | 167.6 |
| QPA-1371 | 220.4 |
| QPA-910 | 105.5 |
| QPA-915 | 50.11 |
| QPA-972 | 32.17 |
| QPA-1034 | 136.9 |
| QPA-1040 | 177.1 |
| QPA-1045 | 64.98 |
| QPA-599 | 164 |
| QPA-606 | 65.45 |
| QPA-625 | 393.6 |
| QPA-642 | 53.55 |
| QPA-644 | 65.81 |
| QPA-648 | 77.34 |
| QPA-650 | 80.25 |
| QPA-652 | 89.47 |
| QPA-655 | 102.9 |
| QPA-656 | 101 |
| QPA-602 | 136.6 |
| QPA-603 | 99.34 |
| QPA-604 | 39.32 |
| QPA-605 | 93.72 |
| QPA-607 | 67.92 |
| QPA-608 | 157.6 |
| QPA-609 | 135.1 |
| QPA-708 | 167.5 |
| QPA-709 | 212.7 |
| QPA-794 | 1116 |
| QPA-795 | 164.6 |

To establish that AON decrease PPM1A expression in ALS motor neurons, 5 PPM1A AONs were tested at 4 dose points in human motor neurons derived from 2 ALS iPSC lines. One line carries a mutation in the TBK1 gene c.992+1 G>A and a second line carries a hexanucleotide repeat in C9orf72. The protocol used to generate spinal motor neurons is a modified version of the published protocol in Du et al. Generation and expansion of highly pure motor neuron progenitors from human pluripotent stem cells, Nat. Commun 6, 6626 (2015). iPSC were dissociated into single cells and seeded onto Matrigel (Corning cat #354277, dilution done following vendor specifications for lot #9280004 and 9273009) coated plates. 24 hour later, neural induction medium was added (1:1 DMEM/F12-Thermofisher 11330057 and Neurobasal-Thermofisher 21103049, lx Glutamax-Thermofisher 35050061, lx NEAA-Thermofisher 11140050, lx penicillin-streptomycin-Thermofisher 15140122, 0.1 mM beta-mercaptoethanol-Thermofisher 21985023, lx B-27 supplement-Thermofisher A35828-01, 1×N2 supplement-Thermofisher 17502048, 0.2 ug/mL ascorbic acid-SIGMA A4403) and supplemented with the GSK3B inhibitor CHIR99021 (3 uM from day 1 to day 6 and then 1 uM from day 7 to 12, R&D systems 4423) in addition to the dual SMAD inhibitors SB431542 (10 uM, from day 1 to 12, R&D Systems1614) and LDN193189 (100 nM from day 1 to 12, REPROCELL 04007402), which drives the iPSC's towards neuroepithelial progenitors (NEPs). These NEPs were differentiated towards motor neuron progenitors by adding retinoic acid (1 uM from day 7 to 21, Sigma R2625) and smoothened agonist SAG (1 uM from day 7 to 21, Millipore 566660). These small molecules drive the rostro-caudal axis and ventral identities, respectively. The addition of the gamma secretase inhibitor DAPT (10 uM from day 16 to 21, R&D Systems 2634) during the last 6 days of differentiation helps with the specification of post-mitotic motor neurons increasing the expression of ISL1 positive cells. The spinal motor neurons in culture were maintained in neuronal maturation medium (Neurobasal-Thermofisher 21103049, lx Glutamax-Thermofisher 35050061, lx NEAA-Thermofisher 11140050, lx B-27 plus supplement-Thermofisher A3582801, 1×N2 supplement-Thermofisher 17502048, 0.2 ug/mL ascorbic acid-SIGMA A4403) that contains the growth factors BDNF, CNTF and GDNF (10 ng/mL BDNF-R&D Systems 248-BDB, 10 ng/mL CNTF R&D 257-N and 10 ng/mL GDNF-R&D Systems 212-GD).

Patient iPSC-derived motor neurons were seeded onto 96 well plate (0.32 cm²/well) at a density of 10,000 cells/well. Motor neurons were maintained in neuronal maturation medium, PPM1A knockdown was established by transfecting patient motor neurons with example AON at 4 dose points (5, 20, 50, 200 nM) together with 6 uM endoporter delivery. Cells were treated with 6 uM endoporter alone for transfection control. siControl and siPPM1A were transfected in RNAiMax as negative and positive controls. Treatment conditions were performed in triplicate wells. siRNA were washed out at 48 hours post-transfection. 72 hours post-transfection, all treatment conditions were quantified for PPM1A RNA levels by qRT-PCR assay as described above. Relative quantity was calculated for each AON compared to endoporter alone (RQ=1.0).

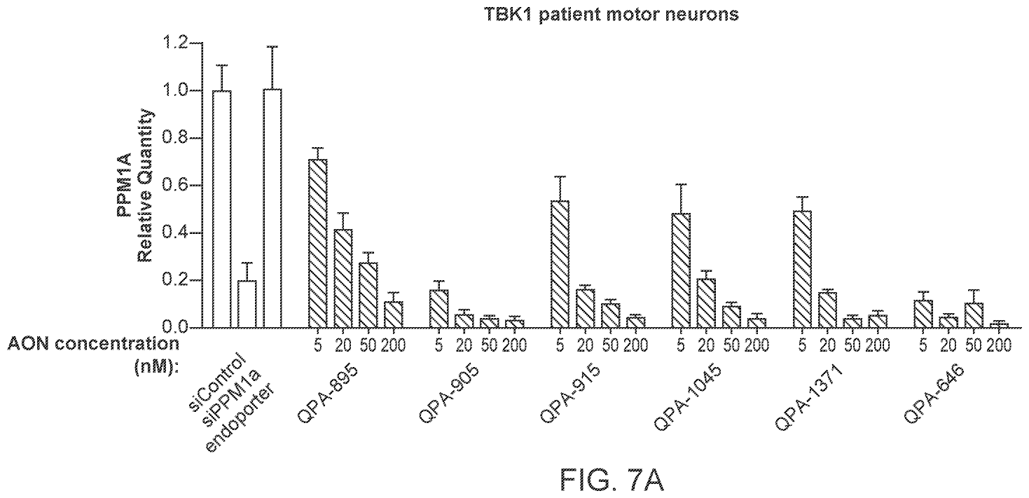

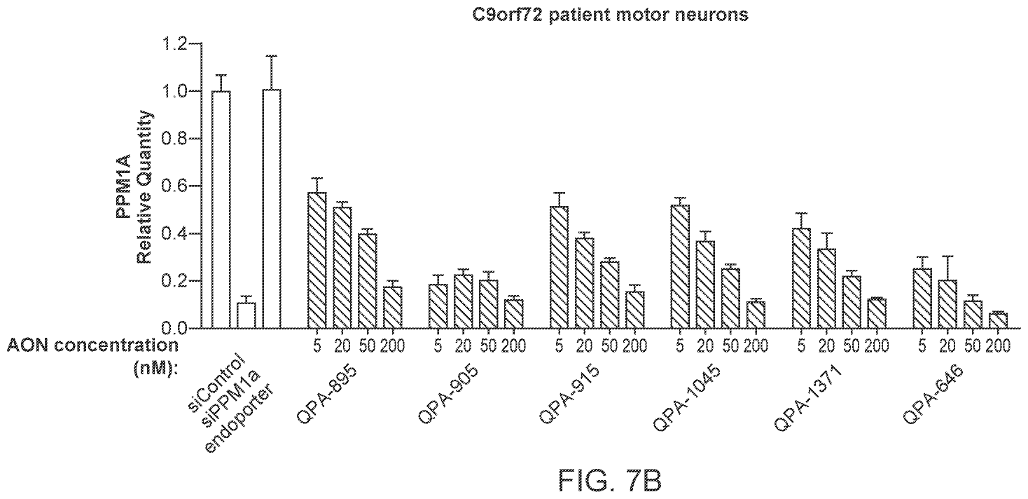

FIGS. 7A and 7B show reduction of PPM1A expression in two ALS iPSC lines (TBK1 and C9orf72) following treatment using PPM1A AONs (QPA-895, QPA-905, QPA-915, QPA-1045, QPA-1371, AND QPA-646). In TBK1 patient motor neurons, PPM1A AON decreased PPM1A RNA in a dose-dependent manner (FIG. 7A, Table 13). 200 nM QPA-895 (SEQ ID NO: 2893) reduced PPM1A RNA to 0.12, 200 nM QPA-905 (SEQ ID NO: 2895) reduced PPM1A RNA to 0.038, 200 nM QPA-915 (SEQ ID NO: 2897) reduced PPM1A RNA to 0.048, 200 nM QPA-1045 (SEQ ID NO: 2907) reduced PPM1A RNA to 0.045, 200 nM QPA-1371 (SEQ ID NO: 2911) reduced PPM1A RNA to 0.057, and 200 nM QPA-646 (SEQ ID NO: 2883) reduced PPM1A RNA to 0.022.

TABLE 13
Relative PPM1A quantities in response to PPM1A AONs in TBK1 patent
motor neurons. Mean +/− Standard deviation

| | Control | 5 nM | 20 nM | 50 nM | 200 nM |
|---|---|---|---|---|---|
| siControl | 1.00 ± 0.10 | | | | |
| siPPM1a | 0.20 ± 0.07 | | | | |
| endoporter | 1.01 ± 0.14 | | | | |
| QPA-895 | | 0.71 ± 0.04 | 0.42 ± 0.07 | 0.28 ± 0.04 | 0.12 ± 0.03 |
| QPA-905 | | 0.16 ± 0.034 | 0.06 ± 0.02 | 0.04 ± 0.01 | 0.04 ± 0.01 |
| QPA-915 | | 0.54 ± 0.10 | 0.17 ± 0.01 | 0.11 ± 0.01 | 0.05 ± 0.01 |
| QPA-1045 | | 0.49 ± 0.12 | 0.21 ± 0.03 | 0.10 ± 0.01 | 0.04 ± 0.01 |
| QPA-1371 | | 0.50 ± 0.05 | 0.15 ± 0.01 | 0.04 ± 0.01 | 0.06 ± 0.02 |
| QPA-646 | | 0.12 ± 0.03 | 0.05 ± 0.01 | 0.11 ± 0.05 | 0.02 ± 0.01 |

In C9orf72 patient motor neurons, PPM1A AON decreased PPM1A RNA in a dose-dependent manner (FIG. 7B, Table 14). 200 nM QPA-895 (SEQ ID NO: 2893) reduced PPM1A RNA to 0.18, 200 nM QPA-905 (SEQ ID NO: 2895) reduced PPM1A RNA to 0.12, 200 nM QPA-915 (SEQ ID NO: 2897) reduced PPM1A RNA to 0.15, 200 nM QPA-1045 (SEQ ID NO: 2907) reduced PPM1A RNA to 0.11, 200 nM QPA-1371 (SEQ ID NO: 2911) reduced PPM1A RNA to 0.12, and 200 nM QPA-646 (SEQ ID NO: 2883) reduced PPM1A RNA to 0.063. These results show example PPM1A AON function to reduce PPM1A transcripts in ALS patient motor neurons.

TABLE 14
Relative PPM1A quantities in response to PPM1A AONs in C9orf72
patent motor neurons. Mean +/− Standard deviation

| | Control | 5 nM | 20 nM | 50 nM | 200 nM |
|---|---|---|---|---|---|
| siControl | 1.00 ± 0.06 | | | | |
| siPPM1a | 0.11 ± 0.03 | | | | |
| endoporter | 1.01 ± .014 | | | | |
| QPA-895 | | 0.57 ± 0.06 | 0.51 ± 0.02 | 0.40 ± 0.02 | 0.18 ± 0.02 |
| QPA-905 | | 0.19 ± 0.03 | 0.22 ± 0.02 | 0.20 ± 0.03 | 0.12 ± 0.01 |
| QPA-915 | | 0.51 ± 0.05 | 0.38 ± 0.02 | 0.28 ± 0.01 | 0.15 ± 0.03 |
| QPA-1045 | | 0.52 ± 0.02 | 0.37 ± 0.04 | 0.25 ± 0.02 | 0.11 ± 0.01 |
| QPA-1371 | | 0.42 ± 0.06 | 0.33 ± 0.06 | 0.22 ± 0.02 | 0.12 ± 0.003 |
| QPA-646 | | 0.25 ± 0.04 | 0.20 ± 0.10 | 0.12 ± 0.02 | 0.06 ± 0.003 |

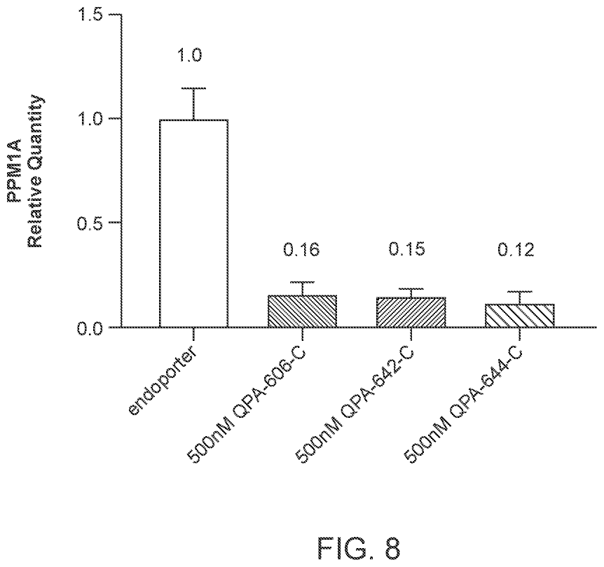

Three PPM1A AON were synthesized with cholesterol conjugated to the 3′ end and tested for function in the PPM1A qRT-PCR assay using iCell human motor neurons in triplicate wells. The three PPM1A AON with a cholesterol conjugate group are shown above in Table 6. 72 hours post-transfection, PPM1A and GAPDH RNA levels were quantified by qRT-PCR. FIG. 8 shows the decreased PPM1A relative quantity in human motor neurons in response to treatment using PPM1A AONs with a cholesterol conjugate group (QPA-606-C, QPA-642-C, QPA-644-C). Results are further shown in Table 15. As compared to endoporter alone (RQ=1.0), 500 nM QPA-606-C(SEQ ID NO: 2876) reduced PPM1A RNA to 0.16, 500 nM QPA-642-C(SEQ ID NO: 2881) reduced PPM1A RNA to 0.15, and 500 nM QPA-644-C(SEQ ID NO: 2882) reduced PPM1A RNA to 0.12. Therefore, cholesterol conjugates of PPM1A AON sequences significantly decrease PPM1A RNA.

TABLE 15
Relative PPM1A quantities in response to PPM1A AONs with cholesterol
conjugate group. Mean +/− Standard deviation

| | PPM1A Relative Quantity |
|---|---|
| endoporter | 1.00 ± 0.14 |
| 500 nM QPA-606-C | 0.16 ± 0.06 |
| 500 nM QPA-642-C | 0.15 ± 0.03 |
| 500 nM QPA-644-C | 0.12 ± 0.05 |

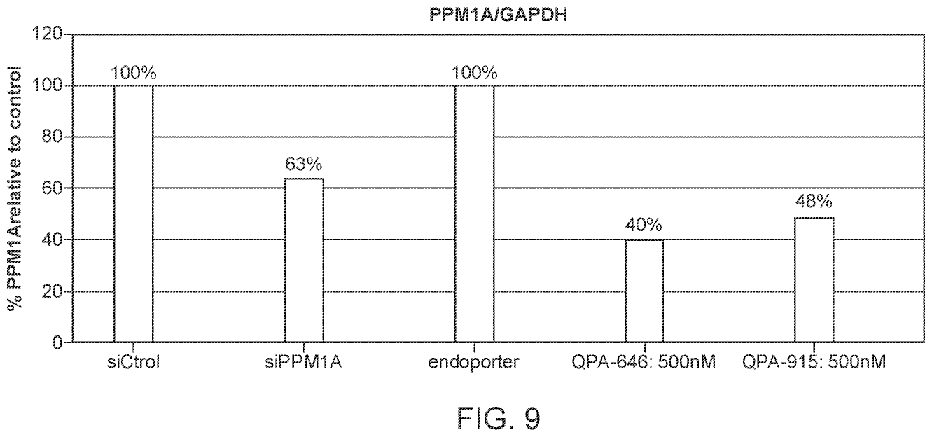

To further test PPM1A AON for ability to inhibit PPM1A expression, PPM1A and downstream target protein levels were quantified following AON transfection of human motor neurons (FIGS. 9-12). Protein levels were quantified by western blot and using the method as follows. Motor neurons derived from wildtype or diseased iPSC-derived motor neurons were seeded onto 6 well plates (9.6 cm²) or 12 well plates (3.5 cm²) at a density of 750,000 cells/well and 400,000 cells/well respectively. Motor neurons were maintained with neuronal maturation media (Neurobasal-Thermofisher 21103049, lx Glutamax-Thermofisher 35050061, lx NEAA-Thermofisher 11140050, lx B-27 plus supplement-Thermofisher A3582801, 1×N2 supplement-Thermofisher 17502048, 0.2 ug/mL ascorbic acid-Sigma A4403 supplemented with growth factors BDNF, CNTF and GDNF (10 ng/mL BDNF-R&D Systems 248-BDB, 10 ng/mL CNTF R&D 257-N and 10 ng/mL GDNF-R&D Systems 212-GD).

Motor neurons were transfected 5 days post-plating in complete neuronal maturation media. The transfection of AONs were done using Endoporter at a final concentration of 6 μM. Cells were incubated for 72 hours and then collected for western blotting. The cell lysis buffer 2% SDS (50 mM Tris pH7, 10% glycerol, 2% SDS) was supplemented with 1×Halt protease inhibitor cocktail (Thermofisher 78425) and 1× Halt phosphatase inhibitor cocktail (Thermofisher 78428). Samples collected using 2% SDS were left in the 95° C. heat block for 10 minutes right after collection followed by a short spin to gather any evaporation accumulated on the lids. Protein quantification was done using a Pierce BCA Protein Assay Kit (Thermofisher 23227) following manufacturer instructions. The plate reading was done using a SpectraMax i3× from Molecular Devices and the data collected using the SoftMax pro. Gels were run using 4-20% Criterion™ TGX Stain-Free™ Protein Gel (Biorad). After running the gels, the membranes were transferred using the Iblot2 transfer system. Membranes were blocked in either 5% BSA (for phosphorylated proteins) or 5% milk for 40 minutes. Membranes were incubated with primary antibodies overnight at 4° C. The following antibodies were used LC3B (Cell Signaling CST2775); PPM1A (Abcam ab14824); NAK/TBK1 (Abcam ab40676); Phospho-TBK1/NAK (Cell Signaling 5483); GAPDH (Proteintech 60004 and Abcam ab181602). The following secondary antibodies were used (Anti-rb Rabbit IgG, HRP linked (Cell Signaling 7074) and Anti-ms IgG, HRP linked (Cell Signaling 7076). Images were obtained using Li-Cor Fc imaging system and the software used for quantification was the Image Studio Lite.

First, PPM1A AON were examined for ability to decrease PPM1A protein levels in TBK1 mutation ALS patient iPSC-derived motor neurons. PPM1A AON were transfected at 500 nM with endoporter and control wells were treated with endoporter alone. Additionally, siControl (siCtrol) and siPPM1A were transfected with RNAiMax and washed out after 48 hours. 72 hours post-transfection, all treatment groups were collected for western blot analysis of PPM1A protein levels. PPM1A band intensity was quantified and normalized to GAPDH. Percent expression of PPM1A was calculated by dividing the PPM1A/GAPDH value by control and multiplying by 100 (SiPPM1A vs. siCtrol; PPM1A AON vs. endoporter).

FIG. 9 and Table 16 shows the reduction in PPM1A protein in response to treatment using PPM1A AONs (QPA-646 and QPA-915). 500 nM QPA-646 (SEQ ID NO: 2883) reduced PPM1A protein to 40% of normal and QPA-915 (SEQ ID NO: 2897) reduced PPM1A protein to 48% of normal. Thus, PPM1A AON decrease PPM1A transcripts leading to reduction of protein expression.

TABLE 16
Relative PPM1A quantities normalized to control
(endoporter) in response to PPM1A AONs.

| | % PPM1A relative to control |
|---|---|
| siCtrol | 100 |
| siPPMIA | 63.463 |
| endoporter | 100 |
| endo + CL | 69.4383 |
| QPA-646: 500 nM | 39.8165 |
| QPA-915: 500 nM | 48.4634 |

Next, PPM1A AON were examined for ability to decrease PPM1A protein levels in wildtype iPSC-derived motor neurons. The following PPM1A AON were evaluated: QPA-642 (SEQ ID NO: 2881), QPA-646 (SEQ ID NO: 2883), QPA-1371 (SEQ ID NO: 2911), QPA-905 (SEQ ID NO: 2895), and QPA-915 (SEQ ID NO: 2897). PPM1A AON were transfected at 50, 250, and 500 nM with endoporter and control wells were treated with endoporter alone. 72 hours post-transfection, all treatment groups were collected for western blot analysis of PPM1A protein levels. PPM1A band intensity was quantified and normalized to GAPDH. Percent expression of PPM1A was calculated by dividing the PPM1A/GAPDH value by control and multiplying by 100 (PPM1A AON vs. endoporter control).

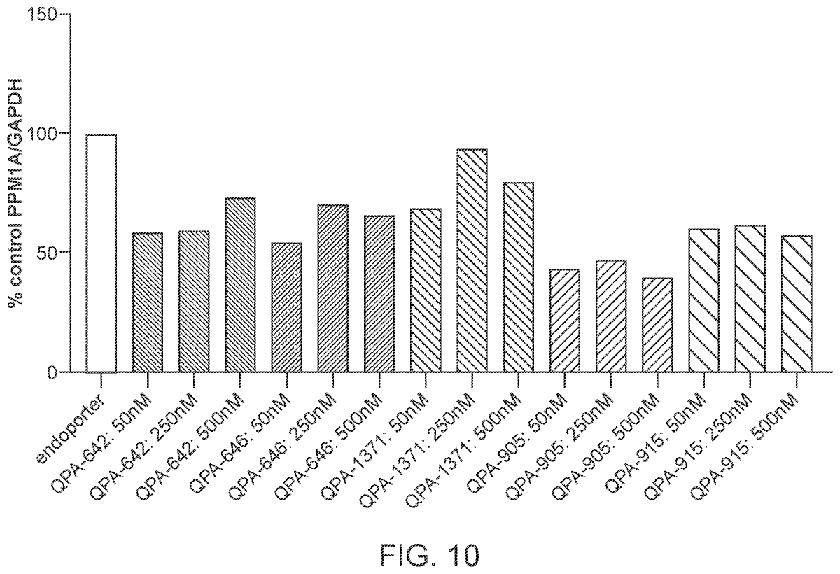

FIG. 10 shows the decrease in PPM1A protein levels in wildtype iPSC-derived motor neurons in response to treatment using PPM1A AONs (QPA-642, QPA-646, QPA-1371, QPA-905, and QPA-915). All PPM1A AONs decreased PPM1A protein to levels between 40-94% of normal by 72 hours (Table 17). Thus, PPM1A AONs decrease PPM1A transcripts leading to reduction of protein expression.

TABLE 17
PPM1A AON decrease PPM1A protein levels at 72 hours

| AON | control | 50 nM | 250 nM | 500 nM |
|---|---|---|---|---|
| Endoporter alone | 100 | | | |
| QPA-642 | | 58.9% | 59.4% | 73.5% |
| QPA-646 | | 54.4% | 70.5% | 65.9% |
| QPA-1371 | | 69.0% | 93.8% | 80.0% |
| QPA-905 | | 43.4% | 47.3% | 39.8% |
| QPA-915 | | 60.3% | 62.0% | 57.5% |

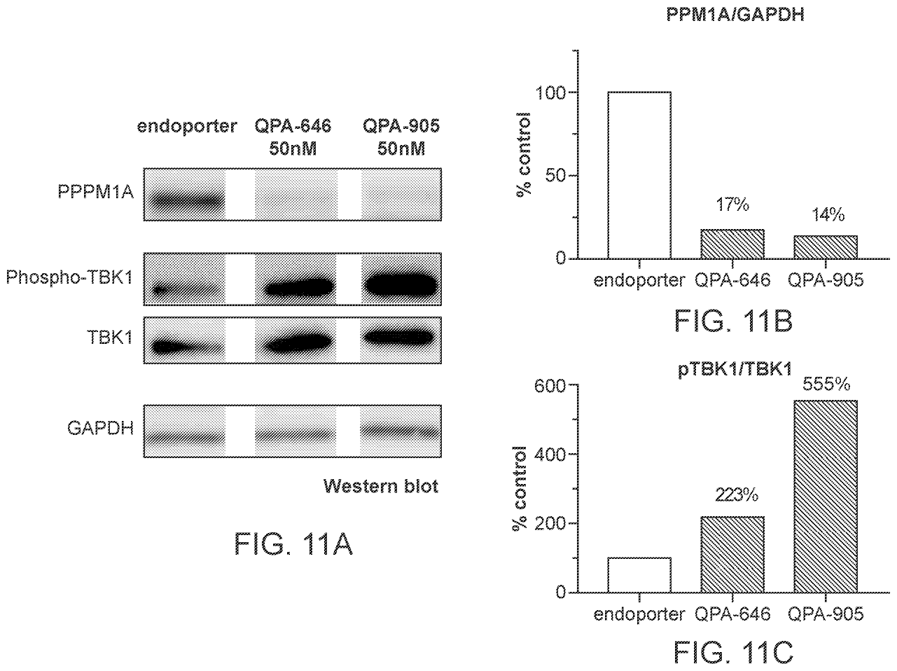

PPM1A functions as a phosphatase and one of the targets it dephosphorylates is the protein TBK1. Therefore, we investigated whether reduction of PPM1A transcripts and protein has a downstream function impact to increase phosphorylation of TBK1. TBK1 is known to be phosphorylated at serine 172, and dephosphorylation controlled by PPM1A activity (Xiang et al, PPM1A silences cytosolic RNA sensing and antiviral defense through direct dephosphorylation of MAVS and TBK1, Science Advances, 2(7), Jul. 1, 2016). Wildtype iPSC-derived human motor neurons were endoporter transfected with 50 nM QPA-646 (SEQ ID NO: 2883), 50 nM QPA-905 (SEQ ID NO: 2895), or treated with endoporter alone (control) according to the methods described above for western blot assay. AON and endoporter was removed and neurons replaced with fresh media after 72 hours. On day 7 post-transfection, motor neurons were treated a second time with AON and endoporter or endoporter alone. On day 14, motor neurons were lysed and analyzed for PPM1A, phosphorylated TBK1 (pTBK1, serine172), TBK1, and GAPDH by western blot for protein levels. FIGS. 11A-11C and Table 17 show the qualitative and quantitative results of the Western blot analysis in human motor neurons treated using PPM1A AONs (QPA-646 and QPA-905). QPA-646 (SEQ ID NO: 2883) decreased PPM1A protein to 17% of control and QPA-905 (SEQ ID NO: 2895) decreased PPM1A protein to 14% of control. QPA-646 (SEQ ID NO: 2883) increased pTBK1 relative to TBK1 to 223% of control and QPA-905 (SEQ ID NO: 2895) increased pTBK1 relative to TBK1 to 555% of control. Both AON showed sustained knockdown of PPM1A at the protein level after 2 weeks of AON treatment leading to an increase in the downstream effector pTBK1.

TABLE 17
PPM1A AON decrease PPM1A protein levels and
increase PTBK1/TBK1 levels at 72 hours.

| | % Control PPM1A/GAPDH | % Control PTBK1/TBK1 |
|---|---|---|
| endoporter | 100 | 100 |
| QPA-646 | 17.2414 | 222.965 |
| QPA-905 | 13.7931 | 554.865 |

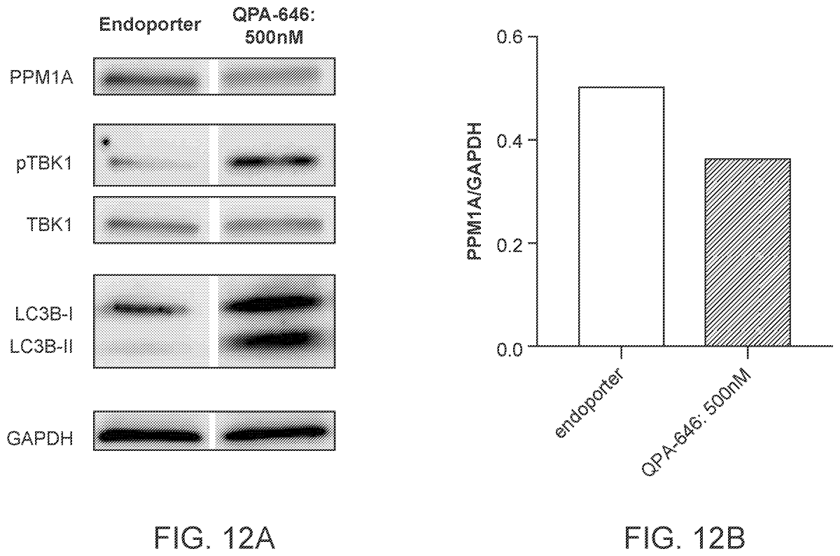

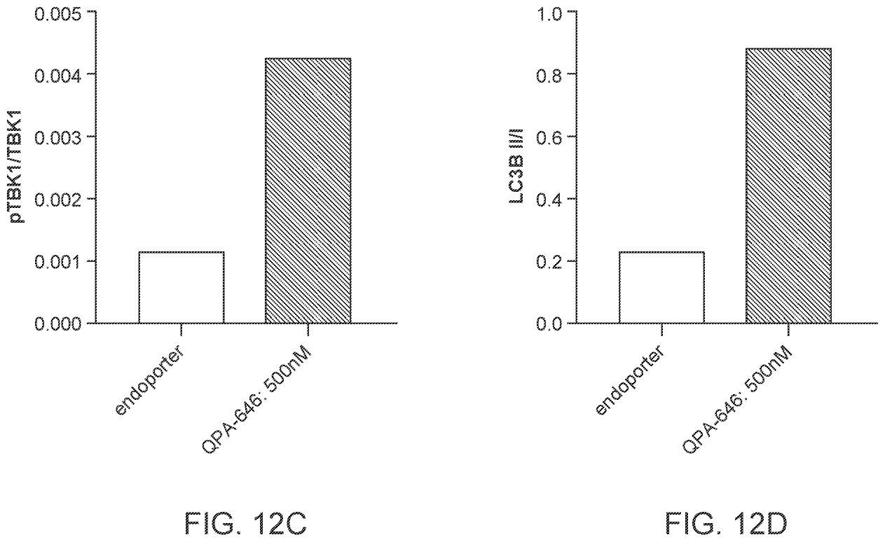

In order to determine whether PPM1A AON can affect additional downstream pathways, induction of autophagy through LC3B was examined. Wildtype iPSC-derived human motor neurons were endoporter transfected with 500 nM QPA-646 (SEQ ID NO: 2883) or treated with endoporter alone (control) according to the methods described above for western blot assay. 72 hours post-transfection, cells were lysed and processed for western blot detection of protein levels. FIGS. 12A-12D and Table 18 show the qualitative and quantitative results of the Western blot analysis in wildtype iPSC-derived human motor neurons treated using PPM1A AON (QPA-646). QPA-646 (SEQ ID NO: 2883) decreased PPM1A protein (0.50 endoporter vs. 0.37 QPA-646), increased pTBK1 relative to TBK1 (0.0011 endoporter vs. 0.0043 QPA-646) and increased LC3B II relative to LC3B 1 (0.23 endoporter vs. 0.88 QPA-646). The ratio of LC3B II to I increases with autophagy induction as more autophagosomes containing the lipidated LC3B (II) are formed. Therefore, PPM1A AON increases downstream pathway activity leading to increased pTBK1 and autophagy.

TABLE 18
PPM1A AON decrease PPM1A protein levels, increase PTBK1/TBK1
levels, and increase LC3B II/I at 72 hours.

| | PPM1A/GAPDH | pTBK1/TBK1 | LC3B II/I |
|---|---|---|---|
| endoporter | 0.50 | 0.001 | 0.23 |
| QPA-646: 500 nM | 0.37 | 0.004 | 0.88 |

Inhibition of the proteasome causes proteotoxic stress leading to cell death. As a model of protein stress and neurodegeneration, we examined whether PPM1A AON rescue cell survival after proteasome inhibition with MG132. SY5Y cells were plated at a density of 5,000 cells/well in a 384-well plate and cultured for 24 hours. SY5Y were then transfected with AON at 200 nM, QPA-905 (SEQ ID NO: 2895), QPA-1045 (SEQ ID NO: 2907), QPA-895 (SEQ ID NO: 2893) for 72 hours. Cells received a 24 hour washout with fresh media. 0.4 uM MG132 (Cat. No. 1748, Tocris) was added to wells treated with AON and also to control wells. Cell survival was measured 16 hours later by the CellTiter-Glo 2.0 cell viability assay (Promega, Madison, Wis.) according to manufacturer's instructions. Cell lysates were quantified for luminescence on the GloMax Luminometer (Promega, Madison, Wis.). All treatment conditions were performed in 7 replicate wells. Luminescence data was normalized so that untreated condition equals 100% response and MG132 treated equals 0% response. Percent rescue of cell survival was calculated for AON and MG132 combination treatment.

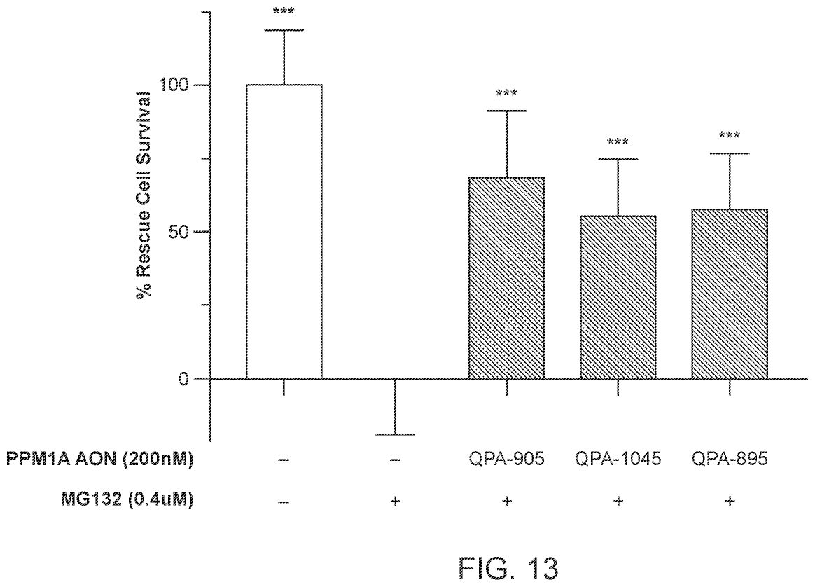

FIG. 13 and Table 19 show the percent rescue of cell survival in a proteotoxic stress neurodegeneration model in response to treatment using PPM1A AONs (QPA-905, QPA-1045, and QPA-895). QPA-905 (SEQ ID NO: 2895) rescued cell survival by 69%, QPA-1045 (SEQ ID NO: 2907) rescued cell survival by 56% and QPA-895 (SEQ ID NO: 2893) rescued cell survival by 58%. QPA-905 (SEQ ID NO: 2895), QPA-1045 (SEQ ID NO: 2907), and QPA-895 (SEQ ID NO: 2893) all significantly increase cell survival (***p<0.0001, one-way ANOVA with Tukey multiple comparisons test vs. MG132 alone). Therefore, AON which decrease PPM1A, lead to increased autophagy capacity that functions to protect cells from neurodegeneration.

TABLE 19
PPM1A AON treatment leads to rescue of cell survival in a proteotoxic
stress degeneration model. Mean +/− Standard deviation

| | % Rescue Cell Survival Relative to Control |
|---|---|
| No Tx | 100 ± 18.72 |
| MG132 | 0 ± 19.13 |
| QPA-905 | 68.7 ± 22.62 |
| QPA-1045 | 55.55 ± 19.46 |
| QPA-895 | 58.37 ± 18.42 |

### INCORPORATION BY REFERENCE

The entire disclosure of each of the patent documents and scientific articles cited herein is incorporated by reference for all purposes.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A compound comprising an oligonucleotide comprising linked nucleosides with a nucleobase sequence that is at least 90% complementary to an equal length portion of a transcript that is transcribed from at least nucleotide 41,932 to nucleotide 42,787 and from nucleotide 44,874 to nucleotide 44,990 of SEQ ID NO: 1, wherein at least one nucleoside linkage of the linked nucleosides is a non-natural linkage.

2. An oligonucleotide comprising linked nucleosides with a nucleobase sequence that is at least 90% complementary to an equal length portion of a transcript that is transcribed from at least nucleotide 41,932 to nucleotide 42,787 and from nucleotide 44,874 to nucleotide 44,990 of SEQ ID NO: 1, wherein at least one nucleoside linkage of the linked nucleosides is a non-natural linkage.

3. The oligonucleotide of claim 1 or 2, wherein the transcript transcribed from nucleotide 41,932 to nucleotide 42,787 and from nucleotide 44,874 to nucleotide 44,990 of SEQ ID NO: 1 comprises a sequence of any of SEQ ID NO: 2864, SEQ ID NO: 2865, or SEQ ID NO: 2866.

4. A compound comprising an oligonucleotide comprising linked nucleosides with a nucleobase sequence that is at least 90% complementary to an equal length portion of a transcript that shares at least 90% identity to SEQ ID NO: 2864, SEQ ID NO: 2865, or SEQ ID NO: 2866, or to a contiguous 15 to 50 nucleobase portion of SEQ ID NO: 2864, SEQ ID NO: 2865, or SEQ ID NO: 2866, wherein at least one nucleoside linkage of the linked nucleosides is a non-natural linkage.

5. An oligonucleotide comprising linked nucleosides with a nucleobase sequence that is at least 90% complementary to an equal length portion of a transcript that shares at least 90% identity to SEQ ID NO: 2864, SEQ ID NO: 2865, or SEQ ID NO: 2866, or to a contiguous 15 to 50 nucleobase portion of SEQ ID NO: 2864, SEQ ID NO: 2865, or SEQ ID NO: 2866, wherein at least one nucleoside linkage of the linked nucleosides is a non-natural linkage.

6. The oligonucleotide of claims 4 or 5, wherein the nucleobase sequence comprises a portion of at least 10 contiguous nucleobases that shares at least 90% identity with an equal length portion of any one of SEQ ID NOs: 2-955, SEQ ID NOs: 1910-2863, SEQ ID NOs: 2868-2913, and SEQ ID NOs: 2914-2959.

7. The oligonucleotide of any one of claims 4-6, wherein the nucleobase sequence comprises a portion of at least 11, 12, 13, 14, 15, 16, 17, 18, 19, or 20 contiguous nucleobases that shares at least 90% identity with an equal length portion of any one of SEQ ID NOs: 2-955, SEQ ID NOs: 1910-2863, SEQ ID NOs: 2868-2913, and SEQ ID NOs: 2914-2959.

8. The oligonucleotide of any one of claims 4-6, wherein the nucleobase sequence comprises a portion of at least 10 contiguous nucleobases that shares at least 90% identity with an equal length portion of any one of SEQ ID NOs: 2868-2913 and SEQ ID NOs: 2914-2959.

9. The oligonucleotide of any one of claims 4-6, wherein the nucleobase sequence comprises a portion of at least 11, 12, 13, 14, 15, 16, 17, 18, 19, or 20 contiguous nucleobases that shares at least 90% identity with an equal length portion of any one of SEQ ID NOs: 2868-2913 and SEQ ID NOs: 2914-2959.

10. The oligonucleotide of claim 4 or 5, wherein the nucleobase sequence comprises a portion of at least 10 contiguous nucleobases that is at least 90% complementary to an equal length portion of nucleobases within any one of positions 457-1410 of SEQ ID NO: 2864.

11. The oligonucleotide of any one of claims 4-5 or 10, wherein the nucleobase sequence comprises a portion of at least 11, 12, 13, 14, 15, 16, 17, 18, 19, or 20 contiguous nucleobases that is at least 90% complementary to an equal length portion of nucleobases within any one of positions 457-1410 of SEQ ID NO: 2864.

12. The oligonucleotide of any one of claims 4-5 or 10, wherein the nucleobase sequence comprises a portion of at least 10 contiguous nucleobases that is at least 90% complementary to an equal length portion of nucleobases within any one of positions 542-814, 895-1006, 1025-1117, or 1361-1407 of SEQ ID NO: 2864.

13. The oligonucleotide of any one of claims 10-12, wherein the nucleobase sequence comprises a portion of at least 11, 12, 13, 14, 15, 16, 17, 18, 19, or 20 contiguous nucleobases that is at least 90% complementary to an equal length portion of nucleobases within any one of positions 542-814, 895-1006, 1025-1117, or 1361-1407 of SEQ ID NO: 2864.

14. The oligonucleotide of claim 10 or 12, wherein the nucleobase sequence comprises a portion of at least 10 contiguous nucleobases that is at least 90% complementary to an equal length portion of nucleobases within any one of positions 542-561, 555-574, 559-578, 599-618, 602-621, 603-622, 604-623, 605-624, 606-625, 607-626, 608-627, 609-628, 625-644, 642-661, 644-663, 646-665, 648-667, 650-669, 652-671, 655-674, 656-675, 708-727, 709-728, 794-813, 795-814, 895-914, 900-919, 905-924, 910-929, 915-934, 962-981, 967-986, 972-991, 977-996, 987-1006, 1025-1044, 1030-1049, 1034-1053, 1040-1059, 1045-1064, 1098-1117, 1361-1380, 1366-1385, 1371-1390, 1378-1397, and 1386-1405 of SEQ ID NO: 2864.

15. The oligonucleotide of any one of claims 10-14, wherein the nucleobase sequence comprises a portion of at least 11, 12, 13, 14, 15, 16, 17, 18, 19, or 20 contiguous nucleobases that is at least 90% complementary to an equal length portion of nucleobases within any one of positions 542-561, 555-574, 559-578, 599-618, 602-621, 603-622, 604-623, 605-624, 606-625, 607-626, 608-627, 609-628, 625-644, 642-661, 644-663, 646-665, 648-667, 650-669, 652-671, 655-674, 656-675, 708-727, 709-728, 794-813, 795-814, 895-914, 900-919, 905-924, 910-929, 915-934, 962-981, 967-986, 972-991, 977-996, 987-1006, 1025-1044, 1030-1049, 1034-1053, 1040-1059, 1045-1064, 1098-1117, 1361-1380, 1366-1385, 1371-1390, 1378-1397, and 1386-1405 of SEQ ID NO: 2864.

16. The oligonucleotide of any one claims 1-15, wherein the oligonucleotide comprises at least one nucleoside linkage selected from the group consisting of a phosphodiester linkage, a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, or any combination(s) thereof.

17. The oligonucleotide of any one of the preceding claims, wherein at least one internucleoside linkage of the nucleotide sequence is a phosphorothioate linkage.

18. The oligonucleotide of claim 17, wherein the phosphorothioate internucleoside linkage is in one of a Rp configuration or a Sp configuration.

19. The oligonucleotide of any one of claims 1-18, wherein the oligonucleotide comprises one or more chiral centers and/or double bonds.

20. The oligonucleotide of claim 19, wherein the oligonucleotide exist as stereoisomers selected from geometric isomers, enantiomers, and diastereomers.

21. The oligonucleotide of any one of the preceding claims, wherein all internucleoside linkages of the nucleotide sequence are phosphorothioate linkages.

22. The oligonucleotide of any one of the preceding claims, wherein the oligonucleotide comprises at least one modified nucleobase.

23. The oligonucleotide of claim 22, wherein the at least one modified nucleobase is 5-methylcytosine, pseudouridine, or 5-methoxyuridine.

24. The oligonucleotide of any one of the preceding claims, wherein the oligonucleotide comprises at least one nucleoside with a modified sugar moiety.

25. The oligonucleotide of claim 24, wherein the modified sugar moiety is one of a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

26. The oligonucleotide of any one of the preceding claims, wherein the oligonucleotide comprises two, three, four, five, six, seven, eight, nine, or ten nucleosides with modified sugar moieties.

27. The oligonucleotide of claim 26, wherein the modified sugar moieties are independently any one of a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

28. The oligonucleotide of any one of the preceding claims, wherein the oligonucleotide comprises ten 2′-O-(2-methoxyethyl) (2′MOE) nucleosides.

29. The oligonucleotide of claim 28, wherein five of the 2′-O-(2-methoxyethyl) (2′MOE) nucleosides are located at the 3′ end of the oligonucleotide, and wherein five of the 2′-O-(2-methoxyethyl) (2′MOE) nucleosides are located at the 5′ end of the oligonucleotide.

30. The oligonucleotide of any one of claims 24-29, wherein the at least one nucleoside with the modified sugar moiety or the nucleosides with modified sugar moieties are ribonucleosides.

31. The oligonucleotide of any one of the preceding claims, wherein the oligonucleotide comprises at least one deoxyribonucleoside.

32. The oligonucleotide of any one of the preceding claims, wherein the oligonucleotide comprises two, three, four, five, six, seven, eight, nine, or ten deoxyribonucleosides.

33. The oligonucleotide of any one of claims 1-17, wherein the oligonucleotide comprises:
e. a gap segment comprising one or more of linked deoxyribonucleosides, 2′-Fluoro Arabino Nucleic Acids (FANA), and Fluoro Cyclohexenyl nucleic acid (F-CeNA);
f. a 5′ wing region comprising linked nucleosides; and
g. a 3′ wing region comprising linked nucleosides;
h. wherein the central region comprises a region of at least 8 contiguous nucleobases having at least 80% identity to an equal length portion of any one of SEQ ID NOs: 2-955, SEQ ID NOs: 1910-2863, or SEQ ID NOs: 2868-2959 positioned between the 5′ wing segment and the 3′ wing segment; wherein the 5′ wing region and the 3′ wing region each comprises at least two linked nucleosides; and wherein at least one nucleoside of each wing region comprises a modified sugar.

34. The oligonucleotide of claim 33, wherein the at least two linked nucleosides of the 5′ wing region are linked through a phosphorothioate internucleoside linkage and/or wherein the at least two linked nucleosides of the 3′ wing region are independently linked through a phosphorothioate internucleoside linkage.

35. The oligonucleotide of claim 33 or 34, wherein every internucleoside linkage of the 5′ wing region and/or every internucleoside linkage of the 3′ wing region, independently are phosphorothioate internucleoside linkages.

36. The oligonucleotide of claim 33 or 34, wherein the 5′ wing region further comprises at least one phosphodiester internucleoside linkage.

37. The oligonucleotide of claim 33 or 34, wherein the 3′ wing region further comprises at least one phosphodiester internucleoside linkage.

38. The oligonucleotide of claim 33, wherein the at least two linked nucleosides of the 5′ wing region are linked through a phosphodiester internucleoside linkage and/or wherein the at least two linked nucleosides of the 3′ wing region are independently linked through a phosphodiester internucleoside linkage.

39. The oligonucleotide of any one of claims 33-38, wherein at least one of the internucleoside linkages of the central region is a phosphodiester linkage.

40. The oligonucleotide of claim 39, wherein at least two, at least three, at least four, at least five, at least six, at least seven, at least eight, or at least nine of the internucleoside linkages of the central region are phosphodiester linkages.

41. The oligonucleotide of any one of claims 33-38, wherein at least one of the internucleoside linkages of the central region is a phosphorothioate internucleoside linkage.

42. The oligonucleotide of claim 41, wherein at least two, at least three, at least four, at least five, at least six, at least seven, at least eight, or at least nine of the internucleoside linkages of the central region are phosphorothioate internucleoside linkages.

43. The oligonucleotide of any one of claims 33-34 or 41-42, wherein all internucleoside linkages of the oligonucleotide are phosphorothioate internucleoside linkages.

44. The oligonucleotide of any one of claims 33-43, wherein any one or all of the phosphorothioate internucleoside linkages are in a Rp configuration, a Sp configuration, or in any combination of Rp and Sp configuration.

45. The oligonucleotide of any one of claims 33-44, wherein the oligonucleotide comprises at least one modified sugar moiety.

46. The oligonucleotide of claim 45, wherein the 5′ wing region or the 3′ wing region comprises the at least one modified sugar moiety.

47. The oligonucleotide of claim 45, wherein the central region comprises the at least one modified sugar moiety.

48. The oligonucleotide of any one of claims 45-47, wherein the at least one modified sugar moiety is any one of a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

49. The oligonucleotide of any one of claims 45-48 wherein the oligonucleotide comprises one or more 2′-MOE nucleosides.

50. The oligonucleotide of claim 49, wherein the 5′ wing region or the 3′ wing region comprise one or more 2′-MOE nucleosides.

51. The oligonucleotide of claim 49 or 50, wherein the 5′ wing region or the 3′ wing region comprise two, three, four, or five 2′-MOE nucleosides.

52. The oligonucleotide of claim 51, wherein every nucleoside of the 5′ wing region or the 3′ wing region is a 2′-MOE nucleoside.

53. The oligonucleotide of claim 49, wherein the central region comprises one or more 2′-MOE nucleosides.

54. The oligonucleotide of claim 53, wherein the central region comprises two, three, four, five, six, seven, eight, nine, or ten 2′-MOE nucleosides.

55. The oligonucleotide of claim 54, wherein every nucleoside of the central region is a 2′-MOE nucleoside.

56. The oligonucleotide of any one of claims 49-55, wherein the one or more 2′-MOE nucleosides are linked through phosphorothioate internucleoside linkages.

57. The oligonucleotide of claim 33, wherein the oligonucleotide comprises sugar modifications in any of the following patterns: eeeee-d10-eeeee, eee-d8-eee, eee-d10-eee, eeee-d10-eeee, and eeee-d8-eeee, wherein e=2′-MOE nucleoside and d=a deoxyribonucleoside.

58. The oligonucleotide of claim 57, wherein the oligonucleotide comprises internucleoside linkages in any of the following patterns: sssssooooooooosssss; ooooosssssssssooooo; oooooooooooooosssss; soossssssssssssssss; ssssssssssssssssoos; sssssoooooooooooooo; sssssssssssssssssss; sssooooooosss; ooosssssssooo; sssssssssssss; sosssssssssos; sosssssssssss; sssssssssssos; ssssssssssooo; ooossssssssss; sssooooooooosss; ooosssssssssooo; sssssssssssssss; ssssssssssssooo; ooossssssssssss; sosssssssssssos; sosssssssssssss; sssssssssssssos; ssssooooooooossss; oooosssssssssoooo; sssssssssssssssss; sssssssssssssoooo; soosssssssssssoos; soossssssssssssss; ssssssssssssssoos; oooosssssssssssss; ssssooooooossss; oooosssssssoooo; sssssssssssoooo; oooosssssssssss; soosssssssssoos; soossssssssssss; ssssssssssssoos; or sssssssssssssss; wherein s=a phosphorothioate linkage, and o=a phosphodiester linkage.

59. The oligonucleotide of claim 57 or 58, wherein the oligonucleotide comprises sugar modification and internucleoside linkage combinations, respectively, in any of the following patterns: ssssooooooooossss
a) eeeee-d10-eeeee and sssssooooooooosssss;
b) eeeee-d10-eeeee and ooooosssssssssooooo;
c) eeeee-d10-eeeee and sssssssssssssssssss;
d) eee-d8-eee and sssooooooosss;
e) eee-d8-eee and ooosssssssooo
f) eee-d8-eee and sssssssssssss;
g) eee-d10-eee and sssooooooooosss;
h) eee-d10-eee and ooosssssssssooo;
i) eee-d10-eee and sssssssssssssss;
j) eeee-d10-eeee and ssssooooooooossss;
k) eeee-d10-eeee and oooosssssssssoooo;
l) eeee-d10-eeee and sssssssssssssssss;
m) eeee-d8-eeee and ssssooooooossss,
n) eeee-d8-eeee and oooosssssssoooo,
o) eeee-d8-eeee and sssssssssssssss,
wherein e=2′-MOE nucleoside and d=a deoxyribonucleoside, and wherein s=a phosphorothioate linkage, and o=a phosphodiester linkage.

60. The oligonucleotide of any one of claims 33-59, wherein the oligonucleotide comprises at least one modified nucleobase.

61. The oligonucleotide of claim 60, wherein the 5′ wing region or the 3′ wing region comprises the at least one modified nucleobase.

62. The oligonucleotide of claim 60, wherein the central region comprises the at least one modified nucleobase.

63. The oligonucleotide of any one of claims 60-62, wherein the at least one modified nucleobase is 5′-methylcytosine, pseudouridine, or 5-methoxyuridine.

64. The oligonucleotide of any one of claims 60-63, wherein every cytosine in the 5′ wing region or the 3′ wing region is a 5′-methylcytosine.

65. The oligonucleotide of any one of claims 60-64, wherein every cytosine in the central region is a 5′-methylcytosine.

66. The oligonucleotide of claim 33, wherein the oligonucleotide comprises sugar modification and internucleoside linkage combination of:
eeeee-d10-eeeee and sssssssssssssssssss, wherein e=2′-MOE nucleoside and d=a deoxyribonucleoside, and wherein s=a phosphorothioate linkage,
wherein each cytosine of the 2′MOE nucleosides is a 5-methylcytosine.

67. The oligonucleotide of any one of the preceding claims, wherein the oligonucleotide further comprises a conjugate moiety.

68. The oligonucleotide of claim 67, wherein the conjugate moiety is a cholesterol conjugate located on the 3′ end of the oligonucleotide.

69. A pharmaceutical composition comprising the oligonucleotide of any one of claims 1-68, or a pharmaceutically acceptable salt thereof, and a pharmaceutically acceptable excipient.

70. A method of treating a neurological disease in a patient in need thereof, the method comprising administering to the patient an oligonucleotide of any one of claims 1-68 or a pharmaceutically acceptable salt thereof, or a pharmaceutical composition of claim 69.

71. The method of claim 70, wherein the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

72. A method of increasing autophagy in a cell, the method comprising exposing the cell to a PPM1A inhibitor.

73. A method of increasing TBK1 ser172 phosphorylation in a cell, the method comprising exposing the cell to a PPM1A inhibitor.

74. A method of increasing TBK1 function in a cell, the method comprising exposing the cell to a PPM1A inhibitor.

75. A method of inhibiting PPM1A in a cell, the method comprising exposing the cell to a PPM1A inhibitor.

76. A method of inhibiting RIPK1 activity in a cell, the method comprising exposing the cell to a PPM1A inhibitor.

77. The method of any one of claims 72-76, wherein the cell is a cell of a patient in need of treatment of a neurological disease.

78. The method of claim 77, wherein the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

79. The method of any one of claims 72-75, wherein the exposing is performed in vivo or ex vivo.

80. The method of any one of claims 72-79, wherein the exposing comprises administering the PPM1A inhibitor to a patient in need thereof.

81. The method of any one of claims 72-80, wherein the PPM1A inhibitor is administered topically, parenterally, intrathecally, intracisternally, orally, rectally, buccally, sublingually, vaginally, pulmonarily, intratracheally, intranasally, transdermally, or intraduodenally.

82. The method of claim 81, wherein the PPM1A inhibitor is administered intrathecally.

83. The method of any one of claims 72-82, wherein a therapeutically effective amount of the PPM1A inhibitor is administered.

84. The method of any one of claims 77-78 or 80-83, wherein the patient is a human.

85. The method of any one of claims 72-84, wherein the PPM1A inhibitor comprises the PPM1A antisense oligonucleotide of any one of claims 1-68, a pharmaceutically acceptable salt thereof, or a pharmaceutical composition of claim 69.

86. The pharmaceutical composition of claim 69, wherein the pharmaceutical composition is suitable for topical, intrathecal, parenteral, oral, pulmonary, intratracheal, intranasal, transdermal, rectal, buccal, sublingual, vaginal, intracisternal, or intraduodenal administration.

87. Use of a PPM1A inhibitor in the manufacture of a medicament for the treatment of neurological disease.

88. The use of claim 87, wherein the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

89. The use of claim 87 or 88, wherein the PPM1A inhibitor is the PPM1A antisense oligonucleotide of any one of claims 1-68.

90. A method of treating a neurological disease in a patient in need thereof, the method comprising administering to a patient in need thereof a therapeutically effective amount of a pharmaceutical composition comprising a PPM1A inhibitor, and a pharmaceutically acceptable excipient.

91. The method of claim 90, wherein the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

92. The method of claim 90 or 91, wherein the PPM1A inhibitor is the PPM1A antisense oligonucleotide of any one of claims 1-68, a pharmaceutically acceptable salt thereof, or a pharmaceutical composition of claim 69.

93. The method of any one of claims 90-92, wherein the pharmaceutical composition is administered topically, parenterally, orally, pulmonarily, rectally, buccally, sublingually, vaginally, intratracheally, intranasally, intrathecally, intracisternally, transdermally, or intraduodenally.

94. The method of any one of claims 90-93, wherein the pharmaceutical composition is administered intrathecally.

95. The method of any one of claims 90-94, wherein the patient is human.

96. A PPM1A antisense oligonucleotide of any one of claims 1-68, or a pharmaceutically acceptable salt thereof, for use as a medicament.

97. A PPM1A antisense oligonucleotide of any one of claims 1-68, or a pharmaceutically acceptable salt thereof, for use in the treatment of a neurological disease.

98. The PPM1A antisense oligonucleotide for use of claim 96 or 97, wherein said neurological disease is selected from the group consisting of amyotrophic lateral sclerosis (ALS), frontotemporal dementia (FTD), ALS with FTD, Alzheimer's disease (AD), Parkinson's disease (PD), Huntington's disease, Brachial plexus injuries, peripheral nerve injuries, progressive supranuclear palsy (PSP), brain trauma, spinal cord injury, corticobasal degeneration (CBD) and/or neuropathies such a chemotherapy induced neuropathy, Spinocerebellar ataxia (SCA), Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), or Gaucher's disease.

99. A Protein Phosphatase 1A (PPM1A) antisense oligonucleotide selected from the group consisting of:
a PPM1A antisense oligonucleotide comprising the nucleotide sequence of any one of SEQ ID NOs: 2-955, SEQ ID NOs: 1910-2863, SEQ ID NOs: 2868-2913, and SEQ ID NOs: 2914-2959, or a pharmaceutically acceptable salt thereof,
wherein at least one nucleoside linkage of the nucleotide sequence is selected from the group consisting of: a phosphodiester linkage, a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage; and/or
wherein at least one nucleoside of the linked nucleosides is substituted with a component selected from the group consisting of a 2′-O-(2-methoxyethyl) (2′-MOE) nucleoside, a 2′-O-methyl nucleoside, a 2′-deoxy-2′-fluoro nucleoside, a 2′-fluoro-β-D-arabinonucleoside, a locked nucleic acid (LNA), constrained methoxyethyl (cMOE), constrained ethyl (cET), and a peptide nucleic acid (PNA).

100. The PPM1A antisense oligonucleotide of claim 99, wherein at least one internucleoside linkage of the nucleotide sequence is a phosphorothioate linkage.

101. The PPM1A antisense oligonucleotide of claim 100, wherein the phosphorothioate internucleoside linkage is in one of a Rp configuration or a Sp configuration.

102. The PPM1A antisense oligonucleotide of claim 99 or 100, wherein all internucleoside linkages of the nucleotide sequence are phosphorothioate linkages.

103. A pharmaceutical composition comprising the antisense oligonucleotide of any one of claims 99-102, or a pharmaceutically acceptable salt thereof, and a pharmaceutically acceptable excipient.

104. The method of any one of claims 70-71, 77-78, and 90-95, wherein the patient for treatment is identified by measuring the presence or level of expression of neurofilament light (NEFL), neurofilament heavy (NEFH), phosphorylated neurofilament heavy chain (pNFH), TDP-43, or p75^{ECD} in the plasma, the spinal cord fluid, the cerebrospinal fluid, the extracellular vesicles (for example, CSF exosomes), the blood, the urine, the lymphatic fluid, fecal matter, or a tissue of the patient.

105. The method of claim 104, wherein the patient for treatment is identified by measuring phosphorylated neurofilament heavy chain (pNFH) in cerebrospinal fluid (CSF).

106. The method of claim 105, wherein the pNFH in the CSF of the patient is used to predict disease status and survival in C9ORF72-associated amyotrophic lateral sclerosis (c9ALS) patients after initial administration and/or during on-going treatment.

107. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising Riluzole (Rilutek), troriluzole, Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO®), ZILUCOPLAN (RA101495), dual AON intrathecal administration (e.g., BIIB067, BIIB078), BIIB100, levodopa/carbidopa, dopaminergic agents (e.g., ropinirole, pramipexole, rotigotine), medroxyprogesterone, KCNQ2/KCNQ3 openers, Pridopidine, PrimeC (combination of ciprofloxacin and Celebrex), lithium, anticonvulsants and psychostimulant agents, breathing care, physical therapy, occupational therapy, speech therapy, and nutritional support.

108. The method of claim 107, wherein the neurological disease is any one of amyotrophic lateral sclerosis (ALS), frontotemporal dementia (FTD), or ALS with FTD.

109. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising Memantine, Rivastigmine, Galantamine, Donepezil, Aricept®, Exelon® (Rivastigmine), Razadyne®, Aducanumab, BAN2401, BIIB091 (gosuranemab), BIIB076, BIIB080 (IONIS-MAPTRx), Elayta (CT1812), MK1942, allogenic hMSC, nilotinib, ABT-957, acitretin, ABT-354, GV1001, Riluzole, CAD106, CNP520, AD-35, Rilapladib, DHP1401, T-817 MA, TC-5619, TPI-287, RVT-101, LY450139, JNJ-54861911, Dapagliflozin, GSK239512, PF-04360365, ASP0777, SB-742457 (a 5-HT6 receptor antagonist), PF-03654746 (an H₃ receptor antagonist), GSK933776 (an Fc-inactivated anti-β amyloid (Aβ) monoclonal antibody (mAb)), Posiphen ((+)-phenserine tartrate), AMX0035 (ELYBRIO®), coenzyme Q10, or any combination thereof.

110. The method of claim 109, wherein the neurological disease is Alzheimer's Disease.

111. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising Levodopa, Carbidopa-levidopa, pramipexole, ropinirole, rotigotine, apomorphine, selegiline, rasagiline, entacapone, tolcapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, or any combination thereof.

112. The method of claim 111 wherein the neurological disease is Parkinson's Disease.

113. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising UCB0107, ABBV-8E12, F-18 AV1451, BIIB092, C2N-8E12, tideglusib, deep transcranial magnetic stimulation, lipoic acid, tolfenamica acid, lithium, AZP2006, Glial Cell Line-Derived Neurotrophic Factor, NBMI, suvorxant, zolpidem, TPI 287, davunetide, pimavanserin, Levodopa, Carbidopa-levidopa, pramipexole, ropinirole, rotigotine, apomorphine, selegiline, rasagiline, entacapone, tolcapone, amantadine, trihexyphenidyl, BIIB054 (cinepanemab), BIIB094, BIIB118, ABBV-0805, zonisamide, deep brain stimulation, brain-derived neurotrophic factor, stem-cell transplant, Niacin, brain stem stimulation, nicotine, nabilone, PF-06649751, DNL201, LRRK2 inhibitors, CK1 inhibitors, isradipine, CLR4001, IRX4204, Yohimbine, coenzyme Q10, OXB-102, duloxetine, pioglitazone, preladenant, or any combination thereof.

114. The method of claim 113 wherein the neurological disease is progressive supranuclear palsy (PSP).

115. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising Tetrabenazine, deutetrabenazine, physical therapy, risperidone, haloperidol, chlorpromazine, clonazepam, diazepam, benzodiazepines, selective serotonin reuptake inhibitors, quetiapine, carbatrol, valproate, lamotrigine, pridopidine, delta-9-tetrahydrocannabinol, cannabidiol, stem-cell therapy, ISIS-443139, nilotinib, resveratrol, neflamapimod, fenofibrate, creatine, RO7234292, SAGE-718, WVE-120102, WVE-120101, dimebon, minocycline, deep brain stimulation, ursodiol, coenzyme Q10, OMS643762, VX15/2503, PF-02545920, BN82451B, SEN0014196, olanzapine, tiapridal (tiapride), or any combination thereof.

116. The method of claim 115, wherein the neurological disease is Huntington's Disease.

117. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising anticoagulants, antidepressants, muscle relaxants, stimulants, anticonvulsants, anti-anxiety medication, erythropoietin, hyperbaric treatment, rehabilitation therapies (e.g., physical, occupational, speech, psychological, or vocational counseling), or any combination thereof.

118. The method of claim 117, wherein the neurological disease is brain trauma.

119. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising AXER-204, glyburide, 5-hydroxytryptophan (5-HTP), L-3,4-dihydroxyphenylalanine (L-DOPA), or rehabilitation therapies (e.g., physical therapy, occupational therapy, recreational therapy, use of assistive devices, improved strategies for exercise and healthy diets), or any combination thereof.

120. The method of claim 119, wherein the neurological disease is spinal cord injury.

121. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising TPI-287, lithium, occupational, physical, and speech therapy, or any combination thereof can be selected as an additional therapy.

122. The method of claim 121, wherein the neurological disease is corticobasal degeneration.

123. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising gabapentin, pregabalin, lamotrigine, carbamazepine, duloxetine, gabapentinoids, tricyclic antidepressants, serotonin-norepinephrine reuptake inhibitors, opioids, neurotoxin, dextromethorphan, nicotinamide riboside, auto-antibodies targeting neuronal antigens (TS-HDS and FGFR3), or any combination thereof.

124. The method of claim 123, wherein the neuropathy is a chemotherapy induced neuropathy.

125. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising troriluzole, BHV-4157, or a combination thereof.

126. The method of claim 125, wherein the neurological disease is spinocerebellar ataxia.

127. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising anti-seizure medications, speech therapy, physical therapy, occupational therapy, Adrabetadex, Arimoclomol, N-Acetyl-L-Leucine, or any combination thereof.

128. The method of claim 127, wherein the neurological disease is Niemann-Pick disease type C.

129. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising physical and occupational therapies, orthopedic surgery, orthopedic devices, PXT3003, or any combination thereof.

130. The method of claim 129, wherein the neurological disease is Charcot-Marie-Tooth Disease (CMT).

131. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising enzyme replacement therapy: idursulfase (Elaprase), surgical intervention (tonsillectomy and/or adenoidectomy), RGX-121 gene therapy, adalimumab, MT2013-31, or any combination thereof.

132. The method of claim 131, wherein the neurological disease is Mucopolysaccharidosis type II (MPSIIA).

133. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising physical, occupational, and speech therapies, contact lenses and artificial tears, genetic counseling, or any combination thereof.

134. The method of claim 133, wherein the neurological disease is Mucolipidosis IV.

135. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising anticonvulsants, physical and occupational therapies, galactosidase, gene delivery of galactosidase, LYS-GM101 gene therapy, or any combination thereof.

136. The method of claim 135, wherein the neurological disease is GM1 gangliosidosis.

137. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising physical and occupational therapies, use of devices such as braces, walkers, wheelchairs, immunosuppressants, BYM338, or any combination thereof.

138. The method of claim 137, wherein the neurological disease is Sporadic inclusion body myositis (sIBM).

139. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising corticosteroids, colchicine, dapsone, azathioprine, or any combination thereof.

140. The method of claim 139, wherein the neurological disease is Henoch-Schonlein purpura (HSP).

141. A method of treating a neurological disease and/or a neuropathy in a patient in need thereof, the method comprising administering to a patient in need thereof a therapeutically effective amount of an oligonucleotide of any one of claims 1-68 or a pharmaceutically acceptable salt thereof, or a pharmaceutical composition of claim 69, in combination with a second therapeutic agent selected from a group comprising enzyme replacement therapy, substrate reduction therapy, N-acetylcysteine, GZ/SAR402671, cerezyme, or any combination thereof.

142. The method of claim 141, wherein the neurological disease is Gaucher's disease.

143. The method of any one of claims 1-3, wherein the transcript comprises a sequence of SEQ ID NO: 2864 and is further transcribed from nucleotides 8,470-8,926, 44,991-45,990, 49,055-49,164, 50,647-50,704, and 51,703-58,336 of SEQ ID NO: 1.

144. The method of any one of claims 1-3, wherein the transcript comprises a sequence of SEQ ID NO: 2865 and is further transcribed from nucleotides 8,470-8,926, 9,629-9,730, and 44,911-47,804 of SEQ ID NO: 1.

145. The method of any one of claims 1-3, wherein the transcript comprises a sequence of SEQ ID NO: 2866 and is further transcribed from nucleotides 4,999-5,295, 49,055-49,164, 50,647-50,704, and 51,703-58,336 of SEQ ID NO: 1.

146. A method of treating a neurological disease in a patient, the method comprising selecting a patient for treatment with an oligonucleotide of any one of claims 1-68 or a pharmaceutically acceptable salt thereof, or a pharmaceutical composition of claim 69, wherein the patient for treatment is selected by a method comprising measuring a presence or level of expression of neurofilament light (NEFL), neurofilament heavy (NEFH), phosphorylated neurofilanent heavy chain (pNFH), TDP-43, or p75^{ECD} in the plasma, the spinal cord fluid, the cerebrospinal fluid, the extracellular vesicles (for example, CSF exosomes), the blood, the urine, the lymphatic fluid, fecal matter, or a tissue of the patient.

147. The method of claim 146, wherein the patient for treatment is identified by measuring phosphorylated neurofilament heavy chain (pNFH) in cerebrospinal fluid (CSF).

148. The method of claim 147, wherein the pNFH in the CSF of the patient is used to predict disease status and survival in C9ORF72-associated amyotrophic lateral sclerosis (c9ALS) patients after initial administration and/or during on-going treatment.

149. A method of treating a neurological disease in a patient, the method comprising selecting a patient for treatment with an oligonucleotide of any one of claims 1-68 or a pharmaceutically acceptable salt thereof, or a pharmaceutical composition of claim 69, wherein the method comprises:
determining whether the patient has a mutation in one or more ALS-associated genes selected from the group comprising TBK1, TARDBP, SQSTM1, VCP, C9orf72, FUS, and CHCHD10;
identifying the patient as a candidate patient for treatment according to the determination; and
optionally administering, to the candidate patient, the oligonucleotide of any one of claims 1-68 or a pharmaceutically acceptable salt thereof, or a pharmaceutical composition of claim 69.

150. A method of treating a neurological disease in a patient, the method comprising administering to the patient an oligonucleotide of any one of claims 1-68 or a pharmaceutically acceptable salt thereof, or a pharmaceutical composition of claim 69, wherein the patient for treatment is selected by a method comprising measuring a presence or level of expression of neurofilament light (NEFL), neurofilament heavy (NEFH), phosphorylated neurofilament heavy chain (pNFH), TDP-43, or p75^{ECD} in the plasma, the spinal cord fluid, the cerebrospinal fluid, the extracellular vesicles (for example, CSF exosomes), the blood, the urine, the lymphatic fluid, fecal matter, or a tissue of the patient.

151. The method of claim 146, wherein the patient for treatment is identified by measuring phosphorylated neurofilament heavy chain (pNFH) in cerebrospinal fluid (CSF).

152. The method of claim 147, wherein the pNFH in the CSF of the patient is used to predict disease status and survival in C9ORF72-associated amyotrophic lateral sclerosis (c9ALS) patients after initial administration and/or during on-going treatment.

153. A method of treating a neurological disease in a patient, the method comprising administering to the patient an oligonucleotide of any one of claims 1-68 or a pharmaceutically acceptable salt thereof, or a pharmaceutical composition of claim 69, wherein the patient is selected for treatment by a method comprising:
determining whether the patient has a mutation in one or more ALS-associated genes selected from the group comprising TBK1, TARDBP, SQSTM1, VCP, C9orf72, FUS, and CHCHD10;
identifying the patient as a candidate patient for treatment according to the determination.
